(19) United States
(12) Patent Application Publication
(10) Pub. No.: US 2023/0027475 A1
(43) Pub. Date: Jan. 26, 2023

(54) ANTI-ONCOLYTIC VIRUS ANTIGEN ANTIBODIES AND METHODS OF USING SAME

(71) Applicants: Admare Therapeutics Society, Vancouver, BC (CA); Provincial Health Services Authority, Vancouver, BC (CA); UVic Industry Partnerships Inc., Vancouver, BC (CA)

(72) Inventors: Emma J. Cummins, Vancouver, BC (CA); Jan Peter Bergqvist, Vancouver, BC (CA); Brad Nelson, Victoria, BC (CA); Kwame Twumasi-Boateng, Hannon, ON (CA); Yin Yu Eunice Kwok, Vancouver, BC (CA); Julian Smazynski, Victoria, BC (CA); Francois Benard, Vancouver, BC (CA); Julie Marie Rousseau, Vancouver, BC (CA); Kuo-Shyan Lin, Richmond, BC (CA)

(21) Appl. No.: 17/642,089

(22) PCT Filed: Sep. 11, 2020
(86) PCT No.: PCT/CA2020/051230

§ 371 Date: Mar. 10, 2022

Related U.S. Application Data
(60) Provisional application No. 62/900,303, filed on Sep. 13, 2019.

(51) Int. Cl. C07K 16/08; A61P 35/00; A61K 51/10; C12N 5/0783

(52) U.S. Cl. CPC C07K 16/081; A61P 35/00; A61K 51/1006; C12N 5/0638; A61K 2039/505

(57) ABSTRACT

Provided are antibodies that specifically bind Vaccinia Virus (VV) A56 or B5 antigen. Also provided are fusion proteins and conjugates that comprise the antibodies. Pharmaceutical compositions and kits that comprise the antibodies, fusion proteins and conjugates are also provided. Aspects of the present disclosure further include methods of using the antibodies, fusion proteins and conjugates, e.g., for therapeutic purposes. In certain embodiments, provided are methods that comprise administering an antibody, fusion protein or conjugate of the present disclosure to an individual having cancer, wherein the individual comprises cancer cells infected with VV, and wherein the antibody, fusion protein or conjugate is targeted to the infected cancer cells by VV antigens expressed on the surface of the infected cancer cells. Aspects of the present disclosure further include methods of targeting an antibody, fusion protein, or conjugate that specifically binds an oncolytic virus (OV) antigen to cancer cells in an individual.

### CROSS-REFERENCE TO RELATED APPLICATIONS

This application claims the benefit of U.S. Provisional Patent Application No. 62/900,303, filed Sep. 13, 2019, which application is incorporated herein by reference in its entirety.

### INTRODUCTION

Immunotherapy has emerged as an effective therapeutic option against multiple malignancies. Oncolytic viruses (OVs), which can be engineered to replicate selectively in and lyse tumor tissues while sparing the normal non-neoplastic host cells and simultaneously restoring antitumor immunity, constitute a next-generation immunotherapeutic approach for the treatment of tumors. The unique ability of OVs to target malignancies without dependence on specific antigen expression patterns makes them an attractive alternative to other immunotherapy approaches. In addition, OVs can promote the recruitment of tumor-infiltrating lymphocytes (TILs), reprogram the immunosuppressive tumor microenvironment (TME), and boost systemic antitumor immunity.

Genetic engineering has enabled the design of live replicating viruses to not only be highly tumor selective through cell entry and transcription targeting but also armed with reporter genes for noninvasive monitoring of the pharmacokinetics of virotherapy, and for enhancing cytotoxic activity or immunogenic cell death, or immune modulators. Three OVs are currently commercially available for the treatment of cancer. These include Rigvir, approved in Latvia, Georgia, and Armenia; Oncorine H101 approved in China; and talimogene laherparepvec (T-VEC) approved in the United States. Rigvir (Riga virus) is an unmodified Enteric Cytopathogenic Human Orphan type 7 (ECHO-7) picornavirus for which limited data has been published to describe its efficacy. Oncorine became the first approved oncolytic virus for clinical use in China, and the world's first recombinant oncolytic virus to gain regulatory approval. It is an attenuated serotype 5 adenoviral vector deleted for viral E1B-55k and with four deletions in viral E3. Oncorine remains the only approved adenovirus for cancer therapy, and only when given in combination with chemotherapy. T-VEC (Imlygic™) was approved by the US Food and Drug Administration (FDA) in 2015 for the treatment of non-resectable metastatic melanoma, and later in the EU for locally advanced or metastatic cutaneous melanoma, making it the most recent oncolytic virus to gain national regulatory approval and the first to gain approval in the United States. T-VEC is a recombinant human herpes simplex virus type 1 (HSV1) deleted for both copies of the HSV1 gamma34.5 and viral ICP47, which accelerates the expression of US11, and encodes 2 copies of human granulocyte-macrophage colony stimulating factor (GM-CSF) under cytomegalovirus (CMV) promoters. T-VEC is currently approved for intratumoral injection into cutaneous high-grade melanoma lesions and exhibits single-agent efficacy for this indication.

OVs in clinical development include measles virus, newcastle disease virus (NDV), rhabdoviruses, adenovirus, vaccinia virus (VV), herpes viruses, coxsackievirus, reovirus, and retrovirus.

Measles virus is a negative strand RNA paramyxovirus which is highly fusogenic and induces extensive cytopathic effects of syncytial formation. Intercellular fusion increases bystander killing of tumor cells and induces immunogenic danger signals which can elicit host mediated cellular antitumor activity. Recombinant Edmonston strain measles virus encoding the sodium iodide symporter (MV-NIS) or soluble carcinoembryonic antigen (MV-CEA) are in Phase I/II clinical testing in patients with relapsed or recurrent cancers including multiple myeloma, ovarian cancer, glioma, breast cancer and mesothelioma.

NDV is an avian paramyxovirus and has been tested as an oncolytic or oncolysate cancer vaccine. NDV strains MTH-68/H, HUJ, and PV701 have been tested clinically. A recombinant NDV based on the mesogenic NDV-73T strain with compromised infection of avian cells but not mammalian cells and encoding GM-CSF (Medimmune, MEDI5395) is in preclinical testing.

Rhabdoviruses are negative sense RNA viruses with rapid, ˜12-hour lytic replication cycles in permissive cells. The best studied oncolytic rhabdovirus Vesicular Stomatitis Virus (VSV) uses the low-density lipoprotein (LDL) receptor for cell entry, allowing VSV to infect nearly all cell types and cause lytic infection in a wide variety of permissive cells.

Adenoviruses are non-enveloped icosahedral double-stranded DNA viruses with long fiber knobs protruding from each capsid vertex. Clinical data has been published for telomelysin in solid tumors, CG0070 in bladder cancer, DNX-2401 in malignant brain tumors.

Vaccinia virus (VV) is a large, enveloped, double-stranded DNA virus with a linear genome approximately 190 kb in length. Attenuation or tumor-specific targeting of these viruses has been accomplished using a variety of deletions and insertional mutations, with loss of thymidine kinase function being a common denominator among the clinical oncolytic vaccinia viruses. JX-594 is deleted for viral thymidine kinase, TG6002 is doubly deleted for thymidine kinase and viral ribonucleotide reductase, and GL-ONC1 has insertional mutations in its thymidine kinase (J2R), hemagglutinin HA (A56R), and F14.5L genes. The TK loss of function limits the virus' ability to replicate in non-dividing cells, and the deletion of viral ribonucleotide reductase further limits this ability. Two clinical vaccinia vectors designed to enhance oncolytic efficacy include transgenes designed to improve tumor cell killing: JX-594, like T-VEC, includes GM-CSF, and TG6002 incorporates a nucleoside analog converting enzyme FCU1, which converts 5-fluorocytosine (5-FC) to 5-FU in infected cells.

HSV1 is a large double stranded DNA virus approximately 152 kb in length. Clinically evaluated gamma34.5-deficient viruses include the now FDA-approved T-VEC, HSV1716 (Seprehvir), G207, and RP1. Attempts to boost the anticancer effects of HSV in metastatic diseases involve the inclusion of therapeutic transgenes used to simultaneously boost anti-cancer and antiviral immunity, with the goal of developing an adaptive anti-tumor response in treated patients.

Coxsackievirus is a single stranded positive RNA picornavirus of approximately 7.4 kb, enclosed in an icosahedral capsid. Oncolytic CVA21 (Viralytics, CAVATAK) is derived from the Kuykendall strain and uses ICAM-1 as the primary receptor for cell entry. Phase I testing of intratumoral CVA21 virus injection in combination with pembrolizumab or ipilimumab is ongoing to enhance the overall efficacy of these drugs.

Reovirus is a double stranded RNA virus, non-enveloped and has an icosahedral capsid composed of an outer and inner protein shell. Reovirus as a monotherapy was investigated in several Phase I trials (Oncolytics Biotech, Reolysin™) as an intratumoral or intravenous administration.

Retroviral replicating vector (Tocagen, Toca-511, vocimagene amiretrorepvec) encodes yeast cytosine deaminase (CD) that converts the prodrug 5-FC to the anticancer drug, 5-FU, thereby enhancing local concentration of 5-FU in the tumor and decreasing overall systemic toxicity of the drug. A phase 1 trial of Toca-511 in patients with recurrent high-grade glioma resulted in overall survival of 13.6 months and was statistically improved relative to an external control.

Despite the recent advances in OV-based therapies, there remains a need for new and improved OV-based methods for the treatment, alleviation, and/or prevention of cancer and for methods of improving survival in subjects with cancer.

### SUMMARY

Provided are antibodies that specifically bind Vaccinia Virus (VV or VACV) A56 or B5 antigen. Also provided are fusion proteins and conjugates that comprise the antibodies. Pharmaceutical compositions and kits that comprise the antibodies, fusion proteins and conjugates are also provided. Aspects of the present disclosure further include methods of using the antibodies, fusion proteins and conjugates, e.g., for therapeutic purposes. In certain embodiments, provided are methods that comprise administering an antibody, fusion protein or conjugate of the present disclosure to an individual having cancer, wherein the individual comprises cancer cells infected with VV, and wherein the antibody, fusion protein or conjugate is targeted to the infected cancer cells by VV antigens expressed on the surface of the infected cancer cells. Aspects of the present disclosure further include methods of targeting an antibody, fusion protein, or conjugate that specifically binds an oncolytic virus (OV) antigen to cancer cells in an individual.

### BRIEF DESCRIPTION OF THE FIGURES

FIG. 1 shows flow cytometry data for antibody binding to A549 and CaOV3 cells infected with VV Western Reserve VVdd (eGFP).

FIG. 2 shows flow cytometry data for antibody binding to A56 or B5 expressing HEK cells (Wyeth VV sequence).

FIG. 3 shows data for antibody binding to HEK-B5 cells. The highest concentration tested in a flow cytometry assay (top panel) or ELISA (bottom panel) for binding to HEK-B5 is plotted, 5 μg/ml for rabbit and murine isotype controls (Rb IgG Isotype and mIgG Isotype, respectively), and the anti-B5 Immunetech antibody, IT anti-B5 IgG, (identified as anti-B5 mIgG in FIG. 3), and 1:1000 for the rabbit polyclonal antibody (c-VV).

FIG. 4 shows binding of A048 antibody, the Immunetech anti-B5 antibody, IT anti-B5 IgG (identified as IT anti-B5 mIgG in FIG. 4), and a human isotype control to VV Western Reserve VVdd (eGFP)-infected A549 cells (top panel) and HT29 cells (bottom panel) measured by flow cytometry.

FIG. 5 shows antibody binding to VV-infected cells. Antibodies were tested for binding to VVdd (eGFP) infected A549 cells by flow cytometry. All formats have similar EC50s, and no binding was observed to uninfected cells.

FIG. 6 shows binding of antibody A049 to virus-infected tumor tissue by immunofluorescence, in particular A049 anti-A56 hIgG1 binding to virus-infected colorectal tumor tissue. The top 2 panels are hematoxylin and eosin (H&E) stained. This is compared to a polyclonal anti-Vaccinia Virus antibody showing some non-specific staining to uninfected tissue.

FIG. 7 provides epitope binning data showing binding of biotinylated antibodies to VVdd (eGFP)-infected cells pre-incubated with non-biotinylated antibodies. Reduced biotinylated antibody binding indicates a similar or overlapping epitope to the test antibody. Antibodies A047 and A049 appear to bind different epitopes on A56. Antibodies A048 and A051 appear to bind different epitopes on B5.

FIG. 8 shows data for antibody binding to virus particles. Virus particles (VVdd (eGFP)) were coated on a plate and incubated with antibodies, or positive control NKp30. All antibodies bound the virus except antibody A054.

FIG. 9 provides data showing virus infection neutralization. Antibodies were added to A549 cells at the same time as VVdd (eGFP) infection. Similar levels of infection were observed to infection without antibody (‘No Ab’).

FIG. 10 shows antibody binding to murine cell lines infected with VVCopenhagen (YFP). Percent YFP expression in each infected cell line indicates the proportion of cells infected with VVcopenhagen (YFP). The anti-A56-PE and anti-B5-PE antibodies bind B16F10, CT26 LacZ, and MC38 cells infected with VVcopenhagen (YFP).

FIG. 11 shows immunohistochemistry data for specific binding of antibodies to HEK cell surface expressed A56 or B5 antigen or VVCopenhagen virus-infected U20S cells. Rabbit/murine chimeric (antibodies A059 and A058) and full rabbit IgG format (antibodies A056 and A073) antibodies specific for A56 or B5 were able to specifically detect target antigen on stably expressed HEK-A56 and HEK-B5 cells, respectively. Both anti-A56 and anti-B5 antibodies were able to detect protein expression on VVCopenhagen-infected U2OS cells.

FIG. 12 shows detection of A56 and B5 from VV-treated tumors by immunohistochemistry. The data shows detection of A56 and B5 proteins on the surface of vaccinia virus-treated tumors.

FIG. 13 Panel A: Representative schematic of an example design of an anti-VV chimeric antigen receptor (CAR) construct. Panel B: Schematic illustration of a therapeutic approach according to embodiments of the present disclosure. As shown, an OV infects a cancer cell, followed by display of OV antigens on the surface of the cancer cell (in this example, A56 or B5 OV antigens, which in some embodiments may be the native A56 or B5 antigen of the OV). Treatment of the infected cancer cell with a CAR T cell comprising a CAR specific for a displayed OV antigen results in destruction of the infected cancer cell. Neighboring cancer cells infected by OVs released by the destroyed cancer cell may in turn be destroyed by a CAR T cell comprising the CAR specific for the displayed OV antigen.

FIG. 14 provides flow cytometry data showing VV CAR detection following lentiviral transduction. The data depicts VV-CAR expression on transduced Jurkat cells. CAR downregulation, indicative of specific target binding, is evident across all VV-CAR constructs tested.

FIG. 15 shows CAR and eGFP expression on activated A56-CAR-06 positive Jurkat cells. The data depicts co-expression of A56-CAR-06 and eGFP on transduced Jurkat cell line. Following culture with target HEK A56 line the activated CAR population (CD69 positive) can be identified within the scFv negative fraction by gating the eGFP positive population.

FIG. 16 provides data showing specific activation of Jurkat cells expressing VV-CARs when co-cultured with target HEK cell lines. All VV-CARs tested show specific on target activation indicated by CD69 upregulation. Minimal cross reactivity/endogenous activation occurred when VV-CAR lines were co-cultured with negative/irrelevant target cell lines.

FIG. 17 shows transduction and enrichment for A56-CAR-06 expression of primary healthy donor T cells. The data depicts stable expression of A56-CAR-06 on primary human T cells following a standard clinical manufacturing protocol. An enriched A56-CAR-06 population was established by cell sorting on CAR expression followed by repeated clinical expansion.

FIG. 18 shows specific activation of human T cells expressing A56-CAR-06 when co-cultured with target expressing HEK-A56 lines. Primary human T cells expressing A56-CAR-06 showed significant upregulation of CD69 and CD137 following co-culture with target HEK-A56 line. Minimal cross-reactivity observed with negative/irrelevant target cell lines.

FIG. 19 provides data demonstrating that primary human T cells expressing A56-CAR-06 show morphological signs of direct tumor killing at 48 h following co-culture with target HEK-A56 cells.

FIG. 20 provides data demonstrating that human T cells expressing B5-CAR-011 show high percent specific cytotoxicity. Primary human T cells expressing B5-CAR-011 exhibited specific cell lysis at 24 h as determined by percent decrease of relative luminescence unit (RLU).

FIG. 21 shows representative maximum intensity projection positron emission tomography (PET) images of the ⁸⁹Zr-DFO-A049 (anti-A56 antibody, DFO: deferoxamine) at 1, 3 and 5 days post-injection for the same mouse. Tumor types: HEK-A56 (expressing A56, on the left shoulder) and the parental HEK (non expressing, on the right shoulder), in addition to the spleen and liver are indicated by arrows. Results are shown as percentage of the injected dose per gram of tissue (% ID/g). Clear preclinical A56-positive tumor PET imaging is shown with efficient and specific accumulation of the ⁸⁹Zr-DFO-A049 in the positive tumor, and expected radioimmunoconjugate pharmacokinetics.

### DETAILED DESCRIPTION

Before the antibodies, compositions and methods of the present disclosure are described in greater detail, it is to be understood that the antibodies, compositions and methods are not limited to particular embodiments described, as such may, of course, vary. It is also to be understood that the terminology used herein is for the purpose of describing particular embodiments only, and is not intended to be limiting, since the scope of the antibodies, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the antibodies, compositions and methods. The upper and lower limits of these smaller ranges may independently be included in the smaller ranges, subject to any specifically excluded limit in the stated range. Where the stated range includes one or both of the limits, ranges excluding either or both of those included limits are also included in the antibodies, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the antibodies, compositions and methods belong. Although any antibodies, compositions and methods similar or equivalent to those described herein can also be used in the practice or testing of the antibodies, compositions and methods, representative illustrative antibodies, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present antibodies, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the antibodies, compositions and methods, which are, for clarity, described in the context of separate embodiments, may also be provided in combination in a single embodiment. Conversely, various features of the antibodies, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present antibodies, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Antibodies

The present disclosure provides antibodies that specifically bind vaccinia virus (VV or VACV) antigens. Vaccinia viruses are members of the poxvirus family characterized by an approximately 192 kb double-stranded DNA genome that encodes numerous viral enzymes and factors that enable the virus to replicate independently from the host cell machinery. VV can stably accommodate up to 25 kb of cloned exogenous DNA. Structurally, it consists of a core region composed of viral DNA and various viral enzymes including RNA polymerase and polyA polymerase encased in a lipoprotein core membrane. The outer layer of the virus consists of double lipid membrane envelope. VV has many inherent characteristics that make VV possess attributes suitable for use in oncolytic viral therapy such as natural tropism for tumors, strong lytic ability, short life cycle with rapid cell-to-cell spread, efficient gene expression and a large cloning capacity. VV has a short life cycle of about 8 hours that takes place in the cytoplasm eliminating the risk of genome integration. Replication typically starts about 2 hours after infection, at which time host cell nucleic acid synthesis shuts down and cellular resources are directed toward viral replication. Cell lysis takes place between 12 and 48 hours releasing packaged viral particles. VV does not depend on host mechanisms for mRNA transcription making it less susceptible to biological changes of the host cell. Unlike other oncolytic viruses (OVs), VV does not require a specific surface receptor for cell entry, allowing it to infect a wide range of cells.

The term “antibody” (also used interchangeably with “immunoglobulin”) encompasses polyclonal (e.g., rabbit polyclonal) and monoclonal antibody preparations where the antibody may be an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein. In some embodiments, the antibody is selected from an IgG, Fv, single chain antibody, scFv, a Fab, a F(ab′)₂, and a F(ab′). The antibodies may be further conjugated to other moieties, such as members of specific binding pairs, e.g., biotin (member of biotin-avidin specific binding pair), and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, Md. (1987); and Lefrancetal. IMGT, the international ImMunoGeneTics Information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, an antibody of the present disclosure comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH—CHI by a disulfide bond. The F(ab)′₂ may be reduced under mild conditions to break the disulfide linkage in the hinge region thereby converting the (Fab′)₂ dimer into an Fab′monomer. The Fab′monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies. In certain embodiments, an antibody of the present disclosure is selected from an IgG, Fv, single chain antibody, scFv, Fab, F(ab′)₂, and Fab′.

According to some embodiments, an antibody of the present disclosure is a monoclonal antibody. “Monoclonal antibody” refers to a composition comprising one or more antibodies obtained from a population of substantially homogeneous antibodies, i.e., a population the individual antibodies of which are identical except for any naturally occurring mutations that may be present in minor amounts. Monoclonal antibodies are highly specific, being directed against a single antigenic site and generally to a single epitope on an antigen. The modifier “monoclonal” indicates the character of the antibody as being obtained from a substantially homogeneous population of antibodies, and does not require that the antibody be produced by any particular method or be the only antibody in the composition.

In certain embodiments, an antibody of the present disclosure is a humanized antibody. As used herein, a humanized antibody is a recombinant polypeptide that is derived from a non-human (e.g., rabbit, rodent, or the like) antibody and has been modified to contain at least a portion of the framework and/or constant regions of a human antibody. Humanized antibodies also encompass chimeric antibodies and CDR-grafted antibodies in which various regions may be derived from different species. Chimeric antibodies may be antibodies that include a variable region from any source linked to a human constant region (e.g., a human Fc domain). Thus, in chimeric antibodies, the variable region can be non-human, and the constant region is human. CDR-grafted antibodies are antibodies that include the CDRs from a non-human “donor” antibody linked to the framework region from a human “recipient” antibody. For example, an antibody of the present disclosure in a form of an scFV may be linked to a human constant region (e.g., Fc domain) to be made into a human immunoglobulin.

In general, humanized antibodies produce a reduced immune response in a human host, as compared to a non-humanized version of the same antibody. Antibodies can be humanized using a variety of techniques including, for example, CDR-grafting, veneering or resurfacing, chain shuffling, and the like. In certain embodiments, framework substitutions are identified by modeling of the interactions of the CDR and framework residues to identify framework residues important for antigen binding and sequence comparison to identify unusual framework residues at particular positions.

Accordingly, any of the antibodies described herein may be humanized using available methods. The substitution of rabbit or mouse CDRs into a human variable domain framework can result in retention of their correct spatial orientation where, e.g., the human variable domain framework adopts the same or similar conformation to the rabbit or mouse variable framework from which the CDRs originated. This can be achieved by obtaining the human variable domains from human antibodies whose framework sequences exhibit a high degree of sequence identity with the rabbit or mouse variable framework domains from which the CDRs were derived. The heavy and light chain variable framework regions can be derived from the same or different human antibody sequences. The human antibody sequences can be the sequences of naturally occurring human antibodies or can be consensus sequences of several human antibodies.

Having identified the complementarity determining regions of the rabbit or mouse donor immunoglobulin and appropriate human acceptor immunoglobulins, the next step is to determine which, if any, residues from these components should be substituted to optimize the properties of the resulting humanized antibody. In general, substitution of human amino acid residues with rabbit or mouse should be minimized, because introduction of rabbit or mouse residues increases the risk of the antibody eliciting a human-anti-rabbit-antibody (HARA) or human-anti-mouse-antibody (HAMA) response in humans. Art-recognized methods of determining immune response can be performed to monitor a HARA or HAMA response in a particular patient or during clinical trials. Patients administered humanized antibodies can be given an immunogenicity assessment at the beginning and throughout the administration of said therapy. The HARA or HAMA response is measured, for example, by detecting antibodies to the humanized therapeutic reagent, in serum samples from the patient using a method known to one in the art, including surface plasmon resonance technology (BIACORE) and/or solid-phase ELISA analysis. In many embodiments, a subject humanized antibody does not substantially elicit a HARA response in a human subject.

Certain amino acids from the human variable region framework residues are selected for substitution based on their possible influence on CDR conformation and/or binding to antigen. The unnatural juxtaposition of rabbit or murine CDR regions with human variable framework region can result in unnatural conformational restraints, which, unless corrected by substitution of certain amino acid residues, lead to loss of binding affinity. The selection of amino acid residues for substitution can be determined, in part, by computer modeling. Computer hardware and software for producing three-dimensional images of immunoglobulin molecules are known in the art. In general, molecular models are produced starting from solved structures for immunoglobulin chains or domains thereof. The chains to be modeled are compared for amino acid sequence similarity with chains or domains of solved three-dimensional structures, and the chains or domains showing the greatest sequence similarity is/are selected as starting points for construction of the molecular model. Chains or domains sharing at least 50% sequence identity are selected for modeling, and preferably those sharing at least 60%, 70%, 80%, 90% sequence identity or more are selected for modeling. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

When framework residues, as defined by, e.g., Kabat, constitute structural loop residues as defined by, e.g., Chothia, the amino acids present in the rabbit or mouse antibody may be selected for substitution into the humanized antibody. Residues which are “adjacent to a CDR region” include amino acid residues in positions immediately adjacent to one or more of the CDRs in the primary sequence of the humanized immunoglobulin chain, for example, in positions immediately adjacent to a CDR as defined by Kabat, or a CDR as defined by Chothia (See e.g., Chothia and Lesk JMB 196:901 (1987)). These amino acids are particularly likely to interact with the amino acids in the CDRs and, if chosen from the acceptor, to distort the donor CDRs and reduce affinity. Moreover, the adjacent amino acids may interact directly with the antigen (Amit et al., Science, 233:747 (1986)) and selecting these amino acids from the donor may be desirable to keep all the antigen contacts that provide affinity in the original antibody. Approaches that may be employed to humanize any of the antibodies described herein include, but are not limited to, those described in Williams, D., Matthews, D. & Jones, T. Humanising Antibodies by CDR Grafting. Antibody Engineering 319-339 (2010) doi:10.1007/978-3-642-01144-3_21; Kuramochi, T., Igawa, T., Tsunoda, H. & Hattori, K. Humanization and simultaneous optimization of monoclonal antibody. Methods Mol. Biol. 1060, 123-37 (2014); Hwang, W. Y., Almagro, J. C., Buss, T. N., Tan, P. & Foote, J. Use of human germline genes in a CDR homology-based approach to antibody humanization. Methods 36, 35-42 (2005); Lo, B. K. Antibody humanization by CDR grafting. Methods Mol. Biol. 248, 135-59 (2004); and Lefranc, M. P. P., Ehrenmann, F., Ginestoux, C., Giudicelli, V. & Duroux, P. Use of IMGT (@) databases and tools for antibody engineering and humanization. Methods Mol. Biol. 907, 3-37 (2012); the disclosures of which are incorporated herein by reference in their entireties for all purposes.

An antibody of the present disclosure specifically binds to the VV A56 or VV B5 antigen. An antibody “specifically binds” or “preferentially binds” to a target if it binds with greater affinity, avidity, more readily, and/or with greater duration than it binds to other substances, e.g., in a sample. In certain embodiments, an antibody “specifically binds” an antigen if it binds to or associates with the antigen with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means the antibody binds to the antigen with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the antigen can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

Whether an antibody of the present disclosure “competes with” a second antibody for binding to the antigen may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is added. One of the two antibodies is labeled. If the labeled antibody and the unlabeled antibody bind to separate and discrete sites on the antigen, the labeled antibody will bind to the same level whether or not the suspected competing antibody is present. However, if the sites of interaction are identical or overlapping, the unlabeled antibody will compete, and the amount of labeled antibody bound to the antigen will be lowered. If the unlabeled antibody is present in excess, very little, if any, labeled antibody will bind.

For purposes of the present disclosure, competing antibodies are those that decrease the binding of an antibody to the antigen by about 50% or more, about 60% or more, about 70% or more, about 80% or more, about 85% or more, about 90% or more, about 95% or more, or about 99% or more. Details of procedures for carrying out such competition assays are known and can be found, for example, in Harlow and Lane, Antibodies, A Laboratory Manual, Cold Spring Harbor Laboratory Press, Cold Spring Harbor, N.Y., 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

Anti-VV A56 Antibodies

According to some embodiments, provided are antibodies that specifically bind a VV A56 antigen (VV A56). While the vaccinia virus A56 protein was originally characterized as a haemagluttinin protein, A56 has other functions as well. The A56 protein is capable of binding two viral proteins, a serine protease inhibitor (K2) and the vaccinia virus complement control protein (VCP), and anchoring them to the surface of infected cells. While both proteins have biologically relevant functions at the cell surface, neither one can locate there on its own. The A56-K2 complex reduces the amount of virus superinfecting an infected cell and also prevents the formation of syncytia by infected cells. The A56-VCP complex can protect infected cells from complement attack. Deletion of the A56R gene results in varying effects on vaccinia virus virulence. Because the gene encoding the A56 protein is non-essential, it may be used as an insertion point for foreign genes and has been deleted in some viruses that are in clinical development as oncolytic agents.

In certain embodiments, an antibody of the present disclosure specifically binds VV A56 and competes for binding to VV A56 with an antibody having one, two, three, four, five, or all six complementarity determining regions (CDRs) of one or more of the anti-VV A56 antibodies designated herein as A047/A057, A049/A059/A056, A050, and A054. According to some embodiments, an antibody of the present disclosure specifically binds VV A56 and comprises one, two, three, four, five, or all six CDRs of the anti-VV A56 antibody designated herein as A047/A057, A049/A059/A056, A050, or A054. The amino acid sequences of the variable heavy chain (V_{H}) polypeptides, the variable light chain (V_{L}) polypeptides, and the CDRs of the A047/A057, A049/A059/A056, A050, and A054 antibodies are provided in Table 1 below. All CDRs and framework regions described throughout the present disclosure are defined according to Kabat unless otherwise indicated.

TABLE 1
Amino Acid Sequences of Example Anti-VV A56 Antibodies

| | |
|---|---|
| A047/A057 V_{H} | QEQLEESGGDLVKPEGSLTLTCTASGFSFSSSYWICWVRQAPG |
| (SEQ ID NO: 1) | KGLEWIACIYAGSGGSTYYATWAKGRFTISKTSSTTVTLQMTSLT |
| | AADTASYFCVRAYSDRSGGYSFNLWGPGTLVTVSS |
| A047/A057 V_{L} | QVLTQTASPVSAAVGGTVTINCQASQSVDNNNYLAWYQQKPGQ |
| (SEQ ID NO: 2) | PPKQLIYSASSLASGVPSRFKGSGSGTQFTLTISDVQCDDAATYY |
| | CLGSYDCSDADCYAFGGGTEVVVK |
| A047/A057 V_{H} CDR1 | SSYWIC |
| (SEQ ID NO: 3) | |
| A047/A057 V_{H} CDR2 | CIYAGSGGSTYYATWAKG |
| (SEQ ID NO: 4) | |
| A047/A057 V_{H} CDR3 | AYSDRSGGYSFNL |
| (SEQ ID NO: 5) | |
| A047/A057 V_{L} CDR1 | QASQSVDNNNYLA |
| (SEQ ID NO: 6) | |
| A047/A057 V_{L} CDR2 | SASSLAS |
| (SEQ ID NO: 7) | |
| A047/A057 V_{L} CDR3 | LGSYDCSDADCYA |
| (SEQ ID NO: 8) | |
| A049/A059/A056 V_{H} | QSLEESGGDLVKPGASLTLTCTASGIDFSDIYYISWVRQAPGKGL |
| (SEQ ID NO: 9) | EWIACTYAGSSGSTYYATWAKGRFTISKASSTTVTLQMTSLTAAD |
| | TATYFCARDRYPGTSGRVYGMDLWGPGTLVTVSS |
| A049/A059/A056 V_{L} | WMTQTPSSVSEPVGGTVTIKCQASQSISDLLSWYQQKPGQPPK |
| (SEQ ID NO: 10) | LLIYSASTLASGVSSRFKGSGSGTEFTLTISDLECADAATYYCQC |
| | NYYSPTYGNGFGGGTEVVVK |
| A049/A059/A056 V_{H} CDR1 | DIYYIS |
| (SEQ ID NO: 11) | |
| A049/A059/A056 V_{H} CDR2 | CTYAGSSGSTYYATWAKG |
| (SEQ ID NO: 12) | |
| A049/A059/A056 V_{H} CDR3 | DRYPGTSGRVYGMDL |
| (SEQ ID NO: 13) | |
| A049/A059/A056 V_{L} CDR1 | QASQSISDLLS |
| (SEQ ID NO: 14) | |
| A049/A059/A056 V_{L} CDR2 | SASTLAS |
| (SEQ ID NO: 15) | |
| A049/A059/A056 V_{L} CDR3 | QCNYYSPTYGNG |
| (SEQ ID NO: 16) | |
| A050 V_{H} | QSLEESGGDLVKPGASLTLTCTASGFSFSSSYWLCWARQAPGK |
| (SEQ ID NO: 17) | GPEWIACIYNGDGSTHYASWAKGRFTISKSSSTTVTLQMTSLTAA |
| | DTATYFCARDYTYNFYTYGFNLWGPGTLVTVSS |
| A050 V_{L} | FELTQTPSSVEAAVGGTVTINCQASQSVNIWASWYQQKPGQPP |
| (SEQ ID N0: 18) | KLLIYKASTLASGVPSRFKGSGSGTEFTLTISDLECADAATYYCQ |
| | GGYPSSSSGWAFGGGTEVVVK |
| A050 V_{H} CDR1 | SSYWLC |
| (SEQ ID NO: 19) | |
| A050 V_{H} CDR2 | CIYNGDGSTHYASWAKG |
| (SEQ ID NO: 20) | |
| A050 V_{H} CDR3 | DYTYNFYTYGFNL |
| (SEQ ID NO: 21) | |
| A050 V_{L} CDR1 | QASQSVNIWAS |
| (SEQ ID NO: 22) | |
| A050 V_{L} CDR2 | KASTLAS |
| (SEQ ID NO: 23) | |
| A050 V_{L} CDR3 | QGGYPSSSSGWA |
| (SEQ ID NO: 24) | |
| A054 V_{H} | QEQVVESGGGLVKPGASLTLTCTASGFTLSSSYWICWVRQAPG |
| (SEQ ID NO: 25) | KGPEWIACTYNGDGSTHYASWAKGRFTISKSSSTTVTLQMTSLT |
| | AADTATYFCARDYTDAFYTYGFNLWGPGTLVTVSS |
| A054 V_{L} | DIVMTQTPASVSEPVGGTVTIKCQASQSTSSYLAWYQQKPGQPP |
| (SEQ ID NO: 26) | KLLIYRASSLASGVPSRFKGSGSATEFTLTISDLECADAATYYCQT |
| | GFYGSSGHTFGGGTEVVVK |
| A054 V_{H} CDR1 | SSYWIC |
| (SEQ ID NO: 27) | |
| A054 V_{H} CDR2 | CTYNGDGSTHYASWAKG |
| (SEQ ID NO: 28) | |
| A054 V_{H} CDR3 | DYTDAFYTYGFNL |
| (SEQ ID NO: 29) | |
| A054 V_{L} CDR1 | QASQSTSSYLA |
| (SEQ ID NO: 30) | |
| A054 V_{L} CDR2 | RASSLAS |
| (SEQ ID NO: 31) | |
| A054 V_{L} CDR3 | QTGFYGSSGHT |
| (SEQ ID NO: 32) | |
| V_{L} of A56-CAR-01, A56- | DVVMTQTPSSVSEPVGGTVTIKCQASQSISDLLSWYQQKPGQPP |
| CAR-02, A56-CAR-05, A56- | KLLIYSASTLASGVSSRFKGSGSGTEFTLTISDLECADAATYYCQ |
| CAR-06, A56-CAR-07, A56- | CNYYSPTYGNGFGGGTEVVVKK |
| CAR-08, A56-CAR-010, | |
| A56-CAR-020, A56-CAR- | |
| 021 | |
| (SEQ ID NO: 62) | |
| V_{L} of A56-CAR-029, A56- | DVVMTQTPSSVSEPVGGTVTIKCQASQSISDLLSWYQQKPGQPP |
| CAR-030 | KLLIYSASTLASGVSSRFKGSGSGTEFTLTISDLECADAATYYCQ |
| (SEQ ID NO: 63) | CNYYSPTYGNGFGGGTEVVVK |

In certain embodiments, an antibody of the present disclosure specifically binds VV A56 and competes for binding to VV A56 with an antibody comprising:
a variable heavy chain (V_{H}) polypeptide comprising
   a V_{H} CDR1 comprising the amino acid sequence SSYWIC (SEQ ID NO: 3),
   a V_{H} CDR2 comprising the amino acid sequence CIYAGSGGSTYYATWAKG (SEQ ID NO: 4), and
   a V_{H} CDR3 comprising the amino acid sequence AYSDRSGGYSFNL (SEQ ID NO: 5); and
a variable light chain (V_{L}) polypeptide comprising
   a V_{L} CDR1 comprising the amino acid sequence QASQSVDNNNYLA (SEQ ID NO: 6),
   a V_{L} CDR2 comprising the amino acid sequence SASSLAS (SEQ ID NO: 7), and
   a V_{L} CDR3 comprising the amino acid sequence LGSYDCSDADCYA (SEQ ID NO: 8).

In certain embodiments, such an antibody comprises the six CDRs set forth in SEQ ID NO: 3, SEQ ID NO: 4, SEQ ID NO: 5, SEQ ID NO: 6, SEQ ID NO: 7, and SEQ ID NO: 8. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 1; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 2; or both.

According to some embodiments, an antibody of the present disclosure specifically binds VV A56 and competes for binding to VV A56 with an antibody comprising:
a variable heavy chain (V_{H}) polypeptide comprising
   a V_{H} CDR1 comprising the amino acid sequence DIYYIS (SEQ ID NO: 11),
   a V_{H} CDR2 comprising the amino acid sequence CTYAGSSGSTYYATWAKG (SEQ ID NO: 12), and
   a V_{H} CDR3 comprising the amino acid sequence DRYPGTSGRVYGMDL (SEQ ID NO: 13); and
a variable light chain (V_{L}) polypeptide comprising
   a V_{L} CDR1 comprising the amino acid sequence QASQSISDLLS (SEQ ID NO: 14),
   a V_{L} CDR2 comprising the amino acid sequence SASTLAS (SEQ ID NO: 15), and
   a V_{L} CDR3 comprising the amino acid sequence QCNYYSPTYGNG (SEQ ID NO: 16).

In certain embodiments, such an antibody comprises the six CDRs set forth in SEQ ID NO: 11, SEQ ID NO: 12, SEQ ID NO: 13, SEQ ID NO: 14, SEQ ID NO: 15, and SEQ ID NO: 16. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 9; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 10; or both. In certain embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 9; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 62; or both. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 9; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 63; or both.

In certain embodiments, an antibody of the present disclosure specifically binds VV A56 and competes for binding to VV A56 with an antibody comprising:
a variable heavy chain (V_{H}) polypeptide comprising
   a V_{H} CDR1 comprising the amino acid sequence SSYWLC (SEQ ID NO: 19),
   a V_{H} CDR2 comprising the amino acid sequence CIYNGDGSTHYASWAKG (SEQ ID NO: 20), and
   a V_{H} CDR3 comprising the amino acid sequence DYTYNFYTYGFNL (SEQ ID NO: 21); and
a variable light chain (V_{L}) polypeptide comprising
   a V_{L} CDR1 comprising the amino acid sequence QASQSVNIWAS (SEQ ID NO: 22),
   a V_{L} CDR2 comprising the amino acid sequence KASTLAS (SEQ ID NO: 23), and
   a V_{L} CDR3 comprising the amino acid sequence QGGYPSSSSGWA (SEQ ID NO: 24).

In certain embodiments, such an antibody comprises the six CDRs set forth in SEQ ID NO: 19, SEQ ID NO: 20, SEQ ID NO: 21, SEQ ID NO: 22, SEQ ID NO: 23, and SEQ ID NO: 24. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 17; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 18; or both.

According to some embodiments, an antibody of the present disclosure specifically binds VV A56 and competes for binding to VV A56 with an antibody comprising:
a variable heavy chain (V_{H}) polypeptide comprising
   a V_{H} CDR1 comprising the amino acid sequence SSYWIC (SEQ ID NO: 27),
   a V_{H} CDR2 comprising the amino acid sequence CTYNGDGSTHYASWAKG (SEQ ID NO: 28), and
   a V_{H} CDR3 comprising the amino acid sequence DYTDAFYTYGFNL (SEQ ID NO: 29); and
a variable light chain (V_{L}) polypeptide comprising
   a V_{L} CDR1 comprising the amino acid sequence QASQSTSSYLA (SEQ ID NO: 30),
   a V_{L} CDR2 comprising the amino acid sequence RASSLAS (SEQ ID NO: 31), and
   a V_{L} CDR3 comprising the amino acid sequence QTGFYGSSGHT (SEQ ID NO: 32).

In certain embodiments, such an antibody comprises the six CDRs set forth in SEQ ID NO: 27, SEQ ID NO: 28, SEQ ID NO: 29, SEQ ID NO: 30, SEQ ID NO: 31, and SEQ ID NO: 32. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 25; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 26; or both.

Suitable VV A56 antigens for determining whether an anti-VV A56 antibody of the present disclosure “competes with” a second antibody for binding to the VV A56 antigen include the Wyeth, Western Reserve and Copenhagen A56 antigens having the amino acid sequences shown in Table 2 below.

TABLE 2
A56 antigen sequences for Wyeth, Western Reserve and Copenhagen VV

| | |
|---|---|
| Wyeth_VV-_A56 | MTRLPILLLLISLVYATPFPQTSKKIGDDATLSCNRNNTNDYVVMS |
| (SEQ ID NO: 180) | AWYKEPNSIILLAAKSDVLYFDNYTKDKISYDSPYDDLVTTITIKSL |
| | TARDAGTYVCAFFMTSPTNDTDKVDYEEYSTELIVNTDSESTIDII |
| | LSGSTHSPETSSEKPDYIDNSNCSSVFEIATPEPITDNVEDHTDTV |
| | TYTSDSINTVSATSGESTTDETPEPITDKEDHTVTDTVSYTTVSTS |
| | SGIVTTKSTTDDTYNDNDTVPPTTVGGSTTSISNYKTKDFVEIFGI |
| | TALIILSAVAIFCITYYICNKRSRKYKTENKV |
| Western_reserve_-_A56 | MTRLPILLLLISLVYATPFPQTSKKIGDDATLSCNRNNTNDYVVMS |
| (Q01218 Uniprot) | AWYKEPNSIILLAAKSDVLYFDNYTKDKISYDSPYDDLVTTITIKSL |
| (SEQ ID NO: 182) | TARDAGTYVCAFFMTSTTNDTDKVDYEEYSTELIVNTDSESTIDIIL |
| | SGSTHSPETSSKKPDYIDNSNCSSVFEIATPEPITDNVEDHTDTVT |
| | YTSDSINTVSASSGESTTDETPEPITDKEDHTVTDTVSYTTVSTSS |
| | GIVTTKSTTDDADLYDTYNDNDTVPPTTVGGSTTSISNYKTKDFV |
| | EIFGITALIILSAVAIFCITYYIYNKRSRKYKTENKV |
| Copenhagen_-_A56 | MTRLPILLLLISLVYATPFPQTSKKIGDDATLSCNRNNTNDYVVMS |
| (P20978 Uniprot) | AWYKEPNSIILLAAKSDVLYFDNYTKDKISYDSPYDDLVTTITIKSL |
| (SEQ ID NO: 184) | TARDAGTYVCAFFMTSPTNDTDKVDYEEYSTELIVNTDSESTIDII |
| | LSGSTHSPETSSEKPDYIDNSNCSSVFEIATPEPITDNVEDHTDTV |
| | TYTSDSINTVSATSGESTTDETPEPITDKEEDHTVTDTVSYTTVST |
| | SSGIVTTKSTTDDADLYDTYNDNDTVPSTTVGSSTTSISNYKTKD |
| | FVEIFGITALIILSAVAIFCITYYICNKRSRKYKTENKV |

Anti-VV B5 Antibodies

According to some embodiments, provided are antibodies that specifically bind a VV B5 antigen (VV B5). VV B5 protein is a 42 kDa type I transmembrane glycoprotein with an extracellular domain composed of four short consensus repeats (SCRs) characteristic of complement control proteins. After the SCRs, B5 has a stalk region before the transmembrane domain and a short cytoplasmic tail (CT). Both the SCRs and CT are dispensable for targeting B5 to the extracellular enveloped virus (EEV) membrane, although the latter affects its transport to the cell surface and recycling via endosomes. B5 is needed for intracellular mature virus (IMV) wrapping to form intracellular enveloped virus (IEV).

In certain embodiments, an antibody of the present disclosure specifically binds VV B5 and competes for binding to VV B5 with an antibody having one, two, three, four, five, or all six complementarity determining regions (CDRs) of one or more of the anti-VV B5 antibodies designated herein as A048/A058/A073 and A051. According to some embodiments, an antibody of the present disclosure specifically binds VV B5 and comprises one, two, three, four, five, or all six CDRs of the anti-VV B5 antibody designated herein as A048/A058/A073 or A051. The amino acid sequences of the variable heavy chain (V_{H}) polypeptides, the variable light chain (V_{L}) polypeptides, and the CDRs of the A048/A058/A073 and A051 antibodies are provided in Table 3 below.

TABLE 3
Amino Acid Sequences of Example Anti-VV B5 Antibodies

| | |
|---|---|
| A048/A058/A073 V_{H} | QEQLEESGGGLVKPEGSLTLTCTASGFSFSSSYYMCWVRQAPG |
| (SEQ ID NO: 33) | RGLEWIACIYTSSGSAYYANWAKGRFTISRTSSTTVTLQMTRLTA |
| | ADTATYFCVRNAVGSSYYLYLWGPGTLVTVSS |
| A048/A058/A073 V_{L} | QVLTQTPSPVSAAVGGTVTISCQASQSVAGNNYLSWYQQKPGQ |
| (SEQ ID NO: 34) | PPNLLIYSVSTLASGVPSRFKGSGSGTQFTLTISDLECDDAATYY |
| | CQGYYNDGIWAFGGGTEVVVK |
| A048/A058/A073 V_{H} CDR1 | SSYYMC |
| (SEQ ID NO: 35) | |
| A048/A058/A073 V_{H} CDR2 | CIYTSSGSAYYANWAKG |
| (SEQ ID NO: 36) | |
| A048/A058/A073 V_{H} CDR3 | NAVGSSYYLYL |
| (SEQ ID NO: 37) | |
| A048/A058/A073 V_{L} CDR1 | QASQSVAGNNYLS |
| (SEQ ID NO: 38) | |
| A048/A058/A073 V_{L} CDR2 | SVSTLAS |
| (SEQ ID NO: 39) | |
| A048/A058/A073 V_{L} CDR3 | QGYYNDGIWA |
| (SEQ ID NO: 40) | |
| A051 V_{H} | QEQVEESGGGLAKPGASLTLTCEASGFTLSSYWMCWVRQAPG |
| (SEQ ID NO: 41) | KGLEWIACIYGGSSGSTYYSNWAKGRFTISKTSSTTVTLQMTSLT |
| | AADTATYFCARDGSTWDYFRLWGPGTLVTVSS |
| A051 V_{L} | YDMTQTPSSVEAAVGGTVTIKCQASQSINTNYLSWYQQKPGQP |
| (SEQ ID NO: 42) | PKLLIYQASTLESGVPSRFKGSGSGTEYTLTISDLECADAATYYC |
| | QGYYTVENIGNPFGGGTEVVVK |
| A051 V_{H} CDR1 | SYWMC |
| (SEQ ID NO: 43) | |
| A051 V_{H} CDR2 | CIYGGSSGSTYYSNWAKG |
| (SEQ ID NO: 44) | |
| A051 V_{H} CDR3 | DGSTWDYFRL |
| (SEQ ID NO: 45) | |
| A051 V_{L} CDR1 | QASQSINTNYLS |
| (SEQ ID NO: 46) | |
| A051 V_{L} CDR2 | QASTLES |
| (SEQ ID NO: 47) | |
| A051 V_{L} CDR3 | QGYYTVENIGNP |
| (SEQ ID NO: 48) | |

In certain embodiments, an antibody of the present disclosure specifically binds VV B5 and competes for binding to WV B5 with an antibody comprising:
a variable heavy chain (V_{H}) polypeptide comprising
   a V_{H} CDR1 comprising the amino acid sequence SSYYMC (SEQ ID NO: 35),
   a V_{H} CDR2 comprising the amino acid sequence CIYTSSGSAYYANWAKG (SEQ ID NO: 36), and
   a V_{H} CDR3 comprising the amino acid sequence NAVGSSYYLYL (SEQ ID NO: 37); and
a variable light chain (V_{L}) polypeptide comprising
   a V_{L} CDR1 comprising the amino acid sequence QASQSVAGNNYLS (SEQ ID NO: 38),
   a V_{L} CDR2 comprising the amino acid sequence SVSTLAS (SEQ ID NO: 39), and
   a V_{L} CDR3 comprising the amino acid sequence QGYYNDGIWA (SEQ ID NO: 40).

In certain embodiments, such an antibody comprises the six CDRs set forth in SEQ ID NO: 35, SEQ ID NO: 36, SEQ ID NO: 37, SEQ ID NO: 38, SEQ ID NO: 39, and SEQ ID NO: 40. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 33; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 34; or both.

According to some embodiments, an antibody of the present disclosure specifically binds VV B5 and competes for binding to VV B5 with an antibody comprising:
a variable heavy chain (V_{H}) polypeptide comprising
   a V_{H} CDR1 comprising the amino acid sequence SYWMC (SEQ ID NO: 43),
   a V_{H} CDR2 comprising the amino acid sequence CIYGGSSGSTYYSNWAKG (SEQ ID NO: 44), and
   a V_{H} CDR3 comprising the amino acid sequence DGSTWDYFRL (SEQ ID NO: 45); and
a variable light chain (V_{L}) polypeptide comprising
   a V_{L} CDR1 comprising the amino acid sequence QASQSINTNYLS (SEQ ID NO: 46),
   a V_{L} CDR2 comprising the amino acid sequence QASTLES (SEQ ID NO: 47), and
   a V_{L} CDR3 comprising the amino acid sequence QGYYTVENIGNP (SEQ ID NO: 48).

In certain embodiments, such an antibody comprises the six CDRs set forth in SEQ ID NO: 43, SEQ ID NO: 44, SEQ ID NO: 45, SEQ ID NO: 46, SEQ ID NO: 47, and SEQ ID NO: 48. According to some embodiments, the antibody comprises: a variable heavy chain (V_{H}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 41; a variable light chain (V_{L}) polypeptide comprising an amino acid sequence having 70% or greater, 75% or greater, 80% or greater, 85% or greater, 90% or greater, 95% or greater, or 100% identity to the amino acid sequence set forth in SEQ ID NO: 42; or both.

Suitable VV B5 antigens for determining whether an anti-VV B5 antibody of the present disclosure “competes with” a second antibody for binding to the VV B5 antigen include the Wyeth, Western Reserve and Copenhagen B5 antigens having the amino acid sequences shown in Table 4 below.

TABLE 4
B5 antigen sequences for Wyeth, Western Reserve and Copenhagen VV

| | |
|---|---|
| Wyeth_VV-_B5 | MKTISWTLLCVLPAVVYSTCTVPTMNNAKLTSTETSFNNNQKVT |
| (SEQ ID NO: 181) | FTCDQGYHSSDPNAVCETDKWKYENPCKKMCTVSDYVSELYDK |
| | PLYEVNSTMTLSCNGETKYFRCEEKNGNTSWNDTVTCPNAECQ |
| | PLQLEHGSCQPVKEKYSFGEYITINCDVGYEVIGASYISCTANSW |
| | NVIPSCQQKCDIPSLSNGLISGSTFSIGGVIHLSCKSGFILTGSPSS |
| | TCIDGKWNPILPTCVRSNEKFDPVDDGPDDETDLSKLSKDWQY |
| | EQEIESLEATYHIIIVALTIMGVIFLISVIVLVCSCDKNNDQYKFHKLL |
| | P |
| Western_reserve_-_B5 | MKTISWTLLCVLPAVVYSTCTVPTMNNAKLTSTETSFNDKQKVT |
| (Q01227 Uniprot) | FTCDQGYHSSDPNAVCETDKWKYENPCKKMCTVSDYISELYNK |
| (SEQ ID NO: 183) | PLYEVNSTMTLSCNGETKYFRCEEKNGNTSWNDTVTCPNAECQ |
| | PLQLEHGSCQPVKEKYSFGEYMTINCDVGYEVIGASYISCTANS |
| | WNVIPSCQQKCDMPSLSNGLISGSTFSIGGVIHLSCKSGFTLTGS |
| | PSSTCIDGKWNPVLPICVRTNEEFDPVDDGPDDETDLSKLSKDV |
| | VQYEQEIESLEATYHIIIVALTIMGVIFLISVIVLVCSCDKNNDQYKF |
| | HKLLP |
| Copenhagen_-_B5 | MKTISWTLLCVLPAVVYSTCTVPTMNNAKLTSTETSFNNNQKVT |
| (P21115 Uniprot) | FTCDQGYHSSDPNAVCETDKWKYENPCKKMCTVSDYISELYNK |
| (SEQ ID NO: 185) | PLYEVNSTMTLSCNGETKYFRCEEKNGNTSWNDTVTCPNAECQ |
| | PLQLEHGSCQPVKEKYSFGEYMTINCDVGYEVIGASYISCTANS |
| | WNVIPSCQQKCDIPSLSNGLISGSTFSIGGVIHLSCKSGFILTGSP |
| | SSTCIDGKWNPVLPICVRTNEEFDPVDDGPDDETDLSKLSKDW |
| | QYEQEIESLEATYHIIIVALTIMGVIFLISVIVLVCSCDKNNDQYKFH |
| | KLLP |

Bispecific Antibodies

Also provided are bispecific antibodies. In certain embodiments, a bispecific antibody of the present disclosure comprises a first antigen-binding domain comprising a V_{H} polypeptide-V_{L} polypeptide pair of any of the anti-VV A56 and anti-VV B5 antibodies of the present disclosure, including any of such antibodies described hereinabove. The bispecific antibody may include a second antigen-binding domain that specifically binds the VV antigen bound by the first antigen-binding domain. In certain embodiments, the bispecific antibody includes a second antigen-binding domain that specifically binds a VV antigen other than the VV antigen bound by the first antigen-binding domain.

According to some embodiments, a bispecific antibody of the present disclosure includes a second antigen-binding domain that specifically binds an antigen other than a VV antigen. In certain embodiments, the antigen other than a VV antigen is an immune cell surface antigen. Non-limiting examples of immune cell surface antigens are immune effector cell surface antigens, e.g., a T cell surface antigen, a natural killer (NK) cell surface antigen, a macrophage cell surface antigen, and the like. Examples of T cell surface antigens that may be bound by the second antigen-binding domain include, but are not limited to, a T cell stimulatory molecule, e.g., CD3, CD28, etc.

Bispecific antibodies of the present disclosure include antibodies having a full-length antibody structure, and bispecific antibody fragments. “Full-length” as used herein refers to an antibody having two full-length antibody heavy chains and two full length antibody light chains. A full-length antibody heavy chain (HC) consists of well-known heavy chain variable and constant domains VH, CH1, CH2, and CH3. A full-length antibody light chain (LC) consists of well-known light chain variable and constant domains VL and CL. The full-length antibody may be lacking the C-terminal lysine in either one or both heavy chains. The term “Fab arm” refers to one heavy chain:light chain pair that specifically binds an antigen.

Full-length bispecific antibodies may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in a cell-free environment or using co-expression. The Fab arm exchange reaction is the result of a disulfide-bond isomerization reaction and dissociation-association of CH3 domains. The heavy chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope.

The “knob-in-hole” strategy (see, e.g., WO 2006/028936) may be used to generate full length bispecific antibodies. Briefly, selected amino acids forming the interface of the CHS domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y7F405A, T366W/F405W, F405W/Y407A, T394W/Y407T, T3945/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US2010/0015133; US2009/0182127; US2010/028637 or US2011/0123532. In other strategies. heterodimerization may be promoted by the following substitutions (expressed as modified position in the first CH3 domain of the first heavy chain/modified position in the second CH3 domain of the second heavy chain): L351 Y_F405A_Y407V T394W, T3661_K392M_T394W/F405A_Y407V, T366L_K392M_T394W/F405A_Y407V, L351 Y_Y407A′T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849.

Also provided are single chain bispecific antibodies. In some embodiments, a single chain bispecific antibody of the present disclosure is a bispecific scFv. Details regarding bispecific scFvs may be found, e.g., in Zhou et al. (2017) J Cancer 8(18):3689-3696.

Approaches that may be employed to produce multispecific (e.g., bispecific) antibodies from the antibodies described herein include, but are not limited to, Ellerman, D. (2019). “Bispecific T-cell engagers: Towards understanding variables influencing the in vitro potency and tumor selectivity and their modulation to enhance their efficacy and safety.” Methods 154: 102-117; Brinkmann, U. and R. E. Kontermann (2017). “The making of bispecific antibodies.” mAbs 9(2): 182-212; and Suurs, F. V., et al. (2019). “A review of bispecific antibodies and antibody constructs in oncology and clinical challenges.” Pharmacol Ther 201: 103-119; the disclosures of which are incorporated herein by reference in their entireties for all purposes.

Fusion Proteins

Also provided are fusion proteins. In certain embodiments, a fusion protein of the present disclosure comprises a chain of any of the anti-VV A56 or anti-VV B5 antibodies of the present disclosure, fused to a heterologous sequence of amino acids. The heterologous sequence of amino acids may be fused to the C-terminus of the chain of the antibody or the N-terminus of the chain of the antibody. In certain embodiments, a fusion protein of the present disclosure includes a heterologous sequence at the C-terminus of the chain of the antibody and a heterologous sequence at the N-terminus of the chain of the antibody, wherein the heterologous sequences may be the same sequence or different sequences. “Heterologous” as used in the context of a nucleic acid or polypeptide generally means that the nucleic acid or polypeptide is from a different origin (e.g., molecule of different sequence, different species origin, and the like) than that with which the nucleic acid or polypeptide is associated or joined, such that the nucleic acid or polypeptide is one that is not found in nature. For example, in a fusion protein, a light chain polypeptide and a reporter polypeptide (e.g., GFP, red fluorescent protein (e.g., mCherry), luciferase, etc.) are said to be “heterologous” to one another. Similarly, a CDR from a mouse antibody and a constant region from a human antibody are “heterologous” to one another.

The chain of the anti-VV A56 or anti-VV B5 antibody may be fused to any heterologous sequence of interest. Heterologous sequences of interest include, but are not limited to, an albumin, a transferrin, XTEN, a homo-amino acid polymer, a proline-alanine-serine polymer, an elastin-like peptide, or any combination thereof. In certain aspects, the heterologous polypeptide increases the stability and/or serum half-life of the antibody upon its administration to an individual in need thereof, as compared to the same antibody which is not fused to the heterologous sequence.

In certain embodiments, a fusion protein of the present disclosure comprises a single chain antibody, e.g., a single chain antibody (e.g., scFv) comprising a V_{H} polypeptide-V_{L} polypeptide pair of any of the anti-VV A56 and anti-VV B5 antibodies of the present disclosure, including any of such antibodies described hereinabove. scFvs of the present disclosure include, but are not limited to, scFvs comprising the six CDRs of an scFv set forth in Table 5 below, which scFv in some embodiments comprises a variable heavy chain (V_{H}) polypeptide comprising an amino acid sequence having 70% or greater, 75% or greater, 80% or greater, 85% or greater, 90% or greater, 95% or greater, or 100% identity to the amino acid sequence of the V_{H} of the scFv set forth in Table 5; and a variable light chain (V_{L}) polypeptide comprising an amino acid sequence having 70% or greater, 75% or greater, 80% or greater, 85% or greater, 90% or greater, 95% or greater, or 100% identity to the amino acid sequence of the V_{L} of the scFv set forth in Table 5. In Table 5, segments/domains of the polypeptides are indicated by alternating underlining, and the identities of the segments/domains are provided in the left column.

TABLE 5
Example scFv Amino Acid Sequences

| | |
|---|---|
| scFv of A56-CAR-01, A56- | QSLEESGGDLVKPGASLTLTCTASGIDFSDIYYISWVRQAPGKGLE |
| CAR-05, A56-CAR-06, A56- | WIACTYAGSSGSTYYATWAKGRFTISKASSTTVTLQMTSLTAADTA |
| CAR-07, A56-CAR-020, A56- | TYFCARDRYPGTSGRVYGMDLWGPGTLVTVSSGSTSGSGKPGSG |
| CAR-021 | EGSTKGDVVMTQTPSSVSEPVGGTVTIKCQASQSISDLLSWYQQK |
| •V_{H} | PGQPPKLLIYSASTLASGVSSRFKGSGSGTEFTLTISDLECADAATY |
| •Linker | YCQCNYYSPTYGNGFGGGTEVVVKK |
| •V_{L} | |
| (SEQ ID NO: 64) | |
| scFv of A56-CAR-02, A56- | DVVMTQTPSSVSEPVGGTVTIKCQASQSISDLLSWYQQKPGQPPK |
| CAR-08, A56-CAR-010 | LLIYSASTLASGVSSRFKGSGSGTEFTLTISDLECADAATYYCQCNY |
| •V_{L} | YSPTYGNGFGGGTEVVVKKGSTSGSGKPGSGEGSTKGQSLEESG |
| •Linker | GDLVKPGASLTLTCTASGIDFSDIYYISWVRQAPGKGLEWIACTYAG |
| •V_{H} | SSGSTYYATWAKGRFTISKASSTTVTLQMTSLTAADTATYFCARDR |
| (SEQ ID NO: 65) | YPGTSGRVYGMDLWGPGTLVTVSS |
| scFv of A56-CAR-27 | QSLEESGGDLVKPGASLTLTCTASGIDFSDIYYISWVRQAPGKGLE |
| •V_{H} | WIACTYAGSSGSTYYATWAKGRFTISKASSTTVTLQMTSLTAADTA |
| •Linker | TYFCARDRYPGTSGRVYGMDLWGPGTLVTVSSGSTSGSGKPGSG |
| •V_{L} | EGSTKGVVMTQTPSSVSEPVGGTVTIKCQASQSISDLLSWYQQKP |
| (SEQ ID NO: 66) | GQPPKLLIYSASTLASGVSSRFKGSGSGTEFTLTISDLECADAATYY |
| | CQCNYYSPTYGNGFGGGTEVVVK |
| scFv of A56-CAR-28 | WMTQTPSSVSEPVGGTVTIKCQASQSISDLLSWYQQKPGQPPKL |
| •V_{L} | LIYSASTLASGVSSRFKGSGSGTEFTLTISDLECADAATYYCQCNYY |
| •Linker | SPTYGNGFGGGTEVVVKGSTSGSGKPGSGEGSTKGQSLEESGGD |
| •V_{H} | LVKPGASLTLTCTASGIDFSDIYYISVWRQAPGKGLEWIACTYAGSS |
| (SEQ ID NO: 67) | GSTYYATWAKGRFTISKASSTTVTLQMTSLTAADTATYFCARDRYP |
| | GTSGRVYGMDLWGPGTLVTVSS |
| scFv of A56-CAR-029 | QSLEESGGDLVKPGASLTLTCTASGIDFSDIYYISWVRQAPGKGLE |
| •V_{H} | WIACTYAGSSGSTYYATWAKGRFTISKASSTTVTLQMTSLTAADTA |
| •Linker | TYFCARDRYPGTSGRVYGMDLWGPGTLVTVSSGSTSGSGKPGSG |
| •V_{L} | EGSTKGDVVMTQTPSSVSEPVGGTVTIKCQASQSISDLLSWYQQK |
| (SEQ ID NO: 68) | PGQPPKLLIYSASTLASGVSSRFKGSGSGTEFTLTISDLECADAATY |
| | YCQCNYYSPTYGNGFGGGTEVVVK |
| scFv of A56-CAR-030 | DVVMTQTPSSVSEPVGGTVTIKCQASQSISDLLSWYQQKPGQPPK |
| •V_{L} | LLIYSASTLASGVSSRFKGSGSGTEFTLTISDLECADAATYYCQCNY |
| •Linker | YSPTYGNGFGGGTEVVVKGSTSGSGKPGSGEGSTKGQSLEESGG |
| •V_{H} | DLVKPGASLTLTCTASGIDFSDIYYISWVRQAPGKGLEWACTYAGS |
| (SEQ ID NO: 69) | SGSTYYATWAKGRFTISKASSTTVTLQMTSLTAADTATYFCARDRY |
| | PGTSGRVYGMDLWGPGTLVTVSS |
| scFv of B5-CAR-03, B5- | QEQLEESGGGLVKPEGSLTLTCTASGFSFSSSYYMCWVRQAPGR |
| CAR-011, B5-CAR-013, B5- | GLEWIACIYTSSGSAYYANWAKGRFTISRTSSTTVTLQMTRLTAAD |
| CAR-019, B5-CAR-022 | TATYFCVRNAVGSSYYLYLWGPGTLVTVSSGSTSGSGKPGSGEG |
| •V_{H} | STKGQVLTQTPSPVSAAVGGTVTISCQASQSVAGNNYLSWYQQKP |
| •Linker | GQPPNLLIYSVSTLASGVPSRFKGSGSGTQFTLTISDLECDDAATYY |
| •V_{L} | CQGYYNDGIWAFGGGTEVVVK |
| (SEQ ID NO: 70) | |
| scFv of B5-CAR-04, B5- | QVLTQTPSPVSAAVGGTVTISCQASQSVAGNNYLSWYQQKPGQP |
| CAR-014, B5-CAR-016 | PNLLIYSVSTLASGVPSRFKGSGSGTQFTLTISDLECDDAATYYCQ |
| •V_{L} | GYYNDGIWAFGGGTEVVVKGSTSGSGKPGSGEGSTKGQEQLEES |
| •Linker | GGGLVKPEGSLTLTCTASGFSFSSSYYMCWVRQAPGRGLEWIACI |
| •V_{H} | YTSSGSAYYANWAKGRFTISRTSSTTVTLQMTRLTAADTATYFCVR |
| (SEQ ID NO: 71) | NAVGSSYYLYLWGPGTLVTVSS |

According to some embodiments, when the fusion protein comprises a single chain antibody (e.g., any of the single chain antibodies of the present disclosure, including any of the scFvs described herein), the fusion protein is a chimeric antigen receptor (CAR) comprising the single chain antibody, a transmembrane domain, and an intracellular signaling domain.

A CAR of the present disclosure may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a serine-glycine linker, such as a serine-glycine linker that includes the amino acid sequence GGGGSGGGGSGGGGS (G₄S)₃ (SEQ ID NO: 49). In certain aspects, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, the antigen binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8α and CD4, which may be wild-type hinge regions from these molecules or variants thereof. In certain aspects, the hinge domain includes a CD8α hinge region. In some embodiments, the hinge is a PD-1 hinge or CD152 hinge.

The “transmembrane domain” (Tm domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a CAR includes a Tm domain derived from CD8α. In certain aspects, a CAR includes a Tm domain derived from CD8α and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3 primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of immune effector cells (e.g., T cells) expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the CAR includes one or more costimulatory signaling domains selected from the group consisting of 4-1BB (CD137), CD28, and CD134, and a CD3 primary signaling domain.

A CAR of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like. Non-limiting examples of such domains that may be included in a CAR of the present disclosure include those provided in Table 6 below. As will be appreciated by one of ordinary skill in the art, the amino acid sequence of one or more of the domains indicated in Table 6 (e.g., linker, hinge, transmembrane, co-stimulatory, signaling, ribosomal skip element-restriction enzyme sequence; reporter protein etc.) may be modified as desired, e.g., for improved functionality, etc. of the CAR.

TABLE 6
Example CAR Domain Amino Acid Sequences
Leader Signal Peptide(s)

| | |
|---|---|
| GM-CSFR alpha (P15509) | MLLLVTSLLLCELPHPAFLLIP |
| (SEQ ID NO: 113) | |

Hinge/Spacer/Linker domain(s)

| | |
|---|---|
| (SEQ ID NO: 114) | GSTSGSGKPGSGEGSTKG |
| (SEQ ID NO: 49) | GGGGSGGGGSGGGGS |
| CD8a hinge | TTTPAPRPPTPAPTIASQPLSLRPEACRPAAGGAVHTRGLDFACD |
| (SEQ ID NO: 115) | |
| CD8a hinge 2 | AKPTTTPAPRPPTPAPTIASQPLSLRPEACRPAAGGAVHTRGLDF |
| (SEQ ID NO: 116) | ACD |
| extended CD8 hinge | GGGGSGGGGSGGGGSGGTTTPAPRPPTPAPTIASQPLSLRPEA |
| (SEQ ID NO: 117) | CRPAAGGAVHTRGLDFACD |
| CD28 hinge | IEVMYPPPYLDNEKSNGTIIHVKGKHLCPSPLFPGPSKP |
| (SEQ ID NO: 118) | |
| GSG linker | GGGSSGGGSG |
| (SEQ ID NO: 119) | |
| IgG4 hinge | ESKYGPPCPSCP |
| (SEQ ID NQ: 120) | |
| IgG4(CH3) | GQPREPQVYTLPPSQEEMTKNQVSLTCLVKGFYPSDIAVEWESN |
| (SEQ ID NO: 121) | GQPENNYKTTPPVLDSDGSFFLYSRLTVDKSRWQEGNVFSCVM |
| | HEALHNHYTQKSLSLSLGK |
| IgG4 (P01861) | ESKYGPPCPSCPAPEFLGGPSVFLFPPKPKDTLMISRTPEVTCVV |
| (SEQ ID NO: 122) | VDVSQEDPEVQFNWYVDGVEVHNAKTKPREEQFNSTYRVVSVLT |
| | VLHQDWLNGKEYKCKVSNKGLPSSIEKTISKAKGQPREPQVYTLP |
| | PSQEEMTKNQVSLTCLVKGFYPSDIAVEWESNGQPENNYKTTPP |
| | VLDSDGSFFLYSRLTVDKSRWQEGNVFSCSVMHEALHNHYTQKS |
| | LSLSLGK |

Transmembrane domain(s)

| | |
|---|---|
| CD8tm (NM_001768) | IYIWAPLAGTCGVLLLSLVITLYC |
| (SEQ ID NO: 123) | |
| CD8tm2 (NM_001768) | IYIWAPLAGTCGVLLLSLVITLY |
| (SEQ ID NO: 124) | |
| CD8tm3 (NM_001768) | IYIWAPLAGTCGVLLLSLVITL |
| (SEQ ID NO: 125) | |
| CD28tm (NM_006139) | FWVLVVVGGVLACYSLLVTVAFIIFW |
| (SEQ ID NO: 126) | |
| CD28tm2 (NM_006139) | MFWVLVVVGGVLACYSLLVTVAFIIFWV |
| (SEQ ID NO: 127) | |
| CD3z (J04132.1) | LCYLLDGILFIYGVILTALFL |
| (SEQ ID NO: 128) | |
| CD4tm (M35160) | MALIVLGGVAGLLLFIGLGIFF |
| (SEQ ID NO: 129) | |
| 4-1BB (NM_001561) | IISFFLALTSTALLFLLFFLTLRFSW |
| (SEQ ID NQ: 130) | |

Costimulatory domain(s)

| | |
|---|---|
| 4-1BB (NM_001561) | KRGRKKLLYIFKQPFMRPVQTTQEEDGCSCRFPEEEEGGCEL |
| (SEQ ID NO: 131) | |
| CD28 (NM_006139) | RSKRSRLLHSDYMNMTPRRPGPTRKHYQPYAPPRDFAAYRS |
| (SEQ ID NO: 132) | |
| CD28gg (NM_006139) | RSKRSRGGHSDYMNMTPRRPGPTRKHYQPYAPPRDFAAYRS |
| (SEQ ID NO: 133) | |
| OX40 (P43489) | ALYLLRRDQRLPPDAHKPPGGGSFRTPIQEEQADAHSTLAKI |
| (SEQ ID NO: 134) | |

CD3ζ domain(s)

| | |
|---|---|
| CD3ζ | RVKFSRSADAPAYQQGQNQLYNELNLGRREEYDVLDKRRGRDP |
| (SEQ ID NO: 135) | EMGGKPRRKNPQEGLYNELQKDKMAEAYSEIGMKGERRRGKGH |
| | DGLYQGLSTATKDTYDALHMQALPPR |

Ribosomal Skip Element(s)

| | |
|---|---|
| E2A | GSGQCTNYALLKLAGDVESNPGP |
| (SEQ ID NO: 136) | |
| T2A | GSGEGRGSLLTCGDVEENPGP |
| (SEQ ID NO: 137) | |

Restriction Enzyme Sequence(s)

| | |
|---|---|
| PacI | LIN |

Reporter Protein(s)

| | |
|---|---|
| eGFP | MVSKGEELFTGVVPILVELDGDVNGHKFSVSGEGEGDATYGKLTL |
| (SEQ ID NO: 138) | KFICTTGKLPVPWPTLVTTLTYGVQCFSRYPDHMKQHDFFKSAMP |
| | EGYVQERTIFFKDDGNYKTRAEVKFEGDTLVNRIELKGIDFKEDGN |
| | ILGHKLEYNYNSHNVYIMADKQKNGIKVNFKIRHNIEDGSVQLADH |
| | YQQNTPIGDGPVLLPDNHYLSTQSALSKDPNEKRDHMVLLEFVTA |
| | AGITLGMDELYK |
| mCherry | MVSKGEEDNMAIIKEFMRFKVHMEGSVNGHEFEIEGEGEGRPYE |
| (SEQ ID NO: 139) | GTQTAKLKVTKGGPLPFAWDILSPQFMYGSKAYVKHPADIPDYLK |
| | LSFPEGFKWERVMNFEDGGVVTVTQDSSLQDGEFIYKVKLRGTN |
| | FPSDGPVMQKKTMGWEASSERMYPEDGALKGEIKQRLKLKDGG |
| | HYDAEVKTTYKAKKPVQLPGAYNVNIKLDITSHNEDYTIVEQYERA |
| | EGRHSTGGMDELYK |

In certain aspects, a CAR of the present disclosure includes a single chain antibody (e.g., any of the scFvs of the present disclosure) that binds to the antigen of interest (e.g., VV A56 antigen or VV B5 antigen); a transmembrane domain from a polypeptide selected from the group consisting of: CD4, CD8α, CD154, and PD-1; one or more intracellular costimulatory signaling domains from a polypeptide selected from the group consisting of: 4-1BB (CD137), CD28, and CD134; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8 alpha hinge.

According to some embodiments, provided are CARs that comprise—from N-terminus to C-terminus—a variable heavy chain (V_{H}) polypeptide of an antibody described herein, a linker, the variable light chain (V_{L}) of the antibody, a CD8 hinge region (which in some embodiments is an extended CD8 hinge region), a CD8 transmembrane domain, a 4-1BB costimulatory domain, and a CD3ζ signaling domain. According to certain embodiments, provided are CARs that comprise—from N-terminus to C-terminus—a variable light chain (V_{L}) polypeptide of an antibody described herein, a linker, the variable heavy chain (V_{H}) of the antibody, a CD8 hinge region (which in some embodiments is an extended CD8 hinge region), a CD8 transmembrane domain, a 4-1 BB costimulatory domain, and a CD3ζ signaling domain. In certain embodiments, provided are CARs that comprise—from N-terminus to C-terminus—a variable heavy chain (V_{H}) polypeptide of an antibody described herein, a linker, the variable light chain (V_{L}) of the antibody, a CD28 hinge region, a CD28 transmembrane domain, a 4-1 BB costimulatory domain, and a CD3ζ signaling domain. According to some embodiments, provided are CARs that comprise—from N-terminus to C-terminus—a variable light chain (V_{L}) polypeptide of an antibody described herein, a linker, the variable heavy chain (V_{H}) of the antibody, a CD28 hinge region, a CD28 transmembrane domain, a 4-1BB costimulatory domain, and a CD3ζ signaling domain. Any of the CARs of the present disclosure may include a domain N-terminal to the V_{H} polypeptide. For example, a leader sequence (e.g., a GM-CSFR leader sequence) may be present at the N-terminus of a CAR of the present disclosure.

The amino acid sequences of example anti-A56 CARs of the present disclosure are provided in Tables 7 and 8 below, where the amino acid sequences of the CARs in Table 7 include an amino acid sequence of an N-terminal leader sequence (here, an N-terminal GM-CSFR leader sequence), and the amino acid sequences of the CARs in Table 8 do not include the amino acid sequence of the leader sequence. Any desired leader sequence (e.g., a GM-CSFR leader sequence) may be present at the N-terminus of such CARs. As will be appreciated by one of ordinary skill in the art, the amino acid sequence of one or more of the domains indicated in Tables 7 and 8 (e.g., linker, hinge, transmembrane, co-stimulatory, signaling, etc.) may be modified as desired, e.g., to improve the functionality, etc. of the CAR. In Tables 7 and 8, segments/domains of the polypeptides are indicated by alternating underlining, and the identities of the segments/domains are provided in the left column.

TABLE 7
CAR Sequences (including leader)

| | |
|---|---|
| A56-CAR-01, A56-CAR-05, | MLLLVTSLLLCELPHPAFLLIPQSLEESGGDLVKPGASLTLTCTASG |
| A56-CAR-06, A56-CAR-020 | IDFSDIYYISWVRQAPGKGLEWIACTYAGSSGSTYYATWAKGRFTI |
| •GM-CSFR Leader | SKASSTTVTLQMTSLTAADTATYFCARDRYPGTSGRVYGMDLWG |
| •VH | PGTLVTVSSGSTSGSGKPGSGEGSTKGDVVMTQTPSSVSEPVG |
| •Linker | GTVTIKCQASQSISDLLSWYQQKPGQPPKLLIYSASTLASGVSSRF |
| •VL | KGSGSGTEFTLTISDLECADAATYYCQCNYYSPTYGNGFGGGTE |
| •CD8 hinge | VVVKKTTTPAPRPPTPAPTIASQPLSLRPEACRPAAGGAVHTRGL |
| •CD8 transmembrane | DFACDIYIWAPLAGTCGVLLLSLVITLYCKRGRKKLLYIFKQPFMRP |
| •4-1BB | VQTTQEEDGCSCRFPEEEEGGCELRVKFSRSADAPAYQQGQNQ |
| •CD3ζ | LYNELNLGRREEYDVLDKRRGRDPEMGGKPRRKNPQEGLYNEL |
| (SEQ ID NO: 72) | QKDKMAEAYSEIGMKGERRRGKGHDGLYQGLSTATKDTYDALH |
| | MQALPPR |
| A56-CAR-07 | MLLLVTSLLLCELPHPAFLLIPQSLEESGGDLVKPGASLTLTCTASG |
| •GM-CSFR Leader | IDFSDIYYISWVRQAPGKGLEWIACTYAGSSGSTYYATWAKGRFTI |
| •VH | SKASSTTVTLQMTSLTAADTATYFCARDRYPGTSGRVYGMDLWG |
| •Linker | PGTLVTVSSGSTSGSGKPGSGEGSTKGDVVMTQTPSSVSEPVG |
| •VL | GTVTIKCQASQSISDLLSWYQQKPGQPPKLLIYSASTLASGVSSRF |
| •Extended CD8 hinge | KGSGSGTEFTLTISDLECADAATYYCQCNYYSPTYGNGFGGGTE |
| •CD8 transmembrane | VVVKKGGGGSGGGGSGGGGSGGTTTPAPRPPTPAPTIASQPLSL |
| •4-1 BB | RPEACRPAAGGAVHTRGLDFACDIYIWAPLAGTCGVLLLSLVITLY |
| •CD3ζ | CKRGRKKLLYIFKQPFMRPVQTTQEEDGCSCRFPEEEEGGCELR |
| (SEQ ID NO: 73) | VKFSRSADAPAYQQGQNQLYNELNLGRREEYDVLDKRRGRDPE |
| | MGGKPRRKNPQEGLYNELQKDKMAEAYSEIGMKGERRRGKGHD |
| | GLYQGLSTATKDTYDALHMQALPPR |
| A56-CAR-021 | MLLLVTSLLLCELPHPAFLLIPQSLEESGGDLVKPGASLTLTCTASG |
| •GM-CSFR Leader | IDFSDIYYISWVRQAPGKGLEWIACTYAGSSGSTYYATWAKGRFTI |
| •VH | SKASSTTVTLQMTSLTAADTATYFCARDRYPGTSGRVYGMDLWG |
| •Linker | PGTLVTVSSGSTSGSGKPGSGEGSTKGDVVMTQTPSSVSEPVG |
| •VL | GTVTIKCQASQSISDLLSWYQQKPGQPPKLLIYSASTLASGVSSRF |
| •CD28 hinge | KGSGSGTEFTLTISDLECADAATYYCQCNYYSPTYGNGFGGGTE |
| •CD28 transmembrane | VVVKKIEVMYPPPYLDNEKSNGTIIHVKGKHLCPSPLFPGPSKPFW |
| •4-1 BB | VLVVVGGVLACYSLLVTVAFIIFWVKRGRKKLLYIFKQPFMRPVQT |
| •CD3ζ | TQEEDGCSCRFPEEEEGGCELRVKFSRSADAPAYQQGQNQLYN |
| (SEQ ID NO: 74) | ELNLGRREEYDVLDKRRGRDPEMGGKPRRKNPQEGLYNELQKD |
| | KMAEAYSEIGMKGERRRGKGHDGLYQGLSTATKDTYDALHMQAL |
| | PPR |
| A56-CAR-02, A56-CAR-08 | MLLLVTSLLLCELPHPAFLLIPDVVMTQTPSSVSEPVGGTVTIKCQ |
| •GM-CSFR Leader | ASQSISDLLSWYQQKPGQPPKLLIYSASTLASGVSSRFKGSGSGT |
| •VL | EFTLTISDLECADAATYYCQCNYYSPTYGNGFGGGTEVVVKKGST |
| •linker | SGSGKPGSGEGSTKGQSLEESGGDLVKPGASLTLTCTASGIDFS |
| •VH | DIYYISWVRQAPGKGLEWIACTYAGSSGSTYYATWAKGRFTISKA |
| •CD8 hinge | SSTTVTLQMTSLTAADTATYFCARDRYPGTSGRVYGMDLWGPGT |
| •CD8 transmembrane | LVTVSSTTTPAPRPPTPAPTIASQPLSLRPEACRPAAGGAVHTRG |
| •4-1BB | LDFACDIYIWAPLAGTCGVLLLSLVITLYCKRGRKKLLYIFKQPFMR |
| •CD3ζ | PVQTTQEEDGCSCRFPEEEEGGCELRVKFSRSADAPAYQQGQN |
| (SEQ ID NO: 75) | QLYNELNLGRREEYDVLDKRRGRDPEMGGKPRRKNPQEGLYNE |
| | LQKDKMAEAYSEIGMKGERRRGKGHDGLYQGLSTATKDTYDALH |
| | MQALPPR |
| A56-CAR-010 | MLLLVTSLLLCELPHPAFLLIPDVVMTQTPSSVSEPVGGTVTIKCQ |
| •GM-CSFR Leader | ASQSISDLLSWYQQKPGQPPKLLIYSASTLASGVSSRFKGSGSGT |
| •VL | EFTLTISDLECADAATYYCQCNYYSPTYGNGFGGGTEVVVKKGST |
| •linker | SGSGKPGSGEGSTKGQSLEESGGDLVKPGASLTLTCTASGIDFS |
| •VH | DIYYISWVRQAPGKGLEWIACTYAGSSGSTYYATWAKGRFTISKA |
| •Extended CD8 hinge | SSTTVTLQMTSLTAADTATYFCARDRYPGTSGRVYGMDLWGPGT |
| •CD8 transmembrane | LVTVSSGGGGSGGGGSGGGGSGGTTTPAPRPPTPAPTIASQPLS |
| •4-1 BB | LRPEACRPAAGGAVHTRGLDFACDIYIWAPLAGTCGVLLLSLVITL |
| •CD3ζ | YCKRGRKKLLYIFKQPFMRPVQTTQEEDGCSCRFPEEEEGGCEL |
| (SEQ ID NO: 76) | RVKFSRSADAPAYQQGQNQLYNELNLGRREEYDVLDKRRGRDP |
| | EMGGKPRRKNPQEGLYNELQKDKMAEAYSEIGMKGERRRGKGH |
| | DGLYQGLSTATKDTYDALHMQALPPR |
| A56-CAR-027 | MLLLVTSLLLCELPHPAFLLIPQSLEESGGDLVKPGASLTLTCTASG |
| •GM-CSFR Leader | IDFSDIYYISWVRQAPGKGLEWIACTYAGSSGSTYYATWAKGRFTI |
| •VH | SKASSTTVTLQMTSLTAADTATYFCARDRYPGTSGRVYGMDLWG |
| •Linker | PGTLVTVSSGSTSGSGKPGSGEGSTKGVVMTQTPSSVSEPVGG |
| •VL | TVTIKCQASQSISDLLSWYQQKPGQPPKLLIYSASTLASGVSSRFK |
| •CD8 hinge | GSGSGTEFTLTISDLECADAATYYCQCNYYSPTYGNGFGGGTEV |
| •CD8 transmembrane | VVKTTTPAPRPPTPAPTIASQPLSLRPEACRPAAGGAVHTRGLDF |
| •4-1 BB | ACDIYIWAPLAGTCGVLLLSLVITLYCKRGRKKLLYIFKQPFMRPVQ |
| •CD3ζ | TTQEEDGCSCRFPEEEEGGCELRVKFSRSADAPAYQQGQNQLY |
| (SEQ ID NO: 77) | NELNLGRREEYDVLDKRRGRDPEMGGKPRRKNPQEGLYNELQK |
| | DKMAEAYSEIGMKGERRRGKGHDGLYQGLSTATKDTYDALHMQ |
| | ALPPR |
| A56-CAR-028 | MLLLVTSLLLCELPHPAFLLIPVVMTQTPSSVSEPVGGTVTIKCQAS |
| •GM-CSFR Leader | QSISDLLSWYQQKPGQPPKLLIYSASTLASGVSSRFKGSGSGTEF |
| •VL | TLTISDLECADAATYYCQCNYYSPTYGNGFGGGTEVVVKGSTSG |
| •linker | SGKPGSGEGSTKGQSLEESGGDLVKPGASLTLTCTASGIDFSDIY |
| •VH | YISWVRQAPGKGLEWIACTYAGSSGSTYYATWAKGRFTISKASST |
| •CD8 hinge | TVTLQMTSLTAADTATYFCARDRYPGTSGRVYGMDLWGPGTLVT |
| •CD8 transmembrane | VSSTTTPAPRPPTPAPTIASQPLSLRPEACRPAAGGAVHTRGLDF |
| •4-1BB | ACDIYIWAPLAGTCGVLLLSLVITLYCKRGRKKLLYIFKQPFMRPVQ |
| •CD3ζ | TTQEEDGCSCRFPEEEEGGCELRVKFSRSADAPAYQQGQNQLY |
| (SEQ ID NO: 78) | NELNLGRREEYDVLDKRRGRDPEMGGKPRRKNPQEGLYNELQK |
| | DKMAEAYSEIGMKGERRRGKGHDGLYQGLSTATKDTYDALHMQ |
| | ALPPR |
| A56-CAR-029 | MLLLVTSLLLCELPHPAFLLIPQSLEESGGDLVKPGASLTLTCTASG |
| •GM-CSFR Leader | IDFSDIYYISWVRQAPGKGLEWIACTYAGSSGSTYYATWAKGRFTI |
| •VH | SKASSTTVTLQMTSLTAADTATYFCARDRYPGTSGRVYGMDLWG |
| •Linker | PGTLVTVSSGSTSGSGKPGSGEGSTKGDVVMTQTPSSVSEPVG |
| •VL | GTVTIKCQASQSISDLLSWYQQKPGQPPKLLIYSASTLASGVSSRF |
| •CD8 hinge | KGSGSGTEFTLTISDLECADAATYYCQCNYYSPTYGNGFGGGTE |
| •CD8 transmembrane | VVVKTTTPAPRPPTPAPTIASQPLSLRPEACRPAAGGAVHTRGLD |
| •4-1 BB | FACDIYIWAPLAGTCGVLLLSLVITLYCKRGRKKLLYIFKQPFMRPV |
| •CD3ζ | QTTQEEDGCSCRFPEEEEGGCELRVKFSRSADAPAYQQGQNQL |
| (SEQ ID NO: 79) | YNELNLGRREEYDVLDKRRGRDPEMGGKPRRKNPQEGLYNELQ |
| | KDKMAEAYSEIGMKGERRRGKGHDGLYQGLSTATKDTYDALHM |
| | QALPPR |
| A56-CAR-030 | MLLLVTSLLLCELPHPAFLLIPDVVMTQTPSSVSEPVGGTVTIKCQ |
| •GM-CSFR Leader | ASQSISDLLSWYQQKPGQPPKLLIYSASTLASGVSSRFKGSGSGT |
| •VL | EFTLTISDLECADAATYYCQCNYYSPTYGNGFGGGTEVVVKGSTS |
| •Linker | GSGKPGSGEGSTKGQSLEESGGDLVKPGASLTLTCTASGIDFSDI |
| •VH | YYISWVRQAPGKGLEWIACTYAGSSGSTYYATWAKGRFTISKASS |
| •CD8 hinge | TTVTLQMTSLTAADTATYFCARDRYPGTSGRVYGMDLWGPGTLV |
| •CD8 transmembrane | TVSSTTTPAPRPPTPAPTIASQPLSLRPEACRPAAGGAVHTRGLD |
| •4-1BB | FACDIYIWAPLAGTCGVLLLSLVITLYCKRGRKKLLYIFKQPFMRPV |
| •CD3ζ | QTTQEEDGCSCRFPEEEEGGCELRVKFSRSADAPAYQQGQNQL |
| (SEQ ID NO: 80) | YNELNLGRREEYDVLDKRRGRDPEMGGKPRRKNPQEGLYNELQ |
| | KDKMAEAYSEIGMKGERRRGKGHDGLYQGLSTATKDTYDALHM |
| | QALPPR |
| B5-CAR-03, B5-CAR-011, | MLLLVTSLLLCELPHPAFLLIPQEQLEESGGGLVKPEGSLTLTCTA |
| B5-CAR-019 | SGFSFSSSYYMCWVRQAPGRGLEWIACIYTSSGSAYYANWAKG |
| •GM-CSFR Leader | RFTISRTSSTTVTLQMTRLTAADTATYFCVRNAVGSSYYLYLWGP |
| •VH | GTLVTVSSGSTSGSGKPGSGEGSTKGQVLTQTPSPVSAAVGGTV |
| •Linker | TISCQASQSVAGNNYLSWYQQKPGQPPNLLIYSVSTLASGVPSRF |
| •VL | KGSGSGTQFTLTISDLECDDAATYYCQGYYNDGIWAFGGGTEVV |
| •CD8 hinge | VKTTTPAPRPPTPAPTIASQPLSLRPEACRPAAGGAVHTRGLDFA |
| •CD8 transmembrane | CDIYIWAPLAGTCGVLLLSLVITLYCKRGRKKLLYIFKQPFMRPVQT |
| •4-1 BB | TQEEDGCSCRFPEEEEGGCELRVKFSRSADAPAYQQGQNQLYN |
| •CD3ζ | ELNLGRREEYDVLDKRRGRDPEMGGKPRRKNPQEGLYNELQKD |
| (SEQ ID NO: 81) | KMAEAYSEIGMKGERRRGKGHDGLYQGLSTATKDTYDALHMQAL |
| | PPR |
| B5-CAR-013 | MLLLVTSLLLCELPHPAFLLIPQEQLEESGGGLVKPEGSLTLTCTA |
| •GM-CSFR Leader | SGFSFSSSYYMCWVRQAPGRGLEWIACIYTSSGSAYYANWAKG |
| •VH | RFTISRTSSTTVTLQMTRLTAADTATYFCVRNAVGSSYYLYLWGP |
| •Linker | GTLVTVSSGSTSGSGKPGSGEGSTKGQVLTQTPSPVSAAVGGTV |
| •VL | TISCQASQSVAGNNYLSWYQQKPGQPPNLLIYSVSTLASGVPSRF |
| •Extended CD8 hinge | KGSGSGTQFTLTISDLECDDAATYYCQGYYNDGIWAFGGGTEVV |
| •CD8 transmembrane | VKGGGGSGGGGSGGGGSGGTTTPAPRPPTPAPTIASQPLSLRP |
| •4-1 BB | EACRPAAGGAVHTRGLDFACDIYIWAPLAGTCGVLLLSLVITLYCK |
| •CD3ζ | RGRKKLLYIFKQPFMRPVQTTQEEDGCSCRFPEEEEGGCELRVK |
| (SEQ ID NO: 82) | FSRSADAPAYQQGQNQLYNELNLGRREEYDVLDKRRGRDPEMG |
| | GKPRRKNPQEGLYNELQKDKMAEAYSEIGMKGERRRGKGHDGL |
| | YQGLSTATKDTYDALHMQALPPR |
| B5-CAR-022 | MLLLVTSLLLCELPHPAFLLIPQEQLEESGGGLVKPEGSLTLTCTA |
| •GM-CSFR Leader | SGFSFSSSYYMCWVRQAPGRGLEWIACIYTSSGSAYYANWAKG |
| •VH | RFTISRTSSTTVTLQMTRLTAADTATYFCVRNAVGSSYYLYLWGP |
| •Linker | GTLVTVSSGSTSGSGKPGSGEGSTKGQVLTQTPSPVSAAVGGTV |
| •VL | TISCQASQSVAGNNYLSWYQQKPGQPPNLLIYSVSTLASGVPSRF |
| •CD28 hinge | KGSGSGTQFTLTISDLECDDAATYYCQGYYNDGIWAFGGGTEVV |
| •CD28 transmembrane | VKIEVMYPPPYLDNEKSNGTIIHVKGKHLCPSPLFPGPSKPFWVLV |
| •4-1BB | VVGGVLACYSLLVTVAFIIFWVKRGRKKLLYIFKQPFMRPVQTTQE |
| •CD3ζ | EDGCSCRFPEEEEGGCELRVKFSRSADAPAYQQGQNQLYNELN |
| (SEQ ID NO: 83) | LGRREEYDVLDKRRGRDPEMGGKPRRKNPQEGLYNELQKDKMA |
| | EAYSEIGMKGERRRGKGHDGLYQGLSTATKDTYDALHMQALPPR |
| B5-CAR-04, B5-CAR-014 | MLLLVTSLLLCELPHPAFLLIPQVLTQTPSPVSAAVGGTVTISCQAS |
| •GM-CSFR Leader | QSVAGNNYLSWYQQKPGQPPNLLIYSVSTLASGVPSRFKGSGSG |
| •VL | TQFTLTISDLECDDAATYYCQGYYNDGIWAFGGGTEVVVKGSTS |
| •Linker | GSGKPGSGEGSTKGQEQLEESGGGLVKPEGSLTLTCTASGFSFS |
| •VH | SSYYMCWVRQAPGRGLEWIACIYTSSGSAYYANWAKGRFTISRT |
| •CD8 hinge | SSTTVTLQMTRLTAADTATYFCVRNAVGSSYYLYLWGPGTLVTVS |
| •CD8 transmembrane | STTTPAPRPPTPAPTIASQPLSLRPEACRPAAGGAVHTRGLDFAC |
| •4-1 BB | DIYIWAPLAGTCGVLLLSLVITLYCKRGRKKLLYIFKQPFMRPVQTT |
| •CD3ζ | QEEDGCSCRFPEEEEGGCELRVKFSRSADAPAYQQGQNQLYNE |
| (SEQ ID NO: 84) | LNLGRREEYDVLDKRRGRDPEMGGKPRRKNPQEGLYNELQKDK |
| | MAEAYSEIGMKGERRRGKGHDGLYQGLSTATKDTYDALHMQALP |
| | PR |
| B5-CAR-016 | MLLLVTSLLLCELPHPAFLLIPQVLTQTPSPVSAAVGGTVTISCQAS |
| •GM-CSFR Leader | QSVAGNNYLSWYQQKPGQPPNLLIYSVSTLASGVPSRFKGSGSG |
| •VL | TQFTLTISDLECDDAATYYCQGYYNDGIWAFGGGTEVVVKGSTS |
| •Linker | GSGKPGSGEGSTKGQEQLEESGGGLVKPEGSLTLTCTASGFSFS |
| •VH | SSYYMCWVRQAPGRGLEWIACIYTSSGSAYYANWAKGRFTISRT |
| •Extended CD8 hinge | SSTTVTLQMTRLTAADTATYFCVRNAVGSSYYLYLWGPGTLVTVS |
| •CD8 transmembrane | SGGGGSGGGGSGGGGSGGTTTPAPRPPTPAPTIASQPLSLRPE |
| •4-1 BB | ACRPAAGGAVHTRGLDFACDIYIWAPLAGTCGVLLLSLVITLYCKR |
| •CD3ζ | GRKKLLYIFKQPFMRPVQTTQEEDGCSCRFPEEEEGGCELRVKF |
| (SEQ ID NO: 85) | SRSADAPAYQQGQNQLYNELNLGRREEYDVLDKRRGRDPEMGG |
| | KPRRKNPQEGLYNELQKDKMAEAYSEIGMKGERRRGKGHDGLY |
| | QGLSTATKDTYDALHMQALPPR |

TABLE 8
CAR Sequences (amino acid sequence of leader not included)

| | |
|---|---|
| A56-CAR-01, A56-CAR-05, | QSLEESGGDLVKPGASLTLTCTASGIDFSDIYYISWVRQAPGKGLE |
| A56-CAR-06, A56-CAR-020 | WIACTYAGSSGSTYYATWAKGRFTISKASSTTVTLQMTSLTAADT |
| •VH | ATYFCARDRYPGTSGRVYGMDLWGPGTLVTVSSGSTSGSGKPG |
| •Linker | SGEGSTKGDVVMTQTPSSVSEPVGGTVTIKCQASQSISDLLSWY |
| •VL | QQKPGQPPKLLIYSASTLASGVSSRFKGSGSGTEFTLTISDLECAD |
| •CD8 hinge | AATYYCQCNYYSPTYGNGFGGGTEVVVKKTTTPAPRPPTPAPTIA |
| •CD8 transmembrane | SQPLSLRPEACRPAAGGAVHTRGLDFACDIYIWAPLAGTCGVLLL |
| •4-1BB | SLVITLYCKRGRKKLLYIFKQPFMRPVQTTQEEDGCSCRFPEEEE |
| •CD3ζ | GGCELRVKFSRSADAPAYQQGQNQLYNELNLGRREEYDVLDKR |
| (SEQ ID NO: 86) | RGRDPEMGGKPRRKNPQEGLYNELQKDKMAEAYSEIGMKGERR |
| | RGKGHDGLYQGLSTATKDTYDALHMQALPPR |
| A56-CAR-07 | QSLEESGGDLVKPGASLTLTCTASGIDFSDIYYISWVRQAPGKGLE |
| •VH | WIACTYAGSSGSTYYATWAKGRFTISKASSTTVTLQMTSLTAADT |
| •Linker | ATYFCARDRYPGTSGRVYGMDLWGPGTLVTVSSGSTSGSGKPG |
| •VL | SGEGSTKGDVVMTQTPSSVSEPVGGTVTIKCQASQSISDLLSWY |
| •Extended CD8 hinge | QQKPGQPPKLLIYSASTLASGVSSRFKGSGSGTEFTLTISDLECAD |
| •CD8 transmembrane | AATYYCQCNYYSPTYGNGFGGGTEVVVKKGGGGSGGGGSGGG |
| •4-1BB | GSGGTTTPAPRPPTPAPTIASQPLSLRPEACRPAAGGAVHTRGLD |
| •CD3ζ | FACDIYIWAPLAGTCGVLLLSLVITLYCKRGRKKLLYIFKQPFMRPV |
| (SEQ ID NO: 87) | QTTQEEDGCSCRFPEEEEGGCELRVKFSRSADAPAYQQGQNQL |
| | YNELNLGRREEYDVLDKRRGRDPEMGGKPRRKNPQEGLYNELQ |
| | KDKMAEAYSEIGMKGERRRGKGHDGLYQGLSTATKDTYDALHM |
| | QALPPR |
| A56-CAR-021 | QSLEESGGDLVKPGASLTLTCTASGIDFSDIYYISWVRQAPGKGLE |
| •VH | WIACTYAGSSGSTYYATWAKGRFTISKASSTTVTLQMTSLTAADT |
| •Linker | ATYFCARDRYPGTSGRVYGMDLWGPGTLVTVSSGSTSGSGKPG |
| •VL | SGEGSTKGDVVMTQTPSSVSEPVGGTVTIKCQASQSISDLLSWY |
| •CD28 hinge | QQKPGQPPKLLIYSASTLASGVSSRFKGSGSGTEFTLTISDLECAD |
| •CD28 transmembrane | AATYYCQCNYYSPTYGNGFGGGTEVVVKKIEVMYPPPYLDNEKS |
| •4-1BB | NGTIIHVKGKHLCPSPLFPGPSKPFWVLVVVGGVLACYSLLVTVAF |
| •CD3ζ | IIFWVKRGRKKLLYIFKQPFMRPVQTTQEEDGCSCRFPEEEEGGC |
| (SEQ ID NO: 88) | ELRVKFSRSADAPAYQQGQNQLYNELNLGRREEYDVLDKRRGR |
| | DPEMGGKPRRKNPQEGLYNELQKDKMAEAYSEIGMKGERRRGK |
| | GHDGLYQGLSTATKDTYDALHMQALPPR |
| A56-CAR-02, A56-CAR-08 | DVVMTQTPSSVSEPVGGTVTIKCQASQSISDLLSWYQQKPGQPP |
| •VH | KLLIYSASTLASGVSSRFKGSGSGTEFTLTISDLECADAATYYCQC |
| •VL | NYYSPTYGNGFGGGTEVVVKKGSTSGSGKPGSGEGSTKGQSLE |
| •linker | ESGGDLVKPGASLTLTCTASGIDFSDIYYISWVRQAPGKGLEWIAC |
| •CD8 hinge | TYAGSSGSTYYATWAKGRFTISKASSTTVTLQMTSLTAADTATYF |
| •CD8 transmembrane | CARDRYPGTSGRVYGMDLWGPGTLVTVSSTTTPAPRPPTPAPTI |
| •4-1BB | ASQPLSLRPEACRPAAGGAVHTRGLDFACDIYIWAPLAGTCGVLL |
| •CD3ζ | LSLVITLYCKRGRKKLLYIFKQPFMRPVQTTQEEDGCSCRFPEEEE |
| (SEQ ID NO: 89) | GGCELRVKFSRSADAPAYQQGQNQLYNELNLGRREEYDVLDKR |
| | RGRDPEMGGKPRRKNPQEGLYNELQKDKMAEAYSEIGMKGERR |
| | RGKGHDGLYQGLSTATKDTYDALHMQALPPR |
| A56-CAR-010 | DVVMTQTPSSVSEPVGGTVTIKCQASQSISDLLSWYQQKPGQPP |
| •VL | KLLIYSASTLASGVSSRFKGSGSGTEFTLTISDLECADAATYYCQC |
| •linker | NYYSPTYGNGFGGGTEVVVKKGSTSGSGKPGSGEGSTKGQSLE |
| •VH | ESGGDLVKPGASLTLTCTASGIDFSDIYYISWVRQAPGKGLEWIAC |
| •Extended CD8 hinge | TYAGSSGSTYYATWAKGRFTISKASSTTVTLQMTSLTAADTATYF |
| •CD8 transmembrane | CARDRYPGTSGRVYGMDLWGPGTLVTVSSGGGGSGGGGSGGG |
| •4-1BB | GSGGTTTPAPRPPTPAPTIASQPLSLRPEACRPAAGGAVHTRGLD |
| •CD3ζ | FACDIYIWAPLAGTCGVLLLSLVITLYCKRGRKKLLYIFKQPFMRPV |
| (SEQ ID NO: 90) | QTTQEEDGCSCRFPEEEEGGCELRVKFSRSADAPAYQQGQNQL |
| | YNELNLGRREEYDVLDKRRGRDPEMGGKPRRKNPQEGLYNELQ |
| | KDKMAEAYSEIGMKGERRRGKGHDGLYQGLSTATKDTYDALHM |
| | QALPPR |
| A56-CAR-027 | QSLEESGGDLVKPGASLTLTCTASGIDFSDIYYISWVRQAPGKGLE |
| •VH | WIACTYAGSSGSTYYATWAKGRFTISKASSTTVTLQMTSLTAADT |
| •Linker | ATYFCARDRYPGTSGRVYGMDLWGPGTLVTVSSGSTSGSGKPG |
| •VL | SGEGSTKGVVMTQTPSSVSEPVGGTVTIKCQASQSISDLLSWYQ |
| •CD8 hinge | QKPGQPPKLLIYSASTLASGVSSRFKGSGSGTEFTLTISDLECADA |
| •CD8 transmembrane | ATYYCQCNYYSPTYGNGFGGGTEVVVKTTTPAPRPPTPAPTIASQ |
| •4-1BB | PLSLRPEACRPAAGGAVHTRGLDFACDIYIWAPLAGTCGVLLLSLV |
| •CD3ζ | ITLYCKRGRKKLLYIFKQPFMRPVQTTQEEDGCSCRFPEEEEGGC |
| (SEQ ID NO: 91) | ELRVKFSRSADAPAYQQGQNQLYNELNLGRREEYDVLDKRRGR |
| | DPEMGGKPRRKNPQEGLYNELQKDKMAEAYSEIGMKGERRRGK |
| | GHDGLYQGLSTATKDTYDALHMQALPPR |
| A56-CAR-028 | VVMTQTPSSVSEPVGGTVTIKCQASQSISDLLSWYQQKPGQPPK |
| •VL | LLIYSASTLASGVSSRFKGSGSGTEFTLTISDLECADAATYYCQCN |
| •linker | YYSPTYGNGFGGGTEVVVKGSTSGSGKPGSGEGSTKGQSLEES |
| •VH | GGDLVKPGASLTLTCTASGIDFSDIYYISWVRQAPGKGLEWIACTY |
| •CD8 hinge | AGSSGSTYYATWAKGRFTISKASSTTVTLQMTSLTAADTATYFCA |
| •CD8 transmembrane | RDRYPGTSGRVYGMDLWGPGTLVTVSSTTTPAPRPPTPAPTIAS |
| •4-1BB | QPLSLRPEACRPAAGGAVHTRGLDFACDIYIWAPLAGTCGVLLLS |
| •CD3ζ | LVITLYCKRGRKKLLYIFKQPFMRPVQTTQEEDGCSCRFPEEEEG |
| (SEQ ID NO: 92) | GCELRVKFSRSADAPAYQQGQNQLYNELNLGRREEYDVLDKRR |
| | GRDPEMGGKPRRKNPQEGLYNELQKDKMAEAYSEIGMKGERRR |
| | GKGHDGLYQGLSTATKDTYDALHMQALPPR |
| A56-CAR-029 | QSLEESGGDLVKPGASLTLTCTASGIDFSDIYYISWVRQAPGKGLE |
| •VH | WIACTYAGSSGSTYYATWAKGRFTISKASSTTVTLQMTSLTAADT |
| •Linker | ATYFCARDRYPGTSGRVYGMDLWGPGTLVTVSSGSTSGSGKPG |
| •VL | SGEGSTKGDVVMTQTPSSVSEPVGGTVTIKCQASQSISDLLSWY |
| •CD8 hinge | QQKPGQPPKLLIYSASTLASGVSSRFKGSGSGTEFTLTISDLECAD |
| •CD8 transmembrane | AATYYCQCNYYSPTYGNGFGGGTEVVVKTTTPAPRPPTPAPTIAS |
| •4-1BB | QPLSLRPEACRPAAGGAVHTRGLDFACDIYIWAPLAGTCGVLLLS |
| •CD3ζ | LVITLYCKRGRKKLLYIFKQPFMRPVQTTQEEDGCSCRFPEEEEG |
| (SEQ ID NO: 93) | GCELRVKFSRSADAPAYQQGQNQLYNELNLGRREEYDVLDKRR |
| | GRDPEMGGKPRRKNPQEGLYNELQKDKMAEAYSEIGMKGERRR |
| | GKGHDGLYQGLSTATKDTYDALHMQALPPR |
| A56-CAR-030 | DVVMTQTPSSVSEPVGGTVTIKCQASQSISDLLSWYQQKPGQPP |
| •VL | KLLIYSASTLASGVSSRFKGSGSGTEFTLTISDLECADAATYYCQC |
| •Linker | NYYSPTYGNGFGGGTEVVVKGSTSGSGKPGSGEGSTKGQSLEE |
| •VH | SGGDLVKPGASLTLTCTASGIDFSDIYYISWVRQAPGKGLEWIACT |
| •CD8 hinge | YAGSSGSTYYATWAKGRFTISKASSTTVTLQMTSLTAADTATYFC |
| •CD8 transmembrane | ARDRYPGTSGRVYGMDLWGPGTLVTVSSTTTPAPRPPTPAPTIA |
| •4-1BB | SQPLSLRPEACRPAAGGAVHTRGLDFACDIYIWAPLAGTCGVLLL |
| •CD3ζ | SLVITLYCKRGRKKLLYIFKQPFMRPVQTTQEEDGCSCRFPEEEE |
| (SEQ ID NO: 94) | GGCELRVKFSRSADAPAYQQGQNQLYNELNLGRREEYDVLDKR |
| | RGRDPEMGGKPRRKNPQEGLYNELQKDKMAEAYSEIGMKGERR |
| | RGKGHDGLYQGLSTATKDTYDALHMQALPPR |
| B5-CAR-03, B5-CAR-011, | QEQLEESGGGLVKPEGSLTLTCTASGFSFSSSYYMCWWRQAPG |
| B5-CAR-019 | RGLEWIACIYTSSGSAYYANWAKGRFTISRTSSTTVTLQMTRLTAA |
| •VH | DTATYFCVRNAVGSSYYLYLWGPGTLVTVSSGSTSGSGKPGSGE |
| •Linker | GSTKGQVLTQTPSPVSAAVGGTVTISCQASQSVAGNNYLSWYQQ |
| •VL | KPGQPPNLLIYSVSTLASGVPSRFKGSGSGTQFTLTISDLECDDAA |
| •CD8 hinge | TYYCQGYYNDGIWAFGGGTEVVVKTTTPAPRPPTPAPTIASQPLS |
| •CD8 transmembrane | LRPEACRPAAGGAVHTRGLDFACDIYIWAPLAGTCGVLLLSLVITL |
| •4-1BB | YCKRGRKKLLYIFKQPFMRPVQTTQEEDGCSCRFPEEEEGGCEL |
| •CD3ζ | RVKFSRSADAPAYQQGQNQLYNELNLGRREEYDVLDKRRGRDP |
| (SEQ ID NO: 95) | EMGGKPRRKNPQEGLYNELQKDKMAEAYSEIGMKGERRRGKGH |
| | DGLYQGLSTATKDTYDALHMQALPPR |
| B5-CAR-013 | QEQLEESGGGLVKPEGSLTLTCTASGFSFSSSYYMCWVRQAPG |
| •VH | RGLEWIACIYTSSGSAYYANWAKGRFTISRTSSTTVTLQMTRLTAA |
| •Linker | DTATYFCVRNAVGSSYYLYLWGPGTLVTVSSGSTSGSGKPGSGE |
| •VL | GSTKGQVLTQTPSPVSAAVGGTVTISCQASQSVAGNNYLSWYQQ |
| •Extended CD8 hinge | KPGQPPNLLIYSVSTLASGVPSRFKGSGSGTQFTLTISDLECDDAA |
| •CD8 transmembrane | TYYCQGYYNDGIWAFGGGTEVVVKGGGGSGGGGSGGGGSGGT |
| •4-1BB | TTPAPRPPTPAPTIASQPLSLRPEACRPAAGGAVHTRGLDFACDIY |
| •CD3ζ | IWAPLAGTCGVLLLSLVITLYCKRGRKKLLYIFKQPFMRPVQTTQE |
| (SEQ ID NO: 96) | EDGCSCRFPEEEEGGCELRVKFSRSADAPAYQQGQNQLYNELN |
| | LGRREEYDVLDKRRGRDPEMGGKPRRKNPQEGLYNELQKDKMA |
| | EAYSEIGMKGERRRGKGHDGLYQGLSTATKDTYDALHMQALPPR |
| B5-CAR-022 | QEQLEESGGGLVKPEGSLTLTCTASGFSFSSSYYMCWVRQAPG |
| •VH | RGLEWIACIYTSSGSAYYANWAKGRFTISRTSSTTVTLQMTRLTAA |
| •Linker | DTATYFCVRNAVGSSYYLYLWGPGTLVTVSSGSTSGSGKPGSGE |
| •VL | GSTKGQVLTQTPSPVSAAVGGTVTISCQASQSVAGNNYLSWYQQ |
| •CD28 hinge | KPGQPPNLLIYSVSTLASGVPSRFKGSGSGTQFTLTISDLECDDAA |
| •CD28 transmembrane | TYYCQGYYNDGIWAFGGGTEVVVKIEVMYPPPYLDNEKSNGTIIH |
| •4-1BB | VKGKHLCPSPLFPGPSKPFWVLVVVGGVLACYSLLVTVAFIIFWVK |
| •CD3ζ | RGRKKLLYIFKQPFMRPVQTTQEEDGCSCRFPEEEEGGCELRVK |
| (SEQ ID NO: 97) | FSRSADAPAYQQGQNQLYNELNLGRREEYDVLDKRRGRDPEMG |
| | GKPRRKNPQEGLYNELQKDKMAEAYSEIGMKGERRRGKGHDGL |
| | YQGLSTATKDTYDALHMQALPPR |
| B5-CAR-04, B5-CAR-014 | QVLTQTPSPVSAAVGGTVTISCQASQSVAGNNYLSWYQQKPGQP |
| •VL | PNLLIYSVSTLASGVPSRFKGSGSGTQFTLTISDLECDDAATYYCQ |
| •Linker | GYYNDGIWAFGGGTEVVVKGSTSGSGKPGSGEGSTKGQEQLEE |
| •VH | SGGGLVKPEGSLTLTCTASGFSFSSSYYMCWVRQAPGRGLEWIA |
| •CD8 hinge | CIYTSSGSAYYANWAKGRFTISRTSSTTVTLQMTRLTAADTATYFC |
| •CD8 transmembrane | VRNAVGSSYYLYLWGPGTLVTVSSTTTPAPRPPTPAPTIASQPLS |
| •4-1BB | LRPEACRPAAGGAVHTRGLDFACDIYIWAPLAGTCGVLLLSLVITL |
| •CD3ζ | YCKRGRKKLLYIFKQPFMRPVQTTQEEDGCSCRFPEEEEGGCEL |
| (SEQ ID NO: 98) | RVKFSRSADAPAYQQGQNQLYNELNLGRREEYDVLDKRRGRDP |
| | EMGGKPRRKNPQEGLYNELQKDKMAEAYSEIGMKGERRRGKGH |
| | DGLYQGLSTATKDTYDALHMQALPPR |
| B5-CAR-016 | QVLTQTPSPVSAAVGGTVTISCQASQSVAGNNYLSWYQQKPGQP |
| •VL | PNLLIYSVSTLASGVPSRFKGSGSGTQFTLTISDLECDDAATYYCQ |
| •Linker | GYYNDGIWAFGGGTEVVVKGSTSGSGKPGSGEGSTKGQEQLEE |
| •VH | SGGGLVKPEGSLTLTCTASGFSFSSSYYMCWVRQAPGRGLEWIA |
| •Extended CD8 hinge | CIYTSSGSAYYANWAKGRFTISRTSSTTVTLQMTRLTAADTATYFC |
| •CD8 transmembrane | VRNAVGSSYYLYLWGPGTLVTVSSGGGGSGGGGSGGGGSGGT |
| •4-1BB | TTPAPRPPTPAPTIASQPLSLRPEACRPAAGGAVHTRGLDFACDIY |
| •CD3ζ | IWAPLAGTCGVLLLSLVITLYCKRGRKKLLYIFKQPFMRPVQTTQE |
| (SEQ ID NO: 99) | EDGCSCRFPEEEEGGCELRVKFSRSADAPAYQQGQNQLYNELN |
| | LGRREEYDVLDKRRGRDPEMGGKPRRKNPQEGLYNELQKDKMA |
| | EAYSEIGMKGERRRGKGHDGLYQGLSTATKDTYDALHMQALPPR |

A CAR of the present disclosure may include one or more additional domains as desired. Non-limiting examples of such additional domains include a ribosomal skip element, an enzymatic domain (e.g., a domain having nuclease activity, e.g., restriction endonuclease activity), a domain that enables detection of the CAR (e.g., a reporter protein domain (e.g., a fluorescent protein (e.g., eGFP, mCherry, or the like), a luminescent protein, and/or the like)), etc. For example, in certain embodiments, provided are CARs that comprise a ribosomal skip element, a restriction enzyme domain, and/or a reporter protein domain. The amino acid sequences of example anti-A56 CARs of the present disclosure having one or more of these features are provided in Table 9 below. As will be appreciated by one of ordinary skill in the art, the amino acid sequence of one or more of the domains indicated in Table 9 (e.g., linker, hinge, transmembrane, co-stimulatory, signaling, restriction enzyme, reporter protein, etc.) may be modified as desired, e.g., to improve functionality, detection, etc. of the CAR. In Table 9, the CAR portion is shown in bold, and segments/domains of the polypeptides are indicated by alternating underlining. Identities of the segments/domains are provided in the left column.

TABLE 9
CAR Sequences (leader and ribosomal skip element, restriction enzyme and/or
reporter protein included)

| | |
|---|---|
| A56-CAR-05 | MLLLVTSLLLCELPHPAFLLIPQSLEESGGDLVKPGASLTLTCTA |
| •GM-CSFR Leader | SGIDFSDIYYISWVRQAPGKGLEWIACTYAGSSGSTYYATWAKG |
| •VH | RFTISKASSTTVTLQMTSLTAADTATYFCARDRYPGTSGRVYGM |
| •Linker | DLWGPGTLVTVSSGSTSGSGKPGSGEGSTKGDVVMTQTPSSVS |
| •VL | EPVGGTVTIKCQASQSISDLLSWYQQKPGQPPKLLIYSASTLASG |
| •CD8 hinge | VSSRFKGSGSGTEFTLTISDLECADAATYYCQCNYYSPTYGNGF |
| •CD8 transmembrane | GGGTEVVVKKTTTPAPRPPTPAPTIASQPLSLRPEACRPAAGGA |
| •4-1BB | VHTRGLDFACDIYIWAPLAGTCGVLLLSLVITLYCKRGRKKLLYIF |
| •CD3ζ | KQPFMRPVQTTQEEDGCSCRFPEEEEGGCELRVKFSRSADAPA |
| •T2A | YQQGQNQLYNELNLGRREEYDVLDKRRGRDPEMGGKPRRKNP |
| •mCherry | QEGLYNELQKDKMAEAYSEIGMKGERRRGKGHDGLYQGLSTAT |
| (SEQ ID NO: 100) | KDTYDALHMQALPPRGSGEGRGSLLTCGDVEENPGPMVSKGEE |
| | DNMAIIKEFMRFKVHMEGSVNGHEFEIEGEGEGRPYEGTQTAKLK |
| | VTKGGPLPFAWDILSPQFMYGSKAYVKHPADIPDYLKLSFPEGFK |
| | WERVMNFEDGGVVTVTQDSSLQDGEFIYKVKLRGTNFPSDGPV |
| | MQKKTMGWEASSERMYPEDGALKGEIKQRLKLKDGGHYDAEVK |
| | TTYKAKKPVQLPGAYNVNIKLDITSHNEDYTIVEQYERAEGRHSTG |
| | GMDELYK |
| A56-CAR-06 | MLLLVTSLLLCELPHPAFLLIPQSLEESGGDLVKPGASLTLTCTA |
| •GM-CSFR Leader | SGIDFSDIYYISWVRQAPGKGLEWIACTYAGSSGSTYYATWAKG |
| •VH | RFTISKASSTTVTLQMTSLTAADTATYFCARDRYPGTSGRVYGM |
| •Linker | DLWGPGTLVTVSSGSTSGSGKPGSGEGSTKGDVVMTQTPSSVS |
| •VL | EPVGGTVTIKCQASQSISDLLSWYQQKPGQPPKLLIYSASTLASG |
| •CD8 hinge | VSSRFKGSGSGTEFTLTISDLECADAATYYCQCNYYSPTYGNGF |
| •CD8 transmembrane | GGGTEVVVKKTTTPAPRPPTPAPTIASQPLSLRPEACRPAAGGA |
| •4-1BB | VHTRGLDFACDIYIWAPLAGTCGVLLLSLVITLYCKRGRKKLLYIF |
| •CD3ζ | KQPFMRPVQTTQEEDGCSCRFPEEEEGGCELRVKFSRSADAPA |
| •PacI | YQQGQNQLYNELNLGRREEYDVLDKRRGRDPEMGGKPRRKNP |
| •E2A | QEGLYNELQKDKMAEAYSEIGMKGERRRGKGHDGLYQGLSTAT |
| •eGFP | KDTYDALHMQALPPRLINGSGQCTNYALLKLAGDVESNPGPMVS |
| (SEQ ID NO: 101) | KGEELFTGWPILVELDGDVNGHKFSVSGEGEGDATYGKLTLKFI |
| | CTTGKLPVPWPTLVTTLTYGVQCFSRYPDHMKQHDFFKSAMPEG |
| | YVQERTIFFKDDGNYKTRAEVKFEGDTLVNRIELKGIDFKEDGNIL |
| | GHKLEYNYNSHNVYIMADKQKNGIKVNFKIRHNIEDGSVQLADHY |
| | QQNTPIGDGPVLLPDNHYLSTQSALSKDPNEKRDHMVLLEFVTAA |
| | GITLGMDELYK |
| A56-CAR-020 | MLLLVTSLLLCELPHPAFLLIPQSLEESGGDLVKPGASLTLTCTA |
| •GM-CSFR Leader | SGIDFSDIYYISWVRQAPGKGLEWIACTYAGSSGSTYYATWAKG |
| •VH | RFTISKASSTTVTLQMTSLTAADTATYFCARDRYPGTSGRVYGM |
| •Linker | DLWGPGTLVTVSSGSTSGSGKPGSGEGSTKGDVVMTQTPSSVS |
| •VL | EPVGGTVTIKCQASQSISDLLSWYQQKPGQPPKLLIYSASTLASG |
| •CD8 hinge | VSSRFKGSGSGTEFTLTISDLECADAATYYCQCNYYSPTYGNGF |
| •CD8 transmembrane | GGGTEVVVKKTTTPAPRPPTPAPTIASQPLSLRPEACRPAAGGA |
| •4-1BB | VHTRGLDFACDIYIWAPLAGTCGVLLLSLVITLYCKRGRKKLLYIF |
| •CD3ζ | KQPFMRPVQTTQEEDGCSCRFPEEEEGGCELRVKFSRSADAPA |
| •T2A | YQQGQNQLYNELNLGRREEYDVLDKRRGRDPEMGGKPRRKNP |
| •eGFP | QEGLYNELQKDKMAEAYSEIGMKGERRRGKGHDGLYQGLSTAT |
| (SEQ ID NO: 102) | KDTYDALHMQALPPRGSGEGRGSLLTCGDVEENPGPMVSKGEE |
| | LFTGVVPILVELDGDVNGHKFSVSGEGEGDATYGKLTLKFICTTGK |
| | LPVPWPTLVTTLTYGVQCFSRYPDHMKQHDFFKSAMPEGYVQER |
| | TIFFKDDGNYKTRAEVKFEGDTLVNRIELKGIDFKEDGNILGHKLEY |
| | NYNSHNVYIMADKQKNGIKVNFKIRHNIEDGSVQLADHYQQNTPI |
| | GDGPVLLPDNHYLSTQSALSKDPNEKRDHMVLLEFVTAAGITLGM |
| | DELYK |
| A56-CAR-07 | MLLLVTSLLLCELPHPAFLLIPQSLEESGGDLVKPGASLTLTCTA |
| •GM-CSFR Leader | SGIDFSDIYYISWVRQAPGKGLEWIACTYAGSSGSTYYATWAKG |
| •VH | RFTISKASSTTVTLQMTSLTAADTATYFCARDRYPGTSGRVYGM |
| •Linker | DLWGPGTLVTVSSGSTSGSGKPGSGEGSTKGDVVMTQTPSSVS |
| •VL | EPVGGTVTIKCQASQSISDLLSWYQQKPGQPPKLLIYSASTLASG |
| •Extended CD8 hinge | VSSRFKGSGSGTEFTLTISDLECADAATYYCQCNYYSPTYGNGF |
| •CD8 transmembrane | GGGTEVVVKKGGGGSGGGGSGGGGSGGTTTPAPRPPTPAPTIA |
| •4-1BB | SQPLSLRPEACRPAAGGAVHTRGLDFACDIYIWAPLAGTCGVLL |
| •CD3ζ | LSLVITLYCKRGRKKLLYIFKQPFMRPVQTTQEEDGCSCRFPEEE |
| •T2A | EGGCELRVKFSRSADAPAYQQGQNQLYNELNLGRREEYDVLDK |
| •mCherry | RRGRDPEMGGKPRRKNPQEGLYNELQKDKMAEAYSEIGMKGE |
| (SEQ ID NO: 103) | RRRGKGHDGLYQGLSTATKDTYDALHMQALPPRGSGEGRGSLL |
| | TCGDVEENPGPMVSKGEEDNMAIIKEFMRFKVHMEGSVNGHEFE |
| | IEGEGEGRPYEGTQTAKLKVTKGGPLPFAWDILSPQFMYGSKAYV |
| | KHPADIPDYLKLSFPEGFKWERVMNFEDGGVVTVTQDSSLQDGE |
| | FIYKVKLRGTNFPSDGPVMQKKTMGWEASSERMYPEDGALKGEI |
| | KQRLKLKDGGHYDAEVKTTYKAKKPVQLPGAYNVNIKLDITSHNE |
| | DYTIVEQYERAEGRHSTGGMDELYK |
| A56-CAR-021 | MLLLVTSLLLCELPHPAFLLIPQSLEESGGDLVKPGASLTLTCTA |
| •GM-CSFR Leader | SGIDFSDIYYISWVRQAPGKGLEWIACTYAGSSGSTYYATWAKG |
| •VH | RFTISKASSTTVTLQMTSLTAADTATYFCARDRYPGTSGRVYGM |
| •Linker | DLWGPGTLVTVSSGSTSGSGKPGSGEGSTKGDVVMTQTPSSVS |
| •VL | EPVGGTVTIKCQASQSISDLLSWYQQKPGQPPKLLIYSASTLASG |
| •CD28 hinge | VSSRFKGSGSGTEFTLTISDLECADAATYYCQCNYYSPTYGNGF |
| •CD28 | GGGTEVVVKKIEVMYPPPYLDNEKSNGTIIHVKGKHLCPSPLFPG |
| transmembrane | PSKPFWVLVVVGGVLACYSLLVTVAFIIFWVKRGRKKLLYIFKQP |
| •4-1BB | FMRPVQTTQEEDGCSCRFPEEEEGGCELRVKFSRSADAPAYQQ |
| •CD3ζ | GQNQLYNELNLGRREEYDVLDKRRGRDPEMGGKPRRKNPQEG |
| •T2A | LYNELQKDKMAEAYSEIGMKGERRRGKGHDGLYQGLSTATKDT |
| •mCherry | YDALHMQALPPRGSGEGRGSLLTCGDVEENPGPMVSKGEEDN |
| (SEQ ID NO: 104) | MAIIKEFMRFKVHMEGSVNGHEFEIEGEGEGRPYEGTQTAKLKVT |
| | KGGPLPFAWDILSPQFMYGSKAYVKHPADIPDYLKLSFPEGFKWE |
| | RVMNFEDGGVVTVTQDSSLQDGEFIYKVKLRGTNFPSDGPVMQK |
| | KTMGWEASSERMYPEDGALKGEIKQRLKLKDGGHYDAEVKTTYK |
| | AKKPVQLPGAYNVNIKLDITSHNEDYTIVEQYERAEGRHSTGGMD |
| | ELYK |
| A56-CAR-08 | MLLLVTSLLLCELPHPAFLLIPDVVMTQTPSSVSEPVGGTVTIKCQ |
| •GM-CSFR Leader | ASQSISDLLSWYQQKPGQPPKLLIYSASTLASGVSSRFKGSGSG |
| •VL | TEFTLTISDLECADAATYYCQCNYYSPTYGNGFGGGTEVVVKKG |
| •linker | STSGSGKPGSGEGSTKGQSLEESGGDLVKPGASLTLTCTASGID |
| •VH | FSDIYYISWVRQAPGKGLEWIACTYAGSSGSTYYATWAKGRFTIS |
| •CD8 hinge | KASSTTVTLQMTSLTAADTATYFCARDRYPGTSGRVYGMDLWG |
| •CD8 transmembrane | PGTLVTVSSTTTPAPRPPTPAPTIASQPLSLRPEACRPAAGGAVH |
| •4-1BB | TRGLDFACDIYIWAPLAGTCGVLLLSLVITLYCKRGRKKLLYIFKQ |
| •CD3ζ | PFMRPVQTTQEEDGCSCRFPEEEEGGCELRVKFSRSADAPAYQ |
| •T2A | QGQNQLYNELNLGRREEYDVLDKRRGRDPEMGGKPRRKNPQE |
| •mCherry | GLYNELQKDKMAEAYSEIGMKGERRRGKGHDGLYQGLSTATKD |
| (SEQ ID NO: 105) | TYDALHMQALPPRGSGEGRGSLLTCGDVEENPGPMVSKGEEDN |
| | MAIIKEFMRFKVHMEGSVNGHEFEIEGEGEGRPYEGTQTAKLKVT |
| | KGGPLPFAWDILSPQFMYGSKAYVKHPADIPDYLKLSFPEGFKWE |
| | RVMNFEDGGVVTVTQDSSLQDGEFIYKVKLRGTNFPSDGPVMQK |
| | KTMGWEASSERMYPEDGALKGEIKQRLKLKDGGHYDAEVKTTYK |
| | AKKPVQLPGAYNVNIKLDITSHNEDYTIVEQYERAEGRHSTGGMD |
| | ELYK |
| A56-CAR-010 | MLLLVTSLLLCELPHPAFLLIPDVVMTQTPSSVSEPVGGTVTIKCQ |
| •GM-CSFR Leader | ASQSISDLLSWYQQKPGQPPKLLIYSASTLASGVSSRFKGSGSG |
| •VL | TEFTLTISDLECADAATYYCQCNYYSPTYGNGFGGGTEVVVKKG |
| •linker | STSGSGKPGSGEGSTKGQSLEESGGDLVKPGASLTLTCTASGID |
| •VH | FSDIYYISWVRQAPGKGLEWIACTYAGSSGSTYYATWAKGRFTIS |
| •Extended CD8 hinge | KASSTTVTLQMTSLTAADTATYFCARDRYPGTSGRVYGMDLWG |
| •CD8 transmembrane | PGTLVTVSSGGGGSGGGGSGGGGSGGTTTPAPRPPTPAPTIAS |
| •4-1BB | QPLSLRPEACRPAAGGAVHTRGLDFACDIYIWAPLAGTCGVLLL |
| •CD3ζ | SLVITLYCKRGRKKLLYIFKQPFMRPVQTTQEEDGCSCRFPEEEE |
| •T2A | GGCELRVKFSRSADAPAYQQGQNQLYNELNLGRREEYDVLDKR |
| •mCherry | RGRDPEMGGKPRRKNPQEGLYNELQKDKMAEAYSEIGMKGER |
| (SEQ ID NO: 106) | RRGKGHDGLYQGLSTATKDTYDALHMQALPPRGSGEGRGSLLT |
| | CGDVEENPGPMVSKGEEDNMAIIKEFMRFKVHMEGSVNGHEFEI |
| | EGEGEGRPYEGTQTAKLKVTKGGPLPFAWDILSPQFMYGSKAYV |
| | KHPADIPDYLKLSFPEGFKWERVMNFEDGGVVTVTQDSSLQDGE |
| | FIYKVKLRGTNFPSDGPVMQKKTMGWEASSERMYPEDGALKGEI |
| | KQRLKLKDGGHYDAEVKTTYKAKKPVQLPGAYNVNIKLDITSHNE |
| | DYTIVEQYERAEGRHSTGGMDELYK |
| B5-CAR-011 | MLLLVTSLLLCELPHPAFLLIPQEQLEESGGGLVKPEGSLTLTCT |
| •GM-CSFR Leader | ASGFSFSSSYYMCWVRQAPGRGLEWIACIYTSSGSAYYANWAK |
| •VH | GRFTISRTSSTTVTLQMTRLTAADTATYFCVRNAVGSSYYLYLW |
| •Linker | GPGTLVTVSSGSTSGSGKPGSGEGSTKGQVLTQTPSPVSAAVG |
| •VL | GTVTISCQASQSVAGNNYLSWYQQKPGQPPNLLIYSVSTLASGV |
| •CD8 hinge | PSRFKGSGSGTQFTLTISDLECDDAATYYCQGYYNDGIWAFGGG |
| •CD8 transmembrane | TEVVVKTTTPAPRPPTPAPTIASQPLSLRPEACRPAAGGAVHTR |
| •4-1BB | GLDFACDIYIWAPLAGTCGVLLLSLVITLYCKRGRKKLLYIFKQPF |
| •CD3ζ | MRPVQTTQEEDGCSCRFPEEEEGGCELRVKFSRSADAPAYQQG |
| •T2A | QNQLYNELNLGRREEYDVLDKRRGRDPEMGGKPRRKNPQEGL |
| •mCherry | YNELQKDKMAEAYSEIGMKGERRRGKGHDGLYQGLSTATKDTY |
| (SEQ ID NO: 107) | DALHMQALPPRGSGEGRGSLLTCGDVEENPGPMVSKGEEDNM |
| | AIIKEFMRFKVHMEGSVNGHEFEIEGEGEGRPYEGTQTAKLKVTK |
| | GGPLPFAWDILSPQFMYGSKAYVKHPADIPDYLKLSFPEGFKWER |
| | VMNFEDGGWTVTQDSSLQDGEFIYKVKLRGTNFPSDGPVMQKK |
| | TMGWEASSERMYPEDGALKGEIKQRLKLKDGGHYDAEVKTTYKA |
| | KKPVQLPGAYNVNIKLDITSHNEDYTIVEQYERAEGRHSTGGMDE |
| | LYK |
| B5-CAR-019 | MLLLVTSLLLCELPHPAFLLIPQEQLEESGGGLVKPEGSLTLTCT |
| •GM-CSFR Leader | ASGFSFSSSYYMCWVRQAPGRGLEWIACIYTSSGSAYYANWAK |
| •VH | GRFTISRTSSTTVTLQMTRLTAADTATYFCVRNAVGSSYYLYLW |
| •Linker | GPGTLVTVSSGSTSGSGKPGSGEGSTKGQVLTQTPSPVSAAVG |
| •VL | GTVTISCQASQSVAGNNYLSWYQQKPGQPPNLLIYSVSTLASGV |
| •CD8 hinge | PSRFKGSGSGTQFTLTISDLECDDAATYYCQGYYNDGIWAFGGG |
| •CD8 transmembrane | TEVVVKTTTPAPRPPTPAPTIASQPLSLRPEACRPAAGGAVHTR |
| •4-1BB | GLDFACDIYIWAPLAGTCGVLLLSLVITLYCKRGRKKLLYIFKQPF |
| •CD3ζ | MRPVQTTQEEDGCSCRFPEEEEGGCELRVKFSRSADAPAYQQG |
| •T2A | QNQLYNELNLGRREEYDVLDKRRGRDPEMGGKPRRKNPQEGL |
| •eGFP | YNELQKDKMAEAYSEIGMKGERRRGKGHDGLYQGLSTATKDTY |
| (SEQ ID NO: 108) | DALHMQALPPRGSGEGRGSLLTCGDVEENPGPMVSKGEELFTG |
| | VVPILVELDGDVNGHKFSVSGEGEGDATYGKLTLKFICTTGKLPVP |
| | WPTLVTTLTYGVQCFSRYPDHMKQHDFFKSAMPEGYVQERTIFF |
| | KDDGNYKTRAEVKFEGDTLVNRIELKGIDFKEDGNILGHKLEYNYN |
| | SHNVYIMADKQKNGIKVNFKIRHNIEDGSVQLADHYQQNTPIGDG |
| | PVLLPDNHYLSTQSALSKDPNEKRDHMVLLEFVTAAGITLGMDEL |
| | YK |
| B5-CAR-013 | MLLLVTSLLLCELPHPAFLLIPQEQLEESGGGLVKPEGSLTLTCT |
| •GM-CSFR Leader | ASGFSFSSSYYMCWVRQAPGRGLEWIACIYTSSGSAYYANWAK |
| •VH | GRFTISRTSSTTVTLQMTRLTAADTATYFCVRNAVGSSYYLYLW |
| •Linker | GPGTLVTVSSGSTSGSGKPGSGEGSTKGQVLTQTPSPVSAAVG |
| •VL | GTVTISCQASQSVAGNNYLSWYQQKPGQPPNLLIYSVSTLASGV |
| •Extended CD8 hinge | PSRFKGSGSGTQFTLTISDLECDDAATYYCQGYYNDGIWAFGGG |
| •CD8 transmembrane | TEVVVKGGGGSGGGGSGGGGSGGTTTPAPRPPTPAPTIASQPL |
| •4-1BB | SLRPEACRPAAGGAVHTRGLDFACDIYIWAPLAGTCGVLLLSLVI |
| •CD3ζ | TLYCKRGRKKLLYIFKQPFMRPVQTTQEEDGCSCRFPEEEEGGC |
| •T2A | ELRVKFSRSADAPAYQQGQNQLYNELNLGRREEYDVLDKRRGR |
| •mCherry | DPEMGGKPRRKNPQEGLYNELQKDKMAEAYSEIGMKGERRRG |
| (SEQ ID NO: 109) | KGHDGLYQGLSTATKDTYDALHMQALPPRGSGEGRGSLLTCGD |
| | VEENPGPMVSKGEEDNMAIIKEFMRFKVHMEGSVNGHEFEIEGE |
| | GEGRPYEGTQTAKLKVTKGGPLPFAWDILSPQFMYGSKAYVKHP |
| | ADIPDYLKLSFPEGFKWERVMNFEDGGWTVTQDSSLQDGEFIYK |
| | VKLRGTNFPSDGPVMQKKTMGWEASSERMYPEDGALKGEIKQR |
| | LKLKDGGHYDAEVKTTYKAKKPVQLPGAYNVNIKLDITSHNEDYTI |
| | VEQYERAEGRHSTGGMDELYK |
| B5-CAR-022 | MLLLVTSLLLCELPHPAFLLIPQEQLEESGGGLVKPEGSLTLTCT |
| •GM-CSFR Leader | ASGFSFSSSYYMCWVRQAPGRGLEWIACIYTSSGSAYYANWAK |
| •VH | GRFTISRTSSTTVTLQMTRLTAADTATYFCVRNAVGSSYYLYLW |
| •Linker | GPGTLVTVSSGSTSGSGKPGSGEGSTKGQVLTQTPSPVSAAVG |
| •VL | GTVTISCQASQSVAGNNYLSWYQQKPGQPPNLLIYSVSTLASGV |
| •CD28 hinge | PSRFKGSGSGTQFTLTISDLECDDAATYYCQGYYNDGIWAFGGG |
| •CD28 | TEVVVKIEVMYPPPYLDNEKSNGTIIHVKGKHLCPSPLFPGPSKP |
| transmembrane | FWVLVWGGVLACYSLLVTVAFIIFWVKRGRKKLLYIFKQPFMRP |
| •4-1BB | VQTTQEEDGCSCRFPEEEEGGCELRVKFSRSADAPAYQQGQN |
| •CD3ζ | QLYNELNLGRREEYDVLDKRRGRDPEMGGKPRRKNPQEGLYN |
| •T2A | ELQKDKMAEAYSEIGMKGERRRGKGHDGLYQGLSTATKDTYDA |
| •mCherry | LHMQALPPRGSGEGRGSLLTCGDVEENPGPMVSKGEEDNMAIIK |
| (SEQ ID NO: 110) | EFMRFKVHMEGSVNGHEFEIEGEGEGRPYEGTQTAKLKVTKGGP |
| | LPFAWDILSPQFMYGSKAYVKHPADIPDYLKLSFPEGFKWERVMN |
| | FEDGGWTVTQDSSLQDGEFIYKVKLRGTNFPSDGPVMQKKTMG |
| | WEASSERMYPEDGALKGEIKQRLKLKDGGHYDAEVKTTYKAKKP |
| | VQLPGAYNVNIKLDITSHNEDYTIVEQYERAEGRHSTGGMDELYK |
| B5-CAR-014 | MLLLVTSLLLCELPHPAFLLIPQVLTQTPSPVSAAVGGTVTISCQA |
| •GM-CSFR Leader | SQSVAGNNYLSWYQQKPGQPPNLLIYSVSTLASGVPSRFKGSG |
| •VL | SGTQFTLTISDLECDDAATYYCQGYYNDGIWAFGGGTEVVVKGS |
| •Linker | TSGSGKPGSGEGSTKGQEQLEESGGGLVKPEGSLTLTCTASGF |
| •VH | SFSSSYYMCWVRQAPGRGLEWIACIYTSSGSAYYANWAKGRFTI |
| •CD8 hinge | SRTSSTTVTLQMTRLTAADTATYFCVRNAVGSSYYLYLWGPGTL |
| •CD8 transmembrane | VTVSSTTTPAPRPPTPAPTIASQPLSLRPEACRPAAGGAVHTRG |
| •4-1BB | LDFACDIYIWAPLAGTCGVLLLSLVITLYCKRGRKKLLYIFKQPFM |
| •CD3ζ | RPVQTTQEEDGCSCRFPEEEEGGCELRVKFSRSADAPAYQQGQ |
| •T2A | NQLYNELNLGRREEYDVLDKRRGRDPEMGGKPRRKNPQEGLY |
| •mCherry | NELQKDKMAEAYSEIGMKGERRRGKGHDGLYQGLSTATKDTYD |
| (SEQ ID NO: 111) | ALHMQALPPRGSGEGRGSLLTCGDVEENPGPMVSKGEEDNMAII |
| | KEFMRFKVHMEGSVNGHEFEIEGEGEGRPYEGTQTAKLKVTKGG |
| | PLPFAWDILSPQFMYGSKAYVKHPADIPDYLKLSFPEGFKWERVM |
| | NFEDGGVVTVTQDSSLQDGEFIYKVKLRGTNFPSDGPVMQKKTM |
| | GWEASSERMYPEDGALKGEIKQRLKLKDGGHYDAEVKTTYKAKK |
| | PVQLPGAYNVNIKLDITSHNEDYTIVEQYERAEGRHSTGGMDELY |
| | K |
| B5-CAR-016 | MLLLVTSLLLCELPHPAFLLIPQVLTQTPSPVSAAVGGTVTISCQA |
| •GM-CSFR Leader | SQSVAGNNYLSWYQQKPGQPPNLLIYSVSTLASGVPSRFKGSG |
| •VL | SGTQFTLTISDLECDDAATYYCQGYYNDGIWAFGGGTEVVVKGS |
| •Linker | TSGSGKPGSGEGSTKGQEQLEESGGGLVKPEGSLTLTCTASGF |
| •VH | SFSSSYYMCWVRQAPGRGLEWIACIYTSSGSAYYANWAKGRFTI |
| •Extended CD8 hinge | SRTSSTTVTLQMTRLTAADTATYFCVRNAVGSSYYLYLWGPGTL |
| •CD8 transmembrane | VTVSSGGGGSGGGGSGGGGSGGTTTPAPRPPTPAPTIASQPLS |
| •4-1BB | LRPEACRPAAGGAVHTRGLDFACDIYIWAPLAGTCGVLLLSLVIT |
| •CD3ζ | LYCKRGRKKLLYIFKQPFMRPVQTTQEEDGCSCRFPEEEEGGCE |
| •T2A | LRVKFSRSADAPAYQQGQNQLYNELNLGRREEYDVLDKRRGRD |
| •mCherry | PEMGGKPRRKNPQEGLYNELQKDKMAEAYSEIGMKGERRRGK |
| (SEQ ID NO: 112) | GHDGLYQGLSTATKDTYDALHMQALPPRGSGEGRGSLLTCGDV |
| | EENPGPMVSKGEEDNMAIIKEFMRFKVHMEGSVNGHEFEIEGEG |
| | EGRPYEGTQTAKLKVTKGGPLPFAWDILSPQFMYGSKAYVKHPA |
| | DIPDYLKLSFPEGFKWERVMNFEDGGWTVTQDSSLQDGEFIYKV |
| | KLRGTNFPSDGPVMQKKTMGWEASSERMYPEDGALKGEIKQRL |
| | KLKDGGHYDAEVKTTYKAKKPVQLPGAYNVNIKLDITSHNEDYTIV |
| | EQYERAEGRHSTGGMDELYK |

In certain embodiments, provided are CARs comprising one or more murine CAR domains. Non-limiting examples of murine CAR domains that may be included in a CAR of the present disclosure include one or more of the murine CAR domains provided in Table 10 below. As will be appreciated by one of ordinary skill in the art, the amino acid sequence of one or more of the domains indicated in Table 10 (e.g., leader sequence, linker, hinge, transmembrane, co-stimulatory, signaling, ribosomal skip element; restriction enzyme sequence; reporter protein; etc.) may be modified as desired.

TABLE 10
Murine CAR Domain Amino Acid Sequences
Leader Signal Peptide(s)

| | |
|---|---|
| CD8 alpha (P01731) | MASPLTRFLSLNLLLLGESIILGSGEA |
| (SEQ ID NO: 144) | |
| GM-CSFR alpha (Q00941) | MTSSHAMNITPLAQLALLFSTLLLPGTQA |
| (SEQ ID NO: 145) | |

Hinge/Spacer/Linker domain(s)

| | |
|---|---|
| (SEQ ID NO: 114) | GSTSGSGKPGSGEGSTKG |
| CD8 alpha Hinge (P01731) | TTKPVLRTPSPVHPTGTSQPQRPEDCRPRGSVKGTGLDFACDIY |
| (SEQ ID NO: 146) | |
| CD28 Hinge (P31041) | FCKIEFMYPPPYLDNERSNGTIIHIKEKHLCHTQSSPKL |
| (SEQ ID NO: 147) | |

Transmembrane domain(s)

| | |
|---|---|
| CD8 alpha TM (P01731) | IWAPLAGICVALLLSLIITL |
| (SEQ ID NO: 148) | |
| CD8 alpha TM + 4 aa | IWAPLAGICVALLLSLIITLICYH |
| (P01731) | |
| (SEQ ID NO: 149) | |
| CD28TM (P31041) | FWALVVVAGVLFCYGLLVTVALCVIWT |
| (SEQ ID NO: 150) | |

Costimulatory domain(s)

| | |
|---|---|
| 41BB (P20334) | KWIRKKFPHIFKQPFKKTTGAAQEEDACSCRCPQEEEGGGGGYEL |
| (SEQ ID NO: 151) | |
| CD28 (P31041) | NSRRNRLLQSDYMNMTPRRPGLTRKPYQPYAPARDFAAYRP |
| (SEQ ID NO: 152) | |

CD3ζ domain(s)

| | |
|---|---|
| CD3z (P24161) | RAKFSRSAETAANLQDPNQLYNELNLGRREEYDVLEKKRARDPEM |
| (SEQ ID NO: 153) | GGKQQRRRNPQEGVYNALQKDKMAEAYSEIGTKGERRRGKGHD |
| | GLYQGLSTATKDTYDALHMQTLAPR |

Ribosomal Skip Element(s)

| | |
|---|---|
| T2A | GSGEGRGSLLTCGDVEENPGP |
| (SEQ ID NO: 137) | |

Reporter Protein(s)

| | |
|---|---|
| Thy1.1 | MNPAISVALLLSVLQVSRGQKVTSLTACLVNQNLRLDCRHENNTKD |
| (SEQ ID NO: 154) | NSIQHEFSLTREKRKHVLSGTLGIPEHTYRSRVTLSNQPYIKVLTLA |
| | NFTTKDEGDYFCELRVSGANPMSSNKSISVYRDKLVKCGGISLLVQ |
| | NTSWMLLLLLSLSLLQALDFISL |

According to some embodiments, provided are murine CARs comprising one or more murine CAR domains (e.g., one or more of any of the domains set forth in Table 10 above). Non-limiting examples of murine CARs of the present disclosure include those provided in Table 11 below. As will be appreciated by one of ordinary skill in the art, the amino acid sequence of one or more of the domains indicated in Table 11 (e.g., leader sequence, V_{H}, linker, V_{L}, hinge, transmembrane, co-stimulatory, signaling, ribosomal skip element, etc.) may be modified as desired. In Table 11, the CAR portion is shown in bold. Segments/domains of the polypeptides are indicated by alternating underlining, and the identities of the segments/domains are provided in the left column.

TABLE 11
Murine CAR Amino Acid Sequences

| | |
|---|---|
| B5-CAR-031 | MASPLTRFLSLNLLLLGESIILGSGEAQEQLEESGGGLVKPEGSL |
| •CD8 Leader | TLTCTASGFSFSSSYYMCWVRQAPGRGLEWIACIYTSSGSAYYA |
| •VH | NWAKGRFTISRTSSTTVTLQMTRLTAADTATYFCVRNAVGSSYY |
| •Linker | LYLWGPGTLVTVSSGSTSGSGKPGSGEGSTKGQVLTQTPSPVS |
| •VL | AAVGGTVTISCQASQSVAGNNYLSWYQQKPGQPPNLLIYSVSTL |
| •CD8 hinge | ASGVPSRFKGSGSGTQFTLTISDLECDDAATYYCQGYYNDGIWA |
| •CD8 transmembrane | FGGGTEVVVKTTKPVLRTPSPVHPTGTSQPQRPEDCRPRGSVK |
| •4-1BB | GTGLDFACDIYIWAPLAGICVALLLSLIITLICYHKWIRKKFPHIFKQ |
| •CD3ζ | PFKKTTGAAQEEDACSCRCPQEEEGGGGGYELRAKFSRSAETA |
| •T2A | ANLQDPNQLYNELNLGRREEYDVLEKKRARDPEMGGKQQRRR |
| •Thy1.1 | NPQEGVYNALQKDKMAEAYSEIGTKGERRRGKGHDGLYQGLST |
| (SEQ ID NO: 140) | ATKDTYDALHMQTLAPRGSGEGRGSLLTCGDVEENPGPMNPAI |
| | SVALLLSVLQVSRGQKVTSLTACLVNQNLRLDCRHENNTKDNSIQ |
| | HEFSLTREKRKHVLSGTLGIPEHTYRSRVTLSNQPYIKVLTLANFT |
| | TKDEGDYFCELRVSGANPMSSNKSISVYRDKLVKCGGISLLVQNT |
| | SWMLLLLLSLSLLQALDFISL |
| B5-CAR-032 | MASPLTRFLSLNLLLLGESIILGSGEAQVLTQTPSPVSAAVGGTV |
| •CD8 Leader | TISCQASQSVAGNNYLSWYQQKPGQPPNLLIYSVSTLASGVPSR |
| •VL | FKGSGSGTQFTLTISDLECDDAATYYCQGYYNDGIWAFGGGTEV |
| •Linker | VVKGSTSGSGKPGSGEGSTKGQEQLEESGGGLVKPEGSLTLTC |
| •VH | TASGFSFSSSYYMCWVRQAPGRGLEWIACIYTSSGSAYYANWA |
| •CD8 hinge | KGRFTISRTSSTTVTLQMTRLTAADTATYFCVRNAVGSSYYLYL |
| •CD8 transmembrane | WGPGTLVTVSSTTKPVLRTPSPVHPTGTSQPQRPEDCRPRGSV |
| •4-1BB | KGTGLDFACDIYIWAPLAGICVALLLSLIITLICYHKWIRKKFPHIFK |
| •CD3ζ | QPFKKTTGAAQEEDACSCRCPQEEEGGGGGYELRAKFSRSAET |
| •T2A | AANLQDPNQLYNELNLGRREEYDVLEKKRARDPEMGGKQQRR |
| •Thy1.1 | RNPQEGVYNALQKDKMAEAYSEIGTKGERRRGKGHDGLYQGL |
| (SEQ ID NO: 141) | STATKDTYDALHMQTLAPRGSGEGRGSLLTCGDVEENPGPMNP |
| | AISVALLLSVLQVSRGQKVTSLTACLVNQNLRLDCRHENNTKDNSI |
| | QHEFSLTREKRKHVLSGTLGIPEHTYRSRVTLSNQPYIKVLTLANF |
| | TTKDEGDYFCELRVSGANPMSSNKSISVYRDKLVKCGGISLLVQN |
| | TSWMLLLLLSLSLLQALDFISL |
| A56-CAR-033 | MASPLTRFLSLNLLLLGESIILGSGEAQSLEESGGDLVKPGASLT |
| •CD8 Leader | LTCTASGIDFSDIYYISWVRQAPGKGLEWIACTYAGSSGSTYYAT |
| •VH | WAKGRFTISKASSTTVTLQMTSLTAADTATYFCARDRYPGTSGR |
| •Linker | VYGMDLWGPGTLVTVSSGSTSGSGKPGSGEGSTKGVVMTQTP |
| •VL | SSVSEPVGGTVTIKCQASQSISDLLSWYQQKPGQPPKLLIYSAST |
| •CD8 hinge | LASGVSSRFKGSGSGTEFTLTISDLECADAATYYCQCNYYSPTY |
| •CD8 transmembrane | GNGFGGGTEVVVKTTKPVLRTPSPVHPTGTSQPQRPEDCRPRG |
| •4-1BB | SVKGTGLDFACDIYIWAPLAGICVALLLSLIITLICYHKWIRKKFPHI |
| •CD3ζ | FKQPFKKTTGAAQEEDACSCRCPQEEEGGGGGYELRAKFSRS |
| •T2A | AETAANLQDPNQLYNELNLGRREEYDVLEKKRARDPEMGGKQ |
| •Thy1.1 | QRRRNPQEGVYNALQKDKMAEAYSEIGTKGERRRGKGHDGLY |
| (SEQ ID NO: 142) | QGLSTATKDTYDALHMQTLAPRGSGEGRGSLLTCGDVEENPGP |
| | MNPAISVALLLSVLQVSRGQKVTSLTACLVNQNLRLDCRHENNTK |
| | DNSIQHEFSLTREKRKHVLSGTLGIPEHTYRSRVTLSNQPYIKVLTL |
| | ANFTTKDEGDYFCELRVSGANPMSSNKSISVYRDKLVKCGGISLL |
| | VQNTSWMLLLLLSLSLLQALDFISL |
| A56-CAR-034 | MASPLTRFLSLNLLLLGESIILGSGEAVVMTQTPSSVSEPVGGTV |
| •CD8 Leader | TIKCQASQSISDLLSWYQQKPGQPPKLLIYSASTLASGVSSRFKG |
| •VL | SGSGTEFTLTISDLECADAATYYCQCNYYSPTYGNGFGGGTEW |
| •Linker | VKGSTSGSGKPGSGEGSTKGQSLEESGGDLVKPGASLTLTCTA |
| •VH | SGIDFSDIYYISWVRQAPGKGLEWIACTYAGSSGSTYYATWAKG |
| •CD8 hinge | RFTISKASSTTVTLQMTSLTAADTATYFCARDRYPGTSGRVYGM |
| •CD8 transmembrane | DLWGPGTLVTVSSTTKPVLRTPSPVHPTGTSQPQRPEDCRPRG |
| •4-1BB | SVKGTGLDFACDIYIWAPLAGICVALLLSLIITLICYHKWIRKKFPHI |
| •CD3ζ | FKQPFKKTTGAAQEEDACSCRCPQEEEGGGGGYELRAKFSRS |
| •T2A | AETAANLQDPNQLYNELNLGRREEYDVLEKKRARDPEMGGKQ |
| •Thy1.1 | QRRRNPQEGVYNALQKDKMAEAYSEIGTKGERRRGKGHDGLY |
| (SEQ ID NO: 143) | QGLSTATKDTYDALHMQTLAPRGSGEGRGSLLTCGDVEENPGP |
| | MNPAISVALLLSVLQVSRGQKVTSLTACLVNQNLRLDCRHENNTK |
| | DNSIQHEFSLTREKRKHVLSGTLGIPEHTYRSRVTLSNQPYIKVLTL |
| | ANFTTKDEGDYFCELRVSGANPMSSNKSISVYRDKLVKCGGISLL |
| | VQNTSWMLLLLLSLSLLQALDFISL |

According to some embodiments, a CAR of the present disclosure is provided by a single polypeptide. In certain embodiments, a CAR of the present disclosure is provided by two or more polypeptides. When the CAR is provided by two or more polypeptides, the CAR may be provided in any useful multi-polypeptide format, including universal CAR formats such as biotin-binding immune receptor (BBIR) format (see, e.g., Urbanska K, Powell D J. Development of a novel universal immune receptor for antigen targeting to infinity and beyond. Oncoimmunology. 2012; 1(5):777-779. doi:10.4161/onci.19730, and Urbanska K, Lanitis E, Poussin M, et al. A universal strategy for adoptive immunotherapy of cancer through use of a novel T cell antigen receptor. 2013; 72(7):1844-1852. doi:10.1158/0008-5472.CAN-11-3890.A); a switchable CAR format with peptide NeoEpitope (PNE) (see, e.g., Kim et al. (2015) J Am Chem Soc. 2015; 137(8):2832-2835; Ma et al. (2016) Proc Natl Acad Sci 113(4):E450-8-; Rodgers et al. (2016) Proc Natl Acad Sci. 113(4): E459-E468; Viaud et al. (2018) Proc Natl Acad Sci 115(46):E10898-E10906); a SUPRA CAR format with leucine zippers (see, e.g., Cho et al. (2108) Cell 173(6):1426-1438.el 1); a CAR-T Adapter Molecule (CAM)-based format with FITC-folic acid (see, e.g., Lee et al. (2019) Cancer Res. 79(2):387-396; and Lu et al. (2019) Front Oncol. 9:151); anti-FITC-folic acid adaptor format (see, e.g., Chu et al. (2018) Biosci Trends. 12(3):298-308); anti-FITC antibody adaptor CAR format (see, e.g., Tamada et al. (2012) Clin Cancer Res. 18(23):6436-6445); Fc-targeting (e.g., anti-CD16) CAR+ anti-tumor antibody format (see, e.g., Kudo et al. (2014) Cancer Res. 74(1):93-103); and the like.

Conjugates

The present disclosure also provides conjugates. According to some embodiments, a conjugate of the present disclosure comprises any of the antibodies or fusion proteins of the present disclosure, and an agent conjugated to the antibody or fusion protein. The term “conjugated” generally refers to a chemical linkage, either covalent or non-covalent, usually covalent, that proximally associates one molecule of interest with a second molecule of interest. In certain embodiments, the agent conjugated to the antibody or fusion protein is selected from a chemotherapeutic agent, a toxin, a radiation-sensitizing agent, a radioactive isotope (e.g., a therapeutic radioactive isotope), a detectable label, and a half-life extending moiety.

According to some embodiments, the agent is a therapeutic agent, e.g., a chemotherapeutic agent. Therapeutic agents of interest include agents capable of affecting the function of a cell/tissue to which the conjugate binds via specific binding of the antibody portion of the conjugate to the antigen. When the function of the cell/tissue is pathological, an agent that reduces the function of the cell/tissue may be employed. In certain aspects, a conjugate of the present disclosure includes an agent that reduces the function of a target cell/tissue by inhibiting cell proliferation and/or killing the cell/tissue. Such agents may vary and include cytostatic agents and cytotoxic agents, e.g., an agent capable of killing a target cell tissue with or without being internalized into a target cell.

In certain embodiments, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to some embodiments, the agent is a toxin, such as a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the agent is a radiation-sensitizing agent. As used herein, a “radiation-sensitizing agent” is an agent that enhances the ability of radiation to kill tumor cells. Non-limiting examples of radiation-sensitizing agents that may be conjugated to the antibody or fusion protein include cisplatin, 5-fluorouracil (5-FU), AZD7762, selumetinib, and the like.

In certain embodiments, the agent is a radioisotope, e.g., useful for therapy and/or detection (e.g., imaging). Non-limiting examples of radioisotopes that may be conjugated to the antibody or fusion protein include but are not limited to ²²⁵Ac, ¹¹¹Ag, ¹¹⁴Ag, ⁷¹As, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ¹³C, ⁵⁵Co, ⁶²Cu, ⁶⁴Cu, ⁶⁷Cu, ¹⁶⁵Dy, ¹⁶⁶Dy, ¹⁶⁹Er, ¹⁸F ¹⁹F, ⁵²Fe, ⁵⁹Fe, ⁶⁶Ga, ⁶⁷Ga, ⁶⁸Ga, ⁷²Ga, ¹⁵⁴⁻¹⁵⁸Gd, ¹⁵⁷Gd, ¹⁵⁹Gd, ¹⁶⁶Ho, ¹²⁰I, 121I, ¹²³I, ¹²⁴I, ¹²⁵I, ¹³¹I, ¹¹⁰In, ¹¹¹In, ^{113m}In, ¹⁹⁴Ir, ^{81m}Kr, ¹⁷⁷Lu, ⁵¹Mn, ⁵²Mn, ⁹⁹Mo, ¹³N, ¹⁵N, ¹⁵O, ¹⁷O, ³²P, ³³P, ²¹¹Pb, ²¹²Pb, ¹⁰⁹Pd, ¹⁴⁹Pm, ¹⁵¹Pm, ¹⁴²Pr, ¹⁴³Pr, ¹⁹¹PT, ^{193m}PT, ^{195m}Pt, ²²³Ra, ¹⁴²Rb, ¹⁸⁶Re, ¹⁸⁸Re, ¹⁸⁹Re, ¹⁰⁵Rh, ⁴⁷Sc, ⁷⁵Se, ¹⁵³Sm, ^{117m}Sn, ¹²¹Sn, ⁸³Sr, ⁸⁹Sr, ¹⁶¹Tb, ⁹⁴Tc, ⁹⁹Tc, ^{99m}Tc, ²²⁷Th, ²⁰¹Tl, ¹⁷²Tm, ¹²⁷Te, ⁹⁰Y, ¹⁶⁹Yb, ¹⁷⁵Yb, ¹³³X, and ⁸⁹Zr.

In certain embodiments, a radioisotope is conjugated to the antibody or fusion protein via a chelator, for example, a bifunctional chelator. A bifunctional chelator may contain a metal chelating moiety that binds the radioisotope in a stable coordination complex and a reactive functional group that is covalently linked to a targeting moiety, such as any of the antibodies or fusion proteins of the present disclosure, so that the radioisotope may be properly directed to the desirable molecular target in vivo. Non-limiting examples of bifunctional chelators that may be employed to conjugate an antibody or fusion protein of the present disclosure to a radioisotope include p-SCN-Bn-DOTA and p-SCN-Bn-deferoxamine. Additional examples of bifunctional chelators that may be employed to conjugate an antibody or fusion protein of the present disclosure to a radioisotope include those described in Price & Orvig (2014) Chem. Soc. Rev. 43:260; and Brechbiel (2008) Q J Nucl Med Mol Imaging 52(2):166-173.

According to some embodiments, the radioisotope is a therapeutic radioisotope. In certain embodiments, the radioisotope is an alpha emitting radioisotope, e.g., ²²⁵Ac, ²¹¹At, ²¹²Bi/²¹²Pb, ²¹³Bi, ²²³Ra, or ²²⁷Th. In other embodiments, the radioisotope is a beta minus emitting radioisotope, e.g., ³²P, ³³P, ⁶⁷Cu, ⁹⁰Y, ¹³¹I or ¹⁷⁷Lu.

According to some embodiments, the agent is a labeling agent. By “labeling agent” (or “detectable label”) is meant the agent detectably labels the antibody or fusion protein, such that the antibody or fusion protein may be detected in an application of interest (e.g., in vitro and/or in vivo research and/or clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain aspects, the antibody or fusion protein is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)±CT imaging, positron emission tomography (PET)±CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 8000 W Fluors. According to some embodiments, the labeling agent is an in vivo imaging agent that finds use in SPECT imaging applications, non-limiting examples of which include ^{99m}Tc, ¹¹¹n, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

For half-life extension, the antibodies and fusion proteins of the present disclosure may be conjugated to an agent that provides for an improved pharmacokinetic profile (e.g., by PEGylation, hyperglycosylation, and the like). Modifications that can enhance serum half-life are of interest. A subject antibody or fusion protein may be “PEGylated”, as containing one or more poly(ethylene glycol) (PEG) moieties. Methods and reagents suitable for PEGylation of a protein are well known in the art and may be found, e.g., in U.S. Pat. No. 5,849,860. PEG suitable for conjugation to a protein is generally soluble in water at room temperature and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject antibody or fusion protein can be linear. The PEG conjugated to the subject antibody or fusion protein may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Where the subject antibody or fusion protein is to be isolated from a source, the antibody or fusion protein may be conjugated to one or more moieties that facilitate purification, such as members of specific binding pairs, e.g., biotin (member of biotin-avidin specific binding pair), a lectin, and the like. The antibody can also be bound to (e.g., immobilized onto) a solid support, including, but not limited to, polystyrene plates or beads, magnetic beads, test strips, membranes, and the like.

Where the antibodies or fusion proteins are to be detected in an assay, the antibodies or fusion proteins may contain a detectable label, e.g., a radioisotope (e.g., ⁸⁹Zr; ¹¹¹In, and the like), an enzyme which generates a detectable product (e.g., luciferase, β-galactosidase, horse radish peroxidase, alkaline phosphatase, and the like), a fluorescent protein, a chromogenic protein, dye (e.g., fluorescein isothiocyanate, rhodamine, phycoerythrin, and the like); fluorescence emitting metals, e.g., ¹⁵²Eu, or others of the lanthanide series, attached to the protein through metal chelating groups such as EDTA; chemiluminescent compounds, e.g., luminol, isoluminol, acridinium salts, and the like; bioluminescent compounds, e.g., luciferin; fluorescent proteins; and the like. Indirect labels include antibodies specific for a subject protein, wherein the antibody may be detected via a secondary antibody; and members of specific binding pairs, e.g., biotin-avidin, and the like.

Any of the above agents may be conjugated to the antibody or fusion protein via a linker. If present, the linker molecule(s) may be of sufficient length to permit the antibody or fusion protein and the linked agent to allow some flexible movement between the antibody or fusion protein and the linked agent. Linker molecules may be, e.g., about 6-50 atoms long. Linker molecules may also be, e.g., aryl acetylene, ethylene glycol oligomers containing 2-10 monomer units, diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G)ₙ, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of an antibody or fusion protein conjugated to any agents described above can include linkers that are all or partially flexible, such that the linker can include a flexible linker as well as one or more portions that confer a less flexible structure.

According to some embodiments, the antibody or fusion protein is conjugated to the agent via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the antibody is conjugated to the agent via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzoyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9(1):33-46; and elsewhere.

Numerous strategies are available for linking agents to an antibody or fusion protein directly, or indirectly via a linker. For example, the agent may be derivatized by covalently attaching a linker to the agent, where the linker has a functional group capable of reacting with a “chemical handle” on the antibody or fusion protein. The functional group on the linker may vary and may be selected based on compatibility with the chemical handle on the antibody or fusion protein. According to one embodiment, the chemical handle on the antibody or fusion protein is provided by incorporation of an unnatural amino acid having the chemical handle into the antibody or fusion protein. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into an antibody of a conjugate of the present disclosure, which unnatural amino acid may be selected to provide a functional group of interest, are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. An unnatural amino acid may be incorporated into an antibody or fusion protein via chemical synthesis or recombinant approaches, e.g., using a suitable orthogonal amino acyl tRNA synthetase-tRNA pair for incorporation of the unnatural amino acid during translation of the antibody or fusion protein in a host cell.

The functional group of an unnatural amino acid present in the antibody or fusion protein may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the antibody or fusion protein and the linker portion of a linker-agent moiety may include an alkyne functional group, such that the antibody or fusion protein and linker-agent moiety are covalently conjugated via azide-alkyne cycloaddition. Conjugation may be carried out using, e.g., a copper-catalyzed azide-alkyne cycloaddition reaction.

In certain embodiments, the chemical handle on the antibody or fusion protein does not involve an unnatural amino acid. An antibody containing no unnatural amino acids may be conjugated to the agent by utilizing, e.g., nucleophilic functional groups of the antibody or fusion protein (such as the N-terminal amine or the primary amine of lysine, or any other nucleophilic amino acid residue) as a nucleophile in a substitution reaction with a moiety bearing a reactive leaving group or other electrophilic group. An example would be to prepare an agent-linker moiety bearing an N-hydroxysuccinimidyl (NHS) ester and allow it to react with the antibody or fusion protein under aqueous conditions at elevated pH (˜10) or in polar organic solvents such as DMSO with an added non-nucleophilic base, such as N,N-diisopropylethylamine.

It will be appreciated that the particular approach for attaching a linker, agent and/or antibody or fusion protein to each other may vary depending upon the particular linker, agent and/or antibody or fusion protein and functional groups selected and employed for conjugating the various components to each other.

Methods of Producing Antibodies

Using the information provided herein, the anti-VV A56 and anti-VV B5 antibodies and fusion proteins of the present disclosure may be prepared using standard techniques well known to those of skill in the art. For example, a nucleic acid sequence(s) encoding the amino acid sequence of an antibody or fusion protein of the present disclosure can be used to express the antibodies or fusion proteins. The polypeptide sequences provided herein (see, e.g., Tables 1, 3, 5-11) can be used to determine appropriate nucleic acid sequences encoding the antibodies or fusion proteins and the nucleic acids sequences then used to express one or more antibodies or fusion proteins specific for VV A56 or VV B5. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject antibody is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the antibodies and fusion proteins of the present disclosure can be achieved by operably linking a nucleic acid encoding the antibody or fusion protein to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing antibodies are available using, for example, E. coli, Bacillus sp. and Salmonella. E. coli systems may also be used.

The antibody gene(s) may also be subcloned into an expression vector that allows for the addition of a tag (e.g., FLAG, hexahistidine, and the like) at the C-terminal end or the N-terminal end of the antibody (e.g., IgG, Fab, scFv, etc.) to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject antibody is isolated and cloned, one can express the nucleic acid in a variety of recombinantly engineered cells known to those of skill in the art. Examples of such cells include bacteria, yeast, filamentous fungi, insect (e.g. those employing baculoviral vectors), and mammalian cells.

Isolation and purification of a subject antibody can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound antibody. The isolated antibody can be further purified by dialysis and other methods normally employed in protein purification methods. In one embodiment, the antibody may be isolated using metal chelate chromatography methods. Antibodies of the present disclosure may contain modifications to facilitate isolation, as discussed above.

The antibodies may be prepared in substantially pure or isolated form (e.g., free from other polypeptides). The protein can be present in a composition that is enriched for the polypeptide relative to other components that may be present (e.g., other polypeptides or other host cell components). Purified antibodies may be provided such that the antibody is present in a composition that is substantially free of other expressed proteins, e.g., less than 90%, usually less than 60% and more usually less than 50% of the composition is made up of other expressed proteins.

The antibodies produced by prokaryotic cells may require exposure to chaotropic agents for proper folding. During purification from E. coli, for example, the expressed protein can be optionally denatured and then renatured. This can be accomplished, e.g., by solubilizing the bacterially produced antibodies in a chaotropic agent such as guanidine HCl. The antibody is then renatured, either by slow dialysis or by gel filtration. Alternatively, nucleic acid encoding the antibodies may be operably linked to a secretion signal sequence such as pelB so that the antibodies are secreted into the periplasm in correctly-folded form.

The present disclosure also provides cells that produce the antibodies of the present disclosure, where suitable cells include eukaryotic cells, e.g., mammalian cells. The cells can be a hybrid cell or “hybridoma” that is capable of reproducing antibodies in vitro (e.g. monoclonal antibodies, such as IgG). For example, the present disclosure provides a recombinant host cell (also referred to herein as a “genetically modified host cell”) that is genetically modified with one or more nucleic acids comprising a nucleotide sequence encoding a heavy and/or light chain of an antibody of the present disclosure.

Techniques for creating recombinant DNA versions of the antigen-binding regions of antibody molecules which bypass the generation of hybridomas are also contemplated herein. DNA is cloned into a bacterial (e.g., bacteriophage), yeast (e.g. Saccharomyces or Pichia), insect or mammalian expression system, for example. One example of a suitable technique uses a bacteriophage lambda vector system having a leader sequence that causes the expressed antibody (e.g. Fab or scFv) to migrate to the periplasmic space (between the bacterial cell membrane and the cell wall) or to be secreted. One can rapidly generate great numbers of functional fragments (e.g. Fab or scFv) for those which bind the antigen of interest.

Antibodies that specifically bind VV A56 and VV B5 can be prepared using a wide variety of techniques known in the art including the use of hybridoma, recombinant, phage display technologies, Selected Lymphocyte Antibody Method (SLAM) (1), or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds VV A56 or VV B5 can be selected or identified with VV A56 or VV B5, e.g., using labeled VV A56 or VV B5 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene Ill or gene VIII protein. The production of high affinity human antibodies by chain shuffling is known, as are combinatorial infection and in vivo recombination as a strategy for constructing large phage libraries. In another embodiment, ribosomal display can be used to replace bacteriophage as the display platform. Cell surface libraries may be screened for antibodies. Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′fragments may be employed using methods known in the art.

Nucleic Acids, Expression Vectors and Cells

In view of the section above regarding methods of producing the antibodies and fusion proteins of the present disclosure, it will be appreciated that the present disclosure also provides nucleic acids, expression vectors and cells.

In certain embodiments, provided is a nucleic acid encoding a variable heavy chain (V_{H}) polypeptide, a variable light chain (V_{L}) polypeptide, or both, of an antibody or fusion protein of the present disclosure, including any of the anti-VV A56 and anti-VV B5 antibodies of the present disclosure, e.g., any of such antibodies described hereinabove. According to some embodiments, the antibody is a single chain antibody (e.g., an scFv), and the nucleic acid encodes the single chain antibody.

According to some embodiments, provided are nucleic acids that encode the variable heavy chain (V_{H}) polypeptide, the variable light chain (V_{L}) polypeptide, or both, of the anti-VV A56 antibodies A047/A057, A049/A059/A056, A050, or A054. Examples of such nucleotide sequences are provided in Table 12 below. Sequences that encode framework regions and CDRs are shown in upper case and lower case, respectively.

TABLE 12
Nucleotide Sequences Encoding Example Anti-VV A56 Antibodies

| | |
|---|---|
| A047/A057 V_{H} | CAGGAGCAACTGGAGGAGTCCGGGGGAGACCTGGTCAAGCC |
| (SEQ ID NO: 50) | TGAGGGATCCCTGACACTCACCTGCACAGCCTCTGGATTCTC |
| | CTTCAGTagcagctactggatatgcTGGGTCCGCCAGGCTCCAGGGA |
| | AGGGGCTGGAGTGGATCGCAtgcatttatgctggtagtggtggtagcacttact |
| | acgcgacctgggcgaaaggcCGATTCACCATCTCCAAAACCTCGTCGA |
| | CCACGGTGACTCTGCAAATGACCAGTCTGACAGCCGCGGACA |
| | CGGCCAGCTATTTCTGTGTCCGCgcatatagtgatagaagtggtggttactc |
| | atttaatttgTGGGGCCCAGGCACCCTGGTCACCGTCTCTTCA |
| A047/A057 V_{L} | CAAGTGCTGACCCAGACTGCATCCCCCGTGTCTGCGGCTGTT |
| (SEQ ID NO: 51) | GGAGGCACAGTCACCATCAATTGCcaggccagtcagagtgttgataataa |
| | caactacttagccTGGTATCAGCAGAAACCAGGGCAGCCTCCCAAA |
| | CAACTGATCTATtctgcatccagtctggcatctGGGGTCCCATCGCGGTT |
| | CAAAGGCAGTGGATCTGGGACACAGTTCACTCTCACCATCAG |
| | CGACGTGCAGTGTGACGATGCTGCCACTTACTACTGTctaggca |
| | gttatgattgtagtgatgctgattgttatgctTTCGGCGGAGGGACCGAGGTGG |
| | TGGTCAAA |
| A049/A059/A056 V_{H} | CAGTCCTTGGAGGAGTCCGGGGGAGACCTGGTCAAGCCTGG |
| (SEQ ID NO: 52) | GGCATCCCTGACACTCACCTGCACAGCCTCTGGAATCGACTT |
| | CAGTgacatttattacatatctTGGGTCCGCCAGGCTCCAGGGAAGGG |
| | GCTGGAGTGGATCGCAtgcacttatgctggtagtagtggtagcacttactacgcg |
| | acctgggcgaaaggcCGATTCACCATCTCCAAAGCCTCGTCGACCA |
| | CGGTGACTCTGCAAATGACCAGTCTGACAGCCGCGGACACG |
| | GCCACCTATTTCTGTGCGAGAgatcgttatcctggtactagtggtagggtctac |
| | ggcatggacctcTGGGGCCCAGGGACCCTCGTCACCGTCTCTTCA |
| A049/A059/A056 V_{L} | GTTGTGATGACCCAGACTCCATCCTCCGTGTCTGAACCTGTG |
| (SEQ ID NO: 53) | GGAGGCACAGTCACCATCAAGTGCcaggccagtcagagcattagcgacc |
| | tcttatccTGGTATCAGCAGAAACCAGGGCAGCCTCCCAAGCTCC |
| | TGATCTATtctgcatccactctggcatctGGGGTCTCATCGCGGTTCAAA |
| | GGCAGTGGATCTGGGACAGAATTCACTCTCACCATCAGCGAC |
| | CTGGAGTGTGCCGATGCTGCCACTTACTACTGTcaatgtaattattat |
| | agtcctacttatgggaatggtTTCGGCGGAGGGACCGAGGTGGTGGTCA |
| | AA |
| A050 V_{H} | CAGTCATTGGAGGAGTCCGGGGGAGACCTGGTCAAGCCTGG |
| (SEQ ID NO: 54) | GGCATCCCTGACACTCACCTGCACAGCCTCTGGATTCTCCTT |
| | CAGTagcagctattggctatgcTGGGCCCGCCAGGCTCCAGGGAAGG |
| | GGCCTGAGTGGATCGCAtgcatttataatggtgatggcagcacacactacgcga |
| | gctgggcgaaaggcCGATTCACCATCTCCAAATCCTCGTCGACCAC |
| | GGTGACTCTGCAAATGACCAGTCTGACAGCCGCGGACACGG |
| | CCACCTATTTCTGTGCGAGAgattatacttataatttttatacttatggttttaatttg |
| | TGGGGCCCAGGCACCCTGGTCACCGTCTCTTCA |
| A050 V_{L} | TTCGAATTGACCCAGACTCCATCCTCCGTGGAGGCAGCTGTG |
| (SEQ ID NO: 55) | GGAGGCACAGTCACCATCAACTGCcaggccagtcagagcgtgaatatttg |
| | ggcatccTGGTACCAGCAGAAACCAGGGCAGCCTCCCAAGCTCC |
| | TGATCTACaaggcatccactctggcatctGGGGTCCCATCGCGGTTCAA |
| | AGGCAGTGGATCTGGGACAGAGTTCACTCTCACCATCAGCGA |
| | CCTGGAGTGTGCCGATGCTGCCACTTACTACTGTcaaggcggttat |
| | cctagtagtagtagtggttgggctTTCGGCGGAGGGACCGAGGTGGTGGT |
| | CAAA |
| A054 V_{H} | CAGGAGCAGGTGGTGGAGTCCGGGGGAGGCCTGGTCAAGCC |
| (SEQ ID NO: 56) | TGGGGCATCCCTGACACTCACCTGCACAGCCTCTGGATTCAC |
| | CCTCAGTagtagttattggatatgcTGGGTCCGCCAGGCTCCAGGGAA |
| | GGGGCCTGAGTGGATCGCAtgcacttataatggtgatggcagcacacactac |
| | gcgagctgggcgaaaggcCGATTCACCATCTCCAAATCCTCGTCGAC |
| | CACGGTGACTCTGCAAATGACCAGTCTGACAGCCGCGGACAC |
| | GGCCACCTATTTCTGTGCGAGAgattatactgatgctttttatacttatggttttaa |
| | tttgTGGGGCCCAGGCACCCTGGTCACCGTCTCTTCA |
| A054 V_{L} | GACATTGTGATGACCCAGACTCCAGCCTCCGTGTCTGAACCT |
| (SEQ ID NO: 57) | GTGGGAGGCACAGTCACCATCAAGTGCcaggccagtcagagcactagt |
| | agctacttagccTGGTATCAGCAGAAACCAGGGCAGCCTCCCAAGC |
| | TCCTGATCTACagggcatccagtctggcatctGGGGTCCCATCGCGGTT |
| | CAAAGGCAGTGGATCTGCGACAGAGTTCACTCTCACCATCAG |
| | CGACCTGGAGTGTGCCGATGCTGCCACTTACTACTGTcaaactg |
| | gtttttatggtagtagtgggcatactTTCGGCGGAGGGACCGAGGTGGTGG |
| | TCAAA |
| A56-CAR-01 V_{L} | GACGTTGTAATGACTCAGACGCCATCCTCCGTTAGCGAACCA |
| (SEQ ID NO: 186) | GTCGGAGGTACAGTGACCATAAAGTGCcaagcctctcagtctatatctga |
| | cctgttgagtTGGTATCAACAGAAACCGGGACAACCCCCAAAGTTG |
| | CTCATCTACtctgcgtcaacactcgcatcaGGCGTCTCAAGCAGATTCA |
| | AAGGGAGTGGGTCCGGTACGGAATTTACTCTTACCATAAGTG |
| | ACCTTGAGTGCGCTGACGCTGCTACTTATTATTGCcagtgtaactac |
| | tattctccgacctacggaaatgggTTCGGAGGCGGAACTGAGGTAGTGG |
| | TTAAAAAG |
| A56-CAR-029 VL | GACGTTGTAATGACTCAGACGCCATCCTCCGTTAGCGAACCA |
| (SEQ ID NO: 187) | GTCGGAGGTACAGTGACCATAAAGTGCcaagcctctcagtctatatctga |
| | cctgttgagtTGGTATCAACAGAAACCGGGACAACCCCCAAAGTTG |
| | CTCATCTACtctgcgtcaacactcgcatcaGGCGTCTCAAGCAGATTCA |
| | AAGGGAGTGGGTCCGGTACGGAATTTACTCTTACCATAAGTG |
| | ACCTTGAGTGCGCTGACGCTGCTACTTATTATTGCcagtgtaactac |
| | tattctccgacctacggaaatgggTTCGGAGGCGGAACTGAGGTAGTGG |
| | TTAAA |

According to some embodiments, provided are nucleic acids that encode the variable heavy chain (V_{H}) polypeptide, the variable light chain (V_{L}) polypeptide, or both, of the anti-VV B5 antibodies A048/A058/A073 or A051. Examples of such nucleotide sequences are provided in Table 13 below. Sequences that encode framework regions and CDRs are shown in upper case and lower case, respectively.

TABLE 13
Nucleotide Sequences Encoding Example Anti-VV B5 Antibodies

| | |
|---|---|
| A048/A058/A073 V_{H} | CAGGAGCAGCTGGAGGAGTCCGGGGGAGGCCTGGTCAAGCC |
| (SEQ ID NO: 58) | TGAGGGATCCCTGACACTCACCTGCACAGCTTCTGGATTCTC |
| | CTTCAGTagcagctactacatgtgcTGGGTCCGCCAGGCTCCAGGGA |
| | GGGGGCTGGAGTGGATCGCAtgcatttatactagtagtggtagcgcttactacg |
| | cgaactgggcgaaaggcCGATTCACCATCTCCAGAACCTCGTCGACC |
| | ACGGTGACTCTGCAAATGACCCGTCTGACAGCCGCGGACACG |
| | GCCACCTATTTCTGTGTGAGAaacgctgttgggagtagttattatttgtatttgT |
| | GGGGCCCAGGCACCCTGGTCACCGTCTCCTCA |
| A048/A058/A073 V_{L} | CAAGTGCTGACCCAGACTCCATCCCCTGTGTCTGCAGCTGTG |
| (SEQ ID NO: 59) | GGAGGCACAGTCACCATCAGTTGCcaggccagtcagagtgttgctggtaa |
| | caactacttatccTGGTATCAGCAGAAACCAGGGCAGCCTCCCAAC |
| | CTCTTGATCTATtctgtatccactctggcctctGGGGTCCCATCGCGGTT |
| | CAAAGGCAGTGGATCTGGGACACAGTTCACTCTCACCATCAG |
| | CGACCTGGAGTGTGACGATGCTGCCACTTACTACTGTcaaggct |
| | attataatgatggaatttgggctTTCGGCGGAGGGACCGAGGTGGTGGTC |
| | AAA |
| A051 V_{H} | CAGGAGCAGGTGGAGGAGTCCGGGGGAGGCCTGGCCAAGC |
| (SEQ ID NO: 60) | CTGGGGCATCTTTGACACTCACCTGCGAAGCCTCTGGATTCA |
| | CCCTCAGTagttactggatgtgcTGGGTCCGCCAGGCTCCAGGGAAG |
| | GGGCTGGAGTGGATCGCAtgcatttatggtgggagtagtggtagcacttactact |
| | cgaactgggcgaaaggcCGATTCACCATCTCCAAAACCTCGTCGACC |
| | ACGGTGACTCTGCAAATGACCAGTCTGACAGCCGCGGACACG |
| | GCCACCTATTTCTGTGCGAGAgatggtagtacctgggactactttaggttgTG |
| | GGGCCCAGGCACCCTGGTCACCGTCTCTTCA |
| A051 V_{L} | TATGATATGACCCAGACTCCATCCTCCGTGGAGGCAGCTGTG |
| (SEQ ID NO: 61) | GGAGGCACAGTCACCATCAAGTGCcaggccagtcagagtattaatactaa |
| | ctacttatccTGGTATCAGCAGAAACCAGGGCAGCCTCCCAAGCTC |
| | CTGATCTACcaggcatccactctggaatctGGGGTCCCATCGCGGTTCA |
| | AAGGCAGTGGATCTGGGACAGAGTACACTCTCACCATCAGCG |
| | ACCTGGAGTGTGCCGATGCTGCCACTTACTACTGTcaaggctattat |
| | actgttgagaatattggtaatcctTTCGGCGGAGGGACCGAGGTGGTGGT |
| | CAAA |

According to some embodiments, provided is a nucleic acid that encodes a CAR of the present disclosure, e.g., a CAR comprising: a single chain antibody comprising a V_{H} polypeptide and a V_{L} polypeptide of an anti-VV A56 or anti-VV B5 antibody of the present disclosure; a transmembrane domain; and an intracellular signaling domain. Examples of such single chain antibodies, transmembrane domains, and intracellular signaling domains are described in detail above.

An example nucleotide sequence that encodes A56-CAR-01 described herein is set forth in SEQ ID NO: 155. An example nucleotide sequence that encodes A56-CAR-02 described herein is set forth in SEQ ID NO: 156. An example nucleotide sequence that encodes A56-CAR-05 described herein is set forth in SEQ ID NO: 157. An example nucleotide sequence that encodes A56-CAR-06 described herein is set forth in SEQ ID NO: 158. An example nucleotide sequence that encodes A56-CAR-07 described herein is set forth in SEQ ID NO: 159. An example nucleotide sequence that encodes A56-CAR-08 described herein is set forth in SEQ ID NO: 160. An example nucleotide sequence that encodes A56-CAR-010 described herein is set forth in SEQ ID NO: 161. An example nucleotide sequence that encodes A56-CAR-020 described herein is set forth in SEQ ID NO: 162. An example nucleotide sequence that encodes A56-CAR-021 described herein is set forth in SEQ ID NO: 163. An example nucleotide sequence that encodes A56-CAR-027 described herein is set forth in SEQ ID NO: 164. An example nucleotide sequence that encodes A56-CAR-028 described herein is set forth in SEQ ID NO: 165. An example nucleotide sequence that encodes A56-CAR-029 described herein is set forth in SEQ ID NO: 166. An example nucleotide sequence that encodes A56-CAR-030 described herein is set forth in SEQ ID NO: 167. An example nucleotide sequence that encodes B5-CAR-03 described herein is set forth in SEQ ID NO: 168. An example nucleotide sequence that encodes B5-CAR-04 described herein is set forth in SEQ ID NO: 169. An example nucleotide sequence that encodes B5-CAR-011 described herein is set forth in SEQ ID NO: 170. An example nucleotide sequence that encodes B5-CAR-013 described herein is set forth in SEQ ID NO: 171. An example nucleotide sequence that encodes B5-CAR-014 described herein is set forth in SEQ ID NO: 172. An example nucleotide sequence that encodes B5-CAR-016 described herein is set forth in SEQ ID NO: 173. An example nucleotide sequence that encodes B5-CAR-019 described herein is set forth in SEQ ID NO: 174. An example nucleotide sequence that encodes B5-CAR-022 described herein is set forth in SEQ ID NO: 175. Examples of nucleotide sequences that encode the murine CARs provided in Table 11 are set forth in SEQ ID NOs: 176-179.

Also provided are expression vectors comprising any of the nucleic acids of the present disclosure. Expression of natural or synthetic nucleic acids encoding the antibodies and fusion proteins of the present disclosure can be achieved by operably linking a nucleic acid encoding the antibody or fusion protein to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. According to some embodiments, a cell of the present disclosure includes a nucleic acid that encodes the V_{H} polypeptide of the antibody and the V_{L} polypeptide of the antibody. In certain such embodiments, the antibody is a single chain antibody (e.g., an scFv), and the nucleic acid encodes the single chain antibody. According to some embodiments, provided is a cell comprising a first nucleic acid encoding a variable heavy chain (V_{H}) polypeptide of an antibody of the present disclosure, and a second nucleic acid encoding a variable light chain (V_{L}) polypeptide of the antibody. In certain embodiments, such as cell comprises a first expression vector comprising the first nucleic acid, and a second expression vector comprising the second nucleic acid.

Also provided are methods of making an antibody or fusion protein of the present disclosure, the methods including culturing a cell of the present disclosure under conditions suitable for the cell to express the antibody or fusion protein, wherein the antibody or fusion protein is produced. The conditions for culturing the cell such that the antibody or fusion protein is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

Chimeric Antigen Receptors that Bind Oncolytic Virus Antigens

In addition to the chimeric antigen receptors (CARs) of the present disclosure described in the Fusion Protein section above, aspects of the present disclosure further include anti-oncolytic virus antigen CARs. In certain embodiments, such a CAR comprises an antigen binding domain that specifically binds an oncolytic virus (OV) antigen, a transmembrane domain, and an intracellular signaling domain. Such CARs find a variety of uses. For example, with the benefit of the present disclosure, it will be appreciated that such CARs find use in methods that comprise administering a pharmaceutical composition comprising an anti-OV antigen CAR to an individual having cancer, wherein cancer cells in the individual are infected with OV and express the OV antigen on their surface, thereby targeting the CAR to the cancer cells to treat the individual's cancer.

As used herein, an “oncolytic virus antigen” or “OV antigen” is an antigen encoded by the genome of an oncolytic virus (OV). In some embodiments, the OV antigen is a native OV antigen, whereby “native” is meant the antigen is a protein encoded by the wild-type genome of the OV.

In some embodiments, the OV antigen is an antigen encoded by the wild-type genome of a virus from the Poxviridae, Herpesviridae, Adenoviridae, Paramyxoviridae, Rhabdoviridae, Reoviridae, Picornaviridae, Parvoviridae, or Coronaviridae family of viruses. Such an OV antigen may be native or heterologous to the OV.

In certain embodiments, the OV antigen is an antigen encoded by the wild-type genome of a virus from a family set forth in Table 14 below, where Table 14 further includes non-limiting examples of virus species/strains within such families as well as non-limiting examples of such antigens.

TABLE 14
Example OV Antigens

| | Virus Species | | |
|---|---|---|---|
| Family | and/or Strain | Genome | Example Antigens |
| Poxviridae | Vaccinia Virus | dsDNA | A33, A34, A36, A56, B5, F12, F13 |
| Herpesviridae | Herpes | dsDNA | gB, gC, gD, gH, gL |
| | simplex virus | | |
| | type 1/2 | | |
| Adenoviridae | Adenovirus | dsDNA | Fiber, Penton, Hexon, IX, IIIa, VI, VII |
| Paramyxoviridae | Measles | ssRNA (−) | Hemagglutinin glycoprotein, |
| | | | Fusion glycoprotein |
| | Newcastle | ssRNA (−) | Hemagglutinin-neuraminidase, |
| | Disease Virus | | Fusion glycoprotein |
| Rhabdoviridae | Vesicular | ssRNA (−) | Glycoprotein G |
| | stomatitis virus | | |
| | Maraba | ssRNA (−) | Glycoprotein G |
| Reoviridae | Reovirus | dsRNA | μ1, σ3, σ1, λ2, λ1 |
| Picornaviridae | Seneca Valley | ssRNA (+) | VP1, VP2, VP3, VP4 |
| | Virus | | |
| | Poliovirus | ssRNA (+) | VP1, VP2, VP3, VP4 |
| | Coxsackievirus | ssRNA (+) | VP1, VP2, VP3, VP4 |
| Parvoviridae | Human | ssDNA | VP2, VP1 |
| | Parvovirus | | |
| Coronaviridae | SARS | ssRNA (+) | Spike (S), Envelope (E), Nucleoprotein (N), |
| | coronavirus 2 | | Membrane (M), ORF3a, ORF7a |

According to some embodiments, the OV antigen is an antigen encoded by the wild-type genome of a virus from the Poxviridae family selected from Amsacta moorei entomopoxvirus, Chironomus luridus entomopoxvirus, Fowlpox virus, Melolontha melolontha entomopoxvirus, Molluscum contagiosum virus, Mule deerpox virus, Myxoma virus, Nile crocodilepox virus, Orf virus, Sheeppox virus, Swinepox virus, Vaccinia virus, Yaba monkey tumor virus, Yokapox virus, Acrobasis zelleri entomopoxvirus, Adoxophyes honmai entomopoxvirus, Aedes aegypti entomopoxvirus, Anomala cuprea entomopoxvirus, Aphodius tasmaniae entomopoxvirus, Arphia conspersa entomopoxvirus, Bovine papular stomatitis virus, Camelpox virus, Camptochironomus tentans entomopoxvirus, Canarypox virus, Chironomus attenuatus entomopoxvirus, Chironomus plumosus entomopoxvirus, Choristoneura biennis entomopoxvirus, Choristoneura conflicta entomopoxvirus, Choristoneura diversuma entomopoxvirus, Choristoneura fumiferana entomopoxvirus, Choristoneura rosaceana entomopoxvirus, Chorizagrotis auxiliaris entomopoxvirus, Cowpox virus, Demodema bonariensis entomopoxvirus, Dermolepida albohirtum entomopoxvirus, Diachasmimorpha entomopoxvirus, Ectromelia virus, Figulus sublaevis entomopoxvirus, Geotrupes sylvaticus entomopoxvirus, Goatpox virus, Goeldichironomus holoprasinus entomopoxvirus, Hare fibroma virus, Heliothis armigera entomopoxvirus, Juncopox virus, Locusta migratoria entomopoxvirus, Lumpy skin disease virus, Melanoplus sanguinipes entomopoxvirus, Monkeypox virus, Mynahpox virus, Mythimna separata entomopoxvirus, Oedaleus senegalensis entomopoxvirus, Operophtera brumata entomopoxvirus, Parapoxvirus of red deer in New Zealand, Pigeonpox virus, Pseudocowpox virus, Psittacinepox virus, Pteropox virus, Quailpox virus, Rabbit fibroma virus, Raccoonpox virus, Schistocerca gregaria entomopoxvirus, Skunkpox virus, Sparrowpox virus, Squirrel fibroma virus, Squirrelpox virus, Starlingpox virus, Tanapox virus, Taterapox virus, Turkeypox virus, Variola virus, and Volepox virus. Such an OV antigen may be native or heterologous to the OV.

In certain embodiments, the OV antigen is an antigen encoded by the wild-type genome of a virus from the Herpesviridae family selected from Gallid alphaherpesvirus 1, Psittacid alphaherpesvirus 1, Gallid alphaherpesvirus 2, Anatid alphaherpesvirus 1, Columbid alphaherpesvirus 1, Gallid alphaherpesvirus 3, Meleagrid alphaherpesvirus 1, Chelonid alphaherpesvirus 5, Human alphaherpesvirus 1, Ateline alphaherpesvirus 1, Bovine alphaherpesvirus 2, Cercopithecine alphaherpesvirus 2, Human alphaherpesvirus 2, Leporid alphaherpesvirus 4, Macacine alphaherpesvirus 1, Macropodid alphaherpesvirus 1, Macropodid alphaherpesvirus 2, Panine alphaherpesvirus 3, Papiine alphaherpesvirus 2, Saimiriine alphaherpesvirus 1, Human betaherpesvirus 5, Aotine betaherpesvirus 1, Cebine betaherpesvirus 1, Cercopithecine betaherpesvirus 5, Macacine betaherpesvirus 3, Panine betaherpesvirus 2, Papiine betaherpesvirus 3, Saimiriine betaherpesvirus 4, Murid betaherpesvirus 1, Murid betaherpesvirus 2, Murid betaherpesvirus 8, Human betaherpesvirus 6A, Human betaherpesvirus 6B, Human betaherpesvirus 7, Elephantid betaherpesvirus 1, Human gammaherpesvirus 4, Callitrichine gammaherpesvirus 3, Cercopithecine gammaherpesvirus 14, Gorilline gammaherpesvirus 1, Macacine gammaherpesvirus 4, Panine gammaherpesvirus 1, Papiine gammaherpesvirus 1, Pongine gammaherpesvirus 2, Saimiriine gammaherpesvirus 2, Ateline gammaherpesvirus 2, Ateline gammaherpesvirus 3, Bovine gammaherpesvirus 4, Cricetid gammaherpesvirus 2, Human gammaherpesvirus 8, Macacine gammaherpesvirus 5, Murid gammaherpesvirus 4, Murid gammaherpesvirus 7, Alcelaphine gammaherpesvirus 1, Alcelaphine gammaherpesvirus 2, Bovine gammaherpesvirus 6, Caprine gammaherpesvirus 2, Hippotragine gammaherpesvirus 1, Ovine gammaherpesvirus 2, Suid gammaherpesvirus 3, Suid gammaherpesvirus 4, Suid gammaherpesvirus 5, Equid gammaherpesvirus 2, Equid gammaherpesvirus 5, and Mustelid gammaherpesvirus 1. Such an OV antigen may be native or heterologous to the OV.

According to some embodiments, the OV antigen is an antigen encoded by the wild-type genome of a virus from the Adenoviridae family selected from Fowl aviadenovirus A, Frog siadenovirus A, Human mastadenovirus C, Ovine atadenovirus D, Sturgeon ichtadenovirus A, Bat mastadenovirus A, Bat mastadenovirus B, Bat mastadenovirus C, Bat mastadenovirus D, Bat mastadenovirus E, Bat mastadenovirus F, Bat mastadenovirus G, Bovine atadenovirus D, Bovine mastadenovirus A, Bovine mastadenovirus B, Bovine mastadenovirus C, Canine mastadenovirus A, Deer atadenovirus A, Deer mastadenovirus B, Dolphin mastadenovirus A, Dolphin mastadenovirus B, Duck atadenovirus A, Duck aviadenovirus B, Equine mastadenovirus A, Equine mastadenovirus B, Falcon aviadenovirus A, Fowl aviadenovirus B, Fowl aviadenovirus C, Fowl aviadenovirus D, Fowl aviadenovirus E, Goose aviadenovirus A, Great tit siadenovirus A, Human mastadenovirus A, Human mastadenovirus B, Human mastadenovirus D, Human mastadenovirus E, Human mastadenovirus F, Human mastadenovirus G, Lizard atadenovirus A, Murine mastadenovirus A, Murine mastadenovirus B, Murine mastadenovirus C, Ovine mastadenovirus A, Ovine mastadenovirus B, Penguin siadenovirus A, Pigeon aviadenovirus A, Pigeon aviadenovirus B, Platyrrhini mastadenovirus A, Porcine mastadenovirus A, Porcine mastadenovirus B, Porcine mastadenovirus C, Possum atadenovirus A, Psittacine atadenovirus A, Psittacine aviadenovirus B, Raptor siadenovirus A, Sea lion mastadenovirus A, Simian mastadenovirus A, Simian mastadenovirus B, Simian mastadenovirus C, Simian mastadenovirus D, Simian mastadenovirus E, Simian mastadenovirus F, Simian mastadenovirus G, Simian mastadenovirus H, Simian mastadenovirus I, Skua siadenovirus A, Skunk mastadenovirus A, Snake atadenovirus A, Squirrel mastadenovirus A, Tree shrew mastadenovirus A, Turkey aviadenovirus B, Turkey aviadenovirus C, Turkey aviadenovirus D, and Turkey siadenovirus A. Such an OV antigen may be native or heterologous to the OV.

In certain embodiments, the OV antigen is an antigen encoded by the wild-type genome of a virus from the Paramyxoviridae family selected from Avian avulavirus 1, Hendra henipavirus, Measles morbillivirus, Mumps rubulavirus, Murine respirovirus, Reptilian ferlavirus, Salmon aquaparamyxovirus, Achimota rubulavirus 1, Achimota rubulavirus 2, Avian avulavirus 10, Avian avulavirus 11, Avian avulavirus 12, Avian avulavirus 13, Avian avulavirus 14, Avian avulavirus 15, Avian avulavirus 16, Avian avulavirus 17, Avian avulavirus 18, Avian avulavirus 19, Avian avulavirus 2, Avian avulavirus 3, Avian avulavirus 4, Avian avulavirus 5, Avian avulavirus 6, Avian avulavirus 7, Avian avulavirus 8, Avian avulavirus 9, Bat mumps rubulavirus, Bovine respirovirus 3, Canine morbillivirus, Cedar henipavirus, Cetacean morbillivirus, Feline morbillivirus, Ghanaian bat henipavirus, Human respirovirus 1, Human respirovirus 3, Human rubulavirus 2, Human rubulavirus 4, Mammalian rubulavirus 5, Mapuera rubulavirus, Menangle rubulavirus, Mojiang henipavirus, Nipah henipavirus, Phocine morbillivirus, Porcine respirovirus 1, Porcine rubulavirus, Rinderpest morbillivirus, Simian rubulavirus, Small ruminant morbillivirus, Sosuga rubulavirus, Teviot rubulavirus, Tioman rubulavirus, Tuhoko rubulavirus 1, Tuhoko rubulavirus 2, and Tuhoko rubulavirus 3. Such an OV antigen may be native or heterologous to the OV.

According to some embodiments, the OV antigen is an antigen encoded by the wild-type genome of a virus from the Rhabdoviridae family selected from Bovine fever ephemerovirus, Carp sprivivirus, Curionopolis curiovirus, Drosophila melanogaster sigmavirus, Durham tupavirus, Flanders hapavirus, Indiana vesiculovirus, Le Dantec ledantevirus, Lettuce big-vein associated varicosavirus, Lettuce necrotic yellows cytorhabdovirus, Niakha sripuvirus, Orchid fleck dichorhavirus, Perch perhabdovirus, Potato yellow dwarf nucleorhabdovirus, Puerto Almendras almendravirus, Rabies lyssavirus, Salmonid novirhabdovirus, Tibrogargan tibrovirus, Adelaide River ephemerovirus, Alagoas vesiculovirus, Alfalfa dwarf cytorhabdovirus, Almpiwar sripuvirus, American bat vesiculovirus, Anguillid perhabdovirus, Aravan lyssavirus, Arboretum almendravirus, Australian bat lyssavirus, Balsa almendravirus, Barley yellow striate mosaic cytorhabdovirus, Barur ledantevirus, Bas-Congo tibrovirus, Beatrice Hill tibrovirus, Berrimah ephemerovirus, Bokeloh bat lyssavirus, Broccoli necrotic yellows cytorhabdovirus, Carajas vesiculovirus, Chaco sripuvirus, Chandipura vesiculovirus, Coastal Plains tibrovirus, Cocal vesiculovirus, Coffee ringspot dichorhavirus, Colocasia bobone disease-associated cytorhabdovirus, Coot Bay almendravirus, Datura yellow vein nucleorhabdovirus, Drosophila affinis sigmavirus, Drosophila ananassae sigmavirus, Drosophila immigrans sigmavirus, Drosophila obscura sigmavirus, Drosophila tristis sigmavirus, Duvenhage lyssavirus, Eggplant mottled dwarf nucleorhabdovirus, Ekpoma 1 tibrovirus, Ekpoma 2 tibrovirus, European bat 1 lyssavirus, European bat 2 lyssavirus, Festuca leaf streak cytorhabdovirus, Fikirini ledantevirus, Fukuoka ledantevirus, Gannoruwa bat lyssavirus, Gray Lodge hapavirus, Hart Park hapavirus, Hirame novirhabdovirus, Ikoma lyssavirus, Iriri curiovirus, Irkut lyssavirus, Isfahan vesiculovirus, Itacaiunas curiovirus, Joinjakaka hapavirus, Jurona vesiculovirus, Kamese hapavirus, Kanyawara ledantevirus, Kern Canyon ledantevirus, Keuraliba ledantevirus, Khujand lyssavirus, Kimberley ephemerovirus, Klamath tupavirus, Kolente ledantevirus, Koolpinyah ephemerovirus, Kotonkan ephemerovirus, Kumasi ledantevirus, La Joya hapavirus, Lagos bat lyssavirus, Landjia hapavirus, Lettuce yellow mottle cytorhabdovirus, Lleida bat lyssavirus, Maize Iranian mosaic nucleorhabdovirus, Maize fine streak nucleorhabdovirus, Maize mosaic nucleorhabdovirus, Malpais Spring vesiculovirus, Manitoba hapavirus, Maraba vesiculovirus, Marco hapavirus, Mokola lyssavirus, Morreton vesiculovirus, Mosqueiro hapavirus, Mossuril hapavirus, Mount Elgon bat ledantevirus, Moussa virus, Muscina stabulans sigmavirus, New Jersey vesiculovirus, Ngaingan hapavirus, Nishimuro ledantevirus, Nkolbisson ledantevirus, Northern cereal mosaic cytorhabdovirus, Obodhiang ephemerovirus, Oita ledantevirus, Ord River hapavirus, Parry Creek hapavirus, Perinet vesiculovirus, Pike fry sprivivirus, Piry vesiculovirus, Piscine novirhabdovirus, Radi vesiculovirus, Rice yellow stunt nucleorhabdovirus, Rio Chico almendravirus, Rochambeau curiovirus, Sea trout perhabdovirus, Sena Madureira sripuvirus, Shimoni bat lyssavirus, Snakehead novirhabdovirus, Sonchus cytorhabdovirus 1, Sonchus yellow net nucleorhabdovirus, Sowthistle yellow vein nucleorhabdovirus, Sripur sripuvirus, Strawberry crinkle cytorhabdovirus, Sweetwater Branch tibrovirus, Taro vein chlorosis nucleorhabdovirus, Tupaia tupavirus, West Caucasian bat lyssavirus, Wheat American striate mosaic cytorhabdovirus, Wongabel hapavirus, Wuhan ledantevirus, Yata ephemerovirus, Yongjia ledantevirus, and Yug Bogdanovac vesiculovirus. Such an OV antigen may be native or heterologous to the OV.

In certain embodiments, the OV antigen is an antigen encoded by the wild-type genome of a virus from the Reoviridae family selected from Aedes pseudoscutellaris reovirus, Aquareovirus A, Banna virus, Bluetongue virus, Colorado tick fever virus, Cypovirus 1, Eriocheir sinensis reovirus, Fiji disease virus, Idnoreovirus 1, Mammalian orthoreovirus, Micromonas pusilla reovirus, Mycoreovirus 1, Rice ragged stunt virus, Rotavirus A, Wound tumor virus, African horse sickness virus, Aquareovirus B, Aquareovirus C, Aquareovirus D, Aquareovirus E, Aquareovirus F, Aquareovirus G, Avian orthoreovirus, Baboon orthoreovirus, Changuinola virus, Chenuda virus, Chobar Gorge virus, Corriparta virus, Cypovirus 10, Cypovirus 11, Cypovirus 12, Cypovirus 13, Cypovirus 14, Cypovirus 15, Cypovirus 16, Cypovirus 2, Cypovirus 3, Cypovirus 4, Cypovirus 5, Cypovirus 6, Cypovirus 7, Cypovirus 8, Cypovirus 9, Echinochloa ragged stunt virus, Epizootic hemorrhagic disease virus, Equine encephalosis virus, Eubenangee virus, Eyach virus, Garlic dwarf virus, Great Island virus, Idnoreovirus 2, Idnoreovirus 3, Idnoreovirus 4, Idnoreovirus 5, Ieri virus, Kadipiro virus, Lebombo virus, Liao ning virus, Mahlapitsi orthoreovirus, Maize rough dwarf virus, Mal de Rio Cuarto virus, Mycoreovirus 2, Mycoreovirus 3, Nelson Bay orthoreovirus, Nilaparvata lugens reovirus, Oat sterile dwarf virus, Orungo virus, Palyam virus, Pangola stunt virus, Peruvian horse sickness virus, Piscine orthoreovirus, Reptilian orthoreovirus, Rice black streaked dwarf virus, Rice dwarf virus, Rice gall dwarf virus, Rotavirus B, Rotavirus C, Rotavirus D, Rotavirus E, Rotavirus F, Rotavirus G, Rotavirus H, Rotavirus I, Southern rice black-streaked dwarf virus, St Croix River virus, Umatilla virus, Wad Medani virus, Wallal virus, Warrego virus, Wongorr virus, and Yunnan orbivirus. Such an OV antigen may be native or heterologous to the OV.

According to some embodiments, the OV antigen is an antigen encoded by the wild-type genome of a virus from the Picornaviridae family selected from Aalivirus A, Aichivirus A, Ampivirus A, Aquamavirus A, Avihepatovirus A, Avisivirus A, Bopivirus A, Cadicivirus A, Cardiovirus A, Cosavirus A, Crohivirus B, Enterovirus C, Erbovirus A, Foot-and-mouth disease virus, Gallivirus A, Harkavirus A, Hepatovirus A, Hunnivirus A, Kunsagivirus A, Limnipivirus A, Megrivirus A, Mischivirus A, Mosavirus A, Orivirus A, Oscivirus A, Parechovirus A, Pasivirus A, Passerivirus A, Potamipivirus A, Rabovirus A, Rosavirus A, Sakobuvirus A, Salivirus A, Sapelovirus A, Senecavirus A, Shanbavirus A, Sicinivirus A, Teschovirus A, Torchivirus A, Tremovirus A, Aichivirus B, Aichivirus C, Aichivirus D, Aichivirus E, Aichivirus F, Avian sapelovirus, Avisivirus B, Avisivirus C, Bovine rhinitis A virus, Bovine rhinitis B virus, Cardiovirus B, Cardiovirus C, Cosavirus B, Cosavirus D, Cosavirus E, Cosavirus F, Crohivirus A, Enterovirus A, Enterovirus B, Enterovirus D, Enterovirus E, Enterovirus F, Enterovirus G, Enterovirus H, Enterovirus I, Enterovirus J, Enterovirus K, Enterovirus L, Equine rhinitis A virus, Hepatovirus B, Hepatovirus C, Hepatovirus D, Hepatovirus E, Hepatovirus F, Hepatovirus G, Hepatovirus H, Hepatovirus I, Kunsagivirus B, Kunsagivirus C, Limnipivirus B, Limnipivirus C, Megrivirus B, Megrivirus C, Megrivirus D, Megrivirus E, Mischivirus B, Mischivirus C, Parechovirus B, Parechovirus C, Parechovirus D, Rhinovirus A, Rhinovirus B, Rhinovirus C, and Sapelovirus B. Such an OV antigen may be native or heterologous to the OV.

In certain embodiments, the OV antigen is an antigen encoded by the wild-type genome of a virus from the Parvoviridae family selected from Adeno-associated dependoparvovirus A, Carnivore amdoparvovirus 1, Decapod hepandensovirus 1, Decapod penstyldensovirus 1, Dipteran brevidensovirus 1, Galliform aveparvovirus 1, Lepidopteran ambidensovirus 1, Lepidopteran iteradensovirus 1, Primate erythroparvovirus 1, Primate tetraparvovirus 1, Rodent protoparvovirus 1, Ungulate bocaparvovirus 1, Ungulate copiparvovirus 1, Adeno-associated dependoparvovirus B, Anseriform dependoparvovirus 1, Asteroid ambidensovirus 1, Avian dependoparvovirus 1, Blattodean ambidensovirus 1, Blattodean ambidensovirus 2, Carnivore amdoparvovirus 2, Carnivore amdoparvovirus 3, Carnivore amdoparvovirus 4, Carnivore bocaparvovirus 1, Carnivore bocaparvovirus 2, Carnivore bocaparvovirus 3, Carnivore bocaparvovirus 4, Carnivore bocaparvovirus 5, Carnivore bocaparvovirus 6, Carnivore protoparvovirus 1, Chiropteran bocaparvovirus 1, Chiropteran bocaparvovirus 2, Chiropteran bocaparvovirus 3, Chiropteran bocaparvovirus 4, Chiropteran dependoparvovirus 1, Chiropteran protoparvovirus 1, Chiropteran tetraparvovirus 1, Decapod ambidensovirus 1, Dipteran ambidensovirus 1, Dipteran brevidensovirus 2, Eulipotyphla protoparvovirus 1, Hemipteran ambidensovirus 1, Hemipteran ambidensovirus 2, Hemipteran ambidensovirus 3, Hymenopteran ambidensovirus 1, Lagomorph bocaparvovirus 1, Lepidopteran iteradensovirus 2, Lepidopteran iteradensovirus 3, Lepidopteran iteradensovirus 4, Lepidopteran iteradensovirus 5, Orthopteran ambidensovirus 1, Orthopteran densovirus 1, Pinniped bocaparvovirus 1, Pinniped bocaparvovirus 2, Pinniped dependoparvovirus 1, Primate bocaparvovirus 1, Primate bocaparvovirus 2, Primate erythroparvovirus 2, Primate erythroparvovirus 3, Primate erythroparvovirus 4, Primate protoparvovirus 1, Primate protoparvovirus 2, Primate protoparvovirus 3, Rodent erythroparvovirus 1, Rodent protoparvovirus 2, Rodent protoparvovirus 3, Squamate dependoparvovirus 1, Ungulate bocaparvovirus 2, Ungulate bocaparvovirus 3, Ungulate bocaparvovirus 4, Ungulate bocaparvovirus 5, Ungulate bocaparvovirus 6, Ungulate copiparvovirus 2, Ungulate erythroparvovirus 1, Ungulate protoparvovirus 1, Ungulate protoparvovirus 2, Ungulate tetraparvovirus 1, Ungulate tetraparvovirus 2, Ungulate tetraparvovirus 3, and Ungulate tetraparvovirus 4. Such an OV antigen may be native or heterologous to the OV.

According to some embodiments, the OV antigen is an antigen encoded by the wild-type genome of a virus from the Coronaviridae family selected from Alphacoronavirus 1, Avian coronavirus, Bulbul coronavirus HKU11, Equine torovirus, Murine coronavirus, White bream virus, Ball python nidovirus 1, Bat coronavirus CDPHE15, Bat coronavirus HKU10, Beluga whale coronavirus SW1, Betacoronavirus 1, Bovine nidovirus 1, Bovine torovirus, Chinook salmon nidovirus 1, Common moorhen coronavirus HKU21, Coronavirus HKU15, Fathead minnow nidovirus 1, Hedgehog coronavirus 1, Human coronavirus 229E, Human coronavirus HKU1, Human coronavirus NL63, Human torovirus, Middle East respiratory syndrome-related coronavirus, Miniopterus bat coronavirus 1, Miniopterus bat coronavirus HKU8, Mink coronavirus 1, Munia coronavirus HKU13, Night heron coronavirus HKU19, Pipistrellus bat coronavirus HKU5, Porcine epidemic diarrhea virus, Porcine torovirus, Rhinolophus bat coronavirus HKU2, Rousettus bat coronavirus HKU9, SARS coronavirus 2, Scotophilus bat coronavirus 512, Severe acute respiratory syndrome-related coronavirus, Thrush coronavirus HKU12, Tylonycteris bat coronavirus HKU4, White-eye coronavirus HKU16, Wigeon coronavirus HKU20, and SARS-CoV-2. Such an OV antigen may be native or heterologous to the OV.

In certain embodiments, the OV antigen is an antigen encoded by the genome of a Vaccinia Virus (e.g., antigen A33, A34, A36, A56, B5, F12, F13, or the like, e.g., from a modified VV strain, JX-594, GL-ONC1, or a VV strain selected from Western Reserve, Wyeth, Lister, Copenhagen, Temple of Heaven, Patwadangar, and Modified Vaccinia Virus Ankara, and the like), adenovirus, HSV, reovirus, vesicular stomatitis virus, New Castle Disease virus, Seneca Valley virus, poliovirus, measles virus, Coxsackie virus, and Maraba virus. Such an OV antigen may be native or heterologous to the OV.

In certain embodiments, the OV antigen is a modified version (or derivative) of a native OV antigen. For example, according to some embodiments, the OV antigen comprises the same or substantially similar epitope of a native antigen, but is modified/engineered to confer a desirable property, e.g., improved infectivity of the virus, or the like. In certain embodiments, the OV antigen is modified/engineered to have a different epitope relative to a native antigen, e.g., such that an existing antibody (e.g., an antibody approved for therapeutic use) binds to the modified/engineered OV antigen whereas the antibody does not bind to the native antigen.

According to some embodiments, the genome of the OV may be modified to encode and express one or more proteins not encoded by the wild-type genome of the OV, where such proteins may be referred to herein as OV antigens “heterologous” or “non-native” to the OV. The genome of an OV may be modified to encode and express any heterologous OV antigen of interest. In certain embodiments, the heterologous OV antigen is a viral antigen (e.g., an antigen encoded by the wild-type genome of any of the viruses described elsewhere herein) heterologous to the OV that expresses the antigen. According to some embodiments, the heterologous OV antigen is a tumor antigen, non-limiting examples of which include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), and trophoblast cell-surface antigen (TROP-2).

Non-limiting examples of antigen-binding domains that specifically bind to tumor antigens which an OV genome may be modified to encode and express, and which antigen-binding domains may be employed in an anti-OV CAR or anti-OV conjugate of the present disclosure, include the antigen-binding domains of the following antibodies: Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or a tumor antigen-binding variant thereof. As used herein, “variant” is meant the antigen binding domain specifically binds to the particular antigen (e.g., HER2 for Trastuzumab) but has fewer or more amino acids than the parental antibody (e.g., is a fragment (e.g., scFv) of the parental antibody), has one or more amino acid substitutions relative to the parental antibody, or a combination thereof.

In some embodiments, the antigen-binding domain of an anti-OV antigen CAR or anti-OV antigen conjugate of the present disclosure is from an antibody approved by the United States Food and Drug Administration and/or the European Medicines Agency (EMA) for use as a therapeutic antibody (e.g., for targeting certain disease-associated cells in a patient, etc.), or a fragment thereof (e.g., a single-chain version of such an antibody, such as an scFv version of the antibody) that retains the ability to specifically bind the target antigen.

The antigen binding domain of a CAR of the present disclosure may be any suitable format, e.g., scFv, or the like. Any suitable transmembrane and intracellular signaling domains may be employed, including any of the transmembrane and intracellular signaling domains, as well as costimulatory domains, linker sequences/spacer domains, etc. described in the Fusion Protein section above.

A CAR of the present disclosure may be provided by a single peptide, or may be provided by two or more polypeptides. When the CAR is provided by two or more polypeptides, the CAR may be provided in any useful multi-polypeptide format, including any such multi-peptide formats described above in the Fusion Protein section of the present disclosure.

Also provided are nucleic acids encoding any of the anti-OV antigen CARs of the present disclosure, expression vectors comprising such nucleic acids, and cells comprising such nucleic acids and expression vectors.

According to some embodiments, provided is a cell that expresses an anti-OV antigen CAR on its surface. Cells of interest include, but are not limited to, immune cells. In certain embodiments, the immune cell is an immune effector cell. Non-limiting examples of immune effector cells that may express an anti-OV antigen CAR of the present disclosure on its surface include T cells (that is—the cell may be a CAR T cell), NK cells, NKT cells, macrophages, and the like. Also provided are pharmaceutical compositions comprising any of the cells of the present disclosure that express an anti-OV antigen CAR on its surface. Such compositions may comprise the cells and a pharmaceutically acceptable carrier. Examples of suitable pharmaceutically acceptable carriers are described in detail below.

Conjugates that Bind Oncolytic Virus Antigens

In addition to the anti-VV A56 and anti-VV B5 antibody and fusion protein conjugates of the present disclosure described in the Conjugates section above, aspects of the present disclosure further include anti-oncolytic virus antigen antibody conjugates. In certain embodiments, such a conjugate comprises an antibody that specifically binds an oncolytic virus (OV) antigen, and an agent conjugated to the antibody. According to some embodiments, the agent conjugated to the antibody is selected from a chemotherapeutic agent, a toxin, a radiation sensitizing agent, and a radioactive isotope (e.g., a therapeutic radioactive isotope). The agent may be any of such agents described in the Conjugates section above.

The anti-OV antibody conjugates of the present disclosure find a variety of uses. For example, with the benefit of the present disclosure, it will be appreciated that such conjugates find use in methods that comprise administering a pharmaceutical composition comprising an anti-OV antigen conjugate to an individual having cancer, wherein cancer cells in the individual are infected with OV and express the OV antigen on their surface, thereby targeting the conjugate to the cancer cells to treat the individual's cancer.

The antibody (or fusion protein comprising same) may be selected such that the conjugate specifically binds an OV antigen of interest. The OV antigen may be a native OV antigen. For example, the antibody portion of the conjugate may specifically bind a native antigen encoded by an OV selected from Vaccinia Virus (e.g., antigen A33, A34, A36, A56, B5, F12, F13, or the like, e.g., from JX-594, GL-ONC1, or a VV strain selected from Western Reserve, Wyeth, Lister, Copenhagen, Temple of Heaven, Patwadangar, and Modified Vaccinia Virus Ankara, and the like), adenovirus, HSV, reovirus, vesicular stomatitis virus, New Castle Disease virus, Seneca Valley virus, poliovirus, measles virus, Coxsackie virus, and Maraba virus.

In certain embodiments, the OV antigen is heterologous to the OV. For example, the antibody (or fusion protein comprising the antibody) may be selected such that the antibody portion of the conjugate specifically binds any of the heterologous OV antigens described in the CAR section above.

The antibody portion of an anti-OV antibody conjugate of the present disclosure may be provided in any desired format, e.g., tetrameric format, single chain (e.g., scFv) format, etc. as described in the sections above relating to the antibodies, and anti-VV A56 and anti-VV B5 antibody and fusion protein conjugates, of the present disclosure.

An anti-OV antibody conjugate of the present disclosure may include the agent conjugated to the antibody (or fusion protein comprising the antibody) via a linker, e.g., any of the non-cleavable or cleavable linkers described in the section above relating to the anti-VV A56 and anti-VV B5 antibody and fusion protein conjugates of the present disclosure.

Also provided are pharmaceutical compositions comprising any of the anti-OV antibody conjugates of the present disclosure. Such compositions may comprise the conjugates and a pharmaceutically acceptable carrier. Examples of suitable pharmaceutically acceptable carriers are described in detail below.

### Compositions

As summarized above, the present disclosure also provides compositions. According to some embodiments, a composition of the present disclosure includes an antibody, fusion protein, or conjugate of the present disclosure. For example, the antibody, fusion protein, or conjugate may be any of the antibodies, fusion proteins, or conjugates described in the Antibodies section hereinabove, which descriptions are incorporated but not reiterated herein for purposes of brevity.

In certain aspects, a composition of the present disclosure includes the antibody, fusion protein, or conjugate present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes an anti-VV A56 antibody or anti-VV B5 antibody of the present disclosure (or conjugate or fusion protein comprising same), and a pharmaceutically acceptable carrier.

The antibodies, fusion proteins, or conjugates can be incorporated into a variety of formulations for therapeutic administration. More particularly, the antibodies, fusion proteins, or conjugates can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the antibodies, fusion proteins, or conjugates for administration to an individual (e.g., suitable for human administration) are generally sterile and may further be free of detectable pyrogens or other contaminants contraindicated for administration to a patient according to a selected route of administration.

In pharmaceutical dosage forms, the antibodies, fusion proteins, or conjugates can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the antibodies, fusion proteins, or conjugates can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The antibodies, fusion proteins, or conjugates can be formulated for parenteral (e.g., intravenous, intra-arterial, intraosseous, intramuscular, intracerebral, intracerebroventricular, intrathecal, subcutaneous, etc.) administration. In certain aspects, the antibodies, fusion proteins, or conjugates are formulated for injection by dissolving, suspending or emulsifying the antibodies, fusion proteins, or conjugates in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the antibodies, fusion proteins, or conjugates may be prepared by mixing the antibodies, fusion proteins, or conjugates having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the antibodies, fusion proteins, or conjugates may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylenesorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the antibody and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the antibody, fusion protein, or conjugate, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

Aspects of the present disclosure further include kits. In certain embodiments, the kits find use in practicing the methods of the present disclosure, e.g., methods comprising administering a pharmaceutical composition of the present disclosure to an individual to target the antibody, fusion protein (e.g., CAR) or conjugate to oncolytic virus (e.g. VV)-infected cancer cells in the individual.

Accordingly, in certain embodiments, a kit of the present disclosure comprises any of the pharmaceutical compositions of the present disclosure, and instructions for administering the pharmaceutical composition to an individual in need thereof. The pharmaceutical composition included in the kit may include any of the antibodies, fusion proteins, and/or conjugates of the present disclosure, e.g., any of the antibodies, fusion proteins, and/or conjugates described hereinabove. As will be appreciated, the kits of the present disclosure may include any of the agents and features described above in the sections relating to the subject antibodies, fusion proteins, conjugates and compositions, which are not reiterated herein for purposes of brevity.

The kits of the present disclosure may include a quantity of the compositions, present in unit dosages, e.g., ampoules, or a multi-dosage format. As such, in certain embodiments, the kits may include one or more (e.g., two or more) unit dosages (e.g., ampoules) of a composition that includes an antibody, fusion protein, and/or conjugate of the present disclosure. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular antibody, fusion protein, and/or conjugate employed, the effect to be achieved, and the pharmacodynamics associated with the antibody, fusion protein, and/or conjugate, in the individual. In yet other embodiments, the kits may include a single multi dosage amount of the composition.

In certain embodiments, a kit of the present disclosure includes instructions for targeting the antibody, fusion protein or conjugate present in the pharmaceutical composition to VV-infected cancer cells in an individual having cancer (e.g., to treat the cancer of the individual), e.g., by administering the pharmaceutical composition to the individual, wherein the individual comprises cancer cells infected with VV, and wherein the antibody, fusion protein or conjugate is targeted to the infected cancer cells by VV antigens expressed on the surface of the infected cancer cells.

According to some embodiments, a kit of the present disclosure may further include pharmaceutical composition comprising VV (e.g., JX-594, GL-ONC1, a strain of VV selected from Western Reserve, Wyeth, Lister, Copenhagen, Temple of Heaven, Patwadangar, and Modified Vaccinia Virus Ankara, etc.). Such a kit may further include instructions for administering to an individual having cancer the pharmaceutical composition comprising VV in an amount effective to infect cancer cells in the individual, e.g., prior to administration of a pharmaceutical composition comprising an antibody, fusion protein or conjugate of the present disclosure.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods

Aspects of the present disclosure include methods of using the antibodies, fusion proteins (e.g., CARs), and conjugates of the present disclosure. The methods are useful in a variety of contexts, including in vitro and/or in vivo research and/or clinical applications.

In certain aspects, provided are methods that comprise administering an effective amount of a pharmaceutical composition comprising any of the anti-VV A56 or anti-VV B5 antibodies of the present disclosure (including any of the fusion proteins or conjugates comprising such antibodies) to an individual having cancer, where the individual comprises cancer cells infected with OV that encode the VV A56 or VV B5 antigen to which the antibodies bind (where the VV A56 or VV B5 antigen may be native or heterologous to the OV), and where the antibody, fusion protein or conjugate is targeted to the infected cancer cells by VV antigens expressed on the surface of the infected cancer cells. According to some embodiments, such methods further comprise, prior to administering the pharmaceutical composition to the individual, infecting the cancer cells by administering an effective amount of the OV to the individual. Such methods find use, e.g., in treating the cancer of the individual.

In certain embodiments, the pharmaceutical composition comprises any of the anti-VV A56 or anti-VV B5 antibody conjugates of the present disclosure. For example, the pharmaceutical composition may comprise a conjugate, where the anti-VV A56 or anti-VV B5 antibody is conjugated to a detectable label or radioactive isotope which is an in vivo imaging agent. Such methods may further comprise imaging the infected cancer cells in the individual using the in vivo imaging agent. The methods in which a conjugate comprising a detectable label or radioactive isotope is administered to the individual find use in imaging the cancer cells in the individual, e.g., for diagnostic, prognostic, and/or anti-cancer therapy monitoring purposes.

According to some aspects, provided are methods of targeting an antibody that specifically binds an oncolytic virus (OV) antigen to cancer cells in an individual. Such methods comprise administering to the individual an effective amount of a pharmaceutical composition comprising an antibody (or fusion protein or conjugate comprising an antibody) that specifically binds the OV antigen, where the cancer cells in the individual are infected with OV and express the OV antigen on their surface. The OV antigen may be any of the OV antigens described elsewhere herein. In certain embodiments, the OV antigen is a native OV antigen. For example, the OV may be any of the viruses described above in the section entitled “Chimeric Antigen Receptors that Bind Oncolytic Virus Antigens”, or an engineered variant thereof (e.g., an engineered variant having one or more of the OV modifications described hereinbelow—e.g., transgenic for human GM-CSF, deletion of the thymidine kinase gene, and/or the like), and the OV antigen is native to the OV. According to some embodiments, the OV antigen is heterologous to the OV, e.g., a viral antigen heterologous to the OV (e.g., any of the viral OV antigens described above in the section entitled “Chimeric Antigen Receptors that Bind Oncolytic Virus Antigens”), a tumor antigen, or any other heterologous OV antigens described elsewhere herein. According to some embodiments, such methods further comprise, prior to administering the pharmaceutical composition to the individual, infecting the cancer cells by administering an effective amount of the OV to the individual. Such methods find use, e.g., in treating the cancer of the individual.

When bound on the cancer cell surface, the antibody (or fusion protein) may induce cytotoxicity, e.g., via antibody-dependent cellular cytotoxicity (ADCC), by recruiting complement in complement dependent cytotoxicity (CDC), via antibody-dependent cellular phagocytosis (ADCP), via epitope spreading, or by some other mechanism. The antibodies may be modified in the Fc region to provide desired or enhanced effector functions. This may be achieved by introducing one or more amino acid substitutions in an Fc region of the antibody. Alternatively, where it is desirable to eliminate or reduce effector function, so as to minimize side effects or therapeutic complications, certain other Fc regions may be used.

In certain embodiments, the pharmaceutical composition may comprise a conjugate, where the anti-VV A56 or anti-VV B5 antibody is conjugated to an agent selected from a chemotherapeutic agent, a toxin, a radiation sensitizing agent, a therapeutic radioactive isotope, and a radioisotope that permits in vivo imaging of the antibody. The agent may be any such agents described in the Conjugates section above.

The antibody (or fusion protein comprising same) may be selected such that the conjugate specifically binds an OV antigen of interest. OV antigens of interest include, but are not limited to, any of the native or heterologous OV antigens described elsewhere herein. In certain embodiments, the OV antigen is a native OV antigen. For example, the OV may be any of the viruses described above in the section entitled “Chimeric Antigen Receptors that Bind Oncolytic Virus Antigens”, or an engineered variant thereof (e.g., an engineered variant having one or more of the OV modifications described hereinbelow—e.g., transgenic for human GM-CSF, deletion of the thymidine kinase gene, and/or the like), and the OV antigen is native to the OV. According to some embodiments, the OV antigen is heterologous to the OV, e.g., a viral antigen heterologous to the OV (e.g., any of the viral OV antigens described above in the section entitled “Chimeric Antigen Receptors that Bind Oncolytic Virus Antigens”), a tumor antigen, or any other heterologous OV antigens described elsewhere herein. In some embodiments, the antigen is a native or heterologous OV antigen the expression of which is under exclusive control of the wild-type transcription regulatory elements of the OV for the antigen. That is, according to such embodiments, the transcription regulatory elements of the OV for the antigen are not modified relative to the wild-type OV. In certain embodiments, the antigen is a native OV antigen the expression levels of which are altered (e.g., increased) relative to expression by the wild-type OV by modification (e.g., replacement, duplication, etc.) of one or more wild-type transcription regulatory elements of the OV. By way of example, the antigen may be a native OV antigen (e.g., native A56, native B5, or any other native OV antigen of interest) the expression of which is increased relative to expression by the wild-type OV by operably coupling the coding region of the native OV antigen to one or more heterologous regulatory elements (e.g., promoter, such as a human promoter) that results in higher expression levels as compared to the wild-type transcription regulatory elements of the OV. Such an OV may be a modified OV, e.g., an OV that includes one or more of the OV modifications described hereinbelow.

In certain embodiments, the OV antigen is heterologous to the OV. For example, the antibody may be selected such that the antibody portion of the conjugate specifically binds any of the heterologous OV antigens described elsewhere herein, e.g., viral antigens heterologous to the OV, tumor antigens, or the like.

The antibody may be provided in any desired format, e.g., tetrameric format, single chain (e.g., scFv) format, etc. as described in the preceding sections of the present disclosure. According to some embodiments, the pharmaceutical composition comprises the antibody conjugated to a detectable label or radioactive isotope. In certain embodiments, the detectable label or radioactive isotope is an in vivo imaging agent. The methods in which the antibody is conjugated to an in vivo imaging agent may further comprise detecting the in vivo imaging agent to image the cancer cells in the individual in vivo, e.g., for diagnostic, prognostic, and/or anti-cancer therapy monitoring purposes.

According to some aspects, provided are methods of targeting a CAR that specifically binds an oncolytic virus (OV) antigen to cancer cells in an individual. Such methods comprise administering to the individual an effective amount of a pharmaceutical composition comprising a CAR comprising an antigen binding domain that specifically binds the OV antigen, where the cancer cells in the individual are infected with OV and express the OV antigen on their surface. The CAR may be expressed on the surface of a cell, e.g., an immune cell, such as an immune effector cell, e.g., a T cell, an NK cell, an NKT cell, a macrophage, or the like. For example, the CAR may be present on the surface of T cells, where the method is a method of targeting CAR T cells to the infected cancer cells in the individual. According to some embodiments, such methods further comprise, prior to administering the pharmaceutical composition to the individual, infecting the cancer cells by administering an effective amount of the OV to the individual. Such methods find use, e.g., in treating the cancer of the individual.

The antigen binding domain of the CAR specifically binds an OV antigen of interest. OV antigens of interest include, but are not limited to, any of the native or heterologous OV antigens described elsewhere herein. In certain embodiments, the OV antigen is a native OV antigen. For example, the OV may be any of the viruses described above in the section entitled “Chimeric Antigen Receptors that Bind Oncolytic Virus Antigens”, or an engineered variant thereof (e.g., an engineered variant having one or more of the OV modifications described hereinbelow—e.g., transgenic for human GM-CSF, deletion of the thymidine kinase gene, and/or the like), and the OV antigen is native to the OV. According to some embodiments, the OV antigen is heterologous to the OV, e.g., a viral antigen heterologous to the OV (e.g., any of the viral OV antigens described above in the section entitled “Chimeric Antigen Receptors that Bind Oncolytic Virus Antigens”), a tumor antigen, or any other heterologous OV antigens described elsewhere herein.

A pharmaceutical composition comprising cells that express a CAR on their surface may be prepared by a variety of methods. In some embodiments, a cell of the present disclosure is produced by transfecting the cell with a viral vector encoding the CAR. In some embodiments, the cell is a T cell, such that provided are methods of producing a CAR T cell. In some embodiments, such methods include activating a population of T cells (e.g., T cells obtained from an individual to whom a CAR T cell therapy will be administered), stimulating the population of T cells to proliferate, and transducing the T cell with a viral vector encoding the CAR. In some embodiments, the T cells are transduced with a retroviral vector, e.g., a gamma retroviral vector, encoding the CAR. In some embodiments, the T cells are transduced with a lentiviral vector encoding the CAR.

Cells of the present disclosure may be autologous/autogenetic (“self”) or non-autologous (“non-self,” e.g., allogeneic, syngeneic or xenogeneic). “Autologous” as used herein, refers to cells from the same individual. “Allogeneic” as used herein refers to cells of the same species that differ genetically from the cell in comparison. “Syngeneic,” as used herein, refers to cells of a different individual that are genetically identical to the cell in comparison. In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{MEM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An example approach for such sorting is described in Wang et al. (2016) Blood 127(24):2980-90.

In some embodiments, a specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the CAR into the T cells.

In some embodiments, T cells are activated and expanded for about 6 hours, about 12 hours, about 18 hours or about 24 hours prior to introduction of a nucleic acid (e.g., expression vector) encoding the CAR into the T cells. In some embodiments, T cells are activated at the same time that a nucleic acid (e.g., an expression vector) encoding the CAR is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

In some embodiments, the nucleic acid (e.g., an expression vector) encoding the CAR is introduced into the cell (e.g., a T cell) by microinjection, transfection, lipofection, heat-shock, electroporation, transduction, gene gun, microinjection, DEAE-dextran-mediated transfer, and the like. In some embodiments, the nucleic acid (e.g., expression vector) encoding the CAR is introduced into the cell (e.g., a T cell) by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some embodiments, the AAV vector comprises ITRs from AAV2 and a serotype from AAV6. In some embodiments, the nucleic acid (e.g., expression vector) encoding the CAR is introduced into the cell (e.g., a T cell) by lentiviral or retroviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed.

In certain aspects, provided are methods of targeting a conjugate that comprises an antibody that specifically binds an oncolytic virus (OV) antigen to cancer cells in an individual. Such methods comprise administering to the individual an effective amount of a pharmaceutical composition comprising an antibody that specifically binds the OV antigen, where the cancer cells in the individual are infected with OV and express the OV antigen on their surface. According to some embodiments, such methods further comprise, prior to administering the pharmaceutical composition to the individual, infecting the cancer cells by administering an effective amount of the OV to the individual. Such methods find use, e.g., in treating the cancer of the individual.

The antibody of the conjugate specifically binds an OV antigen of interest, including but not limited to any of the OV antigens described elsewhere herein. In certain embodiments, the OV antigen is a native OV antigen. For example, the OV may be any of the viruses described above in the section entitled “Chimeric Antigen Receptors that Bind Oncolytic Virus Antigens”, or an engineered variant thereof (e.g., an engineered variant having one or more of the OV modifications described hereinbelow—e.g., transgenic for human GM-CSF, deletion of the thymidine kinase gene, and/or the like), and the OV antigen is native to the OV. According to some embodiments, the OV antigen is heterologous to the OV, e.g., a viral antigen heterologous to the OV (e.g., any of the viral OV antigens described above in the section entitled “Chimeric Antigen Receptors that Bind Oncolytic Virus Antigens”), a tumor antigen, or any other heterologous OV antigens described elsewhere herein.

An OV administered to an individual according to the present disclosure may be a wild-type OV or a modified OV. According to some embodiments, the OV is a modified OV, where the modified OV is a chimeric OV comprising two or more domains from different viruses. In some embodiments, the OV is a modified OV (e.g., a non-chimeric or chimeric OV) that includes one or more modifications in one or more of the following functional categories: infection, replication, tropism, improved safety, reporter genes for imaging, thwarting the host anti-viral immune response, enhancing the host anti-tumor immune response, etc. According to some embodiments, the OV is modified to: encode and express human GM-CSF; have a deletion of the thymidine kinase gene (limiting or substantially limiting viral replication to cells with high levels of thymidine kinase, typically seen in cancer cells with a mutated RAS or p53 gene); encode and express a reporter gene (e.g., Lac Z, luciferase, and/or the like); alter one or more genes encoding ribonucleotide reductase (RR); disrupt the F2L gene (which encodes the viral dUTPase involved in both maintaining the fidelity of DNA replication and providing the precursor for the production of TMP by thymidylate synthase); express a therapeutic protein (e.g., a suicide gene—that is, a gene coding for a protein able to convert a precursor of a drug into a cytotoxic compound, including but not limited to TK, cytosine deaminase, purine nucleoside phosphorylase, uracil phosphoribosyl transferase, thymidylate kinase, and the like); express an immunostimulatory protein, where “immunostimulatory protein” refers to a protein which has the ability to stimulate the immune system, in a specific or non-specific way, including cytokines, chemokines, growth factors, etc.; and any combinations thereof. Any of the modified OVs described above may encode any of the OV antigens described elsewhere herein. For example, the modified OV may be modified from any of the viruses described above in the section entitled “Chimeric Antigen Receptors that Bind Oncolytic Virus Antigens”, and the OV antigen is native to the OV. According to some embodiments, the OV antigen is heterologous to the modified OV, e.g., a viral antigen heterologous to the modified OV (e.g., any of the viral OV antigens described above in the section entitled “Chimeric Antigen Receptors that Bind Oncolytic Virus Antigens”), a tumor antigen, or any other heterologous OV antigens described elsewhere herein.

In certain aspects, provided are methods comprising administering a pharmaceutical composition comprising cells (e.g., cancer cells) infected with OV. The pharmaceutical composition may further include an antibody, conjugate, or fusion protein that specifically binds an OV antigen expressed by the infected cells, e.g., cancer cells. The cells (e.g., cancer cells) may have been removed from the individual during surgery. The cells (e.g., cancer cells) may have been altered (and killed) in the lab to make them more likely to be attacked by the immune system when administered back into the patient. The patient's immune system then attacks the cells and any similar cells still in the body. The antibody, conjugate, or fusion protein of the present disclosure may be employed according to this approach to promote uptake of tumor particles/antigens by Fc receptors on professional APC, leading to an enhanced immune response against the tumor.

The pharmaceutical compositions may be administered to any of a variety of individuals. In certain aspects, the individual is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In certain aspects, the individual is an animal model (e.g., a mouse model, a primate model, or the like) of a cellular proliferative disorder, e.g., cancer.

The individual in need thereof may have a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

The antibodies, fusion proteins and conjugates of the present disclosure may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, or intra-tumoral administration.

The antibodies, fusion proteins and conjugates of the present disclosure may be administered in a pharmaceutical composition in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer, as compared to a control. With respect to cancer, in some embodiments, the therapeutically effective amount is sufficient to slow the growth of a tumor, reduce the size of a tumor, and/or the like. An effective amount can be administered in one or more administrations.

As described above, aspects of the present disclosure include methods for treating a cancer of an individual. By treatment is meant at least an amelioration of one or more symptoms associated with the cancer of the individual, where amelioration is used in a broad sense to refer to at least a reduction in the magnitude of a parameter, e.g. symptom, associated with the cancer being treated. As such, treatment also includes situations where the cancer, or at least one or more symptoms associated therewith, are completely inhibited, e.g., prevented from happening, or stopped, e.g., terminated, such that the individual no longer suffers from the cancer, or at least the symptoms that characterize the cancer.

An antibody, fusion protein, or conjugate of the present disclosure may be administered to the individual alone or in combination with a second agent. Second agents of interest include, but are not limited to, agents approved by the United States Food and Drug Administration and/or the European Medicines Agency (EMA) for use in treating cancer. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When an antibody, fusion protein, or conjugate of the present disclosure is administered with a second agent, the antibody, fusion protein, or conjugate and the second agent may be administered to the individual according to any suitable administration regimen. According to certain embodiments, the antibody, fusion protein, or conjugate and the second agent are administered according to a dosing regimen approved for individual use. In some embodiments, the administration of the antibody, fusion protein, or conjugate permits the second agent to be administered according to a dosing regimen that involves one or more lower and/or less frequent doses, and/or a reduced number of cycles as compared with that utilized when the second agent is administered without administration of the antibody, fusion protein, or conjugate. In certain aspects, the administration of the second agent permits the antibody, fusion protein, or conjugate to be administered according to a dosing regimen that involves one or more lower and/or less frequent doses, and/or a reduced number of cycles as compared with that utilized when the antibody, fusion protein, or conjugate is administered without administration of the second agent.

In some embodiments, one or more doses of the antibody, fusion protein, or conjugate and the second agent are administered concurrently to the individual. By “concurrently” is meant the antibody, fusion protein, or conjugate and the second agent are either present in the same pharmaceutical composition, or the antibody, fusion protein, or conjugate and the second agent are administered as separate pharmaceutical compositions within 1 hour or less, 30 minutes or less, or 15 minutes or less.

In some embodiments, one or more doses of the antibody, fusion protein, or conjugate and the second agent are administered sequentially to the individual.

In some embodiments, the antibody, fusion protein, or conjugate and the second agent are administered to the individual in different compositions and/or at different times. For example, the antibody, fusion protein, or conjugate may be administered prior to administration of the second agent, e.g., in a particular cycle. Alternatively, the second agent may be administered prior to administration of the antibody, fusion protein, or conjugate, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the antibody, fusion protein, or conjugate. In certain aspects, such a regimen “primes” the cancer cells to potentiate the anti-cancer effect of the antibody, fusion protein, or conjugate. Such a period of time separating a step of administering the second agent from a step of administering the antibody, fusion protein, or conjugate is of sufficient length to permit priming of the cancer cells, desirably so that the anti-cancer effect of the antibody, fusion protein, or conjugate is increased.

In some embodiments, administration of one agent is specifically timed relative to administration of the other agent. For example, in some embodiments, the antibody, fusion protein, or conjugate is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models, in some embodiments, such assessment or empirical determination is made in vivo, in a patient population (e.g., so that a correlation is established), or alternatively in a particular individual of interest.

In some embodiments, the antibody, fusion protein, or conjugate and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the antibody, fusion protein, or conjugate and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the antibody, fusion protein, or conjugate is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the antibody, fusion protein, or conjugate is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered, properties (e.g., pharmacokinetic properties) of the antibody, fusion protein, or conjugate, and/or one or more features of the patient's response to therapy with the antibody, fusion protein, or conjugate. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The antibody, fusion protein, or conjugate and the second agent may be administered together or independently via any suitable route of administration. The antibody, fusion protein, or conjugate and the second agent may be administered via a route of administration independently selected from oral, parenteral (e.g., by intravenous, intra-arterial, subcutaneous, intramuscular, or epidural injection), topical, or intra-nasal administration. According to certain embodiments, antibody, fusion protein, or conjugate and the second agent are both administered orally (e.g., in tablet form, capsule form, liquid form, or the like) either concurrently (in the same pharmaceutical composition or separate pharmaceutical compositions) or sequentially.

When the methods further comprise, prior to administering the pharmaceutical composition to the individual, infecting the cancer cells by administering the OV to the individual, any suitable administration regimen may be employed to infect the cancer cells. Poxvirus replication takes place in the cytoplasm, as the virus is sufficiently complex to have acquired all the functions necessary for genome replication. Once in the cell cytoplasm, gene expression is carried out by viral enzymes associated with the core. Expression is divided into 2 phases: early genes: which represent about of 50% genome, and are expressed before genome replication, and late genes, which are expressed after genome replication. The temporal control of expression is provided by the late promoters, which are dependent on DNA replication for activity. Genome replication is believed to involve self-priming, leading to the formation of high molecular weight concatemers, which are subsequently cleaved and repaired to make virus genomes. Viral assembly occurs in the cytoskeleton and probably involves interactions with the cytoskeletal proteins (e.g., actin-binding proteins). Inclusions form in the cytoplasm that mature into virus particles. Vaccinia virus is unique among DNA viruses as it replicates only in the cytoplasm of the host cell. Therefore, the large genome is required to code for various enzymes and proteins needed for viral DNA replication. During replication, vaccinia produces several infectious forms, which differ in their outer membranes: the intracellular mature virion (IMV), the intracellular enveloped virion (IEV), the cell-associated enveloped virion (CEV), and the extracellular enveloped virion (EEV).

To infect the cancer cells with the OV, the OV is administered using a suitable route of administration. The route of administration may vary with the location and nature of the cancer, and may include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation. Intratumoral injection, or injection directly into the tumor vasculature is specifically contemplated for discrete, solid, accessible tumors. Local, regional or systemic administration also may be appropriate. The viral particles may advantageously be contacted by administering multiple injections to the tumor, spaced, for example, at approximately 1 cm intervals. Continuous administration also may be applied where appropriate, for example, by implanting a catheter into a tumor or into tumor vasculature. Such continuous perfusion may take place, for example, for a period of from about 1-2 hours, to about 2-6 hours, to about 6-12 hours, or about 12-24 hours following the initiation of administration. Administration regimens may vary, and often depend on tumor type, tumor location, disease progression, and health and age of the patient. Certain types of tumor will require more aggressive treatment, while at the same time, certain patients cannot tolerate more taxing protocols. The clinician will be best suited to make such decisions.

Injection of nucleic acid constructs may be delivered by syringe or any other method used for injection of a solution, so long as the expression construct can pass through the particular gauge of needle required for injection. An exemplary needleless injection system that may be used for the administration of OV is exemplified in U.S. Pat. No. 5,846,233. This system features a nozzle defining an ampule chamber for holding the solution and an energy device for pushing the solution out of the nozzle to the site of delivery. Another exemplary syringe system is one that permits multiple injections of predetermined quantities of a solution precisely at any depth (U.S. Pat. No. 5,846,225). Mixtures of OV particles or nucleic acids encoding same may be prepared in water suitably mixed with one or more excipients, carriers, or diluents. Dispersions may also be prepared in glycerol, liquid polyethylene glycols, and mixtures thereof and in oils.

A physician may start prescribing doses of OV vector at levels lower than that required in order to achieve the desired therapeutic effect and gradually increase the dosage until the desired effect is achieved. Alternatively, a physician may begin a treatment regimen by administering a dose of OV vector and subsequently administer progressively lower doses until a therapeutic effect is achieved, e.g., a reduction in the volume of one or more tumors.

The following examples are offered by way of illustration and not by way of limitation.

### EXPERIMENTAL

### Example 1—Antibody Generation

Stable CHO cell lines were generated expressing either vaccinia virus A56 (CHO-A56) or B5 capsid protein (CHO—B5), using A56 or B5 sequence from a Wyeth VV strain (SEQ ID NO: 180 and SEQ ID NO: 181, respectively). Antibodies were raised in New Zealand White Rabbits by sequential semi-monthly subcutaneous immunization with a mix of CHO-A56 and CHO—B5 cells (30 million per cell type for first injection, 45 million for subsequent injections)—mixed with aluminum hydroxide (5 mg/injection) and CpG (Cat #ODN1826, 20 μg/injection). One week following the 10^{th} injection, the rabbit was euthanized and the spleen was harvested (Cedarlane). B cells were cultured and isolated as per Babcook et al. (1). Antibodies were identified by screening the B-cell supernatants for binding to OVCAR3 cells (ATCC, Cat #HTB-161) infected with VV Western Reserve strain VVdd (eGFP) (gift from Dr. John Bell, Ottawa Hospital Research Institute (OHRI), SEQ ID NOs: 182 and 183), by flow cytometry (data not shown). In order to clone the antibody from cultured B cells, the frozen cells were thawed and analyzed by A56.hFc or B5.hFc hemolytic plaque assay. The human Fc fusion proteins were generated in-house using A56 or B5 sequence from the Wyeth VV strain as set out in Tables 2 and 4. Isolated single B cells were lysed and antibody V-genes were amplified by RT-PCR. PCR products corresponding to matched antibody heavy and light chains were then cloned into human IgG1 constant region and IgK constant region constructs (pTT5/IgG1, pTT5/Igk). To produce recombinant antibody, VH and VL chain plasmids were transfected into HEK293-6E cells using 293 fectin (Thermo, Cat #12347019). After 96 hours of secretion, the antibody-containing supernatant was cleared of cells by centrifugation and sterile filtration (0.22 μm). Antibodies were purified using HiTrap Mab Select SuRe column (GE, Cat #: 11003495) and HiPrep 26/10 Desalting column (GE, Cat #17508701) on an AKTA Express system. The purity of the antibodies was tested by SDS-PAGE, UPLC-SEC, and LC-MS. The sequences of the antibodies generated are shown in Tables 1 and 3. The antibody ID and formats are set out in Table 15 below.

TABLE 15
Antibody ID and Format

| Antibody | ID | Fc |
|---|---|---|
| Anti-B5 | A048 | hIgG1 |
| Rabbit | A058 | muIgG2a |
| Antibody | A073 | RbIgG |
| Anti-A56 | A049 | hIgG1 |
| Rabbit | A059 | muIgG2a |
| Antibody | A056 | RbIgG |
| Anti-A56 | A047 | hIgG1 |
| Rabbit | A057 | muIgG2a |
| Antibody | | |
| Anti-B5 | A051 | hIgG1 |
| Rabbit | | |
| Antibody | | |
| Anti-A56 | A050 | hIgG1 |
| Rabbit | | |
| Antibody | | |
| Anti-A56 | A054 | hIgG1 |
| Rabbit | | |
| Antibody | | |

### Example 2—Antibody Binding to A56 and B5 Antigens

Stable cell lines were generated using Lenti-x293T (Clontech, Cat #632181) cells, resulting in HEK-A56 (GFP) and HEK-B5 (GFP) cells which express the antigens A56 (Wyeth VV sequence) or B5 (Wyeth VV sequence) on the cell surface. A549 (ATCC, Cat #CCL-185) and CaOv3 (ATCC, Cat #HTB-75) tumor cells were grown to 80-90% confluence in appropriate media (A549—F-12K Nutrient Mixture (Gibco, Cat #21127-022); CaOV3-DMEM (Gibco, Cat #11995-040) supplemented with 10% FBS (Corning, Cat #35-015-CV)). Cells were infected with VVdd (eGFP) at a multiplicity of infection of 0.5 in serum free RPMI (Gibco, Cat #A10491-01) and incubated for 6.5 h, at 37° C. in 5% CO₂. Wells were supplemented with growth media 1:2, and further incubated overnight. A549 cells were trypsinized, and plated, alongside the CaOV3 cells, at approximately 1×10⁵ cells per well, in 96-well v-bottom plates (Sarstedt, Cat #82.1583.001). Corresponding uninfected A549 and CaOV3 cells, and HEK-A56 (GFP) and HEK-B5 (GFP) and HEK-WT cell lines were also plated at 4×10⁴ cells per well. Anti-Vaccinia Virus rabbit polyclonal antibody (CedarLane, Cat #LS-C103289) was used as a positive control.

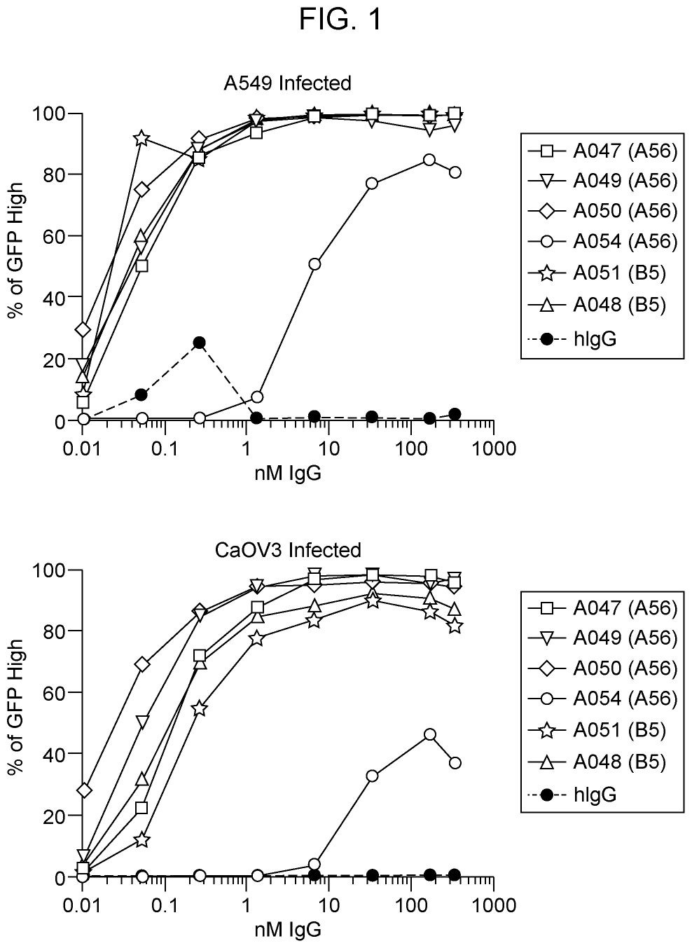

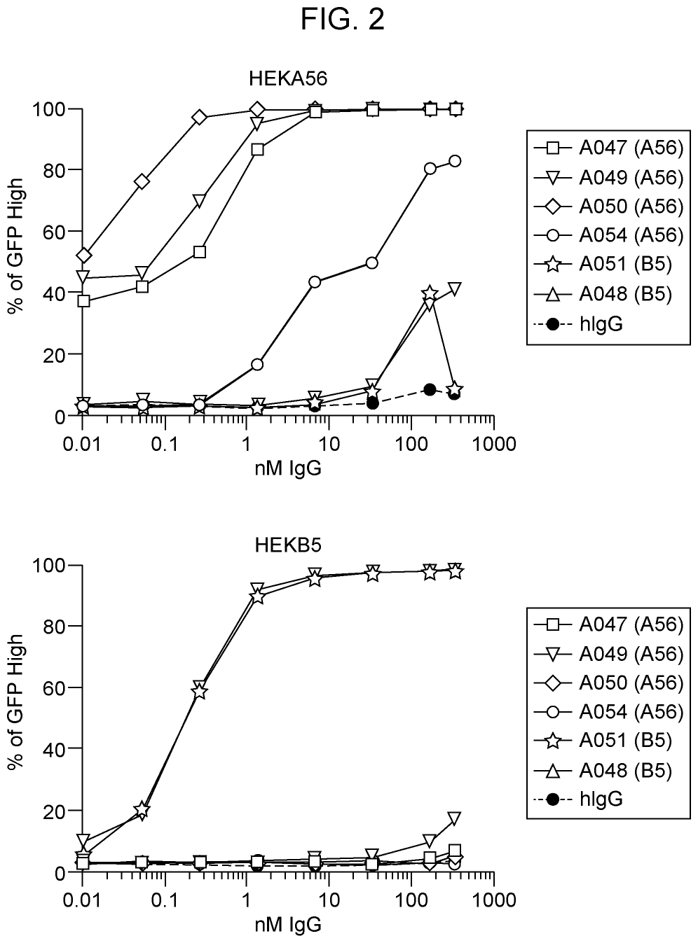

Anti-A56 antibodies (A047, A049, A050, or A054), anti-B5 antibodies (A048 or A051), and human IgG isotype control (Sigma, Cat #15154) were added to cells and incubated for 25 min on ice. Cells were washed twice with PBS, centrifuged at 450 G for 4 min and incubated with 1:100 AlexaFluor 647 conjugated anti-rabbit IgG (Jackson, Cat #115-605-046) or AlexaFluor 647 conjugated anti-human IgG (Jackson, Cat #109-605-098), with 1:1000 fixable viability dye eFluor 780 (eBioscience, Cat #65-0865-14) for 15 min at room temperature (RT). Following two washes with PBS, cells were fixed in IC fixation buffer (eBioscience, Cat #00-8222-49) for 10 min, washed twice with PBS, resuspended in 1% FBS in PBS, and analyzed by flow cytometry on a BD LSRFortessa X-20 HTS system, and data processed with FlowJo V 10.5.3 Software. The data shows binding of antibodies to VV VVdd (eGFP) infected cells (FIG. 1). The anti-A56 antibodies bind HEK-A56 (GFP) cells, and the anti-B5 antibodies bind HEK-B5 (GFP) cells, specifically (FIG. 2). No binding was observed to negative controls—HEK parental cells, uninfected A549 or CaOV3 cells, and an irrelevant VV antigen A33, also expressed in HEK cells (data not shown). Isotype control hIgG showed no binding to cells.

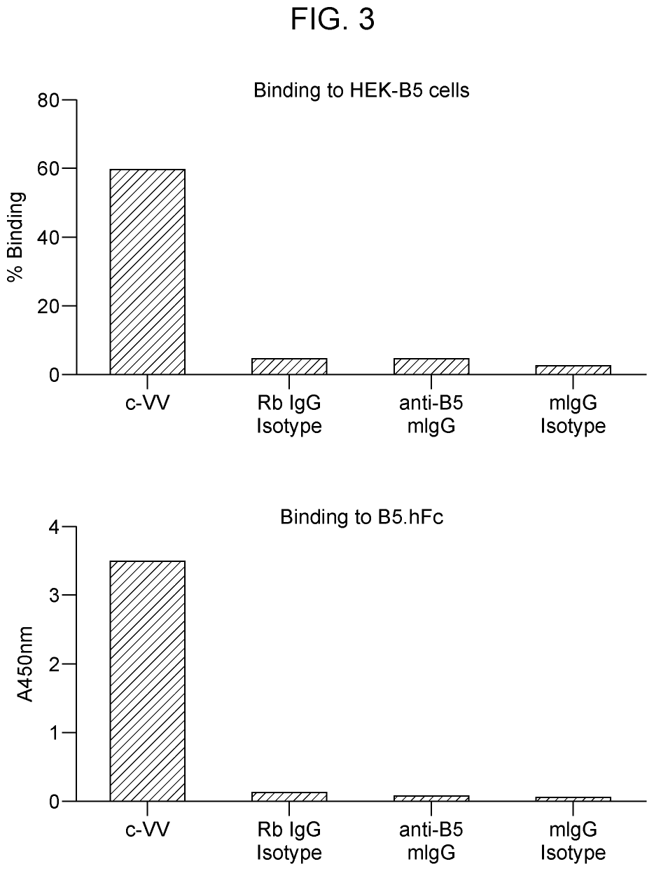

A commercially available anti-B5 antibody (Immunetech, Cat #IT-012-009M1, raised using a VV Western Reserve immunogen) was tested for binding to B5 antigens. By flow cytometry, a dilution series of the anti-Vaccinia rabbit polyclonal antibody was compared to the Immunetech anti-B5 antibody (‘IT anti-B5 IgG’), as well as isotope controls, with similar assay conditions as above. For mouse antibody detection of IT anti-B5 IgG and mouse isotype control, a goat anti-mouse A647 (Jackson, Cat #115-005-146) was used. The IT anti-B5 IgG did not bind to the HEK-B5 cells (FIG. 3, top panel). An ELISA to assess binding to the B5.hFc soluble antigen confirmed this antibody does not bind the B5 reagents made based on the VV Wyeth B5 sequence. In this assay, B5.hFc was coated on a maxisorp plate at 2 μg/ml and incubated overnight at 4° C. Following standard ELISA procedure, the plate was washed, blocked in 5% skim milk powder, the primary antibodies incubated in block for 1 h RT, and washed. Secondary detection was with goat anti-mIgG Fc HRP (Jackson, Cat #115-035-071) or anti-Rb IgG Fc HRP (Jackson ImmunoResearch, Cat #111-035-046), for 1 h at RT, followed by washing, and addition of 50 ul/well TMB K-Blue (Neogen, #308175), and then 50 μl/well 1N HCl. The IT anti-B5 IgG was negative for binding to the B5.hFc soluble antigen by standard ELISA (FIG. 3, bottom panel).

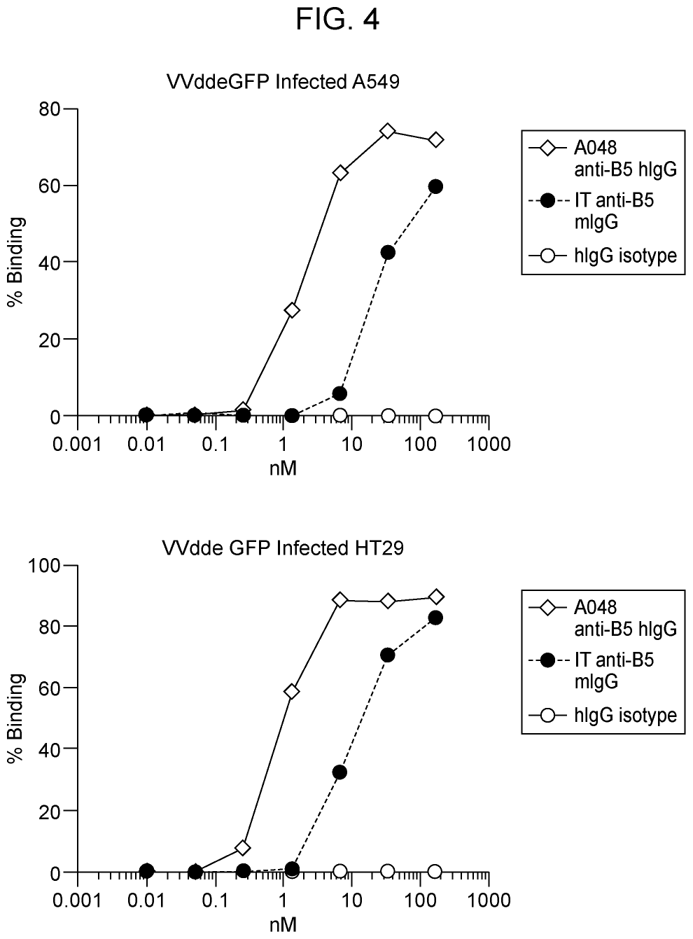

The IT anti-B5 IgG was also tested for binding to Western Reserve VVddeGFP-infected A549 and HT29 cells. A549 and HT29 cells (ATCC cat #HTB-38, grown in McCoy 5A media (Gibco #16600-082)+10% FBS) were cultured to 80-90% confluency. Cells were infected with VVdd (eGFP) at a multiplicity of infection of 1 in serum free RPMI. Cells were incubated for 6.5 h at 37° C. in 5% CO₂, supplemented with growth media 1:2, and further incubated overnight. The cells were trypsinized, and plated at approximately 1×10⁵ cells per well, in 96-well v-bottom plates. Anti-B5 antibodies A048 hIgG1 and IT anti-B5 IgG, and human isotype controls were added to cells and incubated for 25 min on ice. Cells were washed and antibody detected as above, with AlexaFluor 647 conjugated anti-human IgG and anti-murine IgG (Jackson, #115-605-164), followed by 1:1000 fixable viability dye eFluor 780. Following two washes, fixation, and resuspension in 1% FBS in PBS, the samples were analyzed by flow cytometry on a BD LSRFortessa X-20 HTS system. The data show that A048 antibody binds to Western Reserve VVddeGFP-infected A549 and HT29 cells more strongly than IT anti-B5 IgG (FIG. 4). The EC50 for binding of A048 antibody to infected A549 cells and HT29 cells is 6 nM, and 1.1 nM respectively. The EC50 for binding of IT anti-B5 IgG to infected A549 and HT29 cells is 78 nM and 15.8 nM, respectively.

### Example 3—Reformatting to Mouse and Rabbit Antibodies

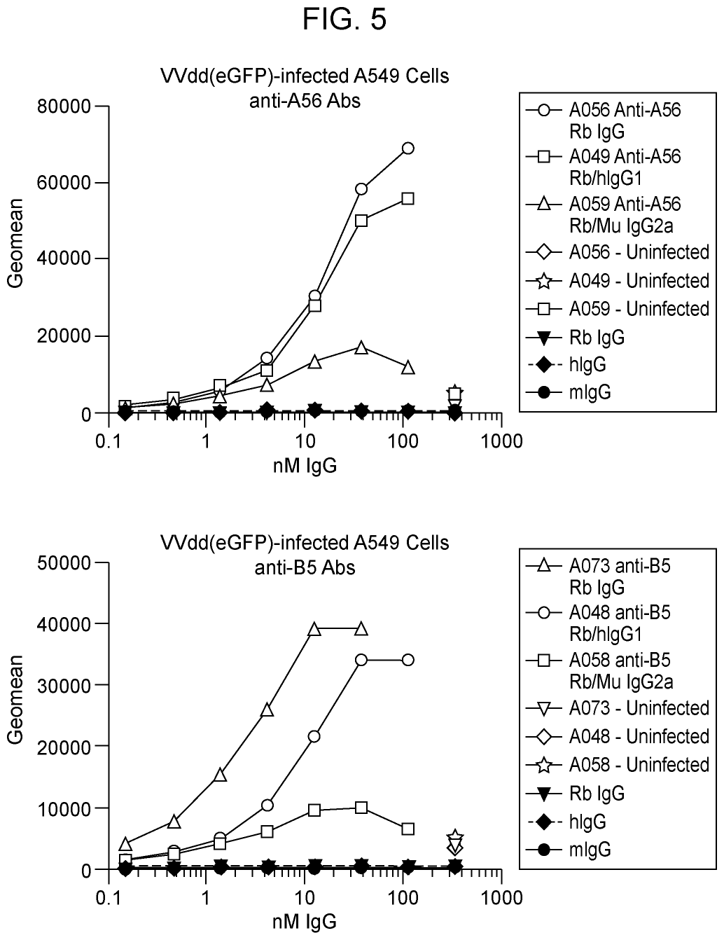

Antibodies were reformatted to full rabbit IgG or chimeric rabbit/mouse IgG2a Fc antibodies, expressed in HEK293 cells, and purified as described previously. A549 cells infected with VVdd (eGFP), as described above, were tested for binding by the antibodies as rabbit IgG, or mouse or human chimeras. Anti-A56 antibodies (A056, A049, A059), and anti-B5 antibodies (A073, A048, A058), as well as rabbit IgG isotype (Jackson, Cat #011-000-003), human IgG isotype (Sigma Aldrich, Cat #15154), and mouse IgG isotype (R&D Biosystems, Cat #MAB004) were added to infected cells and incubated for 25 min on ice. Cells were washed, and incubated with 1:100 AlexaFluor 647 conjugated anti-rabbit IgG (Jackson, Cat #115-605-046), or anti-human IgG (Jackson, Cat #109-605-098), or anti-mouse IgG (Jackson, Cat #115-605-164), with 1:1000 DAPI (BioLegend, Cat #422801) for 15 min at RT. Following washes and fixation, samples were analyzed by flow cytometry. Data confirms binding of all formats to VV VVdd (eGFP)-infected cells with similar EC50s (FIG. 5, Table 16). No binding was observed to uninfected cells, or by isotype controls.

TABLE 16
Antibody format and EC50 for
binding to VV-infected A549 cells

| | | | VVdd (eGFP)- |
|---|---|---|---|
| | | | Infected A549 |
| Antibody | ID | Fc | EC50 nM |
| Anti-B5 | A048 | hIgG1 | 9 nM |
| Rabbit | A058 | muIgG2a | 3 nM |
| Antibody | A073 | RbIgG | 3 nM |
| Anti-A56 | A049 | hIgG1 | 14 nM |
| Rabbit | A059 | muIgG2a | 5 nM |
| Antibody | A056 | RbIgG | 16 nM |

### Example 4—Antibody Binding to Virus-Infected Tissue

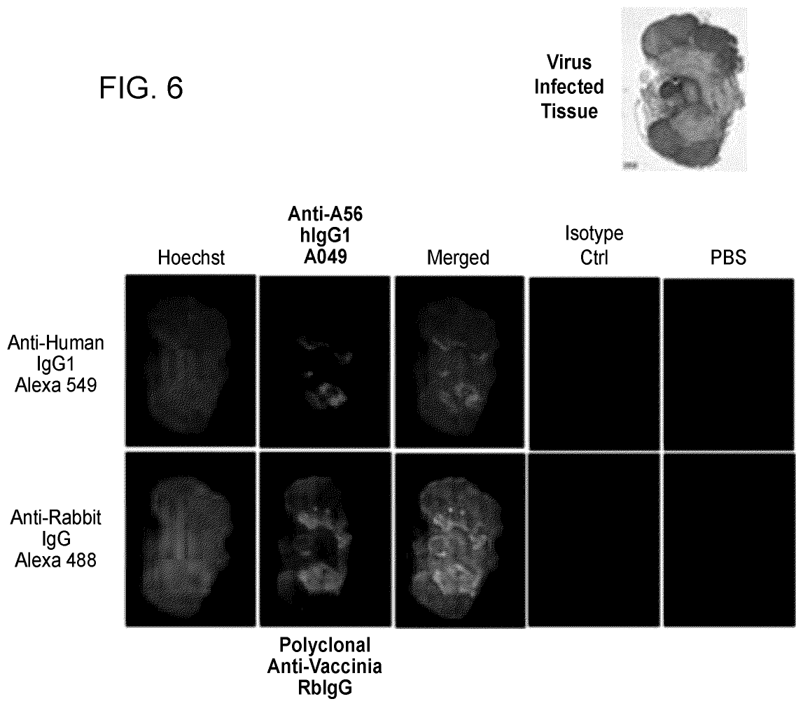

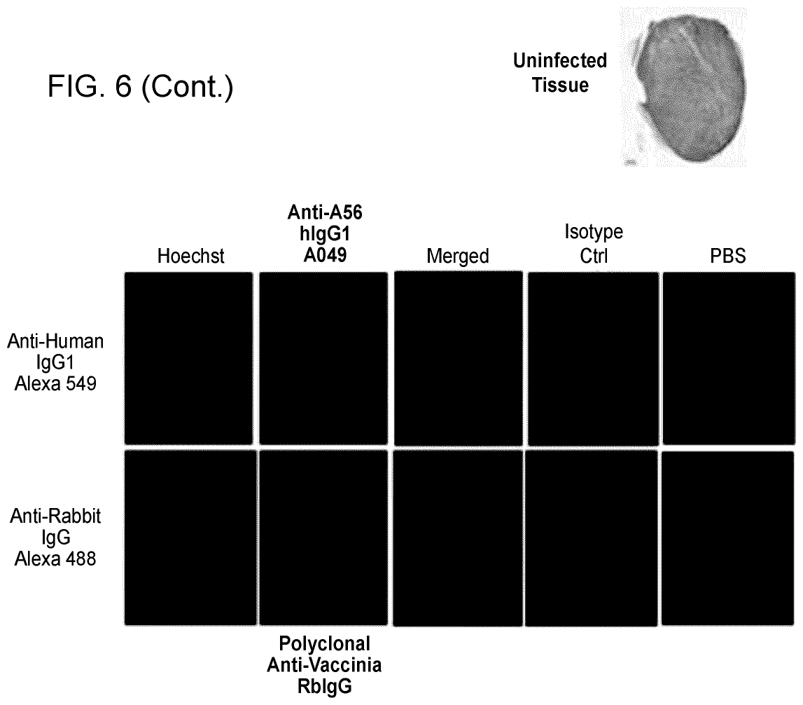

BALB/c female mice were implanted with 1×10⁶ MC38 cells on Day 0. For some mice, VVdd (eGFP) was given IT, 2.5×10⁷ pfu in 100 μl, on Day 11 and Day 13 post MC38 implantation, and mice were euthanized on Day 16. A control mouse bearing an MC38 tumor was treated with PBS in place of VVdd (eGFP) and was euthanized Day 12 post implantation. Tumor tissue was collected in 10% neutral buffered formalin (NBF). Tissues were formalin fixed, paraffin embedded, sectioned and stained. H&E stained images were scanned using the Aperio ScanScope AT2 digital slide scanner. Tissues were deparaffinized and rehydrated according to standard protocols. Tissue was autoclaved in antigen retriever (Sigma, Cat #C999-100ML) for 20 min at 121° C., for heat-induced epitope retrieval. After cooling, slides were rinsed twice with PBS, and the section isolated with a PAP pen to create a hydrophobic barrier. Tissue was kept hydrated with 200 μl wash buffer (0.1% Tween-20, 0.1% BSA in PBS). The tissues were permeabilized at RT for 10 min using 0.2% Triton in PBS, and washed three times. Sections were blocked for 60 min in blocking buffer (5% normal goat serum, 0.05% Tween-20, 0.1% BSA in PBS). Primary antibodies in antibody diluent (1% normal goat serum, 0.05% Tween-20, 0.1% BSA in PBS) were applied to sections, after removal of blocking buffer, and incubated overnight at 4° C. A049 (Anti-A56) hIgG1 was used at 12.5 μg/ml, and a positive control polyclonal rabbit anti-Vaccinia virus antibody (CedarLane, Cat #LS-C103289) was used at 1:100. Human IgG1 (Sigma, Cat #15154) and rabbit IgG (Jackson ImmunoResearch Laboratories, Cat #011-000-003) isotype controls were used at 25 μg/ml. The next day, tissues were washed and secondary antibodies (200 μl) prepped in antibody diluent and incubated for 1.5 h at RT in the dark. Human primary antibody stained tissues were detected with AlexaFluor 549-conjugated goat anti-human IgG (Jackson, Cat #109-585-003) at 1:100, and rabbit primary antibodies were detected with AlexaFluor Plus 488 Goat anti-rabbit IgG (H+L) antibody (Thermo, Cat #A32731) at 10 μg/ml. Following incubation, slides were washed three times, 5 min each. Hoechst stain (Sigma, Cat #B2261) was added to tissues at 1 μg/ml, incubated for 10 min and then excess removed. Cover slips were added with Fluoromont-G (Southern Biotech, Cat #0100-01) and imaged using a Leica SP5 X Laser Scanning Confocal Microscope. Data confirms binding of A049 to VV VVdd (eGFP) infected tumor tissue, with no binding observed to uninfected tumor tissue (FIG. 6). Polyclonal rabbit anti-Vaccinia virus antibody shows non-specific binding to uninfected tumor tissue.

### Example 5—Antibody Affinity to B5 and A56 Protein

Antibody affinity was assessed using a Biacore T200. On a Series S CM5 chip, A048 was immobilized on FC2, and A051 was immobilized on FC3, with FC1 blank immobilized. The sensor surface was activated by injecting EDC and NHS. Ligands were immobilized as follows: anti-B5 A048 hIgG1 (12 μg/mL) prepared in 200 μL sodium acetate, pH 4.5, and A051 (12 μg/mL) in 200 μl sodium acetate pH 5.0. The remaining activated groups were blocked by injecting ethanolamine. Finally, the chip was washed with 50 mM NaOH. Approximately 1000 RU of each antibody was amine coupled, followed by chip equilibration with buffer. All binding studies were performed in freshly prepared and filtered running buffer HBS-EP+ (GE, Cat #BR100188). B5.hFc (MW 150 kDa) was flowed over as analyte in increasing concentration of antigen (1.5 nM, 4.6 nM, 13.8 nM, 41.4 nM and 124 nM) for 200s at 30 μl/min association, followed by a single 30 min dissociation. At the end of each cycle, the IgG capture surfaces were regenerated by injecting pH 1.5, 10 mM glycine for 60s at 30 μl/min. Running buffer without analyte was injected for double reference subtraction. Curves were fit using 1:1 Langmuir binding.

Similar experimental conditions were set up for the A56 antibodies except different concentrations of A56.hFc were flowed over the sensor (0.6 nM, 1.9 nM, 5.6 nM, 16.7 nM, and 50 nM). Apparent KD is listed, as due to use of bivalent antigens B5.hFc and A56.hFc, the interaction with the antibody may not be a simple 1:1 binding (Table 17). Affinity of A051 could not be determined under these conditions, as it was reaching the limits of detection of the Biacore T200.

TABLE 17
Affinity of Antibodies to A56 or B5 proteins

| | | | Apparent |
|---|---|---|---|
| | ka (1/Ms) | kd (1/s) | KD (nM) |
| A047 (A56) | 1.92 × 10⁴ | 4.50 × 10⁻⁵ | 2.34 nM |
| A049 (A56) | 3.95 × 10⁵ | 2.27 × 10⁻⁵ | ≤0.1 nM* |
| A050 (A56) | 9.98 × 10⁴ | 5.07 × 10⁻⁵ | 0.507 nM |
| A054 (A56) | 9.25 × 10⁵ | 1.41 × 10⁻² | 15.32 nM |
| A048 (B5) | 2.967 × 10⁴ | 2.209 × 10⁻⁵ | 0.745 nM |

*Approaching limits of detection

Antibodies were assessed for affinity to A56.hFc or B5.hFc.

### Example 6—Epitope Binning

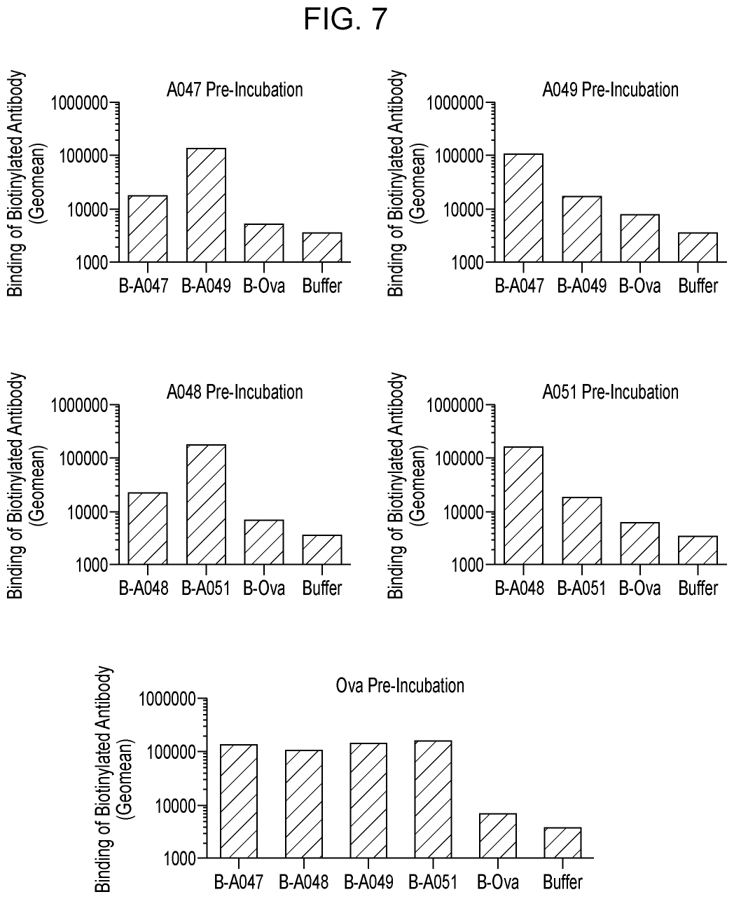

Antibodies were biotinylated using EZ-Link Sulfo-NHS-LC-Biotin (Thermo, Cat #21343) using a 20:1 ratio according to manufacturer's protocols. A549 cells were infected at a MOI of 0.5 using VVdd (eGFP), and fixed on the day before the assay. On the day of the assay, virus-infected A549 cells were pre-incubated with 25 μg/mL of non-biotinylated antibodies and incubated for 1 h. The biotinylated antibodies (10 μg/ml) were then spiked into wells in a 96-well v-bottom plate to a final concentration of 5 μg/ml and allowed incubate for 1 h. Finally, detection was performed using streptavidin-Alexa647 (Jackson, Cat #016-600-084) to detect the bound biotinylated antibodies. Samples were acquired on the Intellicyt HTFC high throughput flow cytometer. An anti-ovalbumin antibody (in-house Centre for Drug Research and Development (CDRD)) was used as an irrelevant control IgG. The data indicates that the anti-A56 antibodies appear to bind different epitopes on A56, as similar binding to the first antibody tested is observed when a 2^{nd} antibody is added to the VAVC coated plate. Similarly, the data indicates that the anti-B5 antibodies appear to bind different epitopes on B5 (FIG. 7).

### Example 7—Antibody Binding to Virus Particles

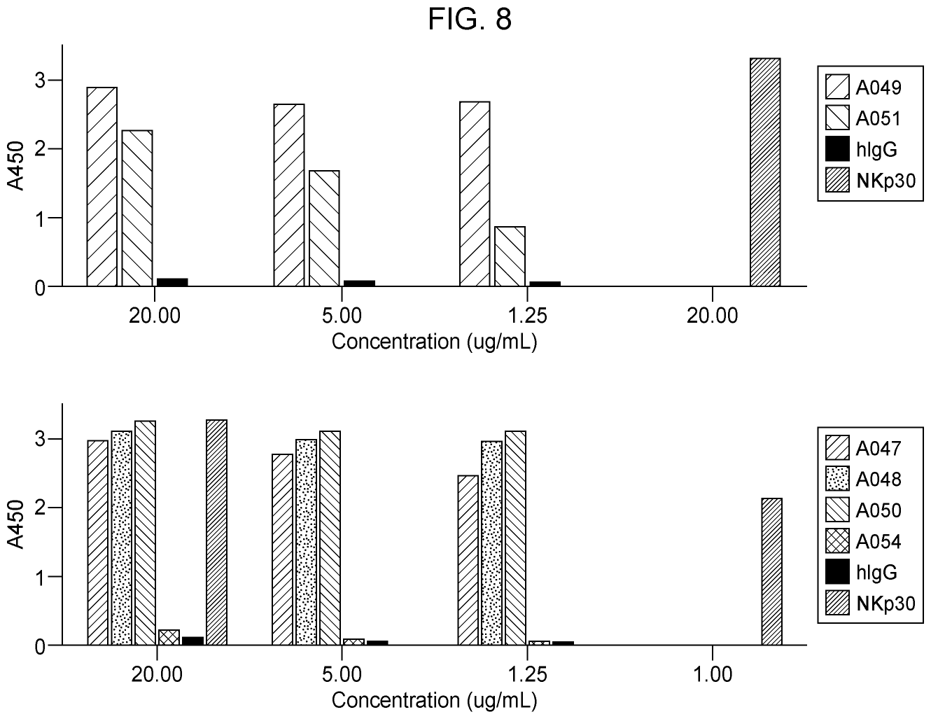

A 96-well plate (Corning, Cat #3368) was coated with 9×10⁶ pfu/mL VVdd (eGFP) in sodium bicarbonate buffer (pH 9.6), 50 μl/well. After overnight incubation at 4° C., the coated wells were washed three times with PBS, and fixation buffer was added and incubated for 10 min. The coated wells were then washed four times with distilled water. The wells were blocked for 1 h at RT with 5% skim milk powder in PBS. Antibodies (A049, A047, A050, A054, A048, A051) were diluted and titrated 1:4 in blocking buffer, from a starting concentration of 20 μg/ml. NKp30, an NK cell cytotoxicity receptor, has been shown to bind VV A56 (2). NKp30-His (in-house) soluble protein was tested at 20 μg/mL or 1 μg/ml as a positive control, and hIgG1 as a negative control. The wells were washed four times with water, and 50 μl/well of antibody was added and incubated for 1 h at RT. Subsequently, the wells were washed with water, and 0.2 μg/mL HRP-conjugated goat anti-hIgG Fc (Jackson, Cat #109-035-098) in blocking buffer was added and incubated for 1 h at RT. After washing, 50 μl/well of TMB (Neogen, Cat #308177) was added and the plate incubated for 30 min at RT. The color development was stopped with 1 M HCl, 50 μl. Absorbance was measured at 450 nm using Spectromax plate reader and data was processed using SoftMaxPro. Data shows all antibodies tested except A054 bind to virus particles of VVdd (eGFP) (FIG. 8).

### Example 8—Effect of Antibodies on Virus Infection

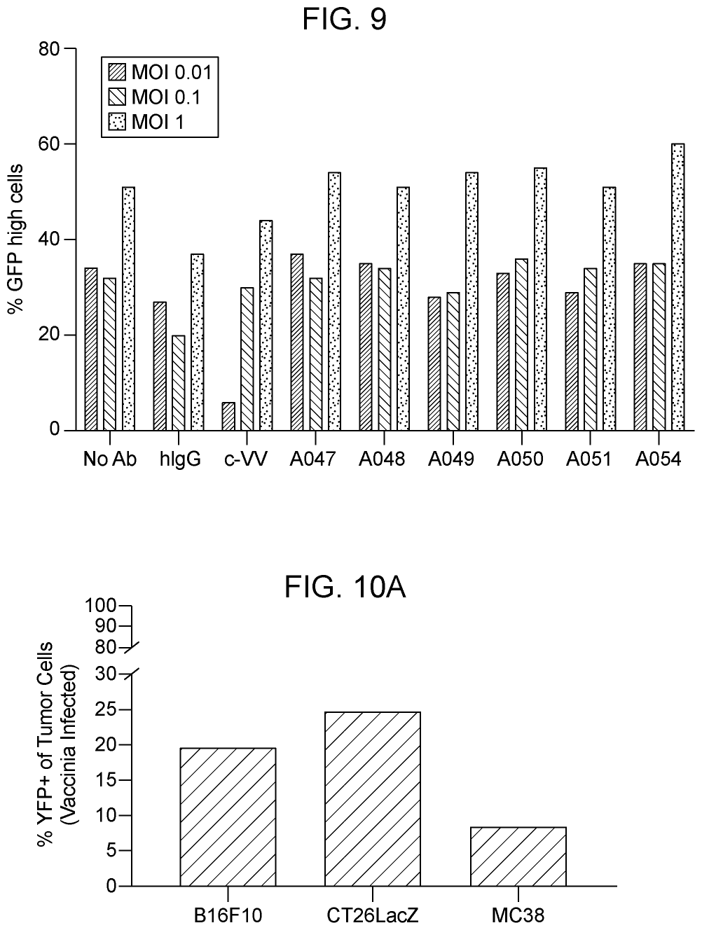

A549 cells were seeded at 5×10⁴ cells per well in 96-well flat bottom plate (Flacon, Cat #353075) and adhered overnight at 37° C., 5% CO₂. VVdd (eGFP) at different MOIs (1.5×10⁶ pfu/mL (MOI 1), 1.5×10⁵ pfu/mL (MOI 0.1) and 1.5×10⁴ pfu/mL (MOI 0.01)) was incubated with equal volume of 20 μg/mL A047, A048, A049, A050, A051, A054, hIgG (Sigma, Cat #15154) or 1:100 vaccinia virus polyclonal antibody (Cedarlane, Cat #LS-C103289) in serum-free F-12K Nutrient Mixture (Gibco, Cat #21127-022), for 15 min at RT. Cells were infected with the VVdd (eGFP)/antibody mixture for 6.5 h at 37° C., 5% CO₂ followed by addition of F-12K Nutrient Mixture (Gibco, Cat #21127-022), supplemented with 10% FBS (Corning 35-015-CV Lot 35015124), at 1:2 dilution and cultured overnight. After incubation, adherent cells were trypsinized and plated alongside non-adherent cells transferred to a 96-well v-bottom plate (Sarstedt, Cat #82.1583.001). Cells were centrifuged at 450 G for 4 min, resuspended in 1:1000 fixable viability dye eFluor 780 (eBioscience, Cat #65-0865-14) and incubated for 15 min, RT. Cells were then washed with PBS, fixed with IC fixation buffer (eBioscience, Cat #00-8222-49) for 10 min RT, washed with PBS, and finally resuspended in 1×PBS with 6000 beads per well of CountBright absolute counting beads (Invitrogen, Cat #C36950). Samples were analyzed by flow cytometry on a BD LSRFortessa X-20 HTS system, and data processed with FlowJo V 10.5.3 Software. Data indicates that addition of these antibodies does not inhibit or reduce VVdd (eGFP) infection of tumor cells (FIG. 9).

### Example 9—Antibody Thermal Stability

Denaturing temperatures (Tₘ) of antibodies were determined from differential scanning fluorimetry (DSF) using Protein Thermo Shift Dye Kit™ (ThermoFisher, Cat #: 4461146). Briefly, 31 μg/mL of antibody was used in each reaction. Melting curves of the antibodies were generated using an Applied Biosystems QuantStudio 7 Flex Real-Time PCR System with the recommended settings stated in the kit manual. The Tₘ's of the antibodies (Table 18) were then determined by using the ThermoFisher Protein Thermal Shift software (v. 1.3). Tₘ1 of the antibodies was determined by DSF.

TABLE 18
Antibody Thermal Stability

| Antibody | Tₘ1 |
|---|---|
| A047 | 71.6 ± 0.2° C. |
| A048 | 70.7 ± 0.1° C. |
| A049 | 73.8 ± 0.4° C. |
| A050 | 70.8 ± 0.2° C. |
| A051 | 71.2 ± 0.2° C. |
| A054 | 68.0 ± 0.9° C. |

### Example 10—Antibody Binding to Murine Cell Lines Infected with Vaccinia Copenhagen

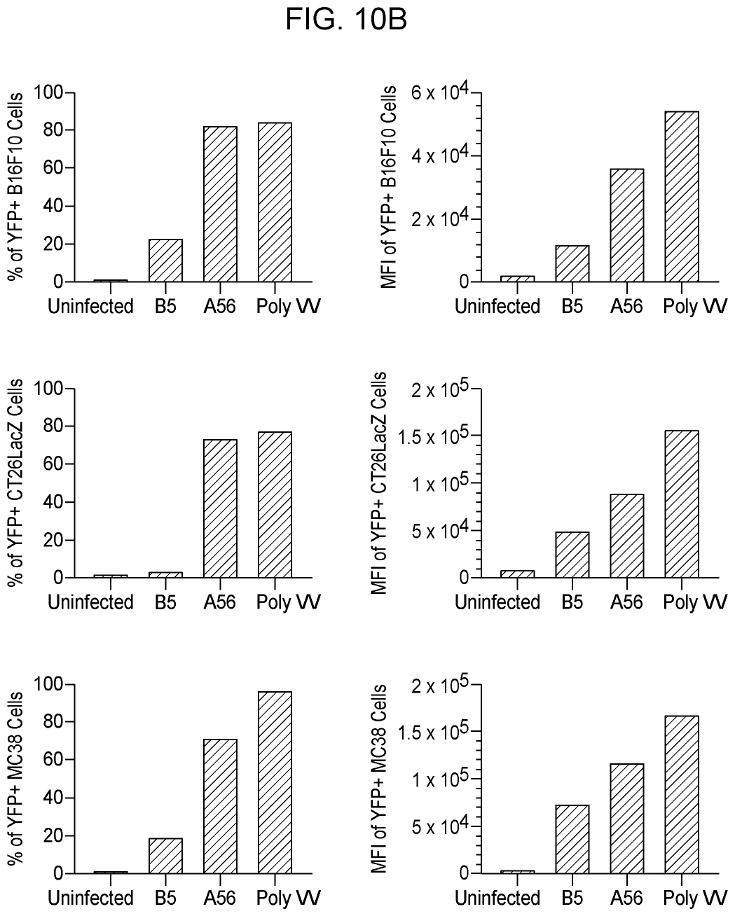

B16F10 (ATCC, Cat #CRL-6475), CT26 LacZ (ATCC, Cat #CRL-2639), and MC38 (gift from Dr. John Bell, OHRI) tumor cells were grown to 80-90% confluence in 10 cm tissue culture plates (Thermo Scientific, Cat #12-556-002) in appropriate media (B16F10—DMEM (Gibco, Cat #11995-040); MC38—DMEM (Gibco, Cat #11995-040); CT26 LacZ—RPMI1640 (Gibco, Cat #11875-093) supplemented with 10% FBS (Gibco, Cat #12483020). Cells were infected with VVcopenhagen (YFP) (gift from Dr. John Bell, OHRI) at a multiplicity of infection of 0.5 in serum-free DMEM (Gibco, Cat #11995-040) and incubated for 6.5 h, at 37° C. in 5% CO₂. Plates were supplemented with growth media at a 1:2 ratio, and further incubated overnight (˜16 hs). B16F10 cells were trypsinized (TrypLE, Cat #12604021) and plated alongside the CT26 LacZ and MC38 cells at approximately 1×10⁵ cells per well, in 96-well U-bottom plates (Sarstedt, Cat #83.3925). Corresponding uninfected B16F10, CT26 LacZ, and MC38 cells were also plated at 1×10⁵ cells per well. Cells were washed twice with PBS 3% FBS (FACs Buffer) with centrifugation at 1800 rpm for 3 min. Cells were resuspended in PBS and incubated with 1:3000 fixable viability dye Zombie NIR (Biolegend, Cat #423106) for 15 min at RT. Following two washes with FACS buffer, cells were resuspended in FACS buffer with mouse Fc block (BD, Cat #553141) and incubated for 5 min at RT. Without further washing, cells were stained directly with either anti-A56-PE (A056, Rb IgG) at 1:100, anti-B5-PE (A073, Rb IgG) at 1:100, or anti-Vaccinia Virus rabbit polyclonal antibody (CedarLane, Cat #LS-C103289) at 1:100 (which served as a positive control) and incubated for 30 min at 4° C. Cells were washed twice with FACS buffer and resuspended. Wells that received anti-vaccinia virus primary antibody were then stained with anti-rabbit IgG-PE (Biolegend, Cat #406421) at 1:100 and incubated for an additional 20 min at 4° C. Following an additional wash with FACs buffer, cells were fixed in IC fixation buffer (eBioscience, Cat #00-8222-49) for 10 min, washed twice with PBS, resuspended in FACS buffer, and analyzed by flow cytometry on a Cytek Aurora flow cytometer. Data was processed using FlowJo V 10.5.3 Software. The data shows percent YFP expression in each infected cell line, indicating the proportion of cells infected with VVcopenhagen (YFP) (FIG. 10A). The anti-A56-PE and anti-B5-PE antibodies were found to bind B16F10, CT26 LacZ, and MC38 cells that, according to their YFP positivity, were infected with VVcopenhagen (YFP) (FIG. 10B). As expected, the anti-Vaccinia Virus rabbit polyclonal antibody also bound to infected (YFP-positive) cells. No binding was observed to uninfected negative control cells.

### Example 11—Immunohistochemistry Detection of Vaccinia A56 and B5

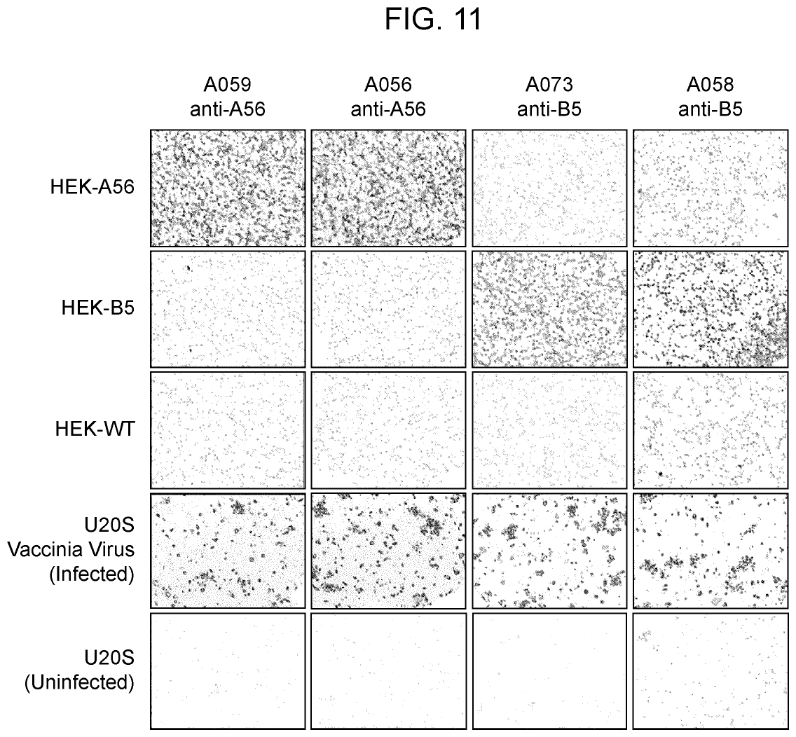

HEK-B5 (GFP) cell lines were grown to 90% confluence in ten 15 cm tissue culture plates (Corning, Cat #353025) in appropriate media (HEK—DMEM (Gibco, Cat #11995-040) supplemented with 10% FBS (Gibco, Cat #12483020)). Additionally, U20S cells (a gift from Dr. John Bell, OHRI) were grown to 90% confluence and infected with VVcopenhagen (YFP) at a multiplicity of infection of 0.025 and incubated for 72 h at 37° C. in 5% CO₂. HEK-A56, HEK-B5, HEK-WT and U20S cells were trypsinized (TrypLE, Cat #12604021), counted, and resuspended to a concentration of 2×10⁸ cells/ml in 15 ml polypropylene tubes (FroggaBio, Cat #TB15-500). Cells were centrifuged at 1500 RPM for 5 min, resuspended in 1 ml of molten histogel (Thermo Scientific, Cat #HG-4000-012), and placed on ice for 10 min to allow the histogel to solidify. Histogel pellets were transferred to 10 ml 10% neutral buffered formalin (Sigma, Cat #HT501128) and left at RT for 24 h. For immunohistochemistry (IHC), histogel sections (4 μm) were incubated overnight at 37° C., deparaffinized, rehydrated using xylene and an ethanol gradient, and subjected to antigen retrieval with Diva decloaker reagent (Biocare, Cat #DV2004) in a Biocare decloaking chamber (110° C. for 15 min), and loaded into an Intellipath FLX Autostainer. Brightfield IHC was performed at room temperature with Biocare reagents. Slides were treated with Peroxidased-1 (Biocare, Cat #PX968) for 5 min and Background Sniper (Biocare, Cat #BS966) for 10 min. Primary antibodies diluted in DaVinci Green (Biocare, Cat #PD900) were applied for 30 min followed by Mach2 HRP Polymer (either mouse or rabbit, based on the species of the primary antibody) (Biocare, Cat #RHRP520 & MHRP520) for 30 min. DAB horseradish peroxidase (Biocare, Cat #IPK5010G80) chromogen substrate was added to the slides. CAT hematoxylin (Biocare, Cat #CATHE) diluted 1:5 with dH₂O was added for 5 min at RT. Stained slides were washed with water, air-dried and cover-slipped using Ecomount (Biocare, Cat #EM897L). Antibody dilutions were as follows: A058 anti-B5, Rb/Mu IgG2a, 1/280 (10 μg/mL); A059 anti-A56, Rb/Mu IgG2a, 1/540 (10 μg/mL); A056 anti-A56, Rb IgG, 1/840 (10 μg/mL); A073 anti-B5, Rb IgG, 1/550 (10 μg/mL). Antibodies specific for A56 or B5 were able to specifically detect target antigen on stably transfected HEK-A56 and HEK-B5 cells, respectively (FIG. 11). No cross reactivity against alternative antigen or HEK-WT cells was observed. Both anti-A56 and anti-B5 antibodies were able to detect protein expression on infected U20S cells, and no background staining was detected on uninfected U20S cells (FIG. 11).

### Example 12—Immunohistochemical Detection of A56 and B5 from VV Treated Tumors

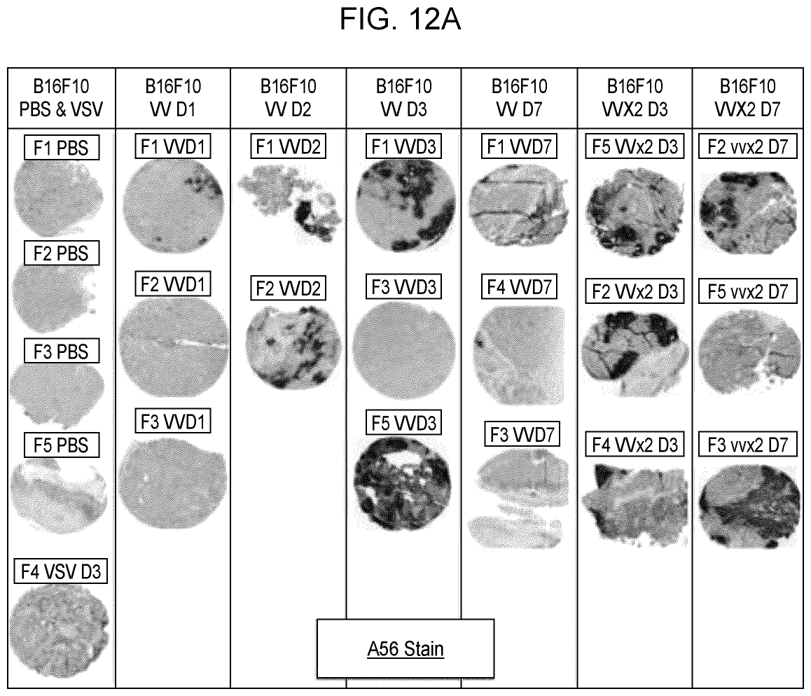

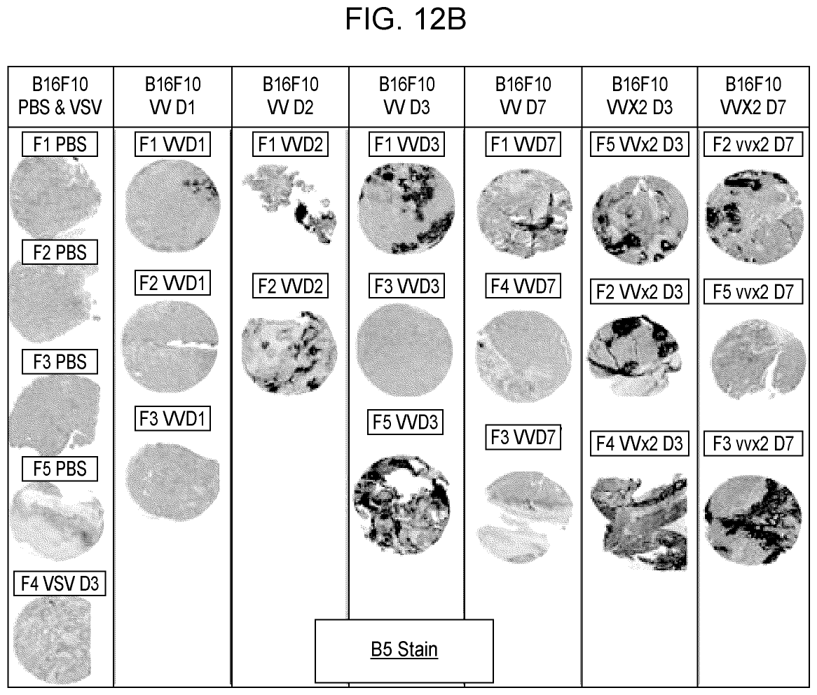

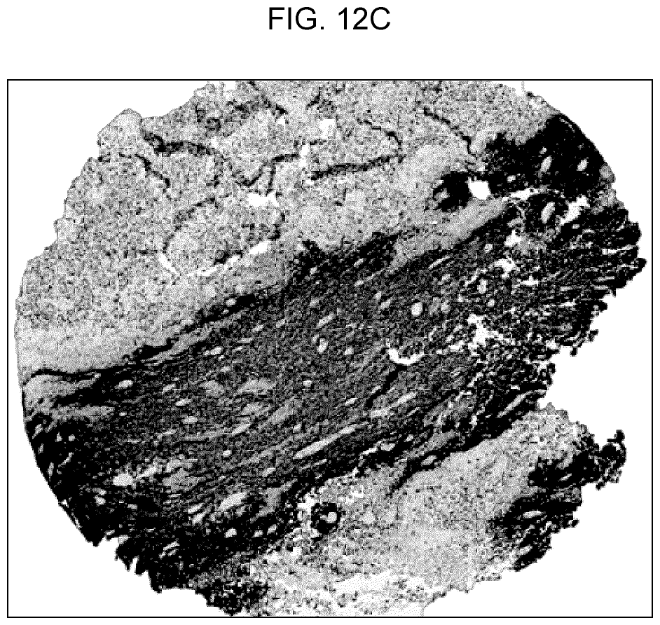

C57BL/6 wild type mice (Jackson Laboratory, Cat #000664) were implanted with 1×10⁶ B16F10 (ATCC, Cat #CRL-6475) tumor cells subcutaneously on the rear right flank. Mice were grouped as follows: 1 dose VVcopenhagen (YFP), 2 doses VVcopenhagen (YFP), PBS, and Vesticular stomatitis Virus (VSV) (gift from Dr. John Bell, OHRI). All injections were at a dose of 1×10⁷ PFU in 100 μl volume and delivered intratumorally when tumors reached a size of 35-40 mm². Animals were euthanized either 1, 2, 3, or 7 days following their first injection. Tumors were collected and fixed in 10% neutral buffered formalin for 24 h. Following fixation tumors were embedded in paraffin wax and 1-52 mm tumor punches were taken from each resected tumor. Tumor punches were arrayed to create a tumor tissue microarray (TMA) and sectioned onto glass slides. For IHC, TMA sections (4 μm) were incubated overnight at 37° C., deparaffinized, rehydrated using xylene and an ethanol gradient, and subjected to antigen retrieval with Diva decloaker reagent (Biocare, Cat #DV2004) in a Biocare decloaking chamber (110° C. for 15 minutes), and loaded into an Intellipath FLX Autostainer. Brightfield IHC was performed at RT with Biocare reagents. Slides were treated with Peroxidazed-1 (Biocare, Cat #PX968) for 5 min and Background Sniper (Biocare, Cat #BS966) for 10 min. Primary antibodies diluted in DaVinci Green (Biocare, Cat #PD900) were applied for 30 min followed by Mach2 HRP Polymer (either murine or rabbit, based on species of primary antibody) (Biocare, Cat #RHRP520 & MHRP520) for 30 min. DAB horseradish peroxidase (Biocare, Cat #1PK5010G80) chromogen substrate was added to the slides. CAT hematoxylin (Biocare, Cat #CATHE) diluted 1/5 with dH₂O was added for 5 min at RT. Stained slides were washed with water, air-dried and cover-slipped using Ecomount (Biocare, Cat #EM897L). Slides were stained with either Anti-A56 (A056) or Anti-B5 (A073) Rb IgG and detected with an anti-rabbit secondary antibody. Antibody dilutions were as follows: A056 anti-A56, Rb IgG, 1/840 (10 μg/mL); A073 anti-B5, Rb IgG, 1/550 (10 μg/mL). DAB staining (dark grey) indicates positive protein detection (FIG. 12). Antibodies for A56 or B5 were able to specifically detect target antigen on the majority of cores taken from treated tumors. Increased protein detection was evident on tumors that received multiple VVcopenhagen (YFP) doses. No cross reactivity against PBS or VSV treated tumors was evident. These findings demonstrate the specific detection and extent of virally derived A56 and B5 protein expression following intratumoral delivery of vaccinia virus.

### Example 13—VV Chimeric Antigen Receptor (CAR) Design and Detection Following Lentiviral Transduction

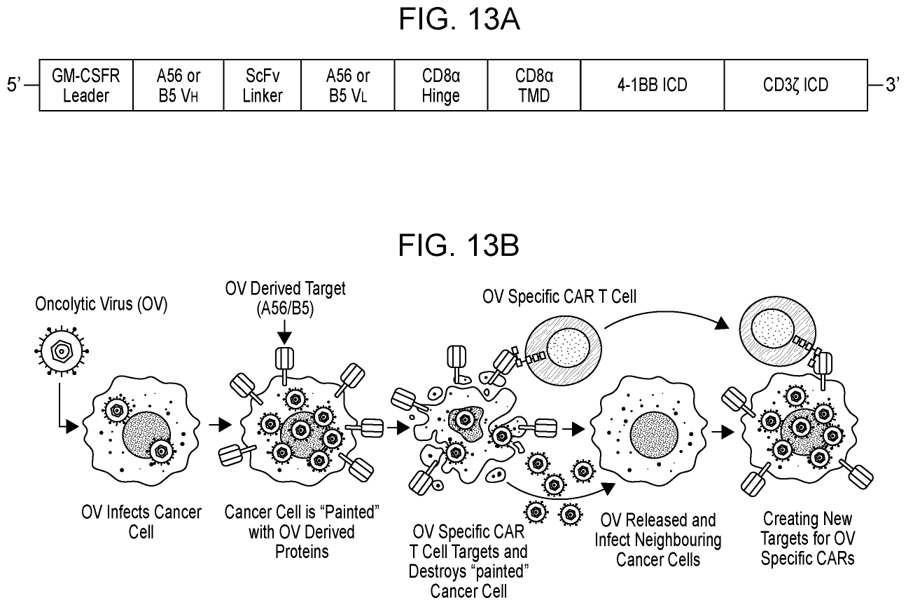

The human VV-CAR constructs all contain a GM-CSFR a leader sequence followed by the A56 or B5 scFv (derived from A56: A049, B5: A048), a human CD8α hinge and transmembrane domain, a human 4-1 BB intracellular signaling domain, and a human CD3ζ intracellular signaling domain (FIG. 13A) (3). Some CAR constructs included a reporter protein (e.g. eGFP) separated by a T2A ribosomal skip sequence. The amino acid sequences of the CARs are provided in Table 7.

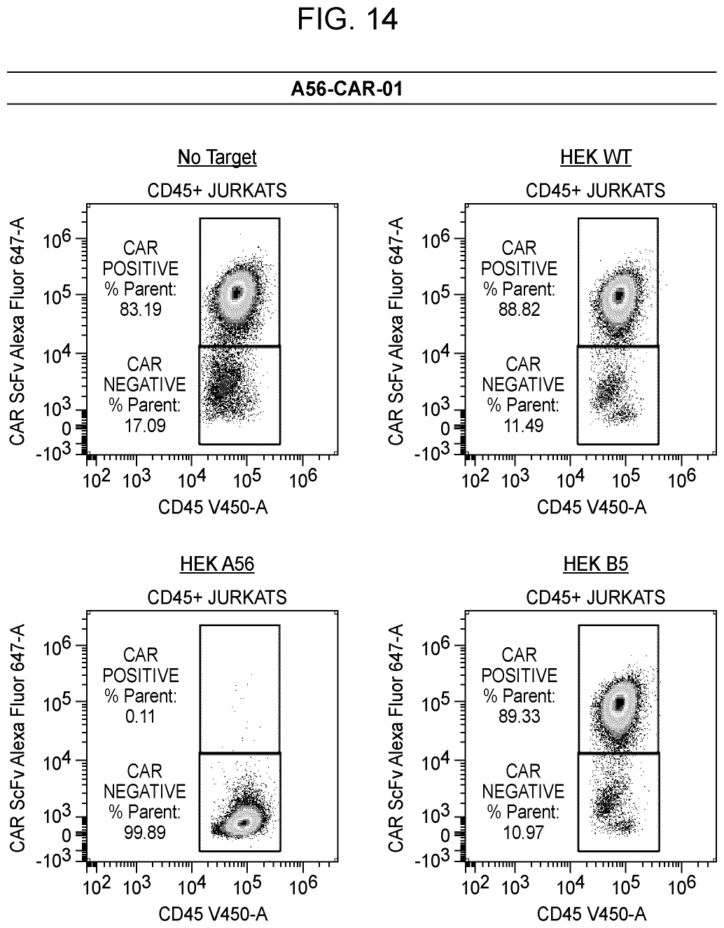

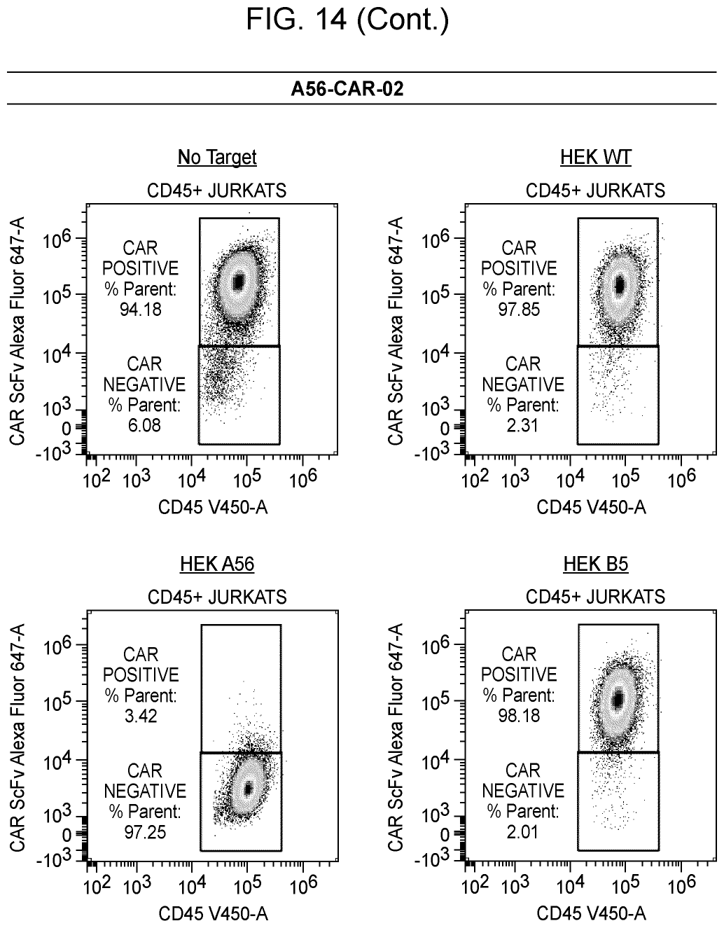

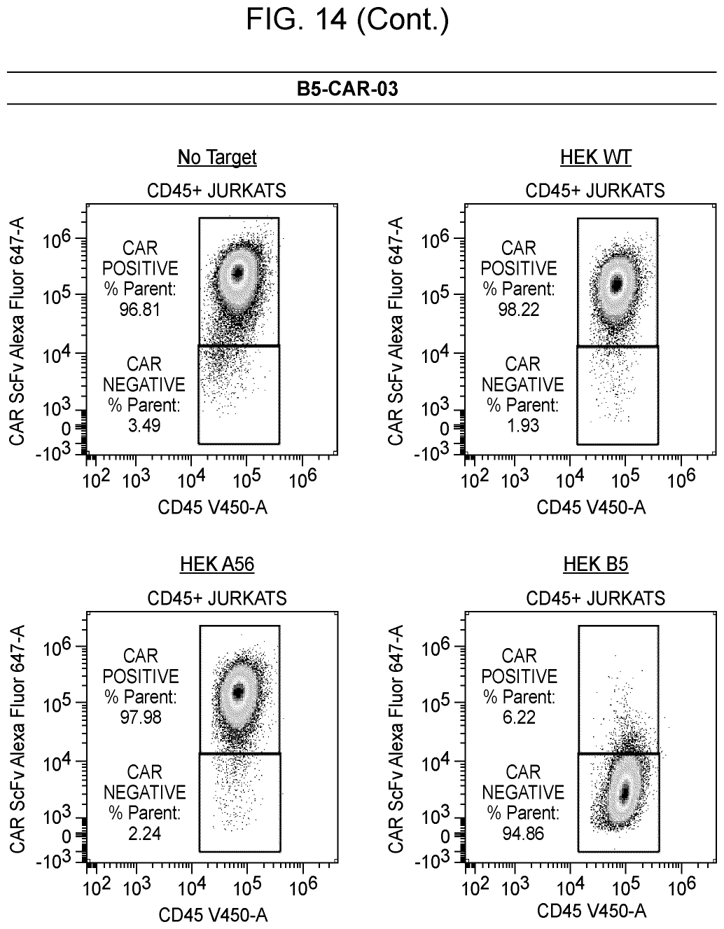

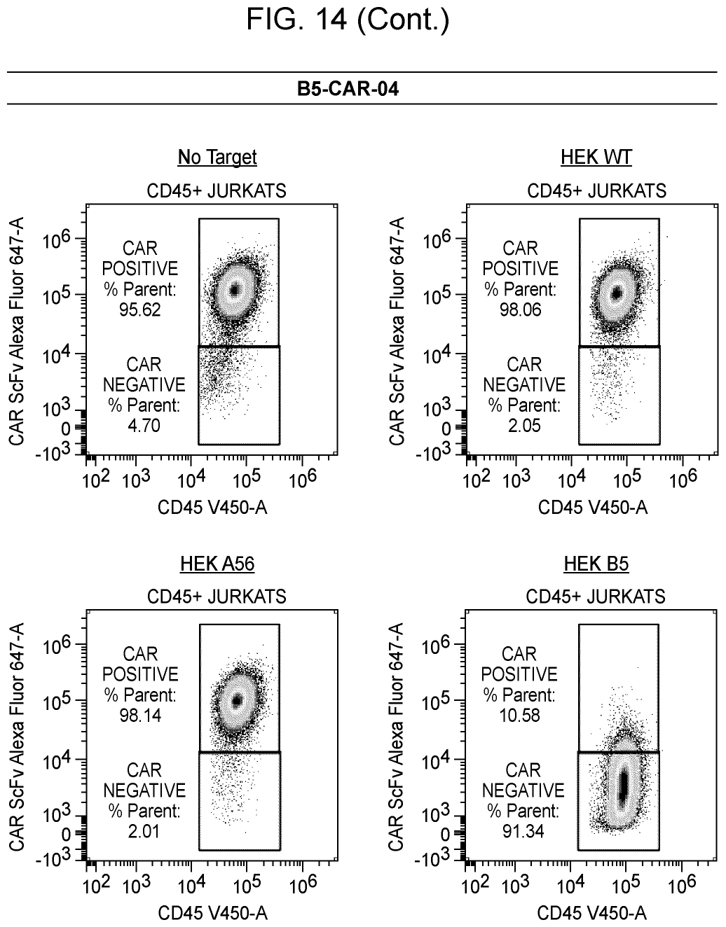

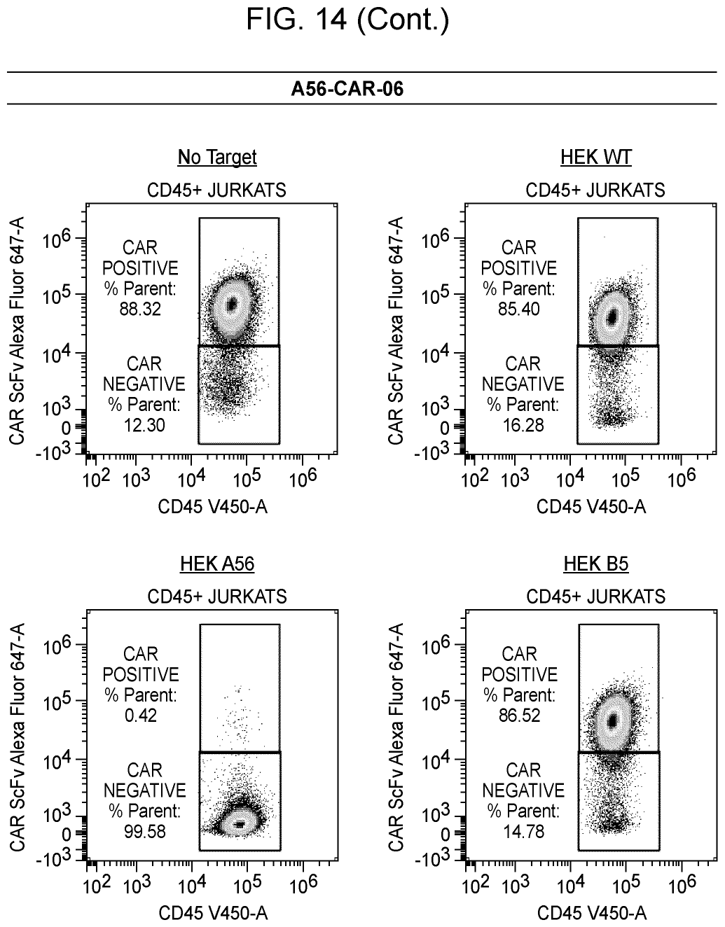

Gene fragments encoding A56-CAR-01, A56-CAR-02, B5-CAR-03, B5-CAR-04 or A56-CAR-06 were synthesized by Twist Biosciences. Gene fragments were cloned into a 2^{nd} generation transfer plasmid (gift from Dr. Robert Holt, BC Cancer). Lentivirus encoding VV-CAR constructs were generated using a standard calcium phosphate transfection protocol (4) and 2^{nd} generation packaging vectors (gift from Dr. Rob Holt BC Cancer). Jurkat T cells (ATCC, Cat #TIB-152) were transduced with 500 μl supernatant from each lentivirus transfection. Jurkat cells were then expanded for 1 week and 1×10⁶ CAR positive cells were sorted based on goat anti-Rb IgG F(ab′)2-Alexa Fluor 647 detection (Jackson Laboratories, Cat #111-605-047). Enriched Jurkat CAR-positive populations were expanded for an additional week and plated in a co-culture assay with HEK-A56, HEK-B5 or HEK-WT cells. Cells were cultured overnight (16 h) at a 1:1 Effector:Target (E:T) ratio (1×10⁵ total cells). The following day cells were washed with PBS, blocked with normal goat serum (Jackson Laboratories, Cat #005-000-001), and stained for CD45 v450 (BD, Cat #560367), CD69 PE-Cy7 (Biolegend, Cat #310912), anti-Rb IgG Alexa Fluor 647. A56-CAR-01, A56-CAR-02, and A56-CAR-06 exhibited downregulation of cell surface CAR molecules when cultured with the target HEK-A56 line, such downregulation of cell surface CAR molecules is indicative of CAR activation (FIG. 14). Similarly, B5-specific CARs, B5-CAR-03, and B5-CAR-04, exhibited downregulation of surface CAR when cultured with the target HEK-B5 line. No CAR downregulation was present when CAR-T cells were cultured with HEK WT lines or irrelevant target antigens. These data demonstrate that effector T cells transduced to express A56- or B5-specific CARs undergo activation (as evidenced by downregulation of cell surface CAR expression) upon encounter with target cells bearing the appropriate antigen (A56 or B5, respectively).

### Example 14—CAR and eGFP Expression on Activated A56-CAR-06 Positive Jurkat T Cells

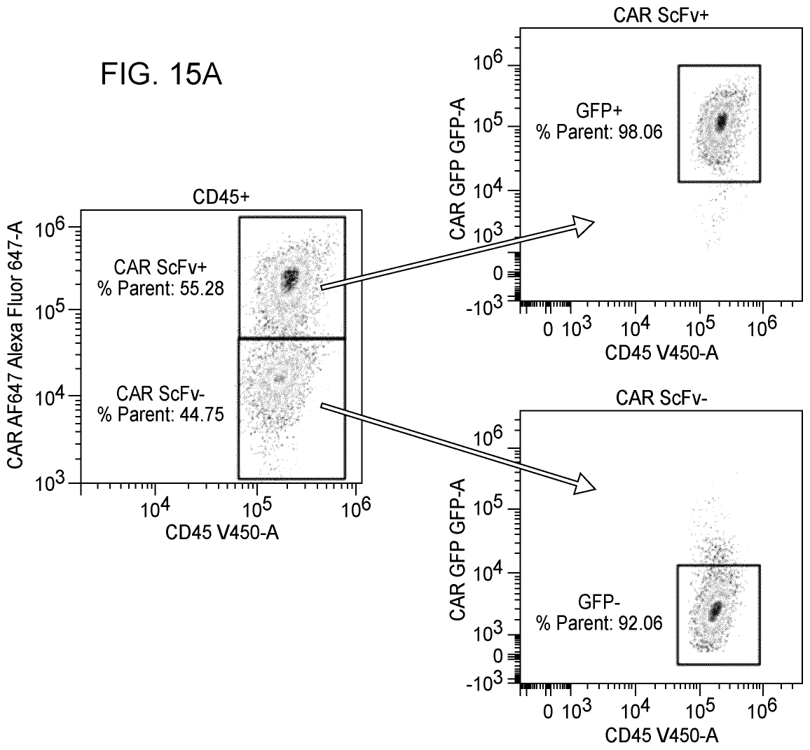

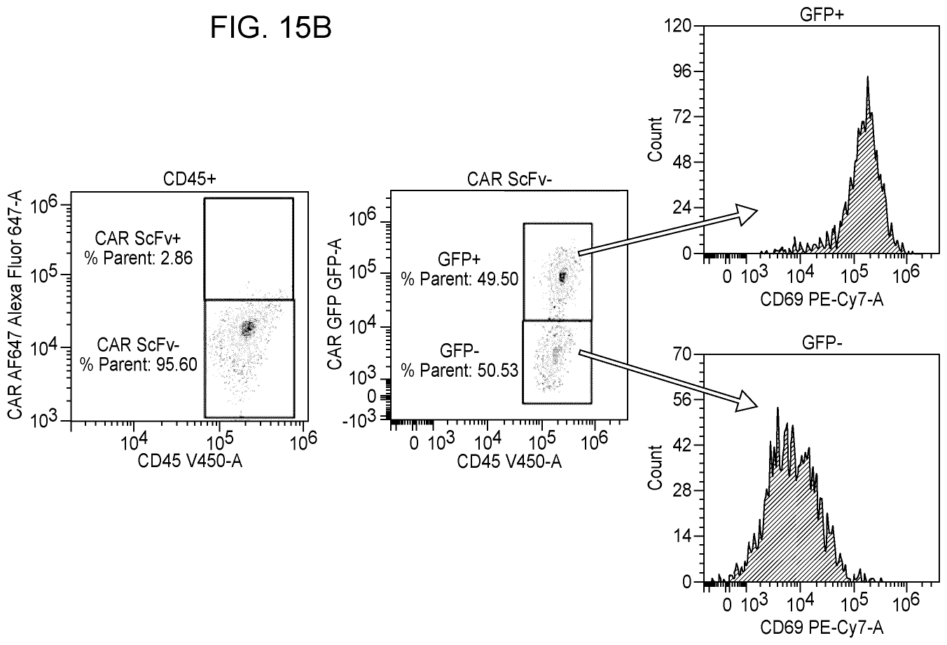

Jurkat A56-CAR-06 lines co-expressing eGFP were cultured with HEK-A56 lines overnight (16 h), and as expected from the results of Example 13, CAR expression was observed to be reduced (FIG. 15). However, within the CAR “negative” population, an eGFP-positive population was observed. Furthermore, the eGFP-positive population exhibited increased expression of CD69, a T cell activation marker, whereas the eGFP-negative population exhibited minimal CD69 expression. CD69 upregulation is indicative of CAR-T cell recognition of target cells bearing the appropriate antigen (A56 in this case). These results show that following encounter with target cells bearing an appropriate antigen (A56 in this case), CAR-T cells exhibit downregulation of cell surface CAR expression yet can still be identified by eGFP and CD69 expression, the latter providing independent evidence of target cell recognition via the CAR.

### Example 15—Jurkat T Cells Expressing VV-CARs Show Specific Activation when Co-Cultured with HEK Cell Lines Bearing the Appropriate Target Antigen

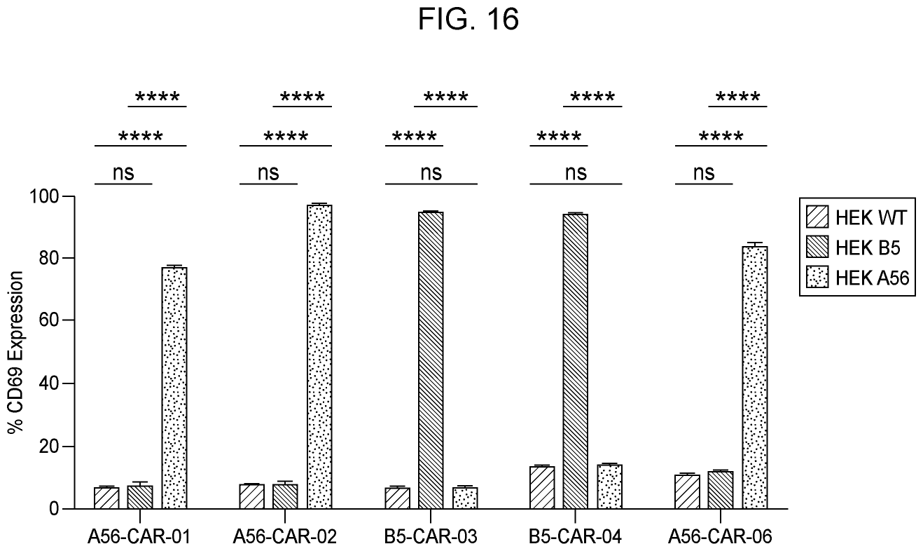

Jurkat T cells expressing either A56-CAR-01, A56-CAR-02, B5-CAR-03, B5-CAR-04 or A56-CAR-06 were co-cultured in triplicate with HEK-WT, HEK-A56, or HEK-B5 stable lines at a 1:1 E:T ratio, 1×10⁵ total cells per condition. Cells were cultured overnight (16 h) at 37° C. in 5% CO₂. The following day cells were washed with PBS, blocked in normal goat serum, and stained for CD45, CD69, anti-RB IgG. CD45+ Jurkat populations were then assessed for CD69 expression. All CARs tested showed increased expression of CD69 when co-cultured with cells expressing the appropriate target antigen but minimal expression of CD69 when co-cultured with HEK WT cells or HEK cells expressing an irrelevant target antigen (FIG. 16). These findings demonstrate that A56- and B5-directed CAR-T cells exhibit specific recognition of target cells expressing the appropriate antigens (A56 and B5, respectively) and show minimal cross-reactivity and/or constitutive activity (tonic signalling).

### Example 16—Primary Healthy Donor T Cells Transduced and Enriched for A56-CAR-06 Expression

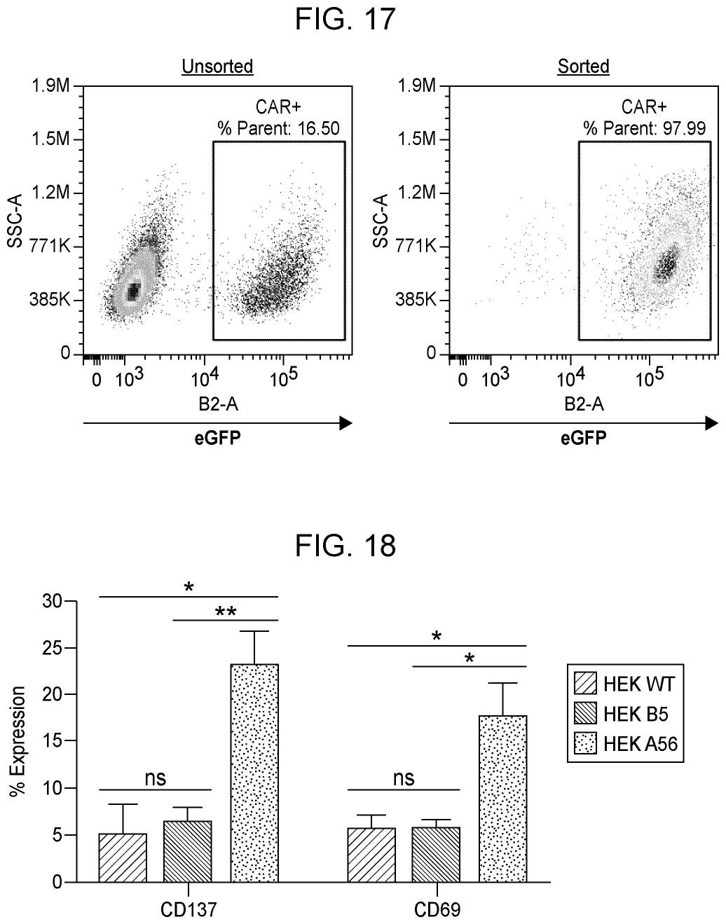

CD4+ and CD8+ T cell populations were isolated from healthy donor PBMC samples. 5×10⁵ healthy donor T cells were activated with Miltenyi TransAct™ (Miltenyi Biotec, Cat #130-111-160) and grown in Miltenyi TexMACS GMP (Miltenyi Biotec, Cat #170-076-309) media supplemented with 3% human serum (Sigma, Cat #H4522), gentamicin sulfate (Sandoz, DIN:02268531) and human interleukin-7 (Miltenyi Biotec, Cat #130-095-367) and interleukin-15 (Miltenyi Biotec, Cat #130-095-764) (10 μg/ml). 24 h after activation, T cells were transduced with A56-CAR-06 lentivirus at an MOI of 0.25. Cells were expanded for the following 12 days at a density less than 1×10⁶ cells per ml. Following expansion a fraction of A56-CAR-06 transduced T cells were sorted on eGFP positivity using a flow cytometer. 2×10⁵ cells were sorted for high purity and plated in a single well of a 96-well round bottom plate. The sorted population was activated as described above and expanded for an additional 12 days, maintaining a cell density below 1×10⁶ cells/ml. The final expanded enriched population was >95% CAR positive by flow cytometry (FIG. 17). These findings show that primary PBMC-derived human T cells can be readily transduced to express VV-CAR constructs and can be enriched and expanded post-transduction.

### Example 17—Human T Cells Expressing A56-CAR-06 Show Specific Activation when Co-Cultured with A56-Expressing HEK293T Lines

Human T cells expressing A56-CAR-06 were co-cultured in triplicate overnight (16 h) with HEK-WT, HEK-A56, or HEK-B5 lines at a 1:1 E:T ratio with 1×10⁵ cells total per condition. The following day cells were washed with PBS, blocked with normal goat serum, and stained to detect expression of CD3 BV510 (Biolegend, Cat #317332), CD45, CD69, CD137 BV650 (Biolegend, Cat #564092), anti-RB IgG. CD3+ eGFP+ T cell populations were assessed by flow cytometry for CD69 and CD137 expression. The A56-CAR-06 transduced T cells showed increased expression of CD69 and CD137 when co-cultured with cells expressing the A56 target antigen but minimal expression of CD69 and CD137 when co-cultured with HEK-WT cells or HEK cells expressing an irrelevant antigen (FIG. 18). These findings demonstrate that primary T cells transduced to express A56-CAR-06 exhibit specific activation when cultured with target cells expressing the A56 antigen on their surface.

### Example 18—Human T Cells Expressing A56-CAR-06 Induce Morphological Signs of Cell Death in A56-Expressing Target Cells

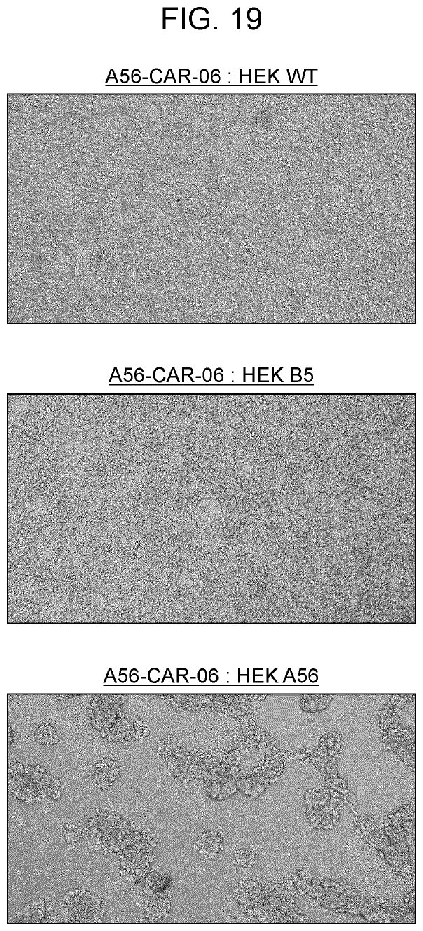

HEK-WT, HEK-A56, or HEK-B5 cells (2×10⁵ each) were seeded in a 24-well tissue culture plate and left overnight to adhere. The following day 1×10⁵ A56-CAR-06 T cells were added to each well, and cells were cultured for an additional 48 h. Images depict distinct morphological changes to the HEK-A56 target cell line when co-cultured with A56-CAR-06-positive T cells, indicative of direct tumor cell killing. In contrast, HEK-WT and HEK-B5 lines remained fully confluent with no morphological changes (FIG. 19). These findings show destruction HEK-A56 cell monolayers following co-culture with A56-CAR-06 T cells, providing evidence of a direct cytopathic effect.

### Example 19—Human T Cells Expressing B5-CAR-011 Demonstrate Cytotoxicity Against B5-Expressing Target Cells

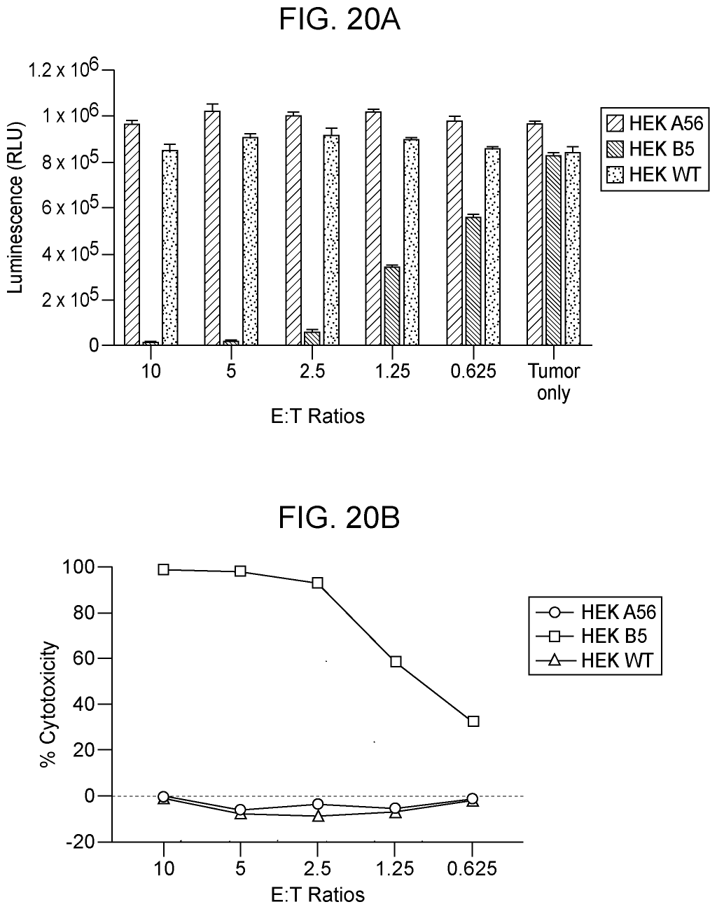

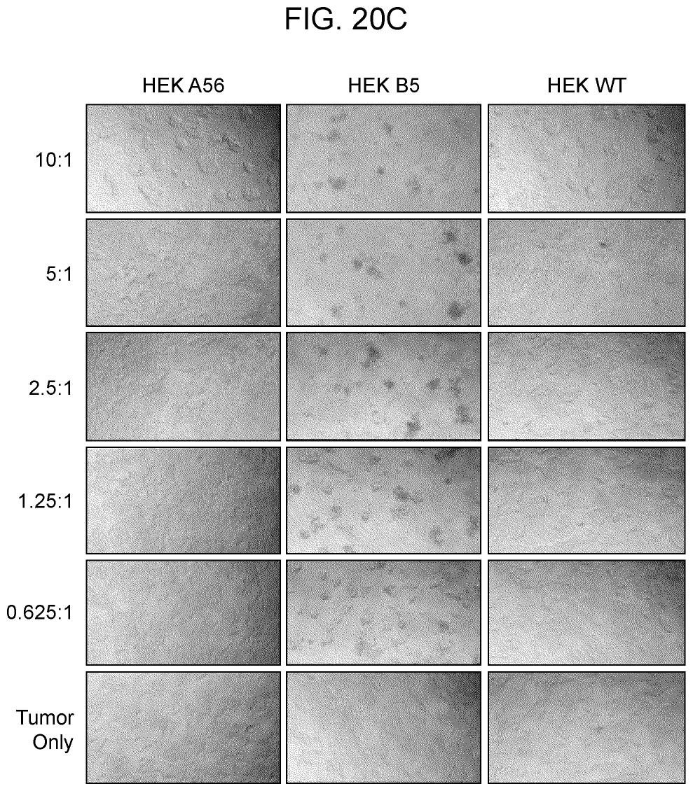

A mammalian expression transfer plasmid encoding firefly luciferase, pCCL Luc Puromycin (gift from Dr. Jonathan Bramson, McMaster University) was used to generate lentivirus, as described previously in Example 13. HEK-WT, A56, and B5 lines were transduced with this luciferase-encoding lentivirus and selected over 2 weeks for resistance to puromycin (1.5 μg/ml, Sigma, Cat #P8833). Puromycin-resistant cell cultures were expanded and used as target populations for VV-CAR killing assays. Luciferase-expressing target HEK lines were seeded at 2×10⁴ cells, 100 μl per well in a 96-well plate (Corning, Cat #3917) and incubated overnight to adhere. A sorted population of previously expanded primary human B5-CAR-011 T cells was plated (100 μl per well) in triplicate in a 96-well plate and split over a 2-fold dilution series to achieve a range of E:T ratios from 10:1 to 0.625:1. The diluted T cell suspensions were then transferred to the adherent HEK cells to achieve a total volume of 200 μl. Additionally, 3 wells of target cells alone and 3 wells of media only were plated to determine maximal and minimal relative luminescence units (RLU). Cells were cultured for 24 h at 37° C. in 5% CO₂. The following day, each well received 22 μl 10× stock of Xenolight™ D-Luciferin (Perkin Elmer, Cat #122799) and incubated for 10 min at RT in the dark. Plates were then scanned on a luminescence plate reader (Perkin Elmer Wallac Envision 2104 Multilabel Reader). FIG. 20A depicts average RLU from triplicate wells at each E:T ratio at 24 h. Triplicate wells were averaged, and the percent specific cytotoxicity was determined by the following equation: percent specific cytotoxicity=100×(Max Luminescence RLU−Test Luminescence RLU)/(Min Luminescence RLU−Max Luminescence RLU). B5-CAR-011 T cells exhibited high percent specific cytotoxicity at multiple E:T ratios (FIG. 20B). Moreover, B5-expressing target cells co-cultured with B5-CAR-011 T cells showed clear morphological signs of cell death at 24 h, whereas B5-CAR-011 T cells showed no signs of cytotoxicity against HEK293 WT or HEK293 A56 cells (FIG. 20C). The results show that B5-CAR-011 T cells efficiently and specifically kill HEK-B5 tumor cells. (FIG. 20). By targeting OV antigens expressed on the surface of tumor cells, target cells can be made susceptible to OV-CAR mediated destruction.

### Example 20—Radiolabeled Anti-A56 Antibody Enables Clear Visualization of A56-Expressing Tumor Cells In Vivo by Positron Emission Tomography

Anti-A56 antibody (A049) was selected and radiolabeled with Zirconium-89 (⁸⁹Zr) for biodistribution studies and microPET imaging, which allows deep tissue imaging in live mice with high sensitivity and high spatial resolution (5, 6). ⁸⁹Zr is well suited for use with mAbs due to its long half-life (78 h) and favourable decay properties (6, 7). Two milligrams of A049 were conjugated with p-SCN-Bn-deferoxamine (1-(4-isothiocyanatophenyl)-3-[6,17-dihydroxy-7,10,18,21-tetraoxo-27-(N-acetylhydroxylamino)-6,11,17,22-tetraazaheptaeicosine]thiourea, DFO, Macrocyclics, Plano, TZ, USA) with a chelator:mAb molecular ratio of 3:1. The reaction was performed at 37° C. for 1 h in PBS pH 9 with a final concentration of 2 mg/mL of antibody. Conjugated antibody (DFO-A049) was then purified from the unconjugated deferoxamine using centrifugal filter units with a 50 kDa molecular weight cutoff (Amicon ultracentrifuge filters, Ultracel-50: regenerated cellulose, Millipore Corp., Billerica, Mass., USA) and washed once with 5 mg/mL 2,5-dihydroxybenzoic acid in 0.25 M sodium acetate solution (Sigma-Aldrich, Oakville, Ontario, Canada). The concentration of the purified immunoconjugate solution was determined by a Bradford assay according to the manufacturer's recommendations (Sigma-Aldrich).

Immunoconjugate Radiolabeling with ⁸⁹Zr

⁸⁹Zr was produced via proton irradiation on an yttrium-89 disk (American Elements, Los Angeles, Calif., USA) in an ACSI (Richmond, Canada) TR-19 cyclotron at 13 MeV as previously described (8). After irradiation, the disc was dissolved in HCl (10 mL, 2 M), and purified using a hydroxamate-based ZR resin supplied by Triskem (Bruz, France). Briefly, the disk solution was loaded on the resin, washed with 10 mL of 2 M HCl solution followed by 10 mL of water, and then eluted off with 0.5 mL of oxalic acid (0.05 M).

For radiolabeling, the immunoconjugate DFO-A049 (0.4 mg) and ⁸⁹Zr solution (0.4 mL, pH 7, 232 MBq) were added to PBS (final volume: 1 mL, pH 7), and the resulting solution was incubated for 1 h at room temperature. The radiochemical yield (RCY) was determined using instant thin layer chromatography silica gel (iTLC-SG, Agilent technologies, Santa Clara, Calif., USA) with 50 mM DTPA pH 7 as a solvent (Sigma-Aldrich). The Rf values of ⁸⁹Zr-labeled antibody and free ⁸⁹Zr were 0 and 1, respectively. Efficient labeling was observed with a RCY of 96%. ⁸⁹Zr-labeled antibodies were then separated from free ⁸⁹Zr by a PD-10 desalting column (GE Healthcare, London, United Kingdom), and concentrated using a 50 kDa molecular weight cut off filter (Millipore Corp.). The specific activity of ⁸⁹Zr-DFO-A049 was determined using a size-exclusion HPLC column (BioSep-SEC-s3000, Phenomenex, Torrance, Calif., USA) on an Agilent HPLC system (Santa Clara, Calif., USA) equipped with a model 1200 quaternary pump, a model 1200 UV absorbance detector, and a Bioscan (Washington D.C., USA) NaI scintillation detector. The HPLC buffer was an isocratic gradient of 0.1 M sodium phosphate monobasic dihydrate, 0.1 M sodium phosphate dibasic dodecahydrate, 0.1 M sodium azide and 0.15 M sodium chloride (pH 6.2-7.0). A specific activity of 0.1 MBq/μg was obtained and was sufficient for in vivo characterization of the radioimmunoconjugate. Final radiochemical purity was determined using iTLC-SG as previously described and was >99.9%.

### Antibody Immunoreactivity

The immunoreactivity fraction of ⁸⁹Zr-DFO-A049 was estimated according to the Lindmo cell-binding method using HEK-293 cells stably modified to express the A56 antigen (Wyeth VV sequence) (HEK-A56). Briefly, HEK-A56 cells were suspended at different concentrations from 1.0 to 24.6×10⁶ cells/mL in PBS pH 7.4. The remaining procedure was performed as previously described (9). Results showed that 52% of the ⁸⁹Zr-DFO-A049 is still efficient to bind to A56 protein.

### Animal Tumor Models and Antibody Injection

All animal experiments were performed at the Animal Resource Centre of the BC Cancer Research Centre in accordance with the institutional guidelines of the University of British Columbia Animal Care Committee (Vancouver, British Columbia, Canada) and under the supervision of authorized investigators. Twelve week old female immunodeficient NOD.Cg-Rag1tm1 Mom II2 rgtm1 Wjl/SzJ (NRG) mice (obtained from an in-house breeding colony) were subcutaneously injected with HEK-A56 cells on the left shoulder or parental non expressing A56 HEK-293 cells on the right shoulder (n=8 mice). For both cell lines, 5×10⁶ cells were injected in matrigel (1:1 ratio, BD Bioscience, Mississauga, Ontario, Canada).

### Positron Emission Tomography (PET)

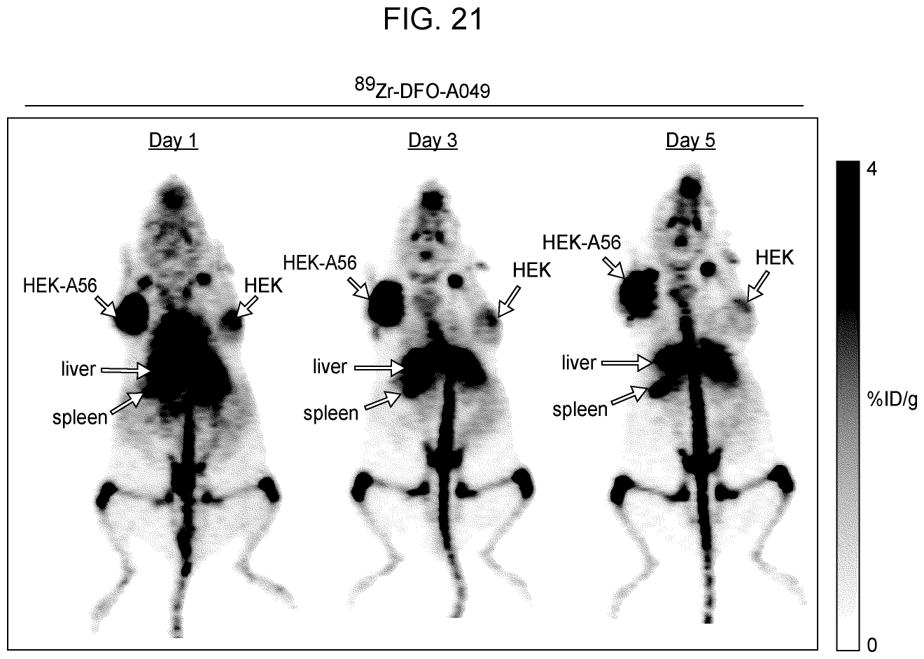

When tumors reached imaging size (˜100 mm³), the mice were anesthetized with 2% isoflurane in oxygen and intravenously injected with ⁸⁹Zr-DFO-A049 (40.9±1.5 μg, 4.1±0.2 MBq). PET images were acquired for one mouse 1, 3 and 5 days post-injection using a Siemens (Knoxville, Tenn., USA) Inveon microPET/CT scanner. The mouse underwent a CT scan for attenuation correction followed by a 20 min static PET acquisition at day 1 and 3, and 30 min scan at day 5. Images were reconstructed using the 3-dimensional ordered-subsets expectation maximization (OSEM3D, 2 iterations) followed by a fast maximum a priori algorithm (FastMAP: 18 iterations). Maximum intensity projection images show expected accumulation in normal organs such as liver and spleen due to antibody processing and metabolism, in addition to bone uptake resulting from known demetalation of ⁸⁹Zr-DFO conjugates in mice (especially in high bone remodeling regions such as long bone epiphysis) (10). More importantly, PET images show higher tumor uptake and tumor-to-background ratios for the HEK-A56 tumor compared to the negative control one (HEK-293), indicating specific and efficient tumor uptake of the ⁸⁹Zr-DFO-A049 antibody (FIG. 21).

### Assessment of ⁸⁹Zr-DFO-A049 Biodistribution

At day 5, all mice were euthanized and organs of interest were collected for biodistribution studies as described previously (9). Results are shown in Table 19 for ⁸⁹Zr-DFO-A049.

TABLE 19
Biodistribution of ⁸⁹Zr-DFO-A049 at
day 5 in A56-expressing HEK (HEK-
A56) and non-expressing HEK-293 tumor-
bearing mice. The values are expressed as
mean ± SD percent injected dose per gram
of tissue (% ID/g); n = 8 per time point.
Statistical difference between HEK-A56
and HEK is shown (**, p < 0.05, t-test).

| Organs | Day 5 |
|---|---|
| Blood | 0.32 ± 0.13 |
| Fat | 0.19 ± 0.07 |
| Uterus | 3.76 ± 1.11 |
| Ovaries | 3.47 ± 1.27 |
| Intestine | 1.13 ± 0.16 |
| Spleen | 16.98 ± 2.05 |
| Liver | 3.52 ± 0.39 |
| Pancreas | 0.51 ± 0.10 |
| Stomach | 0.61 ± 0.09 |
| Adrenal glands | 1.98 ± 0.82 |
| Kidney | 2.02 ± 0.27 |
| Lungs | 3.17 ± 0.76 |
| Heart | 0.58 ± 0.08 |
| HEK-A56 | 7.71 ± 4.52 ** |
| HEK-293 | 1.44 ± 0.25 |
| Muscle | 0.27 ± 0.04 |
| Bone | 4.78 ± 0.53 |
| Brain | 0.22 + 0.39 |

Biodistribution results confirm PET image observations with expected radioimmunoconjugate biodistribution in normal organs. Concerning tumors, a higher uptake of 7.71±4.52% ID/g was obtained in the A56-expressing HEK tumors as compared to 1.44±0.25% ID/g in the non-expressing one (n=8, p<0.05). These in vivo results (PET imaging and biodistribution study) therefore establish that ⁸⁹Zr-DFO-A049 can be effectively and selectively delivered to A56-expressing tumors.

Antibodies targeting oncolytic virus encoded proteins expressed on the surface of OV-infected tumor cells can be used to allow for diagnostic, therapeutic and theranostic applications when labeled with radionuclides, for example, in in vivo imaging of OV infection by PET imaging and localized delivery of therapeutic radioactive isotopes by radioimmunotherapy to kill OV-infected cells and also non-infected neighbouring tumor cells (11,12).

### REFERENCES

1. Babcook, J. S., Leslie, K. B., Olsen, O. A., Salmon, R. A., Schrader, J. W. (1996) A novel strategy for generating monoclonal antibodies from single, isolated lymphocytes producing antibodies of defined specificities. Proc Natl Acad Sci USA 93 (15): p. 7843-8.
2. Jarahian, M., Fiedler, M., Cohnen, A., Djandji, D., Hammerling, G. J., Gati, C., Cerwenka, A., Turner, P. C., Moyer, R. W., Watzl, C., Hengel, H., and Momburg, F. (2011) Modulation of NKp30- and NKp46-mediated natural killer cell responses by poxviral hemagglutinin. PLoS Pathog 7, e1002195.
3. S. Guedan, H. Calderon, A. D. Posey, M. V Maus, Engineering and Design of Chimeric Antigen Receptors, Mol. Ther. Methods Clin. Dev. 12 (2019) 145-156. https://doi.org/10.1016/j.omtm.2018.12.009.
4. Molecular Cloning: A Laboratory Manual 3^{rd} edition (eds. Sambrook, J. & Russell, D. W.) (Cold Spring Harbor Laboratory Press, Cold Spring Harbor, N.Y., USA, 2001).
5. Kim J S. Combination Radioimmunotherapy Approaches and Quantification of Immuno-PET. Nuclear medicine and molecular imaging. 2016; 50(2):104-11.
6. van de Watering F C, Rijpkema M, Perk L, Brinkmann U, Oyen W J, Boerman O C. Zirconium-89 labeled antibodies: a new tool for molecular imaging in cancer patients. BioMed research international. 2014; 2014:203601.
7. Zhang Y, Hong H, Cai W. PET tracers based on Zirconium-89. Current radiopharmaceuticals. 2011; 4(2):131-9.
8. Dias G M, Ramogida C F, Rousseau J, Zacchia N A, Hoehr C, Schaffer P, Lin K S, Bénard F. ⁸⁹Zr for antibody labeling and in vivo studies—A comparison between liquid and solid target production. Nucl Med Biol. 2018 March; 58:1-7.
9. Rousseau J, Zhang Z, Wang X, Zhang C, Lau J, Rousseau E, et al. Synthesis and evaluation of bifunctional tetrahydroxamate chelators for labeling antibodies with (89) Zr for imaging with positron emission tomography. Bioorg Med Chem Lett. 2018; 28(5):899-905.
10. Rousseau J, Zhang Z, Dias G M, Zhang C, Colpo N, Bénard F, Lin K S. Design, synthesis and evaluation of novel bifunctional tetrahydroxamate chelators for PET imaging of ⁸⁹Zr-labeled antibodies. Bioorg Med Chem Lett. 2017; 27(4):708-712.
11. Makvandi M., et al. Alpha-Emitters and Targeted Alpha Therapy in Oncology: from Basic Science to Clinical Investigations. Target Oncol. 2018 April; 13(2):189-203.
12. Kraeber-Bodere F, Bodet-Milin C, Rousseau C, Eugene T, Pallardy A, Frampas E, et al. Radioimmunoconjugates for the treatment of cancer. Seminars in oncology. 2014; 41(5):613-22.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. An antibody that specifically binds to Vaccinia Virus A56 antigen (VV A56) and competes for binding to VV A56 with an antibody comprising:
a variable heavy chain (V_{H}) polypeptide comprising
a V_{H} CDR1 comprising the amino acid sequence SSYWIC (SEQ ID NO: 3),
a V_{H} CDR2 comprising the amino acid sequence CIYAGSGGSTYYATWAKG (SEQ ID NO: 4), and
a V_{H} CDR3 comprising the amino acid sequence AYSDRSGGYSFNL (SEQ ID NO: 5); and
a variable light chain (V_{L}) polypeptide comprising
a V_{L} CDR1 comprising the amino acid sequence QASQSVDNNNYLA (SEQ ID NO: 6),
a V_{L} CDR2 comprising the amino acid sequence SASSLAS (SEQ ID NO: 7), and
a V_{L} CDR3 comprising the amino acid sequence LGSYDCSDADCYA (SEQ ID NO: 8);
a variable heavy chain (V_{H}) polypeptide comprising
a V_{H} CDR1 comprising the amino acid sequence DIYYIS (SEQ ID NO: 11),
a V_{H} CDR2 comprising the amino acid sequence CTYAGSSGSTYYATWAKG (SEQ ID NO: 12), and
a V_{H} CDR3 comprising the amino acid sequence DRYPGTSGRVYGMDL (SEQ ID NO: 13); and
a variable light chain (V_{L}) polypeptide comprising
a V_{L} CDR1 comprising the amino acid sequence QASQSISDLLS (SEQ ID NO: 14),
a V_{L} CDR2 comprising the amino acid sequence SASTLAS (SEQ ID NO: 15), and
a V_{L} CDR3 comprising the amino acid sequence QCNYYSPTYGNG (SEQ ID NO: 16);
a variable heavy chain (V_{H}) polypeptide comprising
a V_{H} CDR1 comprising the amino acid sequence SSYWLC (SEQ ID NO: 19),
a V_{H} CDR2 comprising the amino acid sequence CIYNGDGSTHYASWAKG (SEQ ID NO: 20), and
a V_{H} CDR3 comprising the amino acid sequence DYTYNFYTYGFNL (SEQ ID NO: 21); and
a variable light chain (V_{L}) polypeptide comprising
a V_{L} CDR1 comprising the amino acid sequence QASQSVNIWAS (SEQ ID NO: 22),
a V_{L} CDR2 comprising the amino acid sequence KASTLAS (SEQ ID NO: 23), and
a V_{L} CDR3 comprising the amino acid sequence QGGYPSSSSGWA (SEQ ID NO: 24); or
a variable heavy chain (V_{H}) polypeptide comprising
a V_{H} CDR1 comprising the amino acid sequence SSYWIC (SEQ ID NO: 27),
a V_{H} CDR2 comprising the amino acid sequence CTYNGDGSTHYASWAKG (SEQ ID NO: 28), and
a V_{H} CDR3 comprising the amino acid sequence DYTDAFYTYGFNL (SEQ ID NO: 29); and
a variable light chain (V_{L}) polypeptide comprising
a V_{L} CDR1 comprising the amino acid sequence QASQSTSSYLA (SEQ ID NO: 30),
a V_{L} CDR2 comprising the amino acid sequence RASSLAS (SEQ ID NO: 31), and
a V_{L} CDR3 comprising the amino acid sequence QTGFYGSSGHT (SEQ ID NO: 32).

2. The antibody of claim 1, wherein the antibody comprises:
a variable heavy chain (V_{H}) polypeptide comprising
a V_{H} CDR1 comprising the amino acid sequence SSYWIC (SEQ ID NO: 3),
a V_{H} CDR2 comprising the amino acid sequence CIYAGSGGSTYYATWAKG (SEQ ID NO: 4), and
a V_{H} CDR3 comprising the amino acid sequence AYSDRSGGYSFNL (SEQ ID NO: 5); and
a variable light chain (V_{L}) polypeptide comprising
a V_{L} CDR1 comprising the amino acid sequence QASQSVDNNNYLA (SEQ ID NO: 6),
a V_{L} CDR2 comprising the amino acid sequence SASSLAS (SEQ ID NO: 7), and
a V_{L} CDR3 comprising the amino acid sequence LGSYDCSDADCYA (SEQ ID NO: 8);
a variable heavy chain (V_{H}) polypeptide comprising
a V_{H} CDR1 comprising the amino acid sequence DIYYIS (SEQ ID NO: 11),
a V_{H} CDR2 comprising the amino acid sequence CTYAGSSGSTYYATWAKG (SEQ ID NO: 12), and
a V_{H} CDR3 comprising the amino acid sequence DRYPGTSGRVYGMDL (SEQ ID NO: 13); and
a variable light chain (V_{L}) polypeptide comprising
a V_{L} CDR1 comprising the amino acid sequence QASQSISDLLS (SEQ ID NO: 14),
a V_{L} CDR2 comprising the amino acid sequence SASTLAS (SEQ ID NO: 15), and
a V_{L} CDR3 comprising the amino acid sequence QCNYYSPTYGNG (SEQ ID NO: 16);
a variable heavy chain (V_{H}) polypeptide comprising
a V_{H} CDR1 comprising the amino acid sequence SSYWLC (SEQ ID NO: 19),
a V_{H} CDR2 comprising the amino acid sequence CIYNGDGSTHYASWAKG (SEQ ID NO: 20), and
a V_{H} CDR3 comprising the amino acid sequence DYTYNFYTYGFNL (SEQ ID NO: 21); and
a variable light chain (V_{L}) polypeptide comprising
a V_{L} CDR1 comprising the amino acid sequence QASQSVNIWAS (SEQ ID NO: 22),
a V_{L} CDR2 comprising the amino acid sequence KASTLAS (SEQ ID NO: 23), and
a V_{L} CDR3 comprising the amino acid sequence QGGYPSSSSGWA (SEQ ID NO: 24); or
a variable heavy chain (V_{H}) polypeptide comprising
a V_{H} CDR1 comprising the amino acid sequence SSYWIC (SEQ ID NO: 27),
a V_{H} CDR2 comprising the amino acid sequence CTYNGDGSTHYASWAKG (SEQ ID NO: 28), and
a V_{H} CDR3 comprising the amino acid sequence DYTDAFYTYGFNL (SEQ ID NO: 29); and
a variable light chain (V_{L}) polypeptide comprising
a V_{L} CDR1 comprising the amino acid sequence QASQSTSSYLA (SEQ ID NO: 30),
a V_{L} CDR2 comprising the amino acid sequence RASSLAS (SEQ ID NO: 31), and
a V_{L} CDR3 comprising the amino acid sequence QTGFYGSSGHT (SEQ ID NO: 32).

3. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 1; and
a variable light chain (V_{L}) polypeptide comprising an amino acid sequence having 70% or greater identity to the amino acid sequence set forth in SEQ ID NO: 2.

4. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 9; and
a variable light chain (V_{L}) polypeptide comprising an amino acid sequence having 70% or greater identity to the amino acid sequence set forth in SEQ ID NO: 10.

5. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 17; and
a variable light chain (V_{L}) polypeptide comprising an amino acid sequence having 70% or greater identity to the amino acid sequence set forth in SEQ ID NO: 18.

6. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 25; and
a variable light chain (V_{L}) polypeptide comprising an amino acid sequence having 70% or greater identity to the amino acid sequence set forth in SEQ ID NO: 26.

7. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 9; and
a variable light chain (V_{L}) polypeptide comprising an amino acid sequence having 70% or greater identity to the amino acid sequence set forth in SEQ ID NO: 62.

8. The antibody of claim 1 or claim 2, wherein the antibody comprises:
a variable heavy chain (V_{H}) polypeptide comprising an amino acid sequence having 70% or greater identity to the amino acid sequence set forth in SEQ ID NO: 9; and
a variable light chain (V_{L}) polypeptide comprising an amino acid sequence having 70% or greater identity to the amino acid sequence set forth in SEQ ID NO: 63.

9. An antibody that specifically binds to Vaccinia Virus B5 antigen (VV B5) and competes for binding to VV B5 with an antibody comprising:
a variable heavy chain (V_{H}) polypeptide comprising
a V_{H} CDR1 comprising the amino acid sequence SSYYMC (SEQ ID NO: 35),
a V_{H} CDR2 comprising the amino acid sequence CIYTSSGSAYYANWAKG (SEQ ID NO: 36), and
a V_{H} CDR3 comprising the amino acid sequence NAVGSSYYLYL (SEQ ID NO: 37); and
a variable light chain (V_{L}) polypeptide comprising
a V_{L} CDR1 comprising the amino acid sequence QASQSVAGNNYLS (SEQ ID NO: 38),
a V_{L} CDR2 comprising the amino acid sequence SVSTLAS (SEQ ID NO: 39), and
a V_{L} CDR3 comprising the amino acid sequence QGYYNDGIWA (SEQ ID NO: 40); or
a variable heavy chain (V_{H}) polypeptide comprising
a V_{H} CDR1 comprising the amino acid sequence SYWMC (SEQ ID NO: 43),
a V_{H} CDR2 comprising the amino acid sequence CIYGGSSGSTYYSNWAKG (SEQ ID NO: 44), and
a V_{H} CDR3 comprising the amino acid sequence DGSTWDYFRL (SEQ ID NO: 45); and
a variable light chain (V_{L}) polypeptide comprising
a V_{L} CDR1 comprising the amino acid sequence QASQSINTNYLS (SEQ ID NO: 46),
a V_{L} CDR2 comprising the amino acid sequence QASTLES (SEQ ID NO: 47), and
a V_{L} CDR3 comprising the amino acid sequence QGYYTVENIGNP (SEQ ID NO: 48).

10. The antibody of claim 9, wherein the antibody comprises:
a variable heavy chain (V_{H}) polypeptide comprising
a V_{H} CDR1 comprising the amino acid sequence SSYYMC (SEQ ID NO: 35),
a V_{H} CDR2 comprising the amino acid sequence CIYTSSGSAYYANWAKG (SEQ ID NO: 36), and
a V_{H} CDR3 comprising the amino acid sequence NAVGSSYYLYL (SEQ ID NO: 37); and
a variable light chain (V_{L}) polypeptide comprising
a V_{L} CDR1 comprising the amino acid sequence QASQSVAGNNYLS (SEQ ID NO: 38),
a V_{L} CDR2 comprising the amino acid sequence SVSTLAS (SEQ ID NO: 39), and
a V_{L} CDR3 comprising the amino acid sequence QGYYNDGIWA (SEQ ID NO: 40); or
a variable heavy chain (V_{H}) polypeptide comprising
a V_{H} CDR1 comprising the amino acid sequence SYWMC (SEQ ID NO: 43),
a V_{H} CDR2 comprising the amino acid sequence CIYGGSSGSTYYSNWAKG (SEQ ID NO: 44), and
a V_{H} CDR3 comprising the amino acid sequence DGSTWDYFRL (SEQ ID NO: 45); and
a variable light chain (V_{L}) polypeptide comprising
a V_{L} CDR1 comprising the amino acid sequence QASQSINTNYLS (SEQ ID NO: 46),
a V_{L} CDR2 comprising the amino acid sequence QASTLES (SEQ ID NO: 47), and
a V_{L} CDR3 comprising the amino acid sequence QGYYTVENIGNP (SEQ ID NO: 48).

11. The antibody of claim 9 or claim 10, wherein the antibody comprises:
a variable heavy chain (V_{H}) polypeptide comprising an amino acid sequence having 70% or greater identity to the amino acid sequence set forth in SEQ ID NO: 33; and
a variable light chain (V_{L}) polypeptide comprising an amino acid sequence having 70% or greater identity to the amino acid sequence set forth in SEQ ID NO: 34.

12. The antibody of claim 9 or claim 10, wherein the antibody comprises:
a variable heavy chain (V_{H}) polypeptide comprising an amino acid sequence having 70% or greater identity to the amino acid sequence set forth in SEQ ID NO: 41; and
a variable light chain (V_{L}) polypeptide comprising an amino acid sequence having 70% or greater identity to the amino acid sequence set forth in SEQ ID NO: 42.

13. The antibody of any one of claims 1 to 12, wherein the antibody is a monoclonal antibody.

14. The antibody of any one of claims 1 to 13, wherein the antibody is a humanized antibody.

15. The antibody of any one of claims 1 to 14, wherein the antibody is an IgG.

16. The antibody of claim 15, wherein the antibody comprises a human Fc domain.

17. The antibody of claim 16, wherein the antibody is a human IgG1.

18. The antibody of any one of claims 1 to 14, wherein the antibody is selected from the group consisting of: a Fab, a F(ab′)₂, and a F(ab′).

19. The antibody of any one of claims 1 to 14, wherein the antibody is a single chain antibody.

20. The antibody of claim 19, wherein the single chain antibody is a scFv.

21. The antibody of any one of claims 1 to 20, wherein the antibody is a bispecific antibody comprising a first antigen-binding domain comprising a V_{H} polypeptide-V_{L} polypeptide pair as defined in any one of claims 1 to 12.

22. The antibody of claim 21, wherein the bispecific antibody comprises a second antigen-binding domain that specifically binds an antigen other than a Vaccinia Virus antigen.

23. The antibody of claim 22, wherein the antigen other than a Vaccinia Virus antigen is an immune cell surface antigen.

24. The antibody of claim 23, wherein the immune cell surface antigen is an immune effector cell surface antigen.

25. The antibody of claim 24, wherein the immune cell surface antigen is a T cell surface antigen.

26. The antibody of claim 25, wherein the antigen is a T cell stimulatory molecule.

27. The antibody of claim 26, wherein the T cell stimulatory molecule is CD3 or CD28.

28. The antibody of claim 24, wherein the immune cell surface antigen is a natural killer (NK) cell surface antigen.

29. The antibody of claim 24, wherein the immune cell surface antigen is a macrophage cell surface antigen.

30. A fusion protein, comprising:
a chain of an antibody of any one of claims 1 to 29 fused to a heterologous sequence of amino acids.

31. The fusion protein of claim 30, wherein the heterologous sequence of amino acids is fused to the C-terminus of the chain of the antibody.

32. The fusion protein of claim 30 or claim 31, wherein the antibody is the single chain antibody of claim 19 or 20.

33. The fusion protein of claim 32, wherein the fusion protein is a chimeric antigen receptor (CAR) comprising:
the single chain antibody;
a transmembrane domain; and
an intracellular signaling domain.

34. A conjugate, comprising:
an antibody of any one of claims 1 to 29 or a fusion protein of any one of claims 30 to 33; and
an agent conjugated to the antibody or fusion protein.

35. The conjugate of claim 34, wherein the agent is selected from the group consisting of: a chemotherapeutic agent, a toxin, a radiation sensitizing agent, a radioactive isotope, a detectable label, and a half-life extending moiety.

36. The conjugate of claim 35, wherein the radioactive isotope is a therapeutic radioactive isotope.

37. The conjugate of claim 35, wherein the detectable label is a radiolabel.

38. The conjugate of claim 37, wherein the radiolabel is Zirconium-89 (⁸⁹Zr).

39. The conjugate of any one of claims 34 to 38, wherein the agent is conjugated to the antibody or fusion protein via a non-cleavable linker.

40. The conjugate of any one of claims 34 to 38, wherein the agent is conjugated to the antibody or fusion protein via a cleavable linker.

41. The conjugate of claim 40, wherein the cleavable linker is an enzyme-cleavable linker.

42. The conjugate of claim 41, wherein the linker is cleavable by a lysosomal protease.

43. The conjugate of claim 42, wherein the linker is cleavable by cathepsin or plasmin.

44. A nucleic acid encoding a variable heavy chain (V_{H}) polypeptide, a variable light chain (V_{L}) polypeptide, or both, of an antibody of any one of claims 1 to 29.

45. A nucleic acid encoding the fusion protein of any one of claims 30 to 33.

46. An expression vector comprising the nucleic acid of claim 44 or claim 45.

47. A cell comprising:
the nucleic acid of claim 44 or claim 45; or
the expression vector of claim 46.

48. A cell comprising:
a first nucleic acid encoding the variable heavy chain (V_{H}) polypeptide of an antibody of any one of claims 1 to 18; and
a second nucleic acid encoding the variable light chain (V_{L}) polypeptide of the antibody.

49. The cell of claim 48, comprising:
a first expression vector comprising the first nucleic acid; and
a second expression vector comprising the second nucleic acid.

50. A method of producing the antibody of any one of claims 1 to 28, comprising culturing the cell of any one of claims 47 to 49 under conditions suitable for the cell to express the antibody, wherein the antibody is produced.

51. A pharmaceutical composition, comprising:
the antibody of any one of claims 1 to 28; and
a pharmaceutically acceptable carrier.

52. A pharmaceutical composition, comprising:
the fusion protein of any one of claims 30 to 33; and
a pharmaceutically acceptable carrier.

53. A pharmaceutical composition, comprising:
the conjugate of any one of claims 34 to 43; and
a pharmaceutically acceptable carrier.

54. A kit, comprising:
the pharmaceutical composition of any one of claims 51 to 53; and
instructions for administering the pharmaceutical composition to an individual in need thereof.

55. The kit of claim 54, wherein the pharmaceutical composition is present in one or more unit dosages.

56. The kit of claim 54, wherein the pharmaceutical composition is present in two or more unit dosages.

57. A method, comprising:
administering the pharmaceutical composition of any one of claims 51 to 53 to an individual having cancer, wherein the individual comprises cancer cells infected with Vaccinia Virus (VV), and wherein the antibody, fusion protein or conjugate is targeted to the infected cancer cells by VV antigens expressed on the surface of the infected cancer cells.

58. The method according to claim 57, further comprising, prior to administering the pharmaceutical composition to the individual, infecting the cancer cells by administering VV to the individual.

59. The method according to claim 57 or 58, wherein the method is a method of treating the cancer of the individual.

60. The method according to any one of claims 57 to 59, wherein the pharmaceutical composition of claim 53 is administered to the individual, wherein the conjugate comprises the antibody conjugated to a detectable label or radioactive isotope which is an in vivo imaging agent, and wherein the method comprises imaging the infected cancer cells in the individual using the in vivo imaging agent.

61. A method of targeting an antibody that specifically binds an oncolytic virus (OV) antigen to cancer cells in an individual, comprising:
administering to the individual a pharmaceutical composition comprising an antibody that specifically binds the OV antigen, wherein the cancer cells in the individual are infected with OV and express the OV antigen on their surface.

62. The method according to claim 61, further comprising, prior to administering the pharmaceutical composition to the individual, infecting the cancer cells by administering the OV to the individual.

63. The method according to claim 61 or claim 62, wherein the OV antigen is native to the OV.

64. The method according to claim 61 or claim 62, wherein the OV antigen is heterologous to the OV.

65. The method according to any one of claims 61 to 64, wherein the OV is selected from the group consisting of: Vaccinia Virus, adenovirus, Herpes Simplex Virus (HSV), reovirus, vesicular stomatitis virus, New Castle Disease virus, Seneca Valley virus, poliovirus, measles virus, Coxsackie virus, and Maraba virus.

66. The method according to any one of claims 61 to 64, wherein the OV is Vaccinia Virus (VV).

67. The method according to claim 66, wherein the VV is JX-594 or GL-ONC1.

68. The method according to claim 66, wherein the strain of VV is selected from the group consisting of: Western Reserve, Wyeth, Lister, Copenhagen, Temple of Heaven, Patwadangar, and Modified Vaccinia Virus Ankara.

69. The method according to any one of claims 61 to 68, wherein the antibody is a monoclonal antibody.

70. The method according to any one of claims 61 to 69, wherein the antibody is a humanized antibody.

71. The method according to any one of claims 61 to 70, wherein the antibody is an IgG.

72. The method according to claim 71, wherein the antibody comprises a human Fc domain.

73. The method according to claim 72, wherein the antibody is a human IgG1.

74. The method according to any one of claims 61 to 70, wherein the antibody is selected from the group consisting of: a Fab, a F(ab′)₂, and a F(ab′).

75. The method according to any one of claims 61 to 70, wherein the antibody is a single chain antibody.

76. The method according to claim 75, wherein the single chain antibody is a scFv.

77. The method according to any one of claims 66 to 76, wherein the OV antigen is a VV A56 antigen.

78. The method according to claim 77, wherein the pharmaceutical composition comprises an antibody of any one of claims 1 to 8.

79. The method according to any one of claims 66 to 76, wherein the OV antigen is a VV B5 antigen.

80. The method according to claim 79, wherein the pharmaceutical composition comprises an antibody of any one of claims 9 to 12.

81. The method according to any one of claims 57 to 80, wherein the pharmaceutical composition comprises the antibody conjugated to a detectable label or radioactive isotope.

82. The method according to claim 81, wherein the detectable label or radioactive isotope is an in vivo imaging agent.

83. The method according to claim 82, comprising detecting the in vivo imaging agent to image the cancer cells in vivo.

84. The method according to any one of claims 61 to 83, wherein the method is a method of treating the cancer of the individual.

85. A chimeric antigen receptor (CAR) comprising:
an antigen binding domain that specifically binds an oncolytic virus (OV) antigen;
a transmembrane domain; and
an intracellular signaling domain.

86. The CAR of claim 85, wherein the OV antigen is native to the OV.

87. The CAR of claim 85, wherein the OV antigen is heterologous to the OV.

88. The CAR of any one of claims 85 to 87, wherein the OV is selected from the group consisting of: Vaccinia Virus, adenovirus, Herpes Simplex Virus (HSV), reovirus, vesicular stomatitis virus, New Castle Disease virus, Seneca Valley virus, poliovirus, measles virus, and Maraba virus.

89. The CAR of any one of claims 85 to 87, wherein the OV antigen is an antigen encoded by a Vaccinia Virus (VV).

90. The CAR of claim 89, wherein the VV is JX-594 or GL-ONC1.

91. The CAR of claim 89, wherein the strain of VV is selected from the group consisting of: Western Reserve, Wyeth, Lister, Copenhagen, Temple of Heaven, Patwadangar, and Modified Vaccinia Virus Ankara.

92. The CAR of claim 89, wherein the OV antigen is a VV A56 antigen.

93. The CAR of claim 92, wherein the antigen binding domain comprises a V_{H} polypeptide-V_{L} polypeptide pair of an antibody of any one of claims 1 to 8.

94. The CAR of claim 89, wherein the OV antigen is a VV B5 antigen.

95. The CAR of claim 94, wherein the antigen binding domain comprises a V_{H} polypeptide-V_{L} polypeptide pair of an antibody of any one of claims 9 to 12.

96. The CAR of any one of claims 85 to 95, wherein the antigen-binding domain comprises a scFv.

97. The CAR of any one of 85 to 96, wherein the CAR is provided by a single polypeptide.

98. The CAR of any one of 85 to 96, wherein the CAR is provided by two or more polypeptides.

99. A nucleic acid encoding the CAR of any one of claims 85 to 98.

100. A cell comprising the nucleic acid of claim 99.

101. The cell of claim 100, wherein the cell expresses the CAR on its surface.

102. The cell of claim 100 or 101, wherein the cell is an immune cell.

103. The cell of claim 102, wherein the cell is an immune effector cell.

104. The cell of claim 103, wherein the cell is a T cell.

105. The cell of claim 103, wherein the cell is an NK cell.

106. The cell of claim 103, wherein the cell is a macrophage.

107. A pharmaceutical composition comprising the cell of any one of claims 100 to 105.

108. A method of targeting a CAR that specifically binds an oncolytic virus (OV) antigen to cancer cells in an individual, comprising:
administering to the individual the pharmaceutical composition of claim 107, wherein the cancer cells in the individual are infected with OV and express the OV antigen on their surface.

109. The method according to claim 108, further comprising, prior to administering the pharmaceutical composition to the individual, infecting the cancer cells by administering the OV to the individual.

110. The method according to claim 108 or claim 109, wherein the method is a method of treating the cancer of the individual.

111. A conjugate, comprising:
an antibody that specifically binds an oncolytic virus (OV) antigen; and
an agent conjugated to the antibody, wherein the agent is selected from the group consisting of: a chemotherapeutic agent, a toxin, a radiation sensitizing agent, and a radioactive isotope.

112. The conjugate of claim 111, wherein the radioactive isotope is a therapeutic radioactive isotope.

113. The conjugate of claim 111 or claim 112, wherein the OV antigen is native to the OV.

114. The conjugate of claim 111 or claim 112, wherein the OV antigen is heterologous to the OV.

115. The conjugate of any one of claims 111 to 114, wherein the OV antigen is an antigen encoded by an OV selected from the group consisting of: Vaccinia Virus, adenovirus, HSV, reovirus, vesicular stomatitis virus, New Castle Disease virus, Seneca Valley virus, poliovirus, measles virus, Coxsackie virus, and Maraba virus.

116. The conjugate of any one of claims 111 to 114, wherein the OV antigen is an antigen encoded by a Vaccinia Virus (VV).

117. The conjugate of claim 116, wherein the VV is JX-594 or GL-ONC1.

118. The conjugate of claim 116, wherein the strain of VV is selected from the group consisting of: Western Reserve, Wyeth, Lister, Copenhagen, Temple of Heaven, Patwadangar, and Modified Vaccinia Virus Ankara.

119. The conjugate of claim 116, wherein the OV antigen is a VV A56 antigen.

120. The conjugate of claim 119, wherein the antibody is an antibody of any one of claims 1 to 8.

121. The conjugate of claim 116, wherein the OV antigen is a VV B5 antigen.

122. The conjugate of claim 121, wherein the antibody is an antibody of any one of claims 9 to 12.

123. The conjugate of any one of claims 111 to 122, wherein the antibody is a monoclonal antibody.

124. The conjugate of any one of claims 111 to 123, wherein the antibody is a humanized antibody.

125. The conjugate of any one of claims 111 to 124, wherein the antibody is an IgG.

126. The conjugate of claim 125, wherein the antibody comprises a human Fc domain.

127. The conjugate of claim 126, wherein the antibody is a human IgG1.

128. The conjugate of any one of claims 111 to 124, wherein the antibody is selected from the group consisting of: a Fab, a F(ab′)₂, and a F(ab′).

129. The conjugate of any one of claims 111 to 124, wherein the antibody is a single chain antibody.

130. The conjugate of claim 129, wherein the single chain antibody is a scFv.

131. The conjugate of any one of claims 111 to 130, wherein the agent is conjugated to the antibody via a non-cleavable linker.

132. The conjugate of any one of claims 111 to 130, wherein the agent is conjugated to the antibody via a cleavable linker.

133. The conjugate of claim 132, wherein the cleavable linker is an enzyme-cleavable linker.

134. The conjugate of claim 133, wherein the linker is cleavable by a lysosomal protease.

135. The conjugate of claim 134, wherein the linker is cleavable by cathepsin or plasmin.

136. A pharmaceutical composition comprising the conjugate of any one of claims 111 to 135.

137. A method of targeting a conjugate that comprises an antibody that specifically binds an oncolytic virus (OV) antigen to cancer cells in an individual, comprising:
administering to the individual the pharmaceutical composition of claim 136, wherein the cancer cells in the individual are infected with OV and express the OV antigen on their surface.

138. The method according to claim 137, further comprising, prior to administering the pharmaceutical composition to the individual, infecting the cancer cells by administering the OV to the individual.

139. The method according to claim 137 or claim 138, wherein the method is a method of treating the cancer of the individual.


SEQUENCE LISTING (187 sequences)

SEQ ID NO: 1 - protein, 123 aa, Oryctolagus cuniculus
QEQLEESGGDLVKPEGSLTLTCTASGFSFSSSYWICWVRQAPGKGLEWIACIYAGSGGSTYYATWAKGRFTISKTSSTTVTLQMTSLTAADTASYFCVRAYSDRSGGYSFNLWGPGTLVTVSS

SEQ ID NO: 2 - protein, 112 aa, Oryctolagus cuniculus
QVLTQTASPVSAAVGGTVTINCQASQSVDNNNYLAWYQQKPGQPPKQLIYSASSLASGVPSRFKGSGSGTQFTLTISDVQCDDAATYYCLGSYDCSDADCYAFGGGTEVVVK

SEQ ID NO: 3 - protein, 6 aa, Oryctolagus cuniculus
SSYWIC

SEQ ID NO: 4 - protein, 18 aa, Oryctolagus cuniculus
CIYAGSGGSTYYATWAKG

SEQ ID NO: 5 - protein, 13 aa, Oryctolagus cuniculus
AYSDRSGGYSFNL

SEQ ID NO: 6 - protein, 13 aa, Oryctolagus cuniculus
QASQSVDNNNYLA

SEQ ID NO: 7 - protein, 7 aa, Oryctolagus cuniculus
SASSLAS

SEQ ID NO: 8 - protein, 13 aa, Oryctolagus cuniculus
LGSYDCSDADCYA

SEQ ID NO: 9 - protein, 124 aa, Oryctolagus cuniculus
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSS

SEQ ID NO: 10 - protein, 109 aa, Oryctolagus cuniculus
VVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVK

SEQ ID NO: 11 - protein, 6 aa, Oryctolagus cuniculus
DIYYIS

SEQ ID NO: 12 - protein, 18 aa, Oryctolagus cuniculus
CTYAGSSGSTYYATWAKG

SEQ ID NO: 13 - protein, 15 aa, Oryctolagus cuniculus
DRYPGTSGRVYGMDL

SEQ ID NO: 14 - protein, 11 aa, Oryctolagus cuniculus
QASQSISDLLS

SEQ ID NO: 15 - protein, 7 aa, Oryctolagus cuniculus
SASTLAS

SEQ ID NO: 16 - protein, 12 aa, Oryctolagus cuniculus
QCNYYSPTYGNG

SEQ ID NO: 17 - protein, 121 aa, Oryctolagus cuniculus
QSLEESGGDLVKPGASLTLTCTASGFSFSSSYWLCWARQAPGKGPEWIACIYNGDGSTHYASWAKGRFTISKSSSTTVTLQMTSLTAADTATYFCARDYTYNFYTYGFNLWGPGTLVTVSS

SEQ ID NO: 18 - protein, 109 aa, Oryctolagus cuniculus
FELTQTPSSVEAAVGGTVTINCQASQSVNIWASWYQQKPGQPPKLLIYKASTLASGVPSRFKGSGSGTEFTLTISDLECADAATYYCQGGYPSSSSGWAFGGGTEVVVK

SEQ ID NO: 19 - protein, 6 aa, Oryctolagus cuniculus
SSYWLC

SEQ ID NO: 20 - protein, 17 aa, Oryctolagus cuniculus
CIYNGDGSTHYASWAKG

SEQ ID NO: 21 - protein, 13 aa, Oryctolagus cuniculus
DYTYNFYTYGFNL

SEQ ID NO: 22 - protein, 11 aa, Oryctolagus cuniculus
QASQSVNIWAS

SEQ ID NO: 23 - protein, 7 aa, Oryctolagus cuniculus
KASTLAS

SEQ ID NO: 24 - protein, 12 aa, Oryctolagus cuniculus
QGGYPSSSSGWA

SEQ ID NO: 25 - protein, 122 aa, Oryctolagus cuniculus
QEQVVESGGGLVKPGASLTLTCTASGFTLSSSYWICWVRQAPGKGPEWIACTYNGDGSTHYASWAKGRFTISKSSSTTVTLQMTSLTAADTATYFCARDYTDAFYTYGFNLWGPGTLVTVSS

SEQ ID NO: 26 - protein, 109 aa, Oryctolagus cuniculus
DIVMTQTPASVSEPVGGTVTIKCQASQSTSSYLAWYQQKPGQPPKLLIYRASSLASGVPSRFKGSGSATEFTLTISDLECADAATYYCQTGFYGSSGHTFGGGTEVVVK

SEQ ID NO: 27 - protein, 6 aa, Oryctolagus cuniculus
SSYWIC

SEQ ID NO: 28 - protein, 17 aa, Oryctolagus cuniculus
CTYNGDGSTHYASWAKG

SEQ ID NO: 29 - protein, 13 aa, Oryctolagus cuniculus
DYTDAFYTYGFNL

SEQ ID NO: 30 - protein, 11 aa, Oryctolagus cuniculus
QASQSTSSYLA

SEQ ID NO: 31 - protein, 7 aa, Oryctolagus cuniculus
RASSLAS

SEQ ID NO: 32 - protein, 11 aa, Oryctolagus cuniculus
QTGFYGSSGHT

SEQ ID NO: 33 - protein, 120 aa, Oryctolagus cuniculus
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSS

SEQ ID NO: 34 - protein, 109 aa, Oryctolagus cuniculus
QVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVK

SEQ ID NO: 35 - protein, 6 aa, Oryctolagus cuniculus
SSYYMC

SEQ ID NO: 36 - protein, 17 aa, Oryctolagus cuniculus
CIYTSSGSAYYANWAKG

SEQ ID NO: 37 - protein, 11 aa, Oryctolagus cuniculus
NAVGSSYYLYL

SEQ ID NO: 38 - protein, 13 aa, Oryctolagus cuniculus
QASQSVAGNNYLS

SEQ ID NO: 39 - protein, 7 aa, Oryctolagus cuniculus
SVSTLAS

SEQ ID NO: 40 - protein, 10 aa, Oryctolagus cuniculus
QGYYNDGIWA

SEQ ID NO: 41 - protein, 119 aa, Oryctolagus cuniculus
QEQVEESGGGLAKPGASLTLTCEASGFTLSSYWMCWVRQAPGKGLEWIACIYGGSSGSTYYSNWAKGRFTISKTSSTTVTLQMTSLTAADTATYFCARDGSTWDYFRLWGPGTLVTVSS

SEQ ID NO: 42 - protein, 110 aa, Oryctolagus cuniculus
YDMTQTPSSVEAAVGGTVTIKCQASQSINTNYLSWYQQKPGQPPKLLIYQASTLESGVPSRFKGSGSGTEYTLTISDLECADAATYYCQGYYTVENIGNPFGGGTEVVVK

SEQ ID NO: 43 - protein, 5 aa, Oryctolagus cuniculus
SYWMC

SEQ ID NO: 44 - protein, 18 aa, Oryctolagus cuniculus
CIYGGSSGSTYYSNWAKG

SEQ ID NO: 45 - protein, 10 aa, Oryctolagus cuniculus
DGSTWDYFRL

SEQ ID NO: 46 - protein, 12 aa, Oryctolagus cuniculus
QASQSINTNYLS

SEQ ID NO: 47 - protein, 7 aa, Oryctolagus cuniculus
QASTLES

SEQ ID NO: 48 - protein, 12 aa, Oryctolagus cuniculus
QGYYTVENIGNP

SEQ ID NO: 49 - protein, 15 aa, Oryctolagus cuniculus
GGGGSGGGGSGGGGS

SEQ ID NO: 50 - DNA, 369 bp, Oryctolagus cuniculus
caggagcaactggaggagtccgggggagacctggtcaagcctgagggatccctgacactcacctgcacagcctctggattctccttcagtagcagctactggatatgctgggtccgccaggctccagggaaggggctggagtggatcgcatgcatttatgctggtagtggtggtagcacttactacgcgacctgggcgaaaggccgattcaccatctccaaaacctcgtcgaccacggtgactctgcaaatgaccagtctgacagccgcggacacggccagctatttctgtgtccgcgcatatagtgatagaagtggtggttactcatttaatttgtggggcccaggcaccctggtcaccgtctcttca

SEQ ID NO: 51 - DNA, 336 bp, Oryctolagus cuniculus
caagtgctgacccagactgcatcccccgtgtctgcggctgttggaggcacagtcaccatcaattgccaggccagtcagagtgttgataataacaactacttagcctggtatcagcagaaaccagggcagcctcccaaacaactgatctattctgcatccagtctggcatctggggtcccatcgcggttcaaaggcagtggatctgggacacagttcactctcaccatcagcgacgtgcagtgtgacgatgctgccacttactactgtctaggcagttatgattgtagtgatgctgattgttatgctttcggcggagggaccgaggtggtggtcaaa

SEQ ID NO: 52 - DNA, 372 bp, Oryctolagus cuniculus
cagtccttggaggagtccgggggagacctggtcaagcctggggcatccctgacactcacctgcacagcctctggaatcgacttcagtgacatttattacatatcttgggtccgccaggctccagggaaggggctggagtggatcgcatgcacttatgctggtagtagtggtagcacttactacgcgacctgggcgaaaggccgattcaccatctccaaagcctcgtcgaccacggtgactctgcaaatgaccagtctgacagccgcggacacggccacctatttctgtgcgagagatcgttatcctggtactagtggtagggtctacggcatggacctctggggcccagggaccctcgtcaccgtctcttca

SEQ ID NO: 53 - DNA, 327 bp, Oryctolagus cuniculus
gttgtgatgacccagactccatcctccgtgtctgaacctgtgggaggcacagtcaccatcaagtgccaggccagtcagagcattagcgacctcttatcctggtatcagcagaaaccagggcagcctcccaagctcctgatctattctgcatccactctggcatctggggtctcatcgcggttcaaaggcagtggatctgggacagaattcactctcaccatcagcgacctggagtgtgccgatgctgccacttactactgtcaatgtaattattatagtcctacttatgggaatggtttcggcggagggaccgaggtggtggtcaaa

SEQ ID NO: 54 - DNA, 363 bp, Oryctolagus cuniculus
cagtcattggaggagtccgggggagacctggtcaagcctggggcatccctgacactcacctgcacagcctctggattctccttcagtagcagctattggctatgctgggcccgccaggctccagggaaggggcctgagtggatcgcatgcatttataatggtgatggcagcacacactacgcgagctgggcgaaaggccgattcaccatctccaaatcctcgtcgaccacggtgactctgcaaatgaccagtctgacagccgcggacacggccacctatttctgtgcgagagattatacttataatttttatacttatggttttaatttgtggggcccaggcaccctggtcaccgtctcttca

SEQ ID NO: 55 - DNA, 327 bp, Oryctolagus cuniculus
ttcgaattgacccagactccatcctccgtggaggcagctgtgggaggcacagtcaccatcaactgccaggccagtcagagcgtgaatatttgggcatcctggtaccagcagaaaccagggcagcctcccaagctcctgatctacaaggcatccactctggcatctggggtcccatcgcggttcaaaggcagtggatctgggacagagttcactctcaccatcagcgacctggagtgtgccgatgctgccacttactactgtcaaggcggttatcctagtagtagtagtggttgggctttcggcggagggaccgaggtggtggtcaaa

SEQ ID NO: 56 - DNA, 366 bp, Oryctolagus cuniculus
caggagcaggtggtggagtccgggggaggcctggtcaagcctggggcatccctgacactcacctgcacagcctctggattcaccctcagtagtagttattggatatgctgggtccgccaggctccagggaaggggcctgagtggatcgcatgcacttataatggtgatggcagcacacactacgcgagctgggcgaaaggccgattcaccatctccaaatcctcgtcgaccacggtgactctgcaaatgaccagtctgacagccgcggacacggccacctatttctgtgcgagagattatactgatgctttttatacttatggttttaatttgtggggcccaggcaccctggtcaccgtctcttca

SEQ ID NO: 57 - DNA, 327 bp, Oryctolagus cuniculus
gacattgtgatgacccagactccagcctccgtgtctgaacctgtgggaggcacagtcaccatcaagtgccaggccagtcagagcactagtagctacttagcctggtatcagcagaaaccagggcagcctcccaagctcctgatctacagggcatccagtctggcatctggggtcccatcgcggttcaaaggcagtggatctgcgacagagttcactctcaccatcagcgacctggagtgtgccgatgctgccacttactactgtcaaactggtttttatggtagtagtgggcatactttcggcggagggaccgaggtggtggtcaaa

SEQ ID NO: 58 - DNA, 360 bp, Oryctolagus cuniculus
caggagcagctggaggagtccgggggaggcctggtcaagcctgagggatccctgacactcacctgcacagcttctggattctccttcagtagcagctactacatgtgctgggtccgccaggctccagggagggggctggagtggatcgcatgcatttatactagtagtggtagcgcttactacgcgaactgggcgaaaggccgattcaccatctccagaacctcgtcgaccacggtgactctgcaaatgacccgtctgacagccgcggacacggccacctatttctgtgtgagaaacgctgttgggagtagttattatttgtatttgtggggcccaggcaccctggtcaccgtctcctca

SEQ ID NO: 59 - DNA, 327 bp, Oryctolagus cuniculus
caagtgctgacccagactccatcccctgtgtctgcagctgtgggaggcacagtcaccatcagttgccaggccagtcagagtgttgctggtaacaactacttatcctggtatcagcagaaaccagggcagcctcccaacctcttgatctattctgtatccactctggcctctggggtcccatcgcggttcaaaggcagtggatctgggacacagttcactctcaccatcagcgacctggagtgtgacgatgctgccacttactactgtcaaggctattataatgatggaatttgggctttcggcggagggaccgaggtggtggtcaaa

SEQ ID NO: 60 - DNA, 357 bp, Oryctolagus cuniculus
caggagcaggtggaggagtccgggggaggcctggccaagcctggggcatctttgacactcacctgcgaagcctctggattcaccctcagtagttactggatgtgctgggtccgccaggctccagggaaggggctggagtggatcgcatgcatttatggtgggagtagtggtagcacttactactcgaactgggcgaaaggccgattcaccatctccaaaacctcgtcgaccacggtgactctgcaaatgaccagtctgacagccgcggacacggccacctatttctgtgcgagagatggtagtacctgggactactttaggttgtggggcccaggcaccctggtcaccgtctcttca

SEQ ID NO: 61 - DNA, 330 bp, Oryctolagus cuniculus
tatgatatgacccagactccatcctccgtggaggcagctgtgggaggcacagtcaccatcaagtgccaggccagtcagagtattaatactaactacttatcctggtatcagcagaaaccagggcagcctcccaagctcctgatctaccaggcatccactctggaatctggggtcccatcgcggttcaaaggcagtggatctgggacagagtacactctcaccatcagcgacctggagtgtgccgatgctgccacttactactgtcaaggctattatactgttgagaatattggtaatcctttcggcggagggaccgaggtggtggtcaaa

SEQ ID NO: 62 - protein, 111 aa, Oryctolagus cuniculus
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKK

SEQ ID NO: 63 - protein, 110 aa, Oryctolagus cuniculus
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVK

SEQ ID NO: 64 - protein, 253 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKK

SEQ ID NO: 65 - protein, 253 aa, Artificial sequence
  synthetic sequence
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSS

SEQ ID NO: 66 - protein, 251 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVK

SEQ ID NO: 67 - protein, 251 aa, Artificial sequence
  synthetic sequence
VVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSS

SEQ ID NO: 68 - protein, 252 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVK

SEQ ID NO: 69 - protein, 252 aa, Artificial sequence
  synthetic sequence
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSS

SEQ ID NO: 70 - protein, 247 aa, Artificial sequence
  synthetic sequence
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVK

SEQ ID NO: 71 - protein, 247 aa, Artificial sequence
  synthetic sequence
QVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSS

SEQ ID NO: 72 - protein, 498 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 515 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 495 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 498 aa, Artificial sequence
  synthetic sequence
MLLLVTSLLLCELPHPAFLLIPDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 515 aa, Artificial sequence
  synthetic sequence
MLLLVTSLLLCELPHPAFLLIPDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 496 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 496 aa, Artificial sequence
  synthetic sequence
MLLLVTSLLLCELPHPAFLLIPVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 497 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 497 aa, Artificial sequence
  synthetic sequence
MLLLVTSLLLCELPHPAFLLIPDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 492 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 509 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 489 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 492 aa, Artificial sequence
  synthetic sequence
MLLLVTSLLLCELPHPAFLLIPQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 509 aa, Artificial sequence
  synthetic sequence
MLLLVTSLLLCELPHPAFLLIPQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 476 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 493 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 473 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 476 aa, Artificial sequence
  synthetic sequence
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 493 aa, Artificial sequence
  synthetic sequence
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 474 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 474 aa, Artificial sequence
  synthetic sequence
VVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 475 aa, Artificial sequence
  synthetic sequence
QSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 475 aa, Artificial sequence
  synthetic sequence
DVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 470 aa, Artificial sequence
  synthetic sequence
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 487 aa, Artificial sequence
  synthetic sequence
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 467 aa, Artificial sequence
  synthetic sequence
QEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 470 aa, Artificial sequence
  synthetic sequence
QVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 487 aa, Artificial sequence
  synthetic sequence
QVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 755 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 101 - protein, 763 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLINGSGQCTNYALLKLAGDVESNPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 102 - protein, 758 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 103 - protein, 772 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 104 - protein, 752 aa, Artificial sequence
  synthetic sequence
MLLLVTSLLLCELPHPAFLLIPQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 105 - protein, 755 aa, Artificial sequence
  synthetic sequence
MLLLVTSLLLCELPHPAFLLIPDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 106 - protein, 772 aa, Artificial sequence
  synthetic sequence
MLLLVTSLLLCELPHPAFLLIPDVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 107 - protein, 749 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 108 - protein, 752 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 109 - protein, 766 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 110 - protein, 746 aa, Artificial sequence
  synthetic sequence
MLLLVTSLLLCELPHPAFLLIPQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 111 - protein, 749 aa, Artificial sequence
  synthetic sequence
MLLLVTSLLLCELPHPAFLLIPQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 112 - protein, 766 aa, Artificial sequence
  synthetic sequence
MLLLVTSLLLCELPHPAFLLIPQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGGGGSGGGGSGGGGSGGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 113 - protein, 22 aa, Artificial sequence
  synthetic sequence
MLLLVTSLLLCELPHPAFLLIP

SEQ ID NO: 114 - protein, 18 aa, Artificial sequence
  synthetic sequence
GSTSGSGKPGSGEGSTKG

SEQ ID NO: 115 - protein, 45 aa, Artificial sequence
  synthetic sequence
TTTPAPRPPTPAPTIASQPLSLRPEACRPAAGGAVHTRGLDFACD

SEQ ID NO: 116 - protein, 48 aa, Artificial sequence
  synthetic sequence
AKPTTTPAPRPPTPAPTIASQPLSLRPEACRPAAGGAVHTRGLDFACD

SEQ ID NO: 117 - protein, 62 aa, Artificial sequence
  synthetic sequence
GGGGSGGGGSGGGGSGGTTTPAPRPPTPAPTIASQPLSLRPEACRPAAGGAVHTRGLDFACD

SEQ ID NO: 118 - protein, 39 aa, Artificial sequence
  synthetic sequence
IEVMYPPPYLDNEKSNGTIIHVKGKHLCPSPLFPGPSKP

SEQ ID NO: 119 - protein, 10 aa, Artificial sequence
  synthetic sequence
GGGSSGGGSG

SEQ ID NO: 120 - protein, 12 aa, Artificial sequence
  synthetic sequence
ESKYGPPCPSCP

SEQ ID NO: 121 - protein, 106 aa, Artificial sequence
  synthetic sequence
GQPREPQVYTLPPSQEEMTKNQVSLTCLVKGFYPSDIAVEWESNGQPENNYKTTPPVLDSDGSFFLYSRLTVDKSRWQEGNVFSCVMHEALHNHYTQKSLSLSLGK

SEQ ID NO: 122 - protein, 229 aa, Artificial sequence
  synthetic sequence
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - protein, 24 aa, Artificial sequence
  synthetic sequence
IYIWAPLAGTCGVLLLSLVITLYC

SEQ ID NO: 124 - protein, 23 aa, Artificial sequence
  synthetic sequence
IYIWAPLAGTCGVLLLSLVITLY

SEQ ID NO: 125 - protein, 22 aa, Artificial sequence
  synthetic sequence
IYIWAPLAGTCGVLLLSLVITL

SEQ ID NO: 126 - protein, 27 aa, Artificial sequence
  synthetic sequence
FWVLVVVGGVLACYSLLVTVAFIIFWV

SEQ ID NO: 127 - protein, 28 aa, Artificial sequence
  synthetic sequence
MFWVLVVVGGVLACYSLLVTVAFIIFWV

SEQ ID NO: 128 - protein, 21 aa, Artificial sequence
  synthetic sequence
LCYLLDGILFIYGVILTALFL

SEQ ID NO: 129 - protein, 22 aa, Artificial sequence
  synthetic sequence
MALIVLGGVAGLLLFIGLGIFF

SEQ ID NO: 130 - protein, 27 aa, Artificial sequence
  synthetic sequence
IISFFLALTSTALLFLLFFLTLRFSVV

SEQ ID NO: 131 - protein, 42 aa, Artificial sequence
  synthetic sequence
KRGRKKLLYIFKQPFMRPVQTTQEEDGCSCRFPEEEEGGCEL

SEQ ID NO: 132 - protein, 41 aa, Artificial sequence
  synthetic sequence
RSKRSRLLHSDYMNMTPRRPGPTRKHYQPYAPPRDFAAYRS

SEQ ID NO: 133 - protein, 41 aa, Artificial sequence
  synthetic sequence
RSKRSRGGHSDYMNMTPRRPGPTRKHYQPYAPPRDFAAYRS

SEQ ID NO: 134 - protein, 42 aa, Artificial sequence
  synthetic sequence
ALYLLRRDQRLPPDAHKPPGGGSFRTPIQEEQADAHSTLAKI

SEQ ID NO: 135 - protein, 112 aa, Artificial sequence
  synthetic sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 23 aa, Artificial sequence
  synthetic sequence
GSGQCTNYALLKLAGDVESNPGP

SEQ ID NO: 137 - protein, 21 aa, Artificial sequence
  synthetic sequence
GSGEGRGSLLTCGDVEENPGP

SEQ ID NO: 138 - protein, 239 aa, Artificial sequence
  synthetic sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 139 - protein, 236 aa, Artificial sequence
  synthetic sequence
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 140 - protein, 683 aa, Artificial sequence
  synthetic sequence
MASPLTRFLSLNLLLLGESIILGSGEAQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSGSTSGSGKPGSGEGSTKGQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKTTKPVLRTPSPVHPTGTSQPQRPEDCRPRGSVKGTGLDFACDIYIWAPLAGICVALLLSLIITLICYHKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 141 - protein, 683 aa, Artificial sequence
  synthetic sequence
MASPLTRFLSLNLLLLGESIILGSGEAQVLTQTPSPVSAAVGGTVTISCQASQSVAGNNYLSWYQQKPGQPPNLLIYSVSTLASGVPSRFKGSGSGTQFTLTISDLECDDAATYYCQGYYNDGIWAFGGGTEVVVKGSTSGSGKPGSGEGSTKGQEQLEESGGGLVKPEGSLTLTCTASGFSFSSSYYMCWVRQAPGRGLEWIACIYTSSGSAYYANWAKGRFTISRTSSTTVTLQMTRLTAADTATYFCVRNAVGSSYYLYLWGPGTLVTVSSTTKPVLRTPSPVHPTGTSQPQRPEDCRPRGSVKGTGLDFACDIYIWAPLAGICVALLLSLIITLICYHKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 142 - protein, 687 aa, Artificial sequence
  synthetic sequence
MASPLTRFLSLNLLLLGESIILGSGEAQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSGSTSGSGKPGSGEGSTKGVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKTTKPVLRTPSPVHPTGTSQPQRPEDCRPRGSVKGTGLDFACDIYIWAPLAGICVALLLSLIITLICYHKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 143 - protein, 687 aa, Artificial sequence
  synthetic sequence
MASPLTRFLSLNLLLLGESIILGSGEAVVMTQTPSSVSEPVGGTVTIKCQASQSISDLLSWYQQKPGQPPKLLIYSASTLASGVSSRFKGSGSGTEFTLTISDLECADAATYYCQCNYYSPTYGNGFGGGTEVVVKGSTSGSGKPGSGEGSTKGQSLEESGGDLVKPGASLTLTCTASGIDFSDIYYISWVRQAPGKGLEWIACTYAGSSGSTYYATWAKGRFTISKASSTTVTLQMTSLTAADTATYFCARDRYPGTSGRVYGMDLWGPGTLVTVSSTTKPVLRTPSPVHPTGTSQPQRPEDCRPRGSVKGTGLDFACDIYIWAPLAGICVALLLSLIITLICYHKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGEGRGSLLTCGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 144 - protein, 27 aa, Artificial sequence
  synthetic sequence
MASPLTRFLSLNLLLLGESIILGSGEA

SEQ ID NO: 145 - protein, 29 aa, Artificial sequence
  synthetic sequence
MTSSHAMNITPLAQLALLFSTLLLPGTQA

SEQ ID NO: 146 - protein, 44 aa, Artificial sequence
  synthetic sequence
TTKPVLRTPSPVHPTGTSQPQRPEDCRPRGSVKGTGLDFACDIY

SEQ ID NO: 147 - protein, 39 aa, Artificial sequence
  synthetic sequence
FCKIEFMYPPPYLDNERSNGTIIHIKEKHLCHTQSSPKL

SEQ ID NO: 148 - protein, 20 aa, Artificial sequence
  synthetic sequence
IWAPLAGICVALLLSLIITL

SEQ ID NO: 149 - protein, 24 aa, Artificial sequence
  synthetic sequence
IWAPLAGICVALLLSLIITLICYH

SEQ ID NO: 150 - protein, 27 aa, Artificial sequence
  synthetic sequence
FWALVVVAGVLFCYGLLVTVALCVIWT

SEQ ID NO: 151 - protein, 45 aa, Artificial sequence
  synthetic sequence
KWIRKKFPHIFKQPFKKTTGAAQEEDACSCRCPQEEEGGGGGYEL

SEQ ID NO: 152 - protein, 41 aa, Artificial sequence
  synthetic sequence
NSRRNRLLQSDYMNMTPRRPGLTRKPYQPYAPARDFAAYRP

SEQ ID NO: 153 - protein, 113 aa, Artificial sequence
  synthetic sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 154 - protein, 162 aa, Artificial sequence
  synthetic sequence
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 155 - DNA, 1497 bp, Artificial sequence
  synthetic sequence
atgcttctcctggtgacaagccttctgctctgtgagttaccacacccagcattcctcctgatcccacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcgggagcacgagtggaagcggtaaaccaggttctggagaagggagtacgaaaggagacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaagaccacgacgccagcgccgcgaccaccaacaccggcgcccaccatcgcgtcccaaccacttagtttgaggcctgaagcctgtaggccagcagctggtggtgcagtgcacacgagggggctggacttcgcctgtgatatctacatctgggcgcccttggccgggacttgtggggtccttctcctgtcactggttatcaccctttactgcaaacggggcaggaagaagctgctctacatctttaagcagcctttcatgcgccctgtgcagacgactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 156 - DNA, 1497 bp, Artificial sequence
  synthetic sequence
atgcttctcctggtgacaagccttctgctctgtgagttaccacacccagcattcctcctgatcccagacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaaggggagcacgagtggaagcggtaaaccaggttctggagaagggagtacgaaaggacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcaccacgacgccagcgccgcgaccaccaacaccggcgcccaccatcgcgtcccaaccacttagtttgaggcctgaagcctgtaggccagcagctggtggtgcagtgcacacgagggggctggacttcgcctgtgatatctacatctgggcgcccttggccgggacttgtggggtccttctcctgtcactggttatcaccctttactgcaaacggggcaggaagaagctgctctacatctttaagcagcctttcatgcgccctgtgcagacgactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 157 - DNA, 2268 bp, Artificial sequence
  synthetic sequence
atgctgctcctcgttaccagcttgctcctgtgtgaattgccacatccagcgtttcttctgataccgcaatccctggaagaatccggtggcgatctcgttaagccaggcgcctctcttacgctgacatgtacagcatccggtattgacttctctgacatctattacatttcctgggtgagacaagctccgggtaagggattggaatggatagcctgcacgtacgcgggctcttccgggtcaacatactatgcaacctgggcaaaaggacgttttaccatctccaaagcaagtagcactaccgtcactctccaaatgacgtccctgacagctgcggatacagccacatacttctgtgccagggaccgatatcctggtacttcaggaagggtctatggaatggacctgtggggtcccgggaccctcgttaccgtctcctctgggagcacgagcggatcagggaaacctggcagcggggaaggctcaaccaagggagatgtggttatgactcagacgcctagctctgtgagtgagcccgtcggcgggacagttacgatcaagtgtcaagcctcacaaagcatctcagatctgctgtcctggtatcagcaaaagccggggcaacccccaaaacttcttatctacagcgcctctacgcttgcatcaggagtatcatccagatttaaaggttccggaagcgggaccgaattcactcttacaatctccgatctcgaatgcgctgatgctgcgacttattactgtcaatgtaattactattccccgacgtatgggaacggatttggcggtggcacagaggtcgtcgttaagaagaccactacgcctgccccgagaccgcccactcctgctcccactattgcaagccaacctctgagtctcagaccagaagcttgccgccctgcagcaggtggggcagtgcatacaagagggcttgattttgcttgcgacatttacatatgggctcccctggccggcacttgtggtgttctgctgctttccctcgtgattacactctactgcaagaggggacggaagaaactgctctacatattcaaacaacctttcatgcgacctgtccaaacaactcaggaagaggatgggtgttcctgtagatttcccgaagaagaggaaggcgggtgcgaactgagggtaaaattcagtcgctccgctgacgcacccgcatatcagcaaggccaaaaccagctttataatgaattgaacctcgggcggcgcgaggaatacgacgtgctggacaaacgccgtggtagggatccagaaatgggcggcaaacccaggcgcaagaatccgcaagaaggcctgtataatgaactgcaaaaggataaaatggcagaggcatattcagaaataggtatgaagggcgaacgacggagaggcaagggacatgatggactctaccaaggcttgtccactgccactaaggacacctatgacgcgctgcatatgcaagcgctgcccccgaggggcagcggtgaggggcgcggctctttgcttacgtgcggagatgtagaagaaaatcccgggccgatggtgagcaaaggcgaggaagacaatatggcgattatcaaggaattcatgaggtttaaagtgcacatggaagggtcagtgaatggccatgagtttgaaatagaaggtgagggtgaaggccgaccttacgaaggcacccaaactgcaaagctcaaggtaacaaaaggcggtccgttgccttttgcttgggatatactcagtccgcaattcatgtatggctctaaagcttatgtgaaacacccagctgacattccagattaccttaaattgagctttcccgaaggctttaaatgggaaagagtgatgaattttgaagatggcggagtcgtaaccgttactcaagattcaagtcttcaggacggtgagtttatttacaaagtcaaactccgtgggacaaatttcccttctgatgggccggtgatgcaaaagaaaactatgggctgggaagccagctcagagcgaatgtacccggaagacggcgcgctgaaaggagagattaagcagagactcaaactgaaggacggcggacactacgatgctgaagtcaaaacaacttataaggccaagaagccagtgcaactgcctggggcgtataatgtgaatattaaactggacatcacttctcacaacgaagactataccattgttgaacaatacgaacgtgcagagggtagacatagcacaggcggcatggatgaactctacaagtga

SEQ ID NO: 158 - DNA, 2292 bp, Artificial sequence
  synthetic sequence
atgcttctcctggtgacaagccttctgctctgtgagttaccacacccagcattcctcctgatcccacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcgggagcacgagtggaagcggtaaaccaggttctggagaagggagtacgaaaggagacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaagaccacgacgccagcgccgcgaccaccaacaccggcgcccaccatcgcgtcccaaccacttagtttgaggcctgaagcctgtaggccagcagctggtggtgcagtgcacacgagggggctggacttcgcctgtgatatctacatctgggcgcccttggccgggacttgtggggtccttctcctgtcactggttatcaccctttactgcaaacggggcaggaagaagctgctctacatctttaagcagcctttcatgcgccctgtgcagacgactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcttaattaacggaagcggacagtgtactaattatgctctcttgaaattggctggagatgttgagagcaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 159 - DNA, 2319 bp, Artificial sequence
  synthetic sequence
atgctgctgttggtgacctctctgcttctctgtgagctgcctcatcccgcttttctcttgatcccgcagagcttggaagagtcaggtggtgaccttgtgaagcctggcgcatccctcacattgacctgtacggcatccgggatagactttagcgacatttattacatttcttgggtaaggcaggccccaggcaaagggctggaatggatcgcttgcacttacgctggctcttcaggaagtacctattatgctacttgggcgaagggtcgtttcacgatcagcaaggcatctagcaccaccgtgacactccagatgacttccctgactgctgccgacaccgccacgtatttctgcgccagagaccgctaccctgggacctcaggccgagtctatgggatggacctgtggggccctggcacactggtgactgtctcctctggctcaactagcggatcaggcaagcccgggtctggagagggcagcaccaaaggggatgttgtgatgacccagacgcctagctcagtttcagagcctgtaggtggcacagtgacgatcaaatgtcaggccagtcaatctattagcgacttgctgagctggtatcagcaaaagcctggtcagccccctaagctgctgatatactctgcatccaccctggcctctggcgtgagcagccgatttaaaggctctggatctgggaccgaattcacacttacaatctctgaccttgagtgtgccgatgcggcaacctactactgtcagtgcaactattattctccgacttatggcaatgggttcggcggcggaactgaggtagtggtcaagaagggcggcggcggatcaggtggcggtggatctggcggaggaggctccggtggaacaaccacccctgccccacgtcccccgactcctgcaccgacgattgcatcacagcccttgtctctccgtcctgaggcctgtcgcccggcagcaggtggcgccgtgcatacgagaggactcgatttcgcatgtgatatatatatttgggccccactcgcaggcacttgcggcgtgctgcttctgagcctcgttattacactgtattgtaagcggggaaggaagaagctgttgtacatattcaaacaaccattcatgcgaccagtgcagacaacccaagaggaagatggctgttcatgcagattccccgaggaagaggaaggcgggtgtgagctgcgcgtaaaatttagcaggtccgcagatgcgcccgcttatcagcaaggccagaatcagctctacaatgaactgaatctgggccgcagagaagaatatgacgtccttgacaagaggcgaggtagggaccctgagatgggcggaaagccgagacgcaagaacccacaagagggtctgtacaatgagcttcaaaaggacaagatggccgaggcgtactctgagattggcatgaagggtgaacgtaggcgcggtaaggggcatgacgggctctaccaggggctgtcaacggctaccaaagatacttacgacgccctgcacatgcaggctctcccaccacgcggaagcggcgaagggcgagggagtctcctcacatgcggtgatgtggaagaaaacccaggaccgatggtcagcaaaggggaagaggacaatatggcgataattaaagagtttatgcgattcaaggtacatatggaaggatctgttaatgggcatgaatttgagattgaaggagaaggggaaggacgtccctacgaaggcacacagacagccaaactcaaggttaccaagggcgggcccctgccatttgcatgggacatacttagtccccaattcatgtatggctccaaggcctatgtcaagcaccctgcagatatccctgactatctgaagctgtcattccctgagggatttaagtgggaacgggtaatgaacttcgaagacggcggcgtggtgaccgtgacacaggattcttccctgcaagacggcgagttcatatataaggtaaagctgaggggtacgaacttcccatccgatggccccgtcatgcagaagaaaacaatgggatgggaagcgagtagcgaacgcatgtaccccgaagatggagcgttgaaaggagaaattaaacagcgcctcaaactgaaagacggcggacattacgacgctgaggttaagaccacatataaagccaagaagccggtccaactcccaggggcgtacaatgtcaacatcaaactcgatatcacaagccataatgaagattatactatcgtggagcagtacgaacgagccgagggcaggcactccacaggcggcatggatgaactgtataagtaa

SEQ ID NO: 160 - DNA, 2268 bp, Artificial sequence
  synthetic sequence
atgctcctgctcgtgacctctttgttgctttgtgagctgccccatcctgcgttcctgctgattccggatgttgtgatgacgcaaactccttccagcgtatctgaaccggttggcggtactgtaaccatcaaatgccaggcctcacaaagtatctctgatcttctttcttggtatcaacaaaagccaggtcaaccgcccaaattgctcatttactcagccagtacactcgcgagtggggtcagttcccgcttcaaaggatctggttctggaacagaattcaccctcacaatatccgatttggaatgcgcagatgccgctacttactattgtcaatgtaactattattcaccaacttatgggaatggctttggtggtggcaccgaggtggttgttaagaaggggagcacatccggcagtggcaagccagggtccggggaaggatcaacaaagggtcaatcactggaagaaagcggcggcgaccttgtgaaaccgggagccagcctcacgctcacttgtaccgcatcaggtatcgacttttcagacatctactatatctcctgggtgcgtcaagcgccaggtaaaggcttggaatggatcgcatgtacttacgcaggaagcagtggctctacgtactatgccacatgggcaaaggggagatttacaataagtaaagcatcatctactacagttactcttcaaatgacaagcctgacagcggccgatactgctacgtatttctgtgctagagatcgttacccggggacttcaggcagagtctacggcatggacctctggggacctggaactttggtaacagtcagctccacaacgaccccggctccccggccaccaaccccagcacccactatagcatcccaaccattgtctctgagacctgaggcttgcagaccagcagccggcggcgccgttcacactaggggcttggattttgcatgtgatatttacatttgggccccactggccggcacttgtggcgtgctgcttctcagtctcgtgattactttgtactgcaaaagaggccggaagaagctgctgtatattttcaaacaaccgtttatgcgccctgttcaaacgacgcaagaagaggacggctgttcttgcagattccctgaggaagaagaaggtggatgcgaactcagggttaaattcagcaggagtgcggacgcgccggcgtatcaacagggtcagaatcaactttacaacgagctgaatctgggcagacgggaagagtatgatgttcttgacaagcgcagagggcgcgatccggagatgggtgggaaaccccggcgaaagaatccacaagaaggcctctataacgagctccaaaaggacaaaatggcagaagcttatagcgaaatcggaatgaaaggggagcggagacgtggtaaagggcacgacggcctctatcaagggctttctacagctaccaaagacacttatgacgcccttcacatgcaagcgctcccaccaagaggatcaggcgagggccggggttccctgttgacatgtggggatgtcgaagaaaacccaggcccgatggtgtcaaagggtgaggaagataatatggcgattatcaaagagttcatgagattcaaggttcatatggaaggaagtgtcaatgggcatgaatttgagattgagggagaaggggaaggtcgcccttatgagggcacccaaacagcaaaacttaaagtcaccaagggcggtcctcttccgttcgcgtgggacatcctgtcccctcaattcatgtacggatccaaagcttatgttaagcacccagctgatattcccgattacttgaaactgtccttccccgaaggctttaaatgggagcgcgtgatgaattttgaagatggcggggtagtaaccgttacacaggactcaagtctgcaagatggggaatttatctataaagtcaagcttcgaggcaccaactttccatcagacggaccggttatgcagaagaagactatgggctgggaagccagcagtgaacgtatgtacccagaagatggggcactgaagggagagatcaagcaaaggttgaagttgaaagatggtggacattacgatgccgaggttaagacaacgtataaagccaagaagcctgtccaacttcccggagcatacaacgttaacatcaagttggatatcacatcacataatgaagactatacgatagttgaacaatacgaaagggcagagggaaggcacagtacaggcgggatggatgagctgtataaatga

SEQ ID NO: 161 - DNA, 2319 bp, Artificial sequence
  synthetic sequence
atgttgttgttggtaacatctttgcttctgtgtgagctccctcatcccgccttcctgcttatacccgatgtggtaatgacgcaaactccttcaagtgtcagtgaacccgtaggcggaacggtcaccatcaagtgtcaggcgtctcaaagcattagcgatctcctgtcatggtatcagcaaaagccaggtcagccacccaagctcctcatatattcagcgagcaccctcgcgtcaggagttagcagtcgcttcaaagggtcaggatccggtacggaattcacccttaccataagcgacctcgaatgtgccgatgccgcgacatattattgccaatgtaattattattccccgacatacggtaatggctttgggggggggacagaggtcgtggttaagaaggggtccacgtcaggttccgggaagcccggctccggtgaggggagcactaaaggtcagtcactcgaggaatctggtggagacctcgtgaagccaggagcatccctgacactcacctgcacagcgtcagggattgacttctccgacatatactacattagttgggtccgacaggctccggggaaaggcttggaatggatagcatgcacctatgccgggagctcaggcagtacatactatgccacttgggcgaaaggccgtttcacgatctcaaaagctagctccaccacagtcactttgcaaatgacgtctctcaccgccgccgacacagccacgtatttctgtgccagagatagataccccggaacctctggcagagtctatggtatggatttgtggggtcccggtactctggtcaccgtaagttctggaggaggcggtagtggcggcggcggttccggtggaggcgggtctggcggaacaacaactccggcccccaggccgcctacacctgctccaactatcgccagccaacccttgtcactccgccccgaagcttgcagacctgccgccggtggcgcagttcatacgaggggacttgacttcgcctgtgatatctacatatgggcgccacttgcaggaacttgtggcgttctccttctctctctggtgattaccctctattgtaaacggggccgcaagaaactcctgtatatcttcaagcaaccattcatgaggccagttcagaccacccaagaagaggacggctgctcttgtcggttccctgaggaagaggaaggcggatgtgaactcagagtgaaattctccagatctgcagatgctccggcctaccaacagggccaaaaccaactctataacgaacttaacttggggagacgcgaagaatacgatgtgttggataagcgaagaggtcgggatccagagatgggcggaaaacctagacgcaagaatccgcaggaagggctgtacaatgaactccagaaagacaaaatggcagaagcatacagtgaaattggaatgaaaggagaaagaagaagagggaaaggacacgacggactttatcagggcctctcaaccgcaacaaaagacacttacgacgccctgcacatgcaagctcttccccctcggggctccggagaaggccggggctccctgctgacttgtggcgacgtggaagaaaacccaggacctatggtgagtaaaggggaagaggacaacatggccataattaaagaatttatgaggttcaaagtgcatatggaaggttctgtcaatgggcatgaatttgaaattgagggagaaggggaaggccggccttatgagggtactcaaaccgctaaattgaaagtgacaaagggcggtcctctgccgtttgcatgggatatcttgtctccgcaattcatgtacgggtcaaaagcttacgtcaaacacccggccgatataccagactatttgaaactgtcattcccagagggatttaaatgggagagggtcatgaattttgaggatggcggcgttgttacggtaacccaggattcatcactgcaagacggggaattcatttataaagtcaaactcagagggacaaatttccccagcgatggtcctgtgatgcaaaagaaaacaatgggctgggaagcctcttcagaaagaatgtatcctgaggatggagccctgaagggagagattaaacagcggctgaagctgaaggacggtggtcattacgacgctgaagtaaagacaacatataaggcaaagaaaccagtccaattgccgggcgcatataatgtcaacatcaaactggatataacctctcacaatgaagattacacaatcgtcgagcagtacgagcgggcagaagggagacacagtaccggtggcatggatgagctgtataaatga

SEQ ID NO: 162 - DNA, 2277 bp, Artificial sequence
  synthetic sequence
atgttgttgcttgtcacgtcccttcttctgtgtgagctcccccatcctgcatttctcctcatcccacagagtcttgaggagtcaggcggggacctggtaaaacccggcgcttccttgaccctgacctgcacggcaagtggtattgatttcagcgatatttattatatctcatgggtgcgacaagcacctggaaaaggccttgagtggattgcgtgtacttacgccggtagcagcgggagcacttattacgctacttgggcaaagggccgcttcaccatttccaaggccagtagcacaaccgttactcttcaaatgacctctctcaccgccgccgatacggcgacttatttctgcgcaagagatagataccctggtacatcaggaagagtctatggcatggacctctggggcccggggacactggtcaccgtctcttcaggatccacctcagggagtggaaaaccaggttctggagagggctcaacaaaaggggatgtcgttatgacgcagactccttccagcgtctcagagccagtaggtggcactgtaacgattaaatgtcaggcatctcaatccattagcgatcttctgtcttggtatcaacaaaagcctggtcagccccctaaactcctcatctatagcgcatctacgttggcatcaggggtgtcatctcgctttaaaggctccggaagtggaaccgaatttacacttactatcagtgatttggaatgcgcagacgccgcaacctattattgtcaatgcaattattatagtccaacgtacggcaacgggttcggggggggaactgaagtggttgtcaagaagaccacaaccccggcaccacgtccacctacgcctgcaccaacaattgcctctcagcctcttagcctgcggcctgaagcatgtcgtcccgctgccggtggtgcagtccacacccgcggactggacttcgcctgcgatatttacatatgggctccgctggctgggacctgcggagtgctgctgctcagtcttgtgatcactctgtactgcaaacgaggccgtaagaaactgttgtatattttcaagcaaccgtttatgcgcccagttcagaccacgcaagaggaagacggatgctcttgtcggtttcccgaagaggaagaaggcggctgtgagcttagagtgaaattttcaaggtctgcagatgcccccgcgtaccaacaaggacaaaatcaactgtacaacgagctgaacctggggcggcgcgaggaatatgatgtgctcgacaaaagacgcgggagagaccccgaaatgggcggcaaaccccggcgcaagaatccgcaagaaggtttgtataatgaactccaaaaggataagatggcagaagcatactctgaaattggaatgaaaggggagcggcgcaggggaaaggggcacgacggcctgtatcaggggctgtccacagctactaaggatacatatgacgcgctgcacatgcaggcgttgccgcctcggggttctggcgagggtaggggatcactgcttacttgtggtgacgtggaagagaatcctggtcccatggtttccaagggtgaagagttgttcacaggcgttgtgccgatcctggtggagttggatggagacgttaatggacataagtttagcgtttctggcgaaggagagggcgacgcaacatacggcaaactgaccttgaagttcatttgtacaaccgggaagctgccagtcccgtggccaacactcgtgaccactctgacttacggcgtgcaatgcttttcccgctaccctgaccatatgaagcagcacgatttctttaagtccgccatgccggaagggtatgtccaagagcggacaattttctttaaggatgatggaaactacaagacgagagctgaagtcaagtttgaaggagatacactggttaataggatcgagcttaaaggcatagatttcaaggaagacggaaatattctggggcacaagctcgagtacaactataactcacacaacgtgtatatcatggctgacaagcaaaagaacggcataaaagttaattttaagataagacacaatatcgaagacgggtctgtgcagcttgctgatcattatcaacaaaacactccaataggagatggaccagttctccttcccgataatcactatctcagcacgcaatcagcgctgagtaaagaccccaacgagaagcgagaccacatggtgctcctggaatttgtcactgcagcaggaattacgcttgggatggatgaactgtacaagtga

SEQ ID NO: 163 - DNA, 2259 bp, Artificial sequence
  synthetic sequence
atgctcctcttggtgacatcactccttctgtgtgaactcccacatcctgcttttctgcttattccacaatcactggaagaatctggtggtgacctcgtcaagccaggtgcgtctctcactctgacctgcacagctagtggaattgacttttcagatatttattacatttcctgggtaagacaggccccgggcaagggcttggaatggattgcttgtacatacgcaggttcctcagggtcaacctattatgctacatgggctaaaggtagatttactatcagcaaagcatcttccacgactgttacccttcaaatgaccagcctcacggcggccgacactgctacgtacttttgtgctcgcgacaggtatcctggaaccagcggtcgggtctacgggatggacctttggggtccaggcacccttgttactgtgtcatccggttcaacatccggctccggaaagcctggttccggggaaggtagcaccaagggcgatgtggtaatgactcaaactccaagctctgtctcagaaccggtgggcggtacagtaacaataaaatgtcaagcatcccagagcatctccgacctgttgagttggtatcagcaaaagccaggtcaaccaccgaagttgctcatttacagcgctagcacgctcgccagcggtgtgagcagcaggtttaaaggctccgggtccggaacggaattcactctgactatcagtgacctggagtgcgccgatgcagctacctattattgccaatgtaattattattcacccacttacggcaatggtttcggcggcgggaccgaagtagtagtgaagaaaattgaagtgatgtatcccccgccctatctggataatgagaaaagcaatggtacgatcatccacgttaagggcaagcacttgtgccccagcccactgtttcccggacccagtaaacctttctgggttttggtcgtggttggcggcgttcttgcctgttattccctgcttgtcacggtggcgtttatcatcttctgggtcaagcgaggcaggaagaaactcctctatatctttaaacaaccctttatgcggccagtacaaactacacaagaagaggatggctgttcatgccgtttccccgaagaggaagaaggcggctgcgaactgcgggttaagttctctagaagtgctgacgcaccagcttatcaacaaggacagaatcaactctataatgaactgaatctgggccgacgcgaggaatacgatgttctggacaagagacgcggacgcgacccagagatgggcggaaaaccacgtaggaagaatccacaagaaggcttgtataacgaacttcaaaaggataaaatggccgaggcgtattctgaaataggaatgaagggcgagaggagacgcgggaagggtcatgacgggctgtatcaaggtctgtctacagccaccaaggacacctacgatgccctgcacatgcaagcactcccgccacgcgggagcggtgaagggcgtggatccctgctcacttgcggagatgtggaagaaaacccaggcccgatggttagcaagggcgaagaggataatatggccattatcaaagaatttatgcgttttaaggtccatatggaagggtccgtcaacgggcacgagtttgaaattgaaggggaaggggaagggcggccttacgagggcactcaaacagcgaagctgaaagtgactaaaggcggccctctcccttttgcttgggatattctttctcctcaattcatgtacggaagtaaagcatacgtgaaacatcctgcagatatccctgattaccttaaactctcattccccgaagggttcaagtgggaaagagtgatgaattttgaggatggcggcgtcgttactgtgacgcaagattctagtctccaagacggagagtttatatataaggttaaactgcggggcaccaacttcccctcagatggtcccgtgatgcaaaagaagactatggggtgggaagctagctctgaaaggatgtaccctgaggacggagccctcaagggtgaaattaagcaaaggcttaagctgaaagatggcggccactacgatgccgaagtcaaaacaacatacaaagcgaagaaaccagttcagctgcctggagcttataatgttaacataaaactggatattacctctcacaacgaagactatactatcgtcgaacagtatgagagggctgaagggcggcactctactggcgggatggatgaactttacaagtga

SEQ ID NO: 164 - DNA, 1491 bp, Artificial sequence
  synthetic sequence
atgcttctcctggtgacaagccttctgctctgtgagttaccacacccagcattcctcctgatcccacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcggcagtactagtggtagtggaaagcccggaagcggggaaggctccaccaagggagttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaccaccactccggctccccgaccacctactcctgcccctacaatcgctagccagcctttgagtcttaggcctgaagcttgtaggcctgccgcaggcggggcagtgcacacaaggggccttgacttcgcctgtgatatctacatatgggcacccctggctggaacctgcggagtgctccttttgagtctcgtgattactctgtactgcaagcggggaagaaagaagctgttgtacattttcaagcagccattcatgcgacccgtccagaccacacaagaggaagacggctgtagttgcaggtttcctgaagaggaagagggcggatgcgagctccgcgtgaaattcagtagatccgcagatgcccctgcttaccagcaggggcaaaaccagctgtataacgaattgaatctgggacgccgagaggaatacgacgtgctcgacaagagacgtgggcgtgatccagagatgggtggcaaaccccggcgtaagaaccctcaagaggggctgtacaatgaactccaaaaggataagatggccgaagcttattccgaaattggcatgaaaggggaacgccggcgcgggaaaggccacgacggactttaccagggtcttagcaccgcgacaaaggacacctacgacgcattgcatatgcaggcactcccacccaggtaa

SEQ ID NO: 165 - DNA, 1491 bp, Artificial sequence
  synthetic sequence
atgcttctcctggtgacaagccttctgctctgtgagttaccacacccagcattcctcctgatcccagttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaggcagtactagtggtagtggaaagcccggaagcggggaaggctccaccaagggacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcaccaccactccggctccccgaccacctactcctgcccctacaatcgctagccagcctttgagtcttaggcctgaagcttgtaggcctgccgcaggcggggcagtgcacacaaggggccttgacttcgcctgtgatatctacatatgggcacccctggctggaacctgcggagtgctccttttgagtctcgtgattactctgtactgcaagcggggaagaaagaagctgttgtacattttcaagcagccattcatgcgacccgtccagaccacacaagaggaagacggctgtagttgcaggtttcctgaagaggaagagggcggatgcgagctccgcgtgaaattcagtagatccgcagatgcccctgcttaccagcaggggcaaaaccagctgtataacgaattgaatctgggacgccgagaggaatacgacgtgctcgacaagagacgtgggcgtgatccagagatgggtggcaaaccccggcgtaagaaccctcaagaggggctgtacaatgaactccaaaaggataagatggccgaagcttattccgaaattggcatgaaaggggaacgccggcgcgggaaaggccacgacggactttaccagggtcttagcaccgcgacaaaggacacctacgacgcattgcatatgcaggcactcccacccaggtaa

SEQ ID NO: 166 - DNA, 1494 bp, Artificial sequence
  synthetic sequence
atgcttctcctggtgacaagccttctgctctgtgagttaccacacccagcattcctcctgatcccacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcggcagtactagtggtagtggaaagcccggaagcggggaaggctccaccaagggagacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaccaccactccggctccccgaccacctactcctgcccctacaatcgctagccagcctttgagtcttaggcctgaagcttgtaggcctgccgcaggcggggcagtgcacacaaggggccttgacttcgcctgtgatatctacatatgggcacccctggctggaacctgcggagtgctccttttgagtctcgtgattactctgtactgcaagcggggaagaaagaagctgttgtacattttcaagcagccattcatgcgacccgtccagaccacacaagaggaagacggctgtagttgcaggtttcctgaagaggaagagggcggatgcgagctccgcgtgaaattcagtagatccgcagatgcccctgcttaccagcaggggcaaaaccagctgtataacgaattgaatctgggacgccgagaggaatacgacgtgctcgacaagagacgtgggcgtgatccagagatgggtggcaaaccccggcgtaagaaccctcaagaggggctgtacaatgaactccaaaaggataagatggccgaagcttattccgaaattggcatgaaaggggaacgccggcgcgggaaaggccacgacggactttaccagggtcttagcaccgcgacaaaggacacctacgacgcattgcatatgcaggcactcccacccaggtaa

SEQ ID NO: 167 - DNA, 1494 bp, Artificial sequence
  synthetic sequence
atgcttctcctggtgacaagccttctgctctgtgagttaccacacccagcattcctcctgatcccagacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaggcagtactagtggtagtggaaagcccggaagcggggaaggctccaccaagggacaaagtctggaagagtctgggggcgaccttgtcaagcccggagcttcactgaccctgacctgcacggctagtggaatcgacttctccgacatatattacatcagttgggttcgccaagcgcctgggaagggtctggaatggatagcgtgtacgtatgccgggagtagcgggtccacatattacgctacgtgggcgaaaggtagattcacgatctccaaggctagttcaacaacagtaacattgcagatgacttccctcacagcagccgatacggctacatacttttgtgcgcgggacaggtatccaggaacctcagggagggtatatggaatggacttgtggggaccgggcaccttggttaccgtgagtagcaccaccactccggctccccgaccacctactcctgcccctacaatcgctagccagcctttgagtcttaggcctgaagcttgtaggcctgccgcaggcggggcagtgcacacaaggggccttgacttcgcctgtgatatctacatatgggcacccctggctggaacctgcggagtgctccttttgagtctcgtgattactctgtactgcaagcggggaagaaagaagctgttgtacattttcaagcagccattcatgcgacccgtccagaccacacaagaggaagacggctgtagttgcaggtttcctgaagaggaagagggcggatgcgagctccgcgtgaaattcagtagatccgcagatgcccctgcttaccagcaggggcaaaaccagctgtataacgaattgaatctgggacgccgagaggaatacgacgtgctcgacaagagacgtgggcgtgatccagagatgggtggcaaaccccggcgtaagaaccctcaagaggggctgtacaatgaactccaaaaggataagatggccgaagcttattccgaaattggcatgaaaggggaacgccggcgcgggaaaggccacgacggactttaccagggtcttagcaccgcgacaaaggacacctacgacgcattgcatatgcaggcactcccacccaggtaa

SEQ ID NO: 168 - DNA, 1479 bp, Artificial sequence
  synthetic sequence
atgcttctcctggtgacaagccttctgctctgtgagttaccacacccagcattcctcctgatcccacaggaacagttggaagagtcaggtgggggcttggtcaagcctgaaggtagtctgacactcacctgcactgcatcagggttttcttttagctcatcatattatatgtgctgggtcagacaagcgccggggagaggactggagtggattgcgtgcatttatacttcttctggttcagcctattacgctaattgggctaaagggcggttcacaatatctcgaacttcttctactactgtgacactccagatgacgcgcctgacggctgctgatacagcaacctacttctgtgtacgaaatgctgttgggtccagttactatttgtacctgtgggggcctggaactctcgtgacagtaagtagcgggagcacgagtggaagcggtaaaccaggttctggagaagggagtacgaaaggacaagtcttgactcagaccccaagccctgtgtccgcagcggttggtggtacagtaacaatatcttgccaggcctcacaaagtgtggcaggcaacaactacctttcatggtatcaacaaaaaccagggcagcccccaaacctcctcatctatagcgtgtccaccttggcttccggggttccatcccggtttaagggtagtggaagtggtacacaattcacgttgacaatctcagacttggaatgtgatgacgcagccacatattactgccagggctattacaacgacggcatctgggcttttggtggaggaactgaggtagtggttaaaaccacgacgccagcgccgcgaccaccaacaccggcgcccaccatcgcgtcccaaccacttagtttgaggcctgaagcctgtaggccagcagctggtggtgcagtgcacacgagggggctggacttcgcctgtgatatctacatctgggcgcccttggccgggacttgtggggtccttctcctgtcactggttatcaccctttactgcaaacggggcaggaagaagctgctctacatctttaagcagcctttcatgcgccctgtgcagacgactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 169 - DNA, 1479 bp, Artificial sequence
  synthetic sequence
atgcttctcctggtgacaagccttctgctctgtgagttaccacacccagcattcctcctgatcccacaagtcttgactcagaccccaagccctgtgtccgcagcggttggtggtacagtaacaatatcttgccaggcctcacaaagtgtggcaggcaacaactacctttcatggtatcaacaaaaaccagggcagcccccaaacctcctcatctatagcgtgtccaccttggcttccggggttccatcccggtttaagggtagtggaagtggtacacaattcacgttgacaatctcagacttggaatgtgatgacgcagccacatattactgccagggctattacaacgacggcatctgggcttttggtggaggaactgaggtagtggttaaagggagcacgagtggaagcggtaaaccaggttctggagaagggagtacgaaaggacaggaacagttggaagagtcaggtgggggcttggtcaagcctgaaggtagtctgacactcacctgcactgcatcagggttttcttttagctcatcatattatatgtgctgggtcagacaagcgccggggagaggactggagtggattgcgtgcatttatacttcttctggttcagcctattacgctaattgggctaaagggcggttcacaatatctcgaacttcttctactactgtgacactccagatgacgcgcctgacggctgctgatacagcaacctacttctgtgtacgaaatgctgttgggtccagttactatttgtacctgtgggggcctggaactctcgtgacagtaagtagcaccacgacgccagcgccgcgaccaccaacaccggcgcccaccatcgcgtcccaaccacttagtttgaggcctgaagcctgtaggccagcagctggtggtgcagtgcacacgagggggctggacttcgcctgtgatatctacatctgggcgcccttggccgggacttgtggggtccttctcctgtcactggttatcaccctttactgcaaacggggcaggaagaagctgctctacatctttaagcagcctttcatgcgccctgtgcagacgactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 170 - DNA, 2250 bp, Artificial sequence
  synthetic sequence
atgctcctgctggtgacgtcccttctcctgtgcgaactgccacacccagctttcctgctgataccccaggaacagctcgaggaatcaggtggtggcctggttaaacccgaaggcagcttgactcttacctgcactgctagtggtttctccttctccagctcatactatatgtgttgggttcgtcaagctccaggcaggggcctcgagtggatagcctgtatctacacttcatcaggtagcgcatactacgctaactgggctaagggccgctttactatctctcgaacaagtagcacaactgtcacgttgcagatgacacggcttaccgcggcagatacggcaacctacttttgcgtccggaacgcggtgggctccagctactacctttacctgtggggaccaggcacgctggttactgtatcttctggcagtactagtggtagtggaaagcccggaagcggggaaggctccaccaagggacaggtcctgacccagacaccttccccagtctcagccgccgtgggcggaactgtgacgataagttgtcaagcatctcagagcgtagcaggtaataactatctgtcctggtatcagcaaaagccagggcagccgcccaatctgcttatctattcagtctctacccttgccagcggcgtgccttcccggtttaagggatcaggatcaggcactcagttcacactgactatatccgatctcgaatgtgacgacgccgcaacatactactgtcagggatactataatgatgggatttgggccttcggggggggaaccgaggtcgtggttaaaaccaccactccggctccccgaccacctactcctgcccctacaatcgctagccagcctttgagtcttaggcctgaagcttgtaggcctgccgcaggcggggcagtgcacacaaggggccttgacttcgcctgtgatatctacatatgggcacccctggctggaacctgcggagtgctccttttgagtctcgtgattactctgtactgcaagcggggaagaaagaagctgttgtacattttcaagcagccattcatgcgacccgtccagaccacacaagaggaagacggctgtagttgcaggtttcctgaagaggaagagggcggatgcgagctccgcgtgaaattcagtagatccgcagatgcccctgcttaccagcaggggcaaaaccagctgtataacgaattgaatctgggacgccgagaggaatacgacgtgctcgacaagagacgtgggcgtgatccagagatgggtggcaaaccccggcgtaagaaccctcaagaggggctgtacaatgaactccaaaaggataagatggccgaagcttattccgaaattggcatgaaaggggaacgccggcgcgggaaaggccacgacggactttaccagggtcttagcaccgcgacaaaggacacctacgacgcattgcatatgcaggcactcccacccaggggcagcggtgagggccgcgggagtttgctcacctgcggagacgtggaagaaaacccaggtccgatggtgagcaagggtgaagaggataatatggccattatcaaagagttcatgcgctttaaggttcatatggaaggctccgtgaacggtcacgagtttgagatcgagggagagggtgaaggcaggccttacgaaggaacccaaacagcaaagctgaaagtaaccaaaggcggcccattgcccttcgcatgggacatcctgagtccccagtttatgtacggcagtaaggcctatgtaaagcaccccgcggacatccctgattatttgaaattgtcattcccagagggcttcaaatgggaaagagttatgaactttgaagacggcggcgtcgttaccgtgactcaggacagttctctccaagatggggaatttatttacaaagttaaattgcgaggcacaaacttccctagcgacggcccagtaatgcagaagaaaaccatgggttgggaagcctctagcgaacggatgtacccggaagacggagcactgaagggcgagatcaagcaacggctcaagttgaaggacggcggccattatgatgcagaggtgaaaaccacatacaaggcaaagaagccggtccagcttcccggcgcttacaacgtgaatattaagctggatataacttcccataatgaagactacactatcgttgagcagtatgaaagggcagagggtcgccattccactggtgggatggatgaattgtacaagtag

SEQ ID NO: 171 - DNA, 2301 bp, Artificial sequence
  synthetic sequence
atgctccttctggtgacgagcctcttgctctgcgaattgcctcatcccgcattcctgctcataccccaggagcaattggaagaatcaggtggtggacttgtaaagcctgagggttcactcacccttacgtgcaccgccagcggatttagttttagctcttcctattatatgtgttgggtaagacaagcccctggtaggggtttggaatggatagcctgcatatatacgagctcaggaagtgcttattatgctaactgggcaaagggcaggtttacaatctctcgaaccagctccaccaccgtcacccttcagatgactcgcctcactgcggcagacacagcaacgtatttctgtgtgcggaacgcagtaggatctagctattatttgtacctttggggcccgggaacgctcgttactgtttcttctggaagcaccagcggatcagggaaacccggtagcggcgaaggtagtacgaagggtcaagtcctgacccaaaccccctctcccgtgtccgcagcagtaggcggaactgttactatttcttgccaggcctctcaaagcgtcgctggtaacaactatctcagctggtatcagcaaaagcctggtcagccaccaaacctcttgatttactccgtttccacgcttgcttctggagttccctcacgatttaaagggtctggaagtgggactcagtttaccttgaccattagcgatcttgaatgtgacgacgcggcaacatattattgccaggggtactataatgacggtatctgggcattcggcggcggcactgaggttgtggtgaaaggcggtggcggaagtggcggaggaggctcaggtggcggcggttctggtggaacaaccactcctgcccctcggcccccaacacccgcacctaccatagccagtcaacctttgtctctgcgtcccgaggcttgtagacccgctgctggtggggctgtccatacaaggggtttggactttgcctgcgacatatatatttgggcgcccttggctggtacttgtggagtcctgctgctctctctcgttataacactgtactgcaaaagaggtcggaagaagcttctttatatcttcaagcaaccattcatgcgcccggttcaaaccacccaagaggaagacggctgctcctgccggtttccagaggaagaagagggcgggtgtgaattgagggtcaaatttagtcggagtgccgacgcgcccgcttatcaacaagggcaaaaccagctgtacaatgagttgaatctgggcagacgtgaggagtatgacgttcttgacaagcgtagaggtagggatcctgaaatgggtggcaaaccccggcgtaagaatccacaggaagggctctataacgaactgcagaaggataaaatggcagaagcttactccgagatcggtatgaaaggggaacgacgtcgagggaaaggccatgacgggctctatcaaggcctgagtacggctacaaaggacacctacgatgcccttcatatgcaggctctgccacctcgagggtcaggtgaaggcaggggttctctcttgacttgcggtgatgtagaagaaaatcctgggccaatggtgagcaaaggggaagaggacaatatggctatcatcaaagagttcatgcgattcaaggtacatatggaaggaagtgtcaatggccatgaatttgagatcgaaggcgaaggtgagggacgaccttatgagggtactcaaactgcaaagttgaaagtgaccaaaggtggtcccctgccctttgcgtgggatatcctcagcccacaatttatgtatgggtctaaggcatacgttaagcatcccgccgatatccctgattatctgaaactcagctttcccgagggcttcaaatgggagcgtgtgatgaattttgaggatggcggcgttgtgactgtgacccaggatagctcactccaagacggcgaattcatttacaaagtgaagctgcggggaacaaattttccctcagatggtccagtgatgcaaaagaagacaatgggctgggaagctagttcagaacgcatgtatcctgaggatggtgccctgaagggagaaataaaacaaagattgaaattgaaagatggcggtcactatgacgctgaagtaaagacaacttataaagccaagaagcccgtacaactgccaggcgcctataatgtaaatattaagttggacatcacctcacacaacgaagattataccatcgtcgagcagtacgaaagagcagagggccgtcatagtacaggcgggatggatgaattgtacaaatag

SEQ ID NO: 172 - DNA, 2250 bp, Artificial sequence
  synthetic sequence
atgctgctcttggtaaccagcctgctcctgtgcgagcttcctcatccggcctttctgttgatcccgcaagttctcacccagacaccttcccccgtttctgccgctgtcggcggtacggtcaccatctcttgtcaagcaagtcaaagtgtcgcgggaaataattatctgagctggtatcaacagaaacccggtcaacccccaaaccttctgatctactccgtgtctaccctcgcttctggcgtcccatcaaggtttaaaggctctggctctgggacccaattcacacttacaatctctgatctggagtgtgatgacgctgctacctactactgtcaaggatactacaatgatggaatctgggcatttggtggtgggaccgaagttgtagttaagggatcaacaagtgggagtggtaagccaggcagcggggaagggtccaccaaagggcaagaacaattggaagagagcggaggaggcctggttaaaccagagggctccctcactctgacctgtacagcctcaggatttagcttttcttcatcttattatatgtgttgggtgaggcaagctccgggccgtggtctcgagtggattgcctgtatatacaccagctccggatccgcctattacgcgaactgggccaaaggaaggtttacaatctctaggacgtcctcaactactgtgacacttcaaatgacacgacttacagcggcggacactgcgacatatttctgtgtgcggaatgcggtaggttcatcttactatttgtatctttggggacctggtacactcgtcactgtctcctctactaccactcctgctccaaggcccccaaccccagcccctactatagcttcccaaccactgtcactcaggccagaagcatgtaggcctgcagcgggtggtgctgttcatacgcgtgggctggatttcgcctgtgacatttacatatgggctccgctggcgggcacatgtggtgtgctgctgctgtccctggtcatcaccctctactgtaagaggggacgaaagaagcttctttacatcttcaaacaaccctttatgcgtccggtccaaacaacccaggaagaagatggatgctcctgtcgatttcccgaagaagaagaaggcggttgtgaactgagagttaagttctctcgctcagccgacgctccggcatatcaacaagggcaaaatcagctctacaatgaattgaatctcgggagacgagaggaatatgacgtgttggacaaaaggcgcggtcgtgacccagagatgggtggcaaaccacggcgcaagaaccctcaggaaggactttacaacgagctccaaaaggacaaaatggctgaagcatattccgagataggcatgaagggtgaaaggcgtaggggcaaagggcacgacggcctttatcagggcctctctacggccacgaaggatacgtatgacgcgttgcacatgcaagccttgccgccaaggggtagcggagaaggaagagggtctttgctgacttgtggcgacgtcgaagaaaaccctggaccaatggtaagcaaaggtgaggaagataatatggccatcatcaaagaatttatgagattcaaagtgcacatggaaggcagcgtgaatggccatgaatttgaaattgagggcgagggagaaggccgcccctacgaaggaacccaaactgccaaactcaaagtgactaaaggcgggccgctgccttttgcttgggatattctctccccacaattcatgtatggatccaaggcttatgttaaacatcccgcagatattcccgattacctgaaattgtcatttcccgagggattcaagtgggagagggttatgaattttgaggacggtggggtagttacagttactcaggactcttctcttcaagatggggaatttatctacaaggtcaaattgcggggcacaaattttccctctgatggacccgtgatgcaaaagaagacaatgggatgggaagcaagttctgaacgtatgtatccagaagatggggctttgaaaggcgagataaaacaaaggctgaagctcaaagatggtggacactacgatgcagaggtcaagacaacatataaggctaagaaacctgttcagctccctggggcttacaacgttaatattaaactggacattacttctcacaatgaggattacacgatagtggaacagtacgaaagagctgagggacggcatagtacaggcggcatggacgaactgtataaatga

SEQ ID NO: 173 - DNA, 2301 bp, Artificial sequence
  synthetic sequence
atgctgcttctggtaacctcactgctcctgtgtgaactgcctcatcctgcattcctgcttataccccaagtcctcacgcaaacaccaagtccagtaagcgcagccgtcggtgggactgttaccattagttgtcaggctagtcaatcagtcgctggaaacaattatctctcatggtatcaacaaaagccaggccaaccaccaaatctgctcatttactctgtctccacactggcatccggcgtcccgagtcggtttaagggtagtgggtctggaactcaatttaccctgacaatctcagatctcgagtgcgacgatgctgccacatattattgtcaaggatattataacgatgggatctgggctttcggcggtgggaccgaagtcgtcgtgaaagggtccactagtggttctggtaagcccggttcaggggaaggtagtacaaaagggcaagagcaacttgaagaatccggtggcgggcttgttaaacctgaaggtagcctcaccttgacctgcaccgccagtggcttctcatttagtagttcatactatatgtgctgggttcgccaagcccccgggcgcggactggagtggatcgcttgcatatacacttctagtggaagcgcctactatgccaactgggccaaaggtcggtttactatctcaagaaccagctccactactgtgacccttcaaatgacccggctgacagccgccgatactgccacatacttttgtgtccgcaatgcggtcggcagtagctactatctgtacctctggggaccagggaccttggtcacagtgtcatctggcggggggggttccggtggggggggatctggcggcggcggatctggtggaaccacgaccccagcacccagaccacctactccggctccaaccattgcttcacaaccattgtcactcaggcccgaggcctgccggccagccgcaggcggcgctgtgcatactcggggactggacttcgcatgtgacatttatatttgggcaccgctcgccggaacatgcggggttctgttgctgtccttggttataaccttgtactgtaaaagaggtcgcaagaagctcctttatatattcaaacaaccattcatgcggcccgtccaaacaacccaggaagaggatgggtgcagttgtcgttttccagaagaagaggaaggtggctgcgaattgagagtaaaattctctcgcagcgcagatgcacccgcttaccagcaaggacaaaaccaactttataacgaacttaacctggggcgccgggaagagtacgatgttcttgacaagcggagaggtcgcgacccagaaatgggcggtaagccgcgacggaagaatccccaagaaggtctttataatgagctccaaaaggacaagatggctgaggcatactcagagatagggatgaaaggcgagcggagacggggcaaggggcatgatggcctctaccaaggattgtccacagcaactaaggatacttacgatgctctgcacatgcaagcccttcccccgcgtggctccggcgagggtagagggtctctcctgacttgcggagacgtggaagaaaacccgggacccatggtctctaaaggcgaagaggataacatggccataatcaaggagtttatgcggttcaaggtacatatggaaggtagcgttaatgggcatgaattcgaaatagagggagaaggcgaagggagaccttatgaagggacacaaacagctaaactgaaagtaaccaagggcggacctcttcctttcgcttgggatatccttagccctcaatttatgtatggttccaaggcgtacgttaagcatcccgctgatattcccgactacctcaaactttcatttccagagggctttaaatgggaacgcgttatgaattttgaggacggcggggttgtcactgtgacccaggattcttccctgcaagatggcgaatttatctataaagtcaaactgagaggtacaaactttccttccgacggacctgtgatgcaaaagaaaacgatgggctgggaagcttcttccgagcggatgtatccagaggacggagccctgaagggcgaaataaaacaacgcctcaaactgaaggacggcggccattacgatgcagaggttaagacgacttataaagcaaagaaacctgtccaactgcctggtgcatataacgtcaacataaaactggacatcacatcccataatgaggactataccattgttgaacaatacgaacgggctgaaggcaggcattcaaccggtggcatggatgaactttacaagtga

SEQ ID NO: 174 - DNA, 2259 bp, Artificial sequence
  synthetic sequence
atgctgctcctggtcacttcactcctcttgtgtgaacttccccacccggcatttctcctgatcccacaagagcaactggaagaatccgggggggggctcgtcaagcccgaaggtagcctcactttgacttgtaccgcctcaggctttagtttctcttccagttattatatgtgttgggtccggcaggctcctggaagagggctcgaatggatcgcctgtatctataccagctcagggagcgcctattatgccaactgggcaaaaggccgtttcactatctctaggacttcatcaactactgtgactcttcaaatgacccggctgacagcagctgacactgcaacctacttttgcgtccggaatgcagttggctcctcatactacctctatttgtggggacccggcactctggtaacagtctcaagcgggagcacttccggctcagggaaacctggatccggggaaggaagcactaagggtcaagtgcttacccaaacaccgtctcccgtatcagcggctgtgggcggcactgtgactataagttgtcaggcatcccaatcagtggctggaaacaactatttgagctggtatcaacaaaagcccggtcaaccccctaatctcctgatttattcagtgtccactctcgcttctggcgtgcctagcaggtttaaaggttccgggtcaggtacacagtttaccctgacgatatccgacctcgaatgcgatgacgctgccacctactactgccagggctactataatgacggaatttgggcatttggtggtggcaccgaagtcgtagtcaaaacaaccacgcccgccccaaggccgccgaccccagccccgactatagccagtcaaccactctcattgagacccgaagcatgtcgtcccgctgcgggtggagccgtccacactcgtggccttgacttcgcctgtgatatttatatctgggctccactcgcaggcacctgcggtgtgctgctcctgagtctcgtcattaccctttattgcaagcggggccggaagaagctcttgtatatatttaagcaaccttttatgagacccgtgcaaaccacccaagaggaagacgggtgtagttgccgttttcctgaagaggaagaaggcggatgcgaattgcgagtcaaattttcccggtctgcggatgcccccgcgtatcaacaaggccaaaatcagctgtacaatgaattgaatctgggacggagagaggaatatgatgtacttgacaagcgacgcggccgagatcctgaaatgggcggaaagcctcgccgcaagaacccccaggaaggactctacaatgagctgcagaaggataaaatggcagaagcttatagcgaaattggaatgaaaggcgaaaggcggcgtggtaagggtcatgatggcctttatcagggacttagcaccgctactaaagacacgtatgatgctttgcacatgcaagcacttccaccccgcggctcaggcgagggacggggtagcctgttgacgtgtggcgacgtggaagagaatccagggccaatggtatctaagggagaggaactctttacgggtgtcgtccctatactggtggaactcgatggtgatgttaatggccataagttttctgtgtctggggaaggcgaaggcgacgctacttatggcaaactcaccttgaaatttatttgcactactgggaagttgcccgtcccatggccaaccctcgttaccacattgacgtatggcgtgcaatgtttctccagataccctgaccacatgaagcaacacgacttcttcaagtccgcgatgcccgaaggatacgtacaagaacggacgatcttctttaaagacgatggcaattacaaaacaagagcggaagtgaagtttgaaggggatacgctcgtgaaccgtattgaactgaaaggaatcgactttaaggaagacggtaatattcttggtcataagcttgagtacaactataacagccacaatgtatatatcatggccgataagcagaagaatggcatcaaagtaaattttaaaataaggcataacatagaggacgggagtgtgcaacttgcagatcactatcagcagaatacaccaattggggacgggccagtgctgttgcctgataatcattatctgagcacccaaagcgccctctccaaagaccccaacgaaaagcgcgatcacatggtcctgctggaatttgtcacggctgcaggtatcacactcgggatggacgagttgtataagtga

SEQ ID NO: 175 - DNA, 2241 bp, Artificial sequence
  synthetic sequence
atgttgctcttggtgaccagtcttcttttgtgtgagctgccgcatcccgcatttctgttgatccctcaagagcagctggaagaatcaggcggtggactggttaaacccgaagggtcccttacgcttacatgtactgcatccgggttcagtttctcatcatcatactacatgtgctgggtgagacaagctccagggagaggtctggaatggatcgcctgtatctatacatcctctggaagcgcgtactatgccaattgggctaaaggtcggttcactatttcccgaacatcttctactactgttacgcttcaaatgactagattgacagcggccgacacagcaacctacttttgtgttcgtaacgccgtcggatcaagctactatctgtatttgtggggtcccggcaccctggttactgtcagcagcgggagtacctcaggttcaggaaaacccggcagcggcgagggcagcactaaagggcaggtcctgactcaaacgccctctcctgtctcagcggctgttggtggcaccgtcacaatttcttgccaagcaagtcaaagtgttgcgggtaataattatttgagctggtatcaacaaaagcctggacagccgcctaacctgctcatttactctgtatctacattggcttctggagtgcccagcagatttaaaggaagtgggagtggaactcagtttacgttgacaattagtgatcttgagtgtgatgacgctgcaacctactactgccaaggttattacaatgacggaatttgggcttttggcggcgggacagaagtcgttgtcaaaattgaggttatgtacccaccaccctatctggataatgagaaaagcaatggtactattatccatgtaaagggaaagcatctctgtcctagccctctctttccagggcccagtaaaccattttgggttctcgtcgttgtgggcggcgtgctcgcctgttattcattgctcgtaacagttgcctttattattttctgggtcaagcgcggccggaagaagttgctgtacattttcaaacaaccatttatgcgcccagttcaaactacccaagaagaggatgggtgctcatgtaggttccctgaagaagaagaaggcggatgcgagctcagagtgaaattctcccgctctgcagatgctccggcttatcagcaagggcaaaatcagctgtacaacgagcttaaccttggtaggcgagaagaatatgacgttctggataaaagacgcggcagagacccagagatgggcgggaaaccaaggcgcaagaatccccaagagggtttgtataatgagctgcaaaaggacaaaatggctgaagcttattccgagattggtatgaaaggagaaagacgaagagggaagggccatgatggtttgtaccaaggtttgtcaacagctactaaagacacttacgatgcgcttcatatgcaagcactcccgcctaggggctccggggaaggaaggggaagtctcctcacgtgtggagacgtcgaggagaatccagggccgatggtttcaaagggtgaagaggataacatggctattataaaagagttcatgagatttaaagtgcatatggaaggcagcgtcaacggccatgaattcgaaattgagggtgagggtgaaggtcgtccgtatgaagggactcaaacggcgaaattgaaagttactaaaggcgggccgctcccttttgcgtgggacatattgagcccccagtttatgtatgggtctaaagcttatgtgaaacacccagctgacataccagactatcttaaattgtcctttcctgaaggattcaaatgggaacgcgtcatgaattttgaggatggcggggttgtgacagtgactcaagattcaagtcttcaggatggggaatttatatacaaagtcaagcttcgcggtacaaattttccgtcagatggccccgtcatgcaaaagaagacaatgggatgggaagcgtcaagcgaacgcatgtaccctgaggacggggcattgaaaggcgaaatcaaacaaaggcttaagctcaaggatggcggtcattatgatgcggaagtcaagactacatataaggccaagaagcccgtacagcttcctggcgcttataatgtgaatattaaacttgatatcaccagccacaacgaagattatactatcgtagaacagtatgagagagccgaagggcggcattcaacaggtggtatggacgaattgtacaagtga

SEQ ID NO: 176 - DNA, 2052 bp, Artificial sequence
  synthetic sequence
atggcatctccgctgacgagatttctatcccttaatttgttgctgctaggcgagtcaataattctcgggtcaggcgaagctcaagaacagctggaagaatcaggcggcgggctggttaagccagagggttctctgaccctcacgtgcaccgcctctggatttagcttctccagctcctattatatgtgctgggtccggcaagcccctggccgcggcctagagtggatcgcttgtatctatacgagctctgggtcagcttattatgcgaattgggcaaaaggtcgctttacaatttctagaacttcaagcaccactgtgactcttcaaatgacacgcctaaccgcggccgatacagcaacctatttctgtgtgaggaatgccgtcggttcatcctattacctgtacctgtggggacccggtaccctcgtgactgtcagttctggttctacatccggaagcgggaagcctgggtcaggcgagggaagtactaaaggtcaagtgctgacgcaaacaccgagtcccgtttctgctgccgtaggcggtacagtgaccatctcttgccaggccagtcaatcagtggctgggaacaactacctgtcctggtaccaacaaaagcccggtcaaccgcccaatcttctcatctactccgtgtccacactcgccagcggggtccccagccgcttcaaggggagcggaagcggcacacaatttacgctgacaattagcgacttggaatgcgacgatgcagcgacctattactgtcaaggctattataatgatggtatatgggcttttggtggtggcactgaagtcgtagtcaaaactaccaaaccagtgctgcgcacgccatcccccgtgcaccctactggcacttctcagccccagagacctgaagactgtcgccccaggggcagcgttaagggcactggattggacttcgcatgtgatatttacatctgggcgccactggcgggaatctgtgtagctctcctgctaagccttattattactcttatttgttaccacaagtggatcaggaagaaatttccccatatctttaagcaaccatttaagaaaactacaggagcggcgcaggaagaggatgcctgcagttgtcggtgcccccaagaggaagagggcggcggcggcggatatgaattgagagcaaaattttccaggtcagctgaaacggccgcaaatctacaagatcccaatcaactctataatgagcttaatctaggacgtcgcgaagaatatgatgtccttgagaagaagcgtgccagagaccccgaaatgggcggaaagcaacaacggcggcgcaaccctcaagagggagtctataacgccctgcagaaagacaaaatggccgaagcgtattccgaaatcgggactaagggagaaaggcgcaggggtaaaggccatgacggtctatatcaaggcttgagtacggcaacgaaagacacgtatgacgccctgcatatgcagacacttgcacccagagggtctggtgaagggcgcggcagccttctgacctgcggagatgtggaagaaaatcctggtcctatgaatcctgcaatttccgtcgctttgctgttgagcgtacttcaagtgtctcggggacaaaaggtcacgtctctcaccgcctgcctagtcaatcagaatttgcggctcgattgtagacacgaaaataacacgaaggataactccatccaacatgaattttcactcacccgcgaaaagaggaaacatgtattgagcggcactttgggcatcccagaacatacctatcgatctcgtgtaaccctgagtaatcaaccctatatcaaggttctgactctggccaactttactaccaaggacgaaggagattatttctgtgagctcagggtctcaggagccaacccgatgtccagcaacaagtctatctctgtgtatagagacaagctggtaaaatgtggcgggatctcactgcttgttcaaaacacctcatggatgctactactgctcctgtcactgtctctgctgcaagccctggatttcatctcactctaa

SEQ ID NO: 177 - DNA, 2052 bp, Artificial sequence
  synthetic sequence
atggcttctccactgacaagatttctcagcctcaacttgctcctactcggtgagagtattatcctgggaagcggagaggcgcaagtacttacgcagacaccgtcacccgtgtctgcagccgtgggcggcactgtaaccattagttgccaagcttcccagagtgttgcaggtaataactacctttcttggtaccagcaaaagcctggacaaccgcctaatctactcatctacagcgtttctaccctcgccagtggagtacccagccgtttcaaaggaagcggcagcgggacccaatttactcttactattagtgatctggaatgcgacgatgcggctacctactattgccaaggatattataatgacggaatatgggcgtttggcggcgggacagaggtcgtagtgaaaggatcaacatctggatctggtaagcctggttcaggggaaggctctactaagggacaagagcaactggaagaaagcggcggcggactggttaaacccgaaggaagtctaacgctcacttgcacagctagtgggttctccttctcaagctcatactacatgtgttgggttaggcaagctcctggaaggggactggaatggatcgcctgcatatatacatcttccggatctgcttattacgctaattgggctaaaggacgatttactatcagccgaacctcatctaccacagtgacattgcaaatgacgcgtttgaccgccgctgataccgccacctacttttgtgtccggaatgccgtcggtagttcttattacctttatctctggggacccggcaccctcgtcaccgtcagtagtactacgaaacccgtcttgagaacccctagtccagtgcaccccacgggcacctctcaaccccaacgccccgaagattgtcgcccaagaggctctgtaaagggcaccggccttgacttcgcatgtgatatctatatttgggccccactagccgggatctgtgttgctcttctcctttcactcattatcaccttgatttgttaccacaagtggataagaaagaaatttccacatatctttaaacagccattcaagaagaccaccggcgccgctcaagaagaagatgcatgttcctgtcgttgtccacaagaggaagaaggggggggtggtggatatgagttgcgagctaaatttagtcgctctgcagaaactgcggcaaatcttcaagatccaaatcaactttataatgaacttaatctcggccgccgcgaggagtatgatgtgcttgaaaagaaacgcgctcgcgatcccgagatgggcggaaaacaacaacggcggcggaatccccaagagggtgtctataacgcccttcaaaaggacaagatggccgaggcatactctgaaatagggacgaaaggggagaggcgcagaggaaaaggacatgacggcttgtaccaaggactgagcaccgcaactaaagatacttatgatgctctacacatgcaaacactcgccccacgcggaagcggtgagggaagaggtagcctactgacttgtggagacgtggaagaaaaccccggtcctatgaatccagcgattagtgtggcactcctgttgtccgtcttgcaagtaagccggggccagaaagtaactagcctaacggcgtgcctggtgaaccagaacctgaggctcgactgcagacatgaaaataatacaaaagataactccatacaacatgagttctctctcacccgcgagaagagaaaacatgtcctttctggaactcttgggattccagagcacacctatagaagcagagtcacattgagtaaccaaccatacattaaagtgctgacacttgctaatttcaccacaaaagatgagggcgactatttctgtgaacttagggtaagtggagctaatcccatgagtagcaataaatcaattagcgtgtatcgcgataagctagtcaaatgtggcggtatttcccttcttgttcaaaatacttcatggatgctactcctgctcctctcattgagcttgctgcaagcactcgatttcatcagtttgtag

SEQ ID NO: 178 - DNA, 2064 bp, Artificial sequence
  synthetic sequence
atggcatcccctctcacgagattcctcagcctcaatctgttgttgctcggagaaagcattattttggggagtggtgaggcgcaatccctcgaggaatccggtggtgacctagtaaagccaggagctagcctcaccctgacgtgcactgcatccggaatagatttcagcgatatatattacatatcctgggtccgccaagcacctggtaaaggactcgaatggattgcttgtacgtatgcaggtagctccggcagcacttactacgccacctgggcaaagggccgatttactattagcaaagccagcagcaccacagttacacttcaaatgacatcactgaccgcagctgacaccgcaacctacttctgcgcacgcgatcgatatccaggaacatcaggacgggtgtatggtatggacctctggggtccgggaaccctggtcacggtatcatcaggctcaaccagcggctccggtaaacccgggtctggtgagggaagcactaaaggagttgtcatgacccaaactccatccagcgtctcagagcctgtgggcgggactgtgactatcaaatgtcaagcttcccaaagtatttctgatctcctttcatggtatcagcaaaagccgggacaacccccaaaacttctgatctatagcgcctctactctggccagcggagttagttccagatttaagggaagcggatcaggtacagagtttacgctaactatctctgaccttgaatgtgctgatgcggctacatattattgtcaatgtaactattacagtccaacctacgggaacgggtttggcggcggcacagaagttgtcgtcaaaactacaaagcctgtgctgagaacaccttcacctgtgcacccgaccggaacttcccagccacagaggcccgaagattgccgccctcggggaagcgtaaaagggacgggcctcgattttgcatgtgatatatatatttgggctcctctggcaggcatctgtgttgcactccttctcagccttattatcacgcttatttgctaccataaatggatacgcaagaagtttccacacatctttaagcaaccattcaagaagactacaggcgctgcccaagaagaggatgcttgcagctgtagatgtcctcaagaggaagagggtggtggtggcggatatgaactgagggctaaattcagccgatccgccgagacagccgccaatttgcaagatcccaatcaactatataacgaactaaatctgggaagacgggaagagtatgacgtcctcgaaaagaaacgcgcacgagaccccgaaatgggcgggaagcagcaacggcggcgcaatccccaagaaggggtttataatgcactccaaaaggataaaatggctgaggcctacagcgagatagggactaaaggagaacgacgacgcggcaaaggccatgacggactttaccaagggctttccacagcaacaaaagacacttatgatgccctgcatatgcagacattggctcccagaggaagtggagaggggcgcggaagtcttcttacttgtggcgacgtagaagaaaatccaggcccgatgaatccagccataagcgtggcgttgctgctctctgtgctgcaagtgagccgaggtcaaaaggtaacctcattgaccgcgtgcctcgtgaatcaaaatctgcgccttgactgccgccatgaaaacaataccaaagacaattccatacagcacgaattttcattgacccgcgaaaagcggaaacacgttttgagcggaaccctcggaatacctgagcatacttatcgctcacgcgtcaccctaagcaaccagccatatatcaaggtcttgactttggccaatttcacaacaaaagatgaaggcgattacttttgtgaactccgggtatctggtgctaatcctatgtccagtaataagtccatttccgtgtatcgcgacaaattggtcaaatgtggtggaatctcattgctagtgcaaaacacctcttggatgttgttgcttcttctgagtttgtcactgcttcaagctctggacttcatatctctttag

SEQ ID NO: 179 - DNA, 2064 bp, Artificial sequence
  synthetic sequence
atggccagcccgctaacccgattcctgtcactaaatctgcttctgctcggggaatcaataatcctaggctcaggcgaagcggtagtcatgacccaaactccatcttctgtgtcagaaccagtcggtgggacggttaccataaagtgccaggccagccaaagcatatcagatctcctttcatggtatcaacagaaaccagggcaacccccaaagctcctgatatatagtgcttccaccctggcctcaggagtatcctctaggtttaaaggtagtggaagtggtacggaattcacgctcaccattagtgatctagaatgcgccgacgcagcaacatactactgtcaatgtaactattactcaccaacgtacgggaatggatttggtggtggcaccgaagtggttgtaaagggcagcacttctgggagcgggaaaccaggaagtggagaaggaagcacaaaaggccagtcactggaagaaagtggcggggatctcgtgaagcctggcgcctcactaactctcacttgcaccgcgtcaggcatcgattttagtgatatatactatattagctgggtgcgacaagcaccaggaaaaggtctggaatggattgcctgcacatatgcagggtcttcaggctccacctactacgccacatgggccaaaggcagatttaccatatcaaaagctagctccacaacagtcactctacaaatgacaagcctcaccgcagctgacactgccacttacttctgcgctcgcgatcgttatcctggtacatccggacgggtctatggtatggatctctggggacccggcactcttgttacagtgagctccactacaaaacccgtacttcggacaccaagtccggtccatcctactggtacatctcaaccccagaggcccgaagattgcaggccgcggggttctgtgaaaggtaccgggcttgattttgcgtgcgacatctacatttgggcacctctggcaggaatttgcgtcgcacttttgctctcccttataatcactctgatatgttatcacaaatggattcggaagaagttcccgcacatctttaaacaacctttcaagaagacaacaggggcggcccaagaagaggacgcttgctcatgcagatgcccgcaggaagaagagggtggtggcggtggatatgagctacgcgccaaattttcaagaagtgcggaaacagctgccaatttgcaggacccaaaccaactgtataacgaactgaatttgggtcgaagggaagaatatgatgtacttgaaaagaaaagggccagagatcccgaaatgggcggaaagcagcaacgccgcagaaacccacaagaaggcgtgtacaatgctcttcagaaagacaaaatggccgaagcttattccgaaatcggcactaagggagagagaagacgcggaaaggggcacgatggcctgtatcaaggtttgtctacggcaacaaaagatacttatgacgcactccatatgcaaacacttgccccaaggggatctggtgaaggtcgtggctccctgcttacctgcggagatgtggaagaaaatccaggccctatgaaccctgcgatttctgtggcacttctactgtccgtcctgcaggtgtctagaggacaaaaggttacttctctaacggcttgtctcgtgaaccaaaacctaaggctcgactgccgacacgaaaataatactaaggataattccattcaacatgaattttctcttaccagggagaaacggaagcatgtgctcagcggcacgctaggaattcccgagcacacatatcgatccagggtgacactctcaaatcaaccctacatcaaagtcctcaccctagcaaacttcacaactaaggacgagggcgactacttttgtgagttgcgagtaagtggggcaaacccaatgtcttccaacaagagcattagtgtataccgcgacaaactcgtaaaatgtggtggaatcagccttttggtgcaaaataccagctggatgcttttgctcttgctttccctgtcactactgcaagccctcgactttatcagtctttaa

SEQ ID NO: 180 - protein, 309 aa, Vaccinia virus
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSATSGESTTDETPEPITDKEDHTVTDTVSYTTVSTSSGIVTTKSTTDDTYNDNDTVPPTTVGGSTTSISNYKTKDFVEIFGITALIILSAVAIFCITYYICNKRSRKYKTENKV

SEQ ID NO: 181 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYITINCDVGYEVIGASYISCTANSWNVIPSCQQKCDIPSLSNGLISGSTFSIGGVIHLSCKSGFILTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 182 - protein, 314 aa, Vaccinia virus
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 183 - protein, 317 aa, Vaccinia virus
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 184 - protein, 315 aa, Vaccinia virus
MTRLPILLLLISLVYATPFPQTSKKIGDDATLSCNRNNTNDYVVMSAWYKEPNSIILLAAKSDVLYFDNYTKDKISYDSPYDDLVTTITIKSLTARDAGTYVCAFFMTSPTNDTDKVDYEEYSTELIVNTDSESTIDIILSGSTHSPETSSEKPDYIDNSNCSSVFEIATPEPITDNVEDHTDTVTYTSDSINTVSATSGESTTDETPEPITDKEEDHTVTDTVSYTTVSTSSGIVTTKSTTDDADLYDTYNDNDTVPSTTVGSSTTSISNYKTKDFVEIFGITALIILSAVAIFCITYYICNKRSRKYKTENKV

SEQ ID NO: 185 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 186 - DNA, 333 bp, Artificial sequence
  synthetic sequence
gacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaaaag

SEQ ID NO: 187 - DNA, 330 bp, Artificial sequence
  synthetic sequence
gacgttgtaatgactcagacgccatcctccgttagcgaaccagtcggaggtacagtgaccataaagtgccaagcctctcagtctatatctgacctgttgagttggtatcaacagaaaccgggacaacccccaaagttgctcatctactctgcgtcaacactcgcatcaggcgtctcaagcagattcaaagggagtgggtccggtacggaatttactcttaccataagtgaccttgagtgcgctgacgctgctacttattattgccagtgtaactactattctccgacctacggaaatgggttcggaggcggaactgaggtagtggttaaa